(19) United States
(12) Patent Application Publication
(10) Pub. No.: US 2026/0061082 A1
(43) Pub. Date: Mar. 5, 2026

(54) PROSTATE-SPECIFIC MEMBRANE ANTIGEN TARGETED DEEP-TUMOR PENETRATION OF POLYMER NANODRUGS AND METHODS OF USE THEREOF

(71) Applicants: Daniel V. Santi, San Francisco, CA (US); THE REGENTS OF THE UNIVERSITY OF CALIFORNIA, Oakland, CA (US); PROLYNX INC., San Francisco, CA (US)

(72) Inventors: Robert Flavell, San Francisco, CA (US); Henry VanBrocklin, San Francisco, CA (US); Niranjan Meher, San Francisco, CA (US); Gary W. Ashley, San Francisco, CA (US); Daniel V. Santi, San Francisco, CA (US)

(21) Appl. No.: 19/107,004

(22) PCT Filed: Aug. 30, 2023
(86) PCT No.: PCT/US2023/073195

§ 371 Date: Feb. 26, 2025

Related U.S. Application Data
(60) Provisional application No. 63/402,375, filed on Aug. 30, 2022.

(51) Int. Cl. A61K 51/04; A61K 47/54; A61K 47/60; A61K 51/06; A61P 35/00

(52) U.S. Cl. CPC A61K 51/0453; A61K 47/542; A61K 47/545; A61K 47/547; A61K 47/60; A61K 51/065; A61P 35/00; A61K 2121/00; A61K 2123/00

(57) ABSTRACT

PSMA targeted metal chelate nanodrugs, methods of making these nanodrugs and methods of using them for radiodiagnosis and radiotherapy are provided.

### CROSS-REFERENCE TO RELATED APPLICATIONS

This application claims priority to U.S. Provisional Application No. 63/402,375, filed Aug. 30, 2022, all of which is herein incorporated by reference in its entirety.

### GOVERNMENT RIGHTS

This invention was made with government support under P30CA82103 awarded by the National Institutes of Health and W81XWH-20-1-0292 awarded by US Department of Defense. The Government has certain rights in the invention.

### FIELD OF THE INVENTION

The invention relates generally to compounds with enhanced permeability and retention (EPR) effect for medical imaging and/or therapy.

### BACKGROUND

Prostate cancer is the most prevalent noncutaneous cancer in men in the United States and the second most common cause of cancer deaths in men. Prostate-specific membrane antigen (PSMA) is a highly expressed cell surface enzyme on prostate cancer cells and a valuable clinical biomarker of prostate cancer. A urea-based ligand (ACUPA) with free carboxylic acid groups has been established to bind to the enzymatic domain of PSMA selectively. It has generated considerable excitement by developing various positron emission tomography (PET) and single-photon emission computed tomography (SPECT) theranostic agents for prostate cancer. ¹⁷⁷Lu and ²²⁵Ac based PSMA targeted radiotherapy are currently utilized in the clinic to treat metastatic prostate cancer. Inspired by these promising clinical studies, there has been increased interest in utilizing PSMA targeting for nanoparticle delivery.

Whereas polymer and antibody-based therapeutics have been promising for clinical use, significant recurrence rates have become a major limiting factor. This is due in part to incomplete tumor penetration of the large size macromolecules. Large size nanodrugs can have non-specific tumor accumulation due to the enhanced permeability and retention (EPR) effect. EPR effect is the mechanism by which high-molecular-weight nontargeted drugs tend to accumulate in tissues with defective vasculature and impaired lymphatic drainage. EPR mediated accumulation of nanodrugs in bulk tumor with abnormal vascular architecture has been well proven and a widely accepted strategy for the effective delivery of nanoparticles to the tumour site. However, the magnitude of the EPR effect is governed by various variables including the nanodrug size, in vivo pharmacokinetics, the tumor vasculature, microenvironment, and macrophages. Thus, depending on the tumor phenotype, the nanodrugs often lack deep tumor penetration, limiting drug delivery and therapeutic efficacy. Furthermore, relatively large size nanodrugs with strong target binding affinity may suffer from the binding site barrier (BSB) effect, in which the nanodrugs bind to cells peripheral to blood vessels, preventing their further diffusion into the bulk tumors. Overall, the nanodrug size and the tumor phenotype play potential roles in both passive and active tumor uptake, making it highly challenging to design target specific nanodrugs with long-time retention and deep-tumor penetration in the tumor.

### SUMMARY OF THE INVENTION

In various embodiments, the present invention provides a new class of compounds with enhanced permeability and retention (EPR) effect for medical imaging. Exemplary compounds of this invention comprise a branched PEG moiety linking a prostate-specific membrane antigen (PSMA) targeted ligand and a chelator with a structure according to Formula (I):

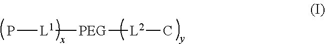

wherein PEG is branched polyethylene glycol; L¹ and L² are independently selected linkers; P is a prostate-specific membrane antigen (PSMA) targeted ligand; C is a chelator; x is an integer from 1 to 8; y is an integer from 1 to 8, such that x+y is less than or equal to 8, wherein each C is independently selected from a chelating agent and a chelating agent binding a radioisotope suitable for positron emission tomography (PET) and/or single-photon emission computed tomography (SPECT).

Exemplary prostate-specific membrane antigen (PSMA) targeted ligands of the present invention include compounds of the structure:

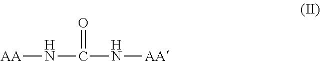

wherein AA and AA′ are independently selected amino acids connected through their NH₂ moieties by a urea linkage. In various embodiments, the PSMA targeted ligand is (S)-2-(3-((S)-5-amino-1-carboxypentyl)ureido)pentanedioic acid (ACUPA).

Exemplary chelators of the present invention include a desferrioxamine, a tetraaza macrocycle, or a multidentate plur-hydroxypyridinone. In various embodiments, the chelator is desferrioxamine-B (DFB, 1,4,7,10-tetraazacyclododecane-1,4,7,10-tetraacetic acid (DOTA), 1,4,7,10-tetraazacyclododececane,1-(glutaric acid)-4,7,10-triacetic acid (DOTAGA), 1,2-hydroxypyridinone (1,2-HOPO), or N,N′-bis[(6-carboxy-2-pyridil)methyl]-4,13-diaza-18-crown-6 (H₂macropa).

Exemplary radioisotopes suitable for positron emission tomography (PET) and/or single photon emission computed tomography (SPECT) of the present invention include ions of ⁸⁹Zr, ^{99m}Tc, ¹⁴⁹Tb, ¹⁵²Tb, ¹⁵⁵Tb, ²⁰³Pb, ¹¹¹In, ¹⁷⁷Lu, ²²⁵Ac, ⁴⁴Sc, ⁶⁸Ga, ⁶⁴Cu, ⁶²Zn, ⁸⁶Y, and ¹³⁴Ce. In various embodiments, the radioisotope is ⁸⁹Zr or ¹⁷⁷Lu.

Compounds of the present invention are particularly useful for imaging solid tumors using positron emission tomography (PET) and/or single photon emission computed tomography (SPECT). As shown in the Examples, compounds of the present invention exhibit cell binding and internalization, enhanced permeability and retention (EPR), targeted accumulation, and deep penetration in prostate-specific membrane antigen (PSMA)-expressing cells and/or tumors relative to cells and/or tumors that do not express PSMA.

Compounds of the present invention may be prodrugs which deliver a chemotherapeutic, for example, a radionuclide such as ¹⁷⁷Lu, ²¹²Bi, ²¹³Bi, ²¹¹At, ⁶⁴Cu, ⁶⁷Cu, ⁹⁰Y, ³²P, ³³P, ⁴⁷Sc, ¹¹¹Ag, ⁶⁷Ga, ¹⁴²Pr, ⁵³Sm, ¹⁴⁹Tb, ¹⁵⁵Tb, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ²¹¹Pb, ⁴⁷Sc, ²¹²Pb, ²²⁵Ac, and ²²⁷Th.

Other embodiments, objects, and advantages of the invention will be apparent from the detailed description following.

### BRIEF DESCRIPTION OF DRAWINGS

FIG. 1 shows how PSMA targeted polymer nanodrugs improve deep-tumor penetration with long tumor retention and background clearance in PSMA+ prostate cancer tumor.

FIG. 2 shows an overview of the synthesis of the azido derivatives and their conjugation to Star-PEG nanodrugs.

FIG. 3 shows the detailed synthetic route to PEG-(DFB)₃(ACUPA)₁.

FIG. 4 shows the detailed synthetic route to PEG-(DFB)₁(ACUPA)₃.

FIG. 5 shows representative chemical structures of ⁸⁹Zr labeled Star-PEG nanodrugs to evaluate the PSMA targeted PET imaging of prostate cancer. [⁸⁹Zr]PEG-(DFB)₄ is previously reported nontargeted nanodrug used in this study as a negative control.

FIG. 6 shows ¹H NMR spectra of Azido-ACUPA-tBu.

FIG. 7 shows ¹H NMR spectra of Azido-ACUPA.

FIG. 8 shows ¹H NMR spectra of Azido-DFB.

FIG. 9 shows ¹H NMR spectra of PEG-(5HCyO)₃(NH₂)₁.

FIG. 10 shows ¹H NMR spectra of PEG-(DFB)₄.

FIG. 11 shows ¹H NMR spectra of PEG-(DFB)₃(ACUPA)₁.

FIG. 12 shows ¹H NMR spectra of PEG-(DFB)₁(ACUPA)₃.

FIG. 13 shows ¹H NMR spectra of PEG-(DFB)₄, PEG-(DFB)₃(ACUPA)₁, and PEG-(DFB)₁(ACUPA)₃.

FIG. 14 shows ¹³C NMR spectra of Azido-ACUPA-tBu.

FIG. 15 shows ³C NMR spectra of Azido-ACUPA.

FIG. 16 shows ¹³C NMR spectra of Azido-DFB.

FIG. 17 shows HRMS of Azido-ACUPA-tBu.

FIG. 18 shows HRMS of Azido-ACUPA.

FIG. 19 shows HRMS of Azido-DFB.

FIG. 20 shows iTLCs of free ⁸⁹Zr and radiolabeled nanodrugs conjugates before and after purification using glass microfiber chromatography paper impregnated with silica gel and developed using 50 mM EDTA solution as mobile phase. Under these conditions, the radiolabeled nanodrugs stay at the origin, while free radiometal moves to the solvent front.

FIG. 21 shows the IC₅₀ of nonradiolabeled Star-PEGs, Azido-ACUPA, and 2-PMPA estimated by ⁶⁸Ga-PSMA-11-based in vitro competitive radioligand binding assay in PSMA+PC3-Pip cells.

FIG. 22 shows K_{d} measurements of ⁸⁹Zr labeled Star-PEGs in the PSMA+PC3-Pip cell line by a saturation binding assay.

FIG. 23 shows a blocking assay of ⁸⁹Zr labeled Star-PEGs (100 nM) in PSMA+PC3-Pip cells by using PSMA-2 as blocking agent at 1 h.

FIG. 24 shows the structure of a previously reported PSMA inhibitor ligand used for the in vitro blocking assay (PSMA-2, Kᵢ=0.24 nM, and IC₅₀=10 nM).

FIG. 25A shows an in vitro blocking assay of ⁸⁹Zr labeled nanodrugs at 10 nM at 1 h incubation in PSMA+PC3-Pip cells by using PSMA-2 as blocking agent.

FIG. 25B shows an in vitro blocking assay of ⁸⁹Zr labeled nanodrugs 1000 nM at 1 h incubation in PSMA+PC3-Pip cells by using PSMA-2 as blocking agent.

FIG. 26 shows the ratio of uptake/blocking at different concentrations in ⁸⁹Zr labeled Star-PEGs in PSMA+PC3-Pip cells by using PSMA-2 as blocking agent at 1 h.

FIG. 27A shows in vitro blocking assay of ⁸⁹Zr labeled nanodrugs at 100 nM concentration for 4 h incubation in both PSMA+PC3-Pip and PSMA-PC3-Flu cells by using PSMA-2 as blocking agent.

FIG. 27B shows in vitro blocking assay of ⁸⁹Zr labeled nanodrugs at 10 nM concentration for 4 h incubation in both PSMA+PC3-Pip and PSMA-PC3-Flu cells by using PSMA-2 as blocking agent.

FIG. 27C shows in vitro blocking assay of ⁸⁹Zr labeled nanodrugs at 100 nM concentration for 24 h incubation in both PSMA+PC3-Pip and PSMA-PC3-Flu cells by using PSMA-2 as blocking agent.

FIG. 27D shows in vitro blocking assay of ⁸⁹Zr labeled nanodrugs at 10 nM concentration for 24 h incubation in both PSMA+PC3-Pip and PSMA-PC3-Flu cells by using PSMA-2 as blocking agent.

FIG. 28 shows Membrane-Bound and Internalization assays of the ⁸⁹Zr labeled Star-PEGs at 1 h in PSMA+PC3-Pip cells. The membrane-bound activity was collected by 5 minutes of acid wash with a cold mixture of 50 mM glycine and 150 mM NaCl.

FIG. 29A shows Membrane Bound assays of the [⁸⁹Zr]Star-PEGs at 1 μM concentration up to 24 h in PSMA+PC3-Pip cells. Membrane bound activity was collected by 5 minute of acid wash with cold mixture of 50 mM glycine and 150 mM NaCl.

FIG. 29B shows Membrane Bound assays of the [⁸⁹Zr]Star-PEGs at 1 μM concentration up to 24 h in PSMA-PC3-Flu cells. Membrane bound activity was collected by 5 minute of acid wash with cold mixture of 50 mM glycine and 150 mM NaCl.

FIG. 29C shows Internalization assays of the [⁸⁹Zr]Star-PEGs at 1 μM concentration up to 24 h in PSMA+PC3-Pip cells.

FIG. 29D shows Internalization assays of the [⁸⁹Zr]Star-PEGs at 1 μconcentration up to 24 h in PSMA-PC3-Flu cells.

FIG. 29E shows total cell uptake assays of the [⁸⁹Zr]Star-PEGs at 1 μM concentration up to 24 h in PSMA-PC3-Pip cells.

FIG. 29F shows total cell uptake assays of the [⁸⁹Zr]Star-PEGs at 1 μM concentration up to 24 h in PSMA-PC3-Flu cells.

FIG. 30A shows a representation of experimental design for in vivo evaluation of the ⁸⁹Zr labeled Star-PEGs in mice bearing PSMA+PC3-Pip and PSMA-PC3-Flu xenograft.

FIG. 30B shows in vivo μPET/CT imaging. Maximum-intensity projection (MIP) μPET/CT, axial μPET/CT, and axial CT images obtained at 216 h following administration of ⁸⁹Zr labeled Star-PEGs reveal high tumor uptake with low background tissue retention of [⁸⁹Zr]PEG-(DFB)₁(ACUPA)₃ overtime.

FIG. 31 shows coronal CT, and coronal μPET/CT fusion images obtained at 24 h, 72 h, 168 h, and 216 h following administration of ⁸⁹Zr labeled nanodrugs reveal high tumor uptake with low background tissue retention of [⁸⁹Zr]PEG-(DFB)₁(ACUPA)₃ over time.

FIG. 32A shows ROI plot on heart up to 216 h (n=4).

FIG. 32B shows ROI plot PC3-Pip tumors up to 216 h (n=4).

FIG. 32C shows ROI PC3-Flu tumors up to 216 h (n=4).

FIG. 33A shows the tumor biodistribution of PSMA+PC3-Pip and PSMA-PC3-Flu Xenograft using the [⁸⁹Zr]Star-PEGs at 216 h post-injection of the nanodrugs.

FIG. 33B shows the ratio of PC3-Pip to PC3-Flu of the tumor biodistribution of the [⁸⁹Zr]Star-PEGs at 216 h post-injection of the nanodrugs.

FIG. 33C shows the ratio of tumor to muscle biodistribution of the [⁸⁹Zr]Star-PEGs at 216 h post-injection of the nanodrugs.

FIG. 33D shows the ratio of tumor to blood biodistribution of the [⁸⁹Zr]Star-PEGs at 216 h post-injection of the nanodrugs.

FIG. 34A shows the organ biodistribution in % ID/g, for ⁸⁹Zr labeled nanodrugs at 216 h postinjection (n=4).

FIG. 34B shows the organ biodistribution in % ID/organ, for ⁸⁹Zr labeled nanodrugs at 216 h postinjection (n=4).

FIG. 35 shows autoradiography images of 20 μm tumor slices of PSMA+PC3-Pip & PSMA-PC3-Flu xenografts collected after 216 h post-injection of ⁸⁹Zr labeled nanodrugs.

FIG. 36 shows representative chemical structures of ¹⁷⁷Lu-labeled StarPEG nanocarriers synthesized to evaluate the PSMA-targeted μSPECT/CT imaging and therapy of prostate cancer.

FIG. 37A-FIG. 37B show synthetic routes to the StarPEG theranostic conjugates PEG-(DOTA)₁ (FIG. 37A) and PEG-(DOTA)₁(ACUPA)₃ (FIG. 37B) evaluated in PSMA+ subcutaneous as well metastatic tumor models.

FIG. 38 shows ¹H NMR spectra of PEG-(5HCyO)₃(NH₂)₁, PEG-(PEG₇)₃(DOTA)₁, and PEG-(DOTA)₁.

FIG. 39 shows ¹H NMR spectra of PEG-(5HCyO)₃(NH₂)₁, PEG-(ACUPA)₃(DOTA)₁, and PEG-(DOTA)₁(ACUPA)₃.

FIG. 40 shows iTLCs of 177Lu radiolabeled nanocarrier conjugates (following the conditions as in Table 9) before and after purification using glass microfiber chromatography paper impregnated with silica gel and developed using 20 mM citric acid solution as mobile phase. Under these conditions, the radiolabeled nanocarriers stay at the origin, while free radiometal moves to the solvent front.

FIG. 41A-FIG. 41D shows in vitro cell-binding assays with StarPEG nanocarriers in PSMA+PC3-Pip and PSMA-PC3-Flu cell lines demonstrate efficient cell binding and uptake of PSMA-targeted theranostic nanocarriers. FIG. 41A show IC₅₀ of nonradiolabeled theranostic StarPEGs, previously evaluated diagnostic nanocarrier PEG-(DFB)₁(ACUPA)₃, and Azido-ACUPA determined by ⁶⁸Ga-PSMA-11-based in vitro competitive radioligand binding assay in PSMA+PC3-Pip cells. FIG. 41B shows Ka measurement of ¹⁷⁷Lu-labeled StarPEGs in the PSMA+PC3-Pip cell line by a saturation binding assay. FIG. 41C shows blocking assay of [¹⁷⁷Lu]PEG-(DOTA)₁(ACUPA)₃ labeled StarPEGs (5 nM) in PSMA+PC3-Pip cells using PSMA-2 (10 μM) as the blocking agent at 1 h, 4 h, and 24 h (% AD=percentage added dose). FIG. 41D shows membrane-bound and internalization assay of the [¹⁷⁷Lu]PEG-(DOTA)₁(ACUPA)₃ at 1 h, 4 h, and 24 h in PSMA+PC3-Pip cells (% AD=percentage added dose). The membrane-bound activity was collected by 5 min of acid wash with a cold mixture of 50 mM glycine and 150 mM NaCl.

FIG. 42A-FIG. 42D show in vitro blocking assay of ¹⁷⁷Lu labeled nanocarriers at 5 nM concentration incubated for 1 h, 4 h, and 24 h with PSMA+PC3-Pip and PSMA-PC3-Flu cells by using PSMA-2 (10 μM) as blocking agent. FIG. 42A shows [¹⁷⁷Lu]PEG-(DOTA)₁ in PC3-Pip. FIG. 42B shows [¹⁷⁷Lu]PEG-(DOTA)₁ in PC3-Flu. FIG. 42C shows [¹⁷⁷Lu]PEG-(DOTA)₁(ACUPA)₃ in PC3-Pip. FIG. 42D shows [¹⁷⁷Lu]PEG-(DOTA)₁(ACUPA)₃ in PC3-Flu.

FIG. 43A-FIG. 43D show membrane Bound, internalized, and total uptake of the 177Lu labeled nanocarriers at 5 nM concentration up to 24 h in PSMA+PC3-Pip and PSMA-PC3-Flu cells. Membrane bound activity was collected by 5 minute of acid wash with cold mixture of 50 mM glycine and 150 mM NaCl. FIG. 43A shows [¹⁷⁷Lu]PEG-(DOTA)₁ in PC3-Pip. FIG. 43B shows [¹⁷⁷Lu]PEG-(DOTA)₁ in PC3-Flu. FIG. 43C shows [¹⁷⁷Lu]PEG-(DOTA)₁(ACUPA)₃ in PC3-Pip. FIG. 43D shows [¹⁷⁷Lu]PEG-(DOTA)₁(ACUPA)₃ in PC3-Flu.

FIG. 44A-FIG. 44B shows colony formation assay of PSMA+PC3-Pip (FIG. 44A) and PSMA-PC3-Flu (FIG. 44B) cells treated with 41 uCi (57 nM) of [¹⁷⁷Lu]PEG-(DOTA)₁(ACUPA)₃ for for 4 h.

FIG. 45A-FIG. 45D show in vivo SPECT/CT imaging demonstrates PSMA-targeted accumulation of StarPEG nanocarriers in PSMA+PC3-Pip subcutaneous tumors. FIG. 45A shows representation of experimental design for in vivo evaluation of the ¹⁷⁷Lu-labeled StarPEGs in mice bearing dual xenografts of PSMA+PC3-Pip (left flank) and PSMA-PC3-Flu (right flank). FIG. 45B shows maximum intensity projection (MIP) μSPECT/CT, and coronal μSPECT/CT images obtained on day 24 h, 72 h, 144 h, and 192 h post-injection of ¹⁷⁷/Lu-labeled StarPEGs reveal high tumor accumulation of targeted nanocarriers in PSMA+PC3-Pip. Quantification of in vivo tumors' accumulation in PC3-Pipp (FIG. 45C) and PC3-Flu (FIG. 45D) by drawing ROIs on the respective tumors at 24 h, 72 h, 144 h, and 192 h post-injection of ¹⁷⁷Lu-labeled StarPEGs.

FIG. 46 shows ROI plot on heart, up to 192 h (n=2).

FIG. 47A-FIG. 47E show ex vivo organ biodistribution of ¹⁷⁷Lu-labeled StarPEG nanocarriers. Organ biodistribution of [¹⁷⁷Lu]PEG-(DOTA)₁ (FIG. 47A) and [¹⁷⁷Lu]PEG-(DOTA)₁(ACUPA)₃ (FIG. 47B) at 72 h, and 192 h post-injection of the nanocarriers (n=3). The ratio of PC3-Pip to PC3-Flu (FIG. 47C), PC3-Pip to the muscle (FIG. 47D), and PC3-Pip to the blood (FIG. 47E) of [¹⁷⁷Lu]StarPEGs at 72 h and 192 h post-injection of the nanocarriers (n=3).

FIG. 48A-FIG. 48B show organ biodistribution presented in % ID/organ for ¹⁷⁷Lu labeled nanocarriers on day 3 and day 8 postinjection (n=3).

FIG. 49A-FIG. 49B show autoradiography images of 20 μm tumor slices of PSMA+PC3-Pip and PSMA-PC3-Flu tumors collected after 72 h and 192 h post-injection of [¹⁷⁷Lu]StarPEG nanocarriers, [¹⁷⁷Lu]PEG-(DOTA)₁ (FIG. 49A) and [¹⁷⁷Lu]PEG-(DOTA)₁(ACUPA)₃ (FIG. 49B).

FIG. 50 shows tumor tissues collected at 72 h and 192 h post-injection which were stained with hematoxylin and eosin (H&E).

FIG. 51A-FIG. 51D show in vivo treatment study of [¹⁷⁷Lu]PEG-(DOTA)₁(ACUPA)₃ in nude mice models bearing PSMA+PC3-Pip tumors. FIG. 51A shows a representation of experimental design for in vivo treatment efficacy and toxicity of 177Lu-labeled StarPEGs in mice models bearing PSMA+PC3-Pip subcutaneous tumors. Tumor volume (FIG. 51B), body weight (FIG. 51C), and survival (FIG. 51D) of mice models bearing PC3-Pip tumors and treated with different doses (0, 125, 250, and 500 μCi) of either [¹⁷⁷Lu]PEG-(DOTA)₁(ACUPA)₃ or 250 μCi of [¹⁷⁷Lu]PSMA-617.

FIG. 52A-FIG. 52G show an in vivo treatment study of [¹⁷⁷Lu]PEG-(DOTA)₁(ACUPA)₃ in nude mice models inoculated with PSMA+PC3-Pip cells in the heart. FIG. 52A shows a representation of experimental design for in vivo treatment study of the 177Lu-labeled StarPEGs in mice bearing PSMA+PC3-Pip metastatic tumors. FIG. 52B shows multiple time points [⁶⁸Ga]PSMA-11 PET/CT imaging of the nude mice bearing PC3-Pip metastatic tumors before and after treatment of 250 μCi [¹⁷⁷Lu]PEG-(DOTA)₁(ACUPA)₃. FIG. 52C shows body weight measurement of the treated and control mice up to 51 days post-injection of 250 μCi [¹⁷⁷Lu]PEG-(DOTA)₁(ACUPA)₃. [⁶⁸Ga]PSMA-11 organ biodistribution of the nude mice bearing PC3-Pip metastatic tumors on 35 (FIG. 52D) and 50 (FIG. 52E) days post-treatment of 250 μCi [¹⁷/Lu]PEG-(DOTA)₁(ACUPA)₃. ⁶⁸Ga-PSMA-11 biodistribution presented in % ID/Organ of mice model bearing PC3-Pip metastatic tumors 35 days (n=4, FIG. 52F) or 50 days (n=8, FIG. 52G) for treated group, n=3 for control group) post treatment of [¹⁷⁷Lu]PEG-(DOTA)₁(ACUPA)₃.

FIG. 53 shows a chronic toxicity study of [¹⁷⁷Lu]PEG-(DOTA)₁(ACUPA)₃ in nude mice: (a) liver and kidney function tests show increased blood urea nitrogen and alkaline phosphatase; (b) blood cell counts showed relatively enhanced WBC and lymphocytes, while relative reduction in monocytes in [¹⁷⁷Lu]PEG-(DOTA)₁(ACUPA)₃ treated mice versus vehicle control.

### DETAILED DESCRIPTION

### Definitions

Unless defined otherwise, all technical and scientific terms used herein generally have the same meaning as commonly understood by one of ordinary skill in the art to which this invention belongs. The nomenclature used herein and the laboratory procedures used in analytical chemistry and organic syntheses described below are those well-known and commonly employed in the art.

The articles “a” and “an” are used herein to refer to one or to more than one (i.e. to at least one) of the grammatical object of the article. By way of example, “an element” means one element or more than one element.

Where substituent groups are specified by their conventional chemical formulae, written from left to right, they optionally equally encompass the chemically identical substituents, which would result from writing the structure from right to left, e.g., —CH₂O— is intended to also recite —OCH₂—.

The term “alkyl”, by itself or as part of another substituent, means a straight or branched chain hydrocarbon, which may be fully saturated, mono- or polyunsaturated and includes mono-, di-, and multivalent radicals. Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds (i.e., alkenyl and alkynyl moieties). Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl” can refer to “alkylene”, which by itself or as part of another substituent means a divalent radical derived from an alkane, as exemplified, but not limited, by —CH₂CH₂CH₂CH₂—. Typically, an alkyl (or alkylene) group will have from 1 to 30 carbon atoms. A “lower alkyl” or “lower alkylene” is a shorter chain alkyl or alkylene group, generally having eight or fewer carbon atoms. In some embodiments, alkyl refers to an alkyl or combination of alkyls selected from C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉ and C₃₀ alkyl. In some embodiments, alkyl refers to C₁-C₂₅ alkyl. In some embodiments, alkyl refers to C₁-C₂₀ alkyl. In some embodiments, alkyl refers to C₁-C₁₅ alkyl. In some embodiments, alkyl refers to C₁-C₁₀ alkyl. In some embodiments, alkyl refers to C₁-C₆ alkyl.

The term “heteroalkyl,” by itself or in combination with another term, means an alkyl in which one or more carbons are replaced with one or more heteroatoms selected from the group consisting of O, N, Si and S, (preferably O, N and S), wherein the nitrogen and sulfur atoms may optionally be oxidized and the nitrogen heteroatom may optionally be quaternized. The heteroatoms O, N, Si and S may be placed at any interior position of the heteroalkyl group or at the position at which the alkyl group is attached to the remainder of the molecule. In some embodiments, depending on whether a heteroatom terminates a chain or is in an interior position, the heteroatom may be bonded to one or more H or substituents such as (C₁, C₂, C₃, C₄, C₅ or C₆) alkyl according to the valence of the heteroatom. Examples of heteroalkyl groups include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, and —CH═CH—N(CH₃)—CH₃. No more than two heteroatoms may be consecutive, as in, for example, —CH₂—NH—OCH₃ and —CH₂—O—Si(CH₃)₃, and in some instances, this may place a limit on the number of heteroatom substitutions. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. The designated number of carbons in heteroforms of alkyl, alkenyl and alkynyl includes the heteroatom count. For example, a (C₁, C₂, C₃, C₄, C₅ or C₆) heteroalkyl will contain, respectively, 1, 2, 3, 4, 5 or 6 atoms selected from C, N, O, Si and S such that the heteroalkyl contains at least one C atom and at least one heteroatom, for example 1-5 C and 1 N or 1-4 C and 2 N. Further, a heteroalkyl may also contain one or more carbonyl groups. In some embodiments, a heteroalkyl is any C₂-C₃₀ alkyl, C₂-C₂₅ alkyl, C₂-C₂₀ alkyl, C₂-C₁₅ alkyl, C₂-C₁₀ alkyl or C₂-C₆ alkyl in any of which one or more carbons are replaced by one or more heteroatoms selected from O, N, Si and S (or from O, N and S). In some embodiments, each of 1, 2, 3, 4 or 5 carbons is replaced with a heteroatom. The terms “alkoxy,” “alkylamino” and “alkylthio” (or thioalkoxy) are used in their conventional sense, and refer to those alkyl and heteroalkyl groups attached to the remainder of the molecule via an oxygen atom, a nitrogen atom (e.g., an amine group), or a sulfur atom, respectively.

The terms “cycloalkyl” and “heterocycloalkyl”, by themselves or in combination with other terms, refer to cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means a polyunsaturated, aromatic substituent that can be a single ring or optionally multiple rings (preferably 1, 2 or 3 rings) that are fused together or linked covalently. In some embodiments, aryl is a 3, 4, 5, 6, 7 or 8 membered ring, which is optionally fused to one or two other 3, 4, 5, 6, 7 or 8 membered rings. The term “heteroaryl” refers to aryl groups (or rings) containing 1, 2, 3 or 4 heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl.

In some embodiments, any of alkyl, heteroalkyl, cycloalkyl, heterocycloalkyl, aryl and heteroaryl is optionally substituted. That is, in some embodiments, any of these groups is substituted or unsubstituted. In some embodiments, substituents for each type of radical are selected from those provided below.

Substituents for the alkyl, heteroalkyl, cycloalkyl and heterocycloalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents”. In some embodiments, an alkyl group substituent is selected from -halogen, —OR′, ═O, ═NR′, ═N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. In one embodiment, R′, R″, R′″ and R″″ are each independently selected from hydrogen, alkyl (e.g., C₁, C₂, C₃, C₄, C₅ and C₆ alkyl). In one embodiment, R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. In one embodiment, R′, R″, R′″ and R″″ are each independently selected from hydrogen, alkyl, heteroalkyl, cycloalkyl, heterocycloalkyl, aryl, heteroaryl, alkoxy, thioalkoxy groups, and arylalkyl. When R′ and R″ are attached to the same nitrogen atom, they can be combined with the nitrogen atom to form a 5-, 6-, or 7-membered ring. For example, —NR′R″ can include 1-pyrrolidinyl and 4-morpholinyl. In some embodiments, an alkyl group substituent is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

Similar to the substituents described for the alkyl radical, substituents for the aryl and heteroaryl groups are generically referred to as “aryl group substituents”. In some embodiments, an aryl group substituent is selected from -halogen, —OR′, =O, ═NR′, ═N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. In some embodiments, R′, R″, R′″ and R″″ are independently selected from hydrogen and alkyl (e.g., C₁, C₂, C₃, C₄, C₅ and C₆ alkyl). In some embodiments, R′, R″, R′″ and R″″ are independently selected from hydrogen, substituted or unsubstituted alkyl, substituted or unsubstituted heteroalkyl, substituted or unsubstituted aryl and substituted or unsubstituted heteroaryl. In some embodiments, R′, R″, R′″ and R″″ are independently selected from hydrogen, alkyl, heteroalkyl, aryl and heteroaryl. In some embodiments, an aryl group substituent is selected from substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛI(CR″R′″)_{d}—, where s and d are independently integers from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

The term “acyl” refers to a species including the moiety —C(O)R, where R has the meaning defined herein. Exemplary species for R include H, halogen, substituted or unsubstituted alkyl, substituted or unsubstituted aryl, substituted or unsubstituted heteroaryl, and substituted or unsubstituted heterocycloalkyl. In some embodiments, R is selected from H and (C₁-C₆)alkyl.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like. In some embodiments, halogen refers to an atom selected from F, Cl and Br.

The term “heteroatom” includes oxygen (O), nitrogen (N), sulfur (S) and silicon (Si). In some embodiments, a heteroatom is selected from N and S. In some embodiments, the heteroatom is O.

Unless otherwise specified, the symbol “R” is a general abbreviation representing a substituent group selected from acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound includes more than one R, R′, R″, R′″ and R″″ group, they are each independently selected.

For groups with solvent exchangeable protons, the ionized form is equally contemplated. For example, —COOH also refers to —COO⁻ and —OH also refers to —O⁻.

Any of the compounds disclosed herein can be made into a pharmaceutically acceptable salt. The term “pharmaceutically acceptable salts” includes salts of compounds prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., Journal of Pharmaceutical Science, 66: 1-19 (1977)). Certain specific compounds of the present invention contain both basic and acidic functionalities allowing the compounds to be converted into either base or acid addition salts. The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides any of the compounds disclosed herein in a prodrug form. Prodrugs of the compounds described herein are those compounds that readily undergo chemical changes under physiological conditions to provide the compounds of the present invention.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

The compounds of the present invention may also contain unnatural proportions of atomic isotopes at one or more of the atoms constituting such compounds. For example, the compounds may be labeled with deuterium (²H) or radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The symbol [symbol] , displayed perpendicular to a bond, indicates the point at which the displayed moiety is attached to the remainder of the molecule.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

Amino acids may be referred to herein by either the commonly known three letter symbols or by the one-letter symbols recommended by the IUPAC-IUB Biochemical Nomenclature Commission.

As used herein, “PSMA-targeting compound”, “PSMA-targeted compound”, “PSMA-targeting ligand”, or “PSMA-targeted ligand” shall include those small molecules, ligands, polypeptides, and proteins that have at least the biological activity of specific binding to prostate-specific membrane antigen (PSMA) or an epitope of PSMA. These compounds include ligands, receptors, peptides, or any amino acid sequence that binds to PSMA or to at least one PSMA epitope.

“Poly(alkylene oxide)” refers to a genus of compounds having a polyether backbone. Poly(alkylene oxide) species of use in the present invention include, for example, straight- and branched-chain species. Moreover, exemplary poly(alkylene oxide) species can terminate in one or more reactive, activatable, or inert groups. For example, poly(ethylene glycol) is a poly(alkylene oxide) consisting of repeating ethylene oxide subunits, which may or may not include additional reactive, activatable or inert moieties at either terminus. Useful poly(alkylene oxide) species include those in which one terminus is “capped” by an inert group, e.g., monomethoxy-poly(alkylene oxide). When the molecule is a branched species, it may include multiple reactive, activatable or inert groups at the termini of the alkylene oxide chains and the reactive groups may be either the same or different. Derivatives of straight-chain poly(alkylene oxide) species that are heterobifunctional are also known in the art. It is understood that such high-molecular weight polymers may have some inherent polydispersity due to the nature of their preparation, such that the given molecular weight represents an average value with a distribution of species about the average. This polydispersity does not affect the utility of these polymers in the present invention. Branched and multi-arm poly(alkylene oxide)s may be formed starting with a number of known core structures, including glycerol (3-arms), pentaerythritol (4-arms), dipentaerythritol (6 arms), and tripentaerythritol or hexaglycerol (8-arms).

A “linker”, “linking member”, or “linking moiety” as used herein is a moiety that joins or potentially joins, covalently or noncovalently, a first moiety to a second moiety. In particular, a linker attaches or could potentially attach a ligand described herein to another molecule, such as a targeting moiety. In some embodiments, a linker attaches or could potentially attach a ligand described herein to a solid support. A linker comprising a reactive functional group that can be further reacted with a reactive functional group on a structure of interest in order to attach the structure of interest to the linker is referred to as a “functionalized linker”. In exemplary embodiments, a linker is a functionalized linker. In exemplary embodiments, a ligand comprises one or more functionalized linkers. In some embodiments, a linker comprises a targeting moiety. In some embodiments, a linker to a targeting moiety comprises a bond to the targeting moiety.

As used herein, “anti-tumor drug” means any agent useful to combat cancer including, but not limited to, cytotoxins and agents such as antimetabolites, alkylating agents, anthracyclines, antibiotics, antimitotic agents, procarbazine, hydroxyurea, asparaginase, corticosteroids, interferons and radioactive agents. As used herein, “a cytotoxin or cytotoxic agent” means any agent that is detrimental to cells. Examples include taxol, cytochalasin B, gramicidin D, ethidium bromide, emetine, mitomycin, etoposide, tenoposide, vincristine, vinblastine, 19olchicine, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof. Other toxins include, for example, ricin, CC-1065 and analogues, the duocarmycins. Still other toxins include diphtheria toxin, and snake venom (e.g., cobra venom).

As used herein, “a radioactive agent” includes any radioisotope that is effective in diagnosing or destroying a tumor. Examples include, but are not limited to, indium-111, cobalt-60 and technetium-99m. Additionally, naturally occurring radioactive elements such as uranium, radium, and thorium, which typically represent mixtures of radioisotopes, are suitable examples of a radioactive agent. The metal ions are typically chelated with an organic chelating moiety.

A “chelator” herein refers to a compound comprised of chelating agents that can form more than one bond with a metal. Numerous chelating agents for binding metal ions are known in the art. Many useful chelating groups, crown ethers, cryptands and the like are known in the art and can be incorporated into the compounds of the invention (e.g. EDTA, DTPA, DOTA, NTA, HDTA, etc. and their phosphonate analogs such as DTPP, EDTP, HDTP, NTP, etc). See, for example, Pitt et al., “The Design of Chelating Agents for the Treatment of Iron Overload,” In, Inorganic Chemistry in Biology and Medicine; Martell, Ed.; American Chemical Society, Washington, D.C., 1980, pp. 279-312; Lindoy, The Chemistry of Macrocyclic Ligand Complexes; Cambridge University Press, Cambridge, 1989; Dugas, Bioorganic Chemistry; Springer-Verlag, New York, 1989, and references contained therein.

Additionally, a manifold of routes allowing the attachment of chelating agents, crown ethers and cyclodextrins to other molecules is available to those of skill in the art. See, for example, Meares et al., “Properties of In Vivo Chelate-Tagged Proteins and Polypeptides.” In, Modification of Proteins: Food, Nutritional, and Pharmacological Aspects;” Feeney, et al., Eds., American Chemical Society, Washington, D.C., 1982, pp. 370-387; Kasina et al., Bioconjugate Chem., 9: 108-117 (1998); Song et al., Bioconjugate Chem., 8: 249-255 (1997).

As used herein, “pharmaceutically acceptable carrier” includes any material, which when combined with the conjugate retains the activity of the conjugate activity and is non-reactive with the subject's immune system. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well-known conventional methods.

As used herein, “administering” means oral administration, administration as a suppository, topical contact, intravenous, intraperitoneal, intramuscular, intralesional, intranasal or subcutaneous administration, intrathecal administration, or the implantation of a slow-release device e.g., a mini-osmotic pump, to the subject.

In some embodiments, the definition of terms used herein is according to IUPAC.

### The Embodiments

### Prostate-Specific Membrane Antigen (PSMA) Targeting Ligand

Small molecule inhibitors of prostate-specific membrane antigen (PSMA) have shown the potential to be good agents for prostate cancer imaging. PSMA is a cell surface protein that is internalized in a process analogous to endocytosis observed with cell surface receptors, such as vitamin receptors. PSMA is a type II membrane protein with a very short intracellular domain connected by a single transmembrane helix to a large extracellular domain (Israeli et al., Cancer Res. (1993) 53(2):227-230). PSMA was first identified as the molecular target of the 7E11-C5 antibody which selectively binds LNCaP cells. In addition to its normal expression in the central nervous system, urogenital system, and small bowel, PSMA is over-expressed on prostate cancer cells and tumor neo-vasculature. A simple, easy to synthesize, and yet potent, urea-based small molecule inhibitor of PSMA was first published in 2001 (Kozikowski et al., J Med Chem (2001) 44(3):298-301). During the last decade, the simple di-amino acid urea compounds first made by Kozikowski et al. have evolved into a myriad of imaging agents for single photon emission tomography (SPECT) and positron emission tomography (PET).

PSMA-targeting compounds suitable for use in the present invention can be selected, for example, based on the following criteria, which are not intended to be exclusive: binding to live cells expressing PSMA; binding to neo-vasculature expressing PSMA; high affinity of binding to PSMA; binding to a unique epitope on PSMA (to eliminate the possibility that antibodies with complimentary activities when used in combination would compete for binding to the same epitope); opsonization of cells expressing PSMA; mediation of growth inhibition, phagocytosis and/or killing of cells expressing PSMA in the presence of effector cells; modulation (inhibition or enhancement) of NAALADase, folate hydrolase, dipeptidyl peptidase IV and/or γ-glutamyl hydrolase activities; growth inhibition, cell cycle arrest and/or cytotoxicity in the absence of effector cells; internalization of PSMA; binding to a conformational epitope on PSMA; minimal cross-reactivity with cells or tissues that do not express PSMA; and preferential binding to dimeric forms of PSMA rather than monomeric forms of PSMA.

PSMA-targeting compounds, PSMA antibodies and antigen-binding fragments thereof provided herein typically meet one or more, and in some instances, more than five of the foregoing criteria. In some embodiments, the PSMA-targeting compounds of the present invention meet six or more of the foregoing criteria. In some embodiments, the PSMA-targeting compounds of the present invention meet seven or more of the foregoing criteria. In some embodiments, the PSMA-targeting compounds of the present invention meet eight or more of the foregoing criteria. In some embodiments, the PSMA-targeting compounds of the present invention meet nine or more of the foregoing criteria. In some embodiments, the PSMA-targeting compounds of the present invention meet ten or more of the foregoing criteria. In some embodiments, the PSMA-targeting compounds of the present invention meet all of the foregoing criteria.

Examples of tumors that can be imaged with the PSMA-targeted compounds of the present invention, include any tumor expressing PSMA such as, e.g., prostate, bladder, pancreas, lung, colon, kidney, melanomas and sarcomas. A tumor expressing PSMA includes tumors with neo-vasculature expressing PSMA.

In some embodiments, the PSMA-targeting compounds bind to a conformational epitope within the extracellular domain of the PSMA molecule. In other embodiments, a PSMA-targeting compound binds to a dimer-specific epitope on PSMA. Generally, the compound that binds to a dimer-specific epitope preferentially binds the PSMA dimer rather than the PSMA monomer. In some embodiments of the present invention, the PSMA-targeting compound preferentially binds to the PSMA dimer. In some embodiments of the present invention, the PSMA-targeting compound has a low affinity for the monomeric PSMA protein.

In exemplary embodiments, the PSMA-targeted compound is chosen from the group consisting of a small molecule, a ligand, or a derivative thereof. In some embodiments, the PSMA-targeted compound is a ligand. In various embodiments, the PSMA-targeting compound is a ligand.

In exemplary embodiments, the invention provides a PSMA-targeted compound having the structure:

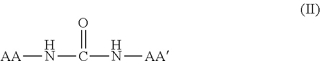

wherein AA and AA′ are independently selected amino acids connected through their NH₂ moieties by a urea linkage. In some embodiments, the amino acids are independently selected from naturally occurring amino acids. In various embodiments, the amino acids are independently selected from lysine and glutamic acid.

In some embodiments, the PSMA-targeting compound is (S)-2-(3-((S)-5-amino-1-carboxypentyl)ureido)pentanedioic acid (ACUPA). In some embodiments, the PSMA-targeting compound is ACUPA or derivative of ACUPA, ligand, inhibitor, or agonist that binds to PSMA-expressing live cells. In some embodiments compounds of the present invention have a binding affinity to PSMA that is similar to the binding affinity of ACUPA. In some embodiments compounds of the present invention are highly selective for targeting to a tumor cell.

In exemplary embodiments, the present invention provides conjugates of prostate specific membrane antigen (PSMA) targeted ligands and methods for their therapeutic and diagnostic use. More specifically, this invention provides compounds and methods for diagnosing and treating diseases associated with cells expressing prostate specific membrane antigen (PSMA), such as prostate cancer and related diseases. The invention further provides methods and compositions for making and using the compounds, methods incorporating the compounds, and kits incorporating the compounds. It has been discovered that a PSMA-targeted ligands, such as ACUPA or conjugating PSMA-targeting ligand to a polymer via a linker (L) is useful in the imaging, diagnosis, and/or treatment of prostate cancer, and related diseases that involve pathogenic cell populations expressing or over-expressing PSMA.

### Linkers

In some embodiments, the linker is a linker to a functional moiety, or a linker to a branched polyethylene glycol (PEG), or a linker to a chelator, or a linker to a PSMA-targeting ligand. In some embodiments, the functional moiety is a reactive functional group, or a protected functional group, or a protected reactive functional group. In some embodiments, the linker is a linker to a reactive functional group, or a linker to a PEG moiety, or a linker to a chelator, or a linker to a PSMA-targeting ligand.

A linker can be any useful structure joining a ligand to a reactive functional group or a targeting moiety, such as an antibody. Examples of a linker include 0-order linkers (i.e., a bond), substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. Further exemplary linkers include substituted or unsubstituted (C₁, C₂, C₃, C₄, C₅, C₆, C₇, C₈, C₉ or C₁₀) alkyl, substituted or unsubstituted heteroalkyl, —C(O)—, —C(O)NR′—, —C(O)O—, —C(O)S—, and —C(O)CR′R″, wherein R′ and R″ are members independently selected from H, substituted or unsubstituted alkyl, substituted or unsubstituted heteroalkyl, substituted or unsubstituted aryl, substituted or unsubstituted heteroaryl and substituted or unsubstituted heterocycloalkyl. In some embodiments, a linker includes at least one heteroatom. Exemplary linkers also include —C(O)NH—, —C(O), —NH—, —S—, —O—, and the like. In various embodiments, a linker is comprised one or more of a bond, —C(O)—, —C(O)O—, (C₁-C₈) alkyl, triazole, (C₃-C₈) cycloalkyl, heteroalkyl, and aryl, unsubstituted or substituted with one or more reactive functional groups.

In some embodiments, a linker is comprised of the structure F^{y}-L^{a}-F^{x}, wherein L^{a} is selected from a bond, acyl, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl; and F^{x} and F^{y} are independently selected from a reactive functional group, a protected functional group, and a targeting moiety. In some embodiments, the linker is comprised of more than one linker comprised of the structure F^{y}-L^{a}-F^{x}. In some embodiments, the linker is comprised of two linkers comprised of the structure F^{y}-L^{a}-F^{x}. In some embodiments, the linker is comprised of three linkers comprised of the structure F^{y}-L^{a}-F^{x}. In some embodiments, the linker is comprised of four linkers comprised of the structure F^{y}-L^{a}-F^{x}.

In some embodiments, L^{a} is selected from substituted or unsubstituted alkyl, substituted or unsubstituted heteroalkyl. In some embodiments, L^{a} is heteroalkyl. In some embodiments, L^{a} is (C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉ or C₂₀) alkyl in which 1, 2 or 3 atoms are replaced with a heteroatom, such as nitrogen or oxygen. In some embodiments, L^{a} comprises a modifying moiety.

In various embodiments, F^{x} and F^{y} are independently selected reactive functional groups. In some embodiments, F^{x} and F^{y} are independently selected from one or more of —NH₂, —C(O)OH, —N₃, alkyl ester (e.g., methyl ester), carbonate, carbamate, substituted or unsubstituted cyclooctyne, N-hydroxysuccinimide (NHS) ester, sulfo-NHS ester, isothiocyanate, urea, thiourea, and maleimide.

In various embodiments, F^{y}-L^{a}-F^{x} is selected from:

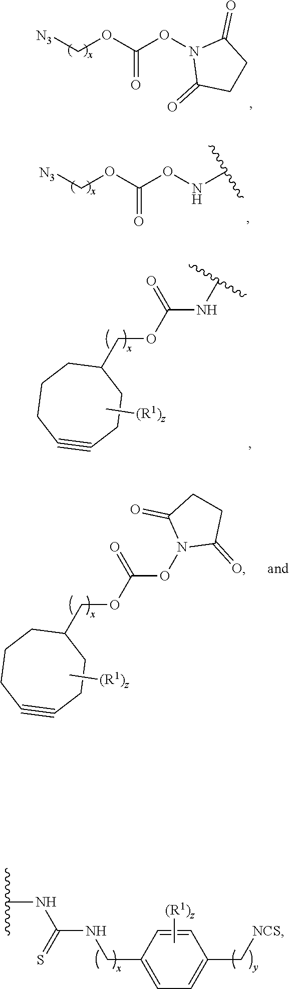

wherein x and y are integers independently selected from 0, 1, 2, 3, 4, 5, 6, 7, 8, 9, 10, 11, and 12; z is an integer selected from 0, 1, 2, 3, 4, and 5; and each R¹ is independently selected from —OR′, =O, ═NR′, ═N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, wherein R′, R″, R′″ and R″″ are each Independently selected from H, substituted or unsubstituted alkyl, substituted or unsubstituted acyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

In various embodiments, F^{y}-L^{a}-F^{x} is selected from:

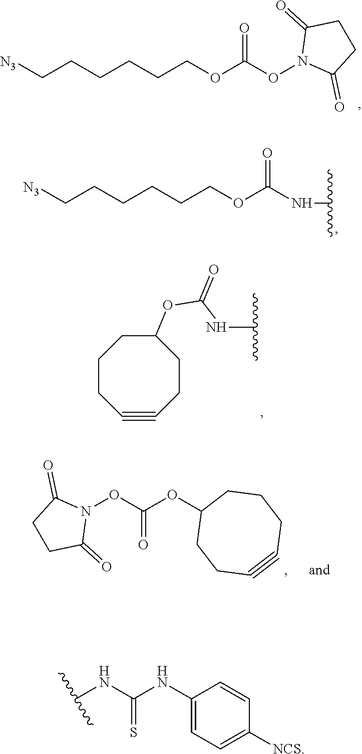

In exemplary embodiments, the linker is selected from:

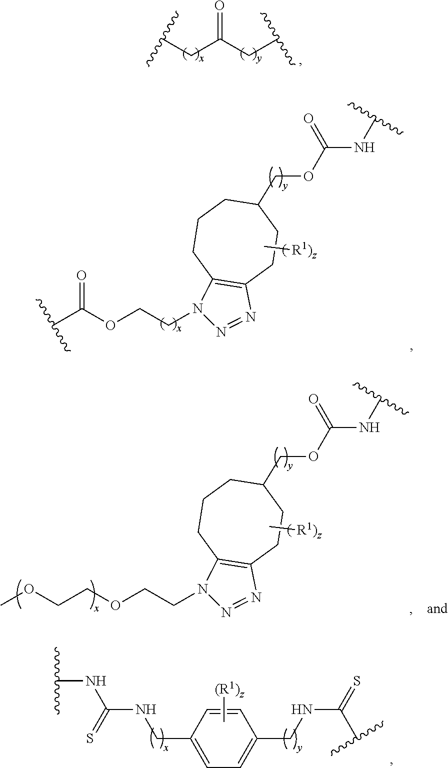

wherein x and y are integers independently selected from 0, 1, 2, 3, 4, 5, 6, 7, 8, 9, 10, 11, and 12; z is an integer selected from 0, 1, 2, 3, 4, and 5; and each R¹ is independently selected from —OR′, =O, ═NR′, ═N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″) ═NR″″, —NR—C(NR′R″)═NR′, —S(O)R′, —S(O)₂R′, —S(O)₂NR′R″, —NRSO₂R′, —CN and —NO₂, wherein R′, R″, R and R″″ are each independently selected from H, substituted or unsubstituted alkyl, substituted or unsubstituted acyl, substituted or unsubstituted heteroalkyl, substituted or unsubstituted cycloalkyl, substituted or unsubstituted heterocycloalkyl, substituted or unsubstituted aryl and substituted or unsubstituted heteroaryl.

In some embodiments, the linker is selected from:

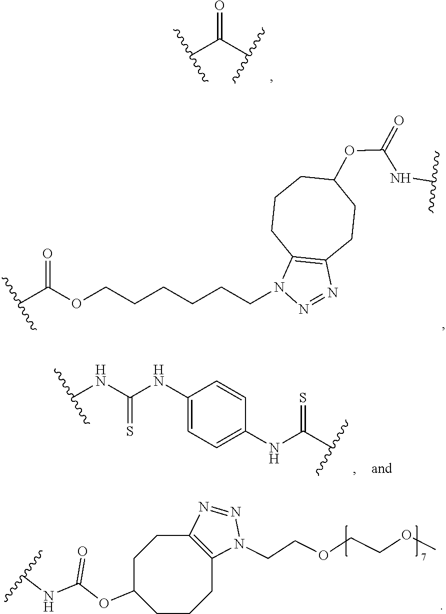

In some embodiments, e.g., according to paragraphs [0129] or [0130], any implied hydrogens can be selected from substituted or unsubstituted alkyl, and substituted or unsubstituted heteroalkyl of 1, 2, 3, 4, 5, 6, 7, 8, 9 members selected from C or a heteroatom.

In a linker with multiple reactive functional groups, a particular functional group can be chosen such that it does not participate in, or interfere with, the reaction controlling the attachment of the functionalized linker component to another ligand component. Alternatively, the reactive functional group can be protected from participating in the reaction by the presence of a protecting group masking the reactive functional group. Those of skill in the art understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

### Reactive Functional Groups

In one embodiment, a linker comprises a reactive functional group (or a “reactive functional moiety”, used synonymously), which can be further reacted to covalently attach the linker to a targeting moiety. Reactive functional groups and classes of reactions useful in practicing the present invention are generally those that are well known in the art of bioconjugate chemistry. Currently favored classes of reactions available with reactive functional groups of the invention are those which proceed under relatively mild conditions. These include, but are not limited to nucleophilic substitutions (e.g., reactions of amines and alcohols with acyl halides and activated esters), electrophilic substitutions (e.g., enamine reactions) and additions to carbon-carbon and carbon-heteroatom multiple bonds (e.g., Michael reactions and Diels-Alder reactions). These and other useful reactions are discussed, for example, in March, Advanced Organic Chemistry (3^{rd} Ed., John Wiley & Sons, New York, 1985); Hermanson, Bioconjugate Techniques (Academic Press, San Diego, 1996); and Feeney et al., Modification of Proteins, Advances in Chemistry Series, Vol. 198 (American Chemical Society, Washington, D.C., 1982).

In some embodiments, a reactive functional group refers to a group selected from olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application or modification for a particular purpose is within the ability of one of skill in the art (see, for example, Sandler and Karo, eds., Organic Functional Group Preparations, (Academic Press, San Diego, 1989)).

A reactive functional group can be chosen according to a selected reaction partner. As an example, an activated ester, such as an NHS ester will be useful to label a protein via lysine residues. Sulfhydryl reactive groups, such as maleimides can be used to label proteins via amino acid residues carrying an SH-group (e.g., cysteine). Antibodies may be labeled by first oxidizing their carbohydrate moieties (e.g., with periodate) and reacting resulting aldehyde groups with a hydrazine containing ligand.

The reactive functional groups can be chosen such that they do not participate in, or interfere with, the reactions necessary to assemble the reactive ligand. Alternatively, a reactive functional group can be protected from participating in the reaction by means of a protecting group. Those of skill in the art understand how to protect a particular functional group so that it does not interfere with a chosen set of reaction conditions. For examples of useful protecting groups, see, for example, Greene et al., PROTECTIVE GROUPS IN ORGANIC SYNTHESIS, John Wiley & Sons, New York, 1991.

### Other Reactive Functional Groups

Other exemplary reactive functional groups include:
(i) carboxyl groups and various derivatives thereof including, but not limited to, N-hydroxybenztriazole esters, acid halides, acyl imidazoles, thioesters, p-nitrophenyl esters, alkyl, alkenyl, alkynyl and aromatic esters;
(ii) hydroxyl groups, which can be converted to esters, ethers, aldehydes, etc.;
(iii) haloalkyl groups, wherein the halide can be displaced with a nucleophilic group such as, for example, an amine, a carboxylate anion, thiol anion, carbanion, or an alkoxide ion, thereby resulting in the covalent attachment of a new group at the site of the halogen atom;
(iv) dienophile groups, which are capable of participating in Diels-Alder reactions such as, for example, maleimido groups;
(v) aldehyde or ketone groups, such that subsequent derivatization is possible via formation of carbonyl derivatives such as, for example, imines, hydrazones, semicarbazones or oximes, or via such mechanisms as Grignard addition or alkyllithium addition;
(vi) alkenes, which can undergo, for example, cycloadditions, acylation, Michael addition, etc;
(vii) epoxides, which can react with, for example, amines and hydroxyl groups;
(ix) phosphoramidites and other standard functional groups useful in nucleic acid synthesis and
(x) any other functional group useful to form a covalent bond between the functionalized ligand and a molecular entity or a surface.
   Functional Groups with Non-Specific Reactivities

In addition to the use of site-specific reactive moieties, the present invention contemplates the use of non-specific reactive groups to attach linking groups. Non-specific groups include photoactivatable groups, for example.

Photoactivatable groups are ideally inert in the dark and are converted to reactive species in the presence of light. In one embodiment, photoactivatable groups are selected from precursors of nitrenes generated upon heating or photolysis of azides. Electron-deficient nitrenes are extremely reactive and can react with a variety of chemical bonds including N—H, O—H, C—H, and C═C. Although three types of azides (aryl, alkyl, and acyl derivatives) may be employed, arylazides are presently preferred. The reactivity of arylazides upon photolysis is better with N—H and O—H than C—H bonds. Electron-deficient arylnitrenes rapidly ring-expand to form dehydroazepines, which tend to react with nucleophiles, rather than form C—H insertion products. The reactivity of arylazides can be increased by the presence of electron-withdrawing substituents such as nitro or hydroxyl groups in the ring. Such substituents push the absorption maximum of arylazides to longer wavelength. Unsubstituted arylazides have an absorption maximum in the range of 260-280 nm, while hydroxy and nitroarylazides absorb significant light beyond 305 nm. Therefore, hydroxy and nitroarylazides are most preferable since they allow to employ less harmful photolysis conditions for the affinity component than unsubstituted arylazides.

In some embodiments, the linker is a linker to a functional moiety, or a linker to a branched polyethylene glycol (PEG), or a linker to a chelator, or a linker to a PSMA-targeting ligand. In some embodiments, the functional moiety is a reactive functional group, or a protected functional group, or a protected reactive functional group. In some embodiments, the linker is a linker to a reactive functional group, or a linker to a PEG moiety, or a linker to a chelator, or a linker to a PSMA-targeting ligand.

Any linker described herein may be a linker comprising a reactive functional group that could react with a reactive functional group on a PEG moiety, chelator moiety, or PSMA-targeting ligand moiety to join the linker to the PEG moiety, chelator moiety, or PSMA-targeting ligand moiety. Any linker described herein may be a linker comprising a bond to a targeting moiety. The term “targeting moiety” refers to a moiety serves to target or direct the molecule to which it is attached (e.g., a compound of Formula (T)) to a particular location or molecule. Thus, for example, a targeting moiety may be used to target a molecule to a specific target protein or enzyme, or to a particular cellular location, to a particular cell type or to a diseased tissue. As will be appreciated by those in the art, the localization of proteins within a cell is a simple method for increasing effective concentration. For example, shuttling an imaging agent and/or therapeutic into the nucleus confines them to a smaller space thereby increasing concentration. Finally, the physiological target may simply be localized to a specific compartment, and the agents must be localized appropriately.

### Metals

In exemplary embodiments, a chelator of the present invention binds a metal. In some embodiments, the metal is an alkali metal. In some embodiments, the alkali metal is rubidium (Rb). In some embodiments, the metal is an alkaline earth metal. In some embodiments, the alkaline earth metal is selected from radium (Ra) and strontium (Sr). In some embodiments, the metal is a transition metal. In some embodiments, the transition metal is selected from gold (Au) zirconium (Zr), lutetium (Lu), technetium (Tc), copper (Cu), zinc (Zn), scandium (Sc), iron (Fe), yttrium (Y), manganese (Mn), cobalt (Co), rhodium (Rh), rhenium (Re), and niobium (Nb). In some embodiments, the metal is a post-transition metal. In some embodiments, the post-transition metal is selected from astatine (At), bismuth (Bi), indium (In), lead (Pb), and gallium (Ga). In some embodiments, the metal is a metalloid. In some embodiments, the metalloid is selected from arsenic (As) and germanium (Ge). In some embodiments, the metal is a lanthanide. In some embodiments, the lanthanide is selected from cerium (Ce), samarium (Sm), terbium (Tb), and holmium (Ho), promethium (Pm). In some embodiments, the metal is an actinide. In some embodiments, the actinide is actinium (Ac) or thorium (Th). In some embodiments, the metal is a metal ion. In some embodiments, the metal is a radionuclide. In some embodiments, the metal ion is a radionuclide. In some embodiments, the metal and/or metal ion is a therapeutic radionuclide.

In exemplary embodiments, a chelator of the present invention binds a metal. In some embodiments, the metal and/or metal ion of the invention is ⁸⁹Zr. In some embodiments, the metal and/or metal ion of the invention is ^{99m}Tc. In some embodiments, the metal and/or metal ion of the invention is ¹¹¹In. In some embodiments, the metal and/or metal ion of the invention is ¹³⁴Ce. In some embodiments, the metal and/or metal ion of the invention is ⁸¹Rb. In some embodiments, the metal and/or metal ion of the invention is ⁶⁸Ga. In some embodiments, the metal and/or metal ion of the invention is ⁶¹Cu. In some embodiments, the metal and/or metal ion of the invention is ⁶⁴Cu. In some embodiments, the metal and/or metal ion of the invention is ⁶⁷Cu. In some embodiments, the metal and/or metal ion of the invention is ⁶²Zn. In some embodiments, the metal and/or metal ion of the invention is ⁴⁷Sc. In some embodiments, the metal and/or metal ion of the invention is ⁴⁴Sc. In some embodiments, the metal and/or metal ion of the invention is ⁵²Fe. In some embodiments, the metal and/or metal ion of the invention is ⁸⁶Y. In some embodiments, the metal and/or metal ion of the invention is ⁹⁰Y. In some embodiments, the metal and/or metal ion of the invention is ⁵²Mn. In some embodiments, the metal and/or metal ion of the invention is ⁵⁵Co. In some embodiments, metal and/or metal ion of the invention is ¹⁴⁹Tb. In some embodiments, the metal and/or metal ion of the invention is ¹⁵²Tb. In some embodiments, metal and/or metal ion of the invention is ¹⁵⁵Tb. In some embodiments, metal and/or metal ion of the invention is ¹⁶¹Tb. In some embodiments, the metal and/or metal ion of the invention is ⁹⁰Nb. In some embodiments, the metal and/or metal ion of the invention is ⁷²As. In some embodiments, metal and/or metal ion of the invention is ⁶⁹Ge. In some embodiments, metal and/or metal ion of the invention is ¹⁷⁷Lu. In some embodiments, metal and/or metal ion of the invention is ¹⁶⁶Ho. In some embodiments, metal and/or metal ion of the invention is ¹⁸⁶Re. In some embodiments, metal and/or metal ion of the invention is ¹⁸⁸Re. In some embodiments, metal and/or metal ion of the invention is ¹⁸⁶Re. In some embodiments, metal and/or metal ion of the invention is ¹⁴⁹Pm. In some embodiments, metal and/or metal ion of the invention is ¹⁹⁹Au. In some embodiments, metal and/or metal ion of the invention is ⁵³Sm. In some embodiments, metal and/or metal ion of the invention is ¹⁰⁵Rh. In some embodiments, metal and/or metal ion of the invention is ⁸⁹Sr. In some embodiments, metal and/or metal ion of the invention is ²¹³Bi. In some embodiments, metal and/or metal ion of the invention is ²²³Ra. In some embodiments, metal and/or metal ion of the invention is ²²⁵Ac. In some embodiments, metal and/or metal ion of the invention is ²¹¹At. In some embodiments, metal and/or metal ion of the invention is ²⁰³Pb. In some embodiments, metal and/or metal ion of the invention is ²¹²Pb. In some embodiments, metal and/or metal ion of the invention is ²²⁷Th. In some embodiments, the metal is a radionuclide. In some embodiments, the metal ion is a radionuclide. In some embodiments, the metal and/or metal ion is a therapeutic radionuclide.

Any metal disclosed herein can be in various oxidation states, for example, +1, +2, +3, +4, +5, and so on.

### Radionuclides

The chelating moieties disclosed herein can be used to bind metal ions, in particular, a radionuclide. The term “radionuclide” or “radioisotope” refers to a radioactive isotope or element with an unstable nucleus that tends to undergo radioactive decay. Numerous decay modes are known in the art and include alpha decay, proton emission, neutron emission, double proton emission, spontaneous fission, cluster decay, β⁻ decay, positron emission (β⁺ decay), electron capture, bound state beta decay, double beta decay, double electron capture, electron capture with positron emission, double positron emission, isomeric transition and internal conversion. Radionuclides of the present invention have diagnostic and/or therapeutic value. Radionuclides useful in medical imaging (e.g., positron emission tomography (PET), single-photon emission computed tomography (SPECT)) and/or therapeutics (e.g., cancer treatment) in the context of the present invention are known in the art.

Exemplary radionuclides include alpha-emitters, which emit alpha particles during decay (e.g., bismuth-213, radium-223, actinium-225, astatine-211). Exemplary radionuclides include beta-emitters, which emit beta particles during decay (e.g., lutetium-177, holmium-166, rhenium-186, rhenium-188, copper-67, promethium-149, gold-199, bromine-77, samarium-153, rhodium-105, strontium-89, yttrium-90, iodine-131). In some embodiments, a radionuclide is an emitter of a gamma ray, or a particle selected from an alpha particle, an electron, and a positron.

Of particular use in the complexes provided herein are radionuclides selected from isotopes of actinium (Ac), arsenic (As), astatine (At), gold (Au), bismuth (Bi), cerium (Ce), cobalt (Co), copper (Cu), gallium (Ga), germanium (Ge), holmium (Ho), indium (In), iron (Fe), lead (Pb), lutetium (Lu), manganese (Mn), niobium (Nb), promethium (Pm), radium (Ra), rhenium (Re), rhodium (Rh), rubidium (Rb), samarium (Sm), scandium (Sc), strontium (Sr), terbium (Tb), thorium (Th), technetium (Tc), yttrium (Y), and zirconium (Zr). In some embodiments, a radionuclide is selected from actinium-225, arsenic-72, astatine-211, bismuth-213, bromine-77, cerium-134, cobalt-55, copper-61, copper-64, copper-67, gallium-68, germanium-69, gold-199, holmium-166, indium-111, iron-52, lead-203, lead-212, lutetium-177, manganese-52, niobium-90, promethium-149, radium-223, rhenium-186, rhenium-188, rhodium-105, rubidium-81, samarium-153, scandium-47, strontium-89, technetium-99m, terbium-149, terbium-152, terbium-155, terbium-161, thorium-227, yttrium-86, yttrium-90, zinc-62, and zirconium-89.

Other radionuclides with diagnostic and therapeutic value that can be used with the compounds disclosed herein can be found, for example, in U.S. Pat. Nos. 5,482,698 and 5,601,800; and Boswell and Brechbiel, Nuclear Medicine and Biology, 2007 October, 34(7): 757-778.

### Chelators

The present invention provides numerous chelators and metal ion complexes thereof. Exemplary chelators useful in the invention include the chelators disclosed herein as well as other chelators known in the art. These chelating agents include catechols, hydroxypyridinones, hydroxyphthalamides, and salicylamides bound together via a linking structure. See U.S. Pat. Nos. 4,181,654; 4,309,305; 4,442,305; 4,543,213; 4,698,431; 4,939,254; 5,010,191; 5,049,280; 5,624,901; 5,892,029; 6,406,297; 6,515,113; 6,846,915; 6,864,103; 7,018,850; 7,404,912; and 7,442,558; US/2008/0213917; WO/2008/008797; and US/2008/0213780.

Generally, a chelator comprises a plurality of chelating agents linked together by way of one or more scaffold moieties. Chelating agents bound together by one scaffold moiety can be referred to as open chelators, while those bound together by two scaffold moieties such that at least one closed ring is formed can be referred to as closed chelators, macrocycles, or macrocyclic chelators.

A chelator can comprise numerous chelating agents. Particularly useful chelators contain a number of chelating agents sufficient to provide, for example, 6, 8 or 10 heteroatoms such as oxygen that coordinate with a metal ion to form a complex. The heteroatoms such as oxygen provide electron density for forming coordinate bonds with a positively charged ion, and such heteroatoms can thus be considered “donors”. In some embodiments, the plurality of chelating agents of a chelator comprises a plurality of oxygen donors and a radioisotope is chelated to the chelator via at least one of the oxygen donors. In some embodiments, a chelator comprises a plurality of oxygen donors and a radioisotope is chelated to the chelator via a plurality or all of the oxygen donors.

Chelators of the present invention include two classes used in current radioimmunotherapy practice: diethylenetriamine pentaacetic acid (DTPA) (i.e., acyclic/open chain, linear chelators designed to provide fast kinetics with strong binding affinity to the metal) and 1,4,7,10-tetraazacyclododecane-1,4,7,10-tetraacetic acid (DOTA) (i.e., macrocyclic chelators designed to provide high stability and high structural pre-organization), and/or derivatives thereof.

Exemplary chelators of the present invention include a multidentate chelator comprised of a multiplicity of hydroxypyridinones, HOPO (abbreviated “multidentate plur-hydroxypyridinone”). For example, 1,2-hydroxypyridinone (1,2-HOPO) is a useful chelating moiety. HOPO units are more acidic than catecholates and hydroxamic acids. They are powerful, selective chelators for “hard” metal ions, ionized at physiological pH. See also, Ma et al., Dalton Trans (2015) 44:4884-4900 and Deri, J Med Chem (2014) 57:4849-4860, herein incorporated by reference.

Chelators of the present invention include a cyclic or acyclic desferrioxamine, e.g., desferrioxamine B, desferrioxamine (e.g., Deferoxamine; DFO), and/or derivatives thereof.

In exemplary embodiments, the chelator is selected from a desferrioxamine, a tetraaza macrocycle, or a multidentate plur-hydroxypyridinone. In some embodiments, the chelator is selected from a derivative of a desferrioxamine, a tetraaza macrocycle, or a multidentate plur-hydroxypyridinone. In some embodiments, the chelator is 1,4,7,10-tetraazacyclododecane-1,4,7,10-tetraacetic acid (DOTA), and/or a derivative thereof. In various embodiments, the chelator is desferrioxamine-B (DFB), and/or a derivative thereof. In various embodiments, the chelator is 1,4,7,10-tetraazacyclododececane,1-(glutaric acid)-4,7,10-triacetic acid (DOTAGA), and/or a derivative thereof. In various embodiments, the chelator is 1,2-hydroxypyridinone, and/or derivative thereof. In various embodiments, the chelator is N,N′-bis[(6-carboxy-2-pyridil)methyl]-4,13-diaza-18-crown-6 (H₂macropa), and/or a derivative thereof.

### Imaging Agents

The present invention provides a new class of compounds leveraging off the enhanced permeability and retention (EPR) effect for medical imaging and/or radionuclide therapy. An exemplary compound (e.g., imaging agent and/or therapeutic) of the invention comprises a branched PEG moiety linking a prostate-specific membrane antigen (PSMA) targeted ligand and a chelator with a structure of:

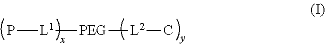

wherein PEG is branched polyethylene glycol; L¹ and L² are independently selected linkers; P is a prostate-specific membrane antigen (PSMA) targeted ligand; C is a chelator; x is an integer from 1 to 8; y is an integer from 1 to 8, such that x+y is less than or equal to 8, wherein each C is independently selected from a chelating agent and a chelating agent binding a radioisotope suitable for positron emission tomography (PET) and/or single-photon emission computed tomography (SPECT).

In exemplary embodiments, PEG is a poly(ethylene glycol) of about 2 kD to about 100 kD average molecular weight. In various embodiments, PEG is a polyethylene glycol of about 2 kD to about 60 kD. In some embodiments, PEG is about 2 to about 40 kD. In some embodiments, PEG is about 40 kD to about 60 kD. In some embodiments, PEG is about 40 kD to about 45 kD. In some embodiments, PEG is about 2 kD, or about 3 kD, or about 4 kD, or about 5 kD, or about 6 kD, or about 7 kD, or about 8 kD, or about 9 kD, or about 10 kD, or about 11 kD, or about 12 kD, or about 13 kD, or about 14 kD, or about 15 kD, or about 16 kD, or about 17 kD, or about 18 kD, or about 19 kD, or about 20 kD, or about 21 kD, or about 22 kD, or about 23 kD, or about 24 kD, or about 25 kD, or about 26 kD, or about 27 kD, or about 28 kD, or about 29 kD, or about 30 kD, or about 31 kD, or about 32 kD, or about 33 kD, or about 34 kD, or about 35 kD, or about 36 kD, or about 37 kD, or about 38 kD, or about 39 kD, or about 40 kD, or about 41 kD, or about 42 kD, or about 43 kD, or about 44 kD, or about 45 kD, or about 46 kD, or about 47 kD, or about 48 kD, or about 49 kD or about 50 kD, or about 51 kD, or about 52 kD, or about 53 kD, or about 54 kD, or about 55 kD, or about 56 kD, or about 57 kD, or about 58 kD, or about 59 kD, or about 60 kD, or about 61 kD, or about 62 kD, or about 63 kD, or about 64 kD, or about 65 kD, or about 67 kD, or about 68 kD, or about 69 kD, or about 70 kD, or about 71 kD, or about 72 kD, or about 73 kD, or about 74 kD, or about 75 kD, or about 76 kD, or about 77 kD, or about 78 kD, or about 79 kD, or about 80 kD, or about 81 kD, or about 82 kD, or about 83 kD, or about 84 kD, or about 85 kD, or about 86 kD, or about 87 kD, or about 88 kD, or about 89 kD, or about 90 kD, or about 91 kD, or about 92 kD, or about 93 kD, or about 94 kD, or about 95 kD, or about 96 kD, or about 97 kD, or about 98 kD, or about 99 kD, or about 100 kD. In various embodiments, PEG is about 40 kD.

In exemplary embodiments, x is an integer from 1 to 8. In some embodiments, x is an integer of 1 or 2 or 3 or 4 or 5 or 6 or 7 or 8. In various embodiments, x is 1. In various embodiments, x is 3.

In exemplary embodiments, y is an integer from 1 to 8. In some embodiments, y is an integer of 1 or 2 or 3 or 4 or 5 or 6 or 7 or 8. In various embodiments, y is 1. In various embodiments, y is 3.

In exemplary embodiments, x+y is less than or equal to 8. In some embodiments, x+y is less than or equal to 7. In some embodiments, x+y is less than or equal to 6. In some embodiments, x+y is less than or equal to 5. In some embodiments, x+y is less than or equal to 4. In some embodiments, x+y is less than or equal to 3. In some embodiments, x+y is less than or equal to 2. In some embodiments, x+y is 2. In some embodiments, x+y is 3. In some embodiments, x+y is 4. In some embodiments, x+y is 5. In some embodiments, x+y is 6. In some embodiments, x+y is 7. In some embodiments, x+y is 8.

The present invention provides numerous chelators and metal ion complexes thereof. Numerous chelating agents for binding metal ions are known in the art. In exemplary embodiments, the chelator is selected from a desferrioxamine, a tetraaza macrocycle, or a multidentate plur-hydroxypyridinone. In some embodiments, the chelator is selected from a derivative of a desferrioxamine, a tetraaza macrocycle, or a multidentate plur-hydroxypyridinone. In some embodiments, the chelator is 1,4,7,10-tetraazacyclododecane-1,4,7,10-tetraacetic acid (DOTA), and/or a derivative thereof. In various embodiments, the chelator is desferrioxamine-B (DFB), and/or a derivative thereof. In exemplary embodiments, the radioisotope suitable for positron emission tomography (PET) and/or single-photon emission computed tomography (SPECT) is an ion selected from ⁸⁹Zr, ^{99m}Tc, ¹¹¹In, ¹⁷⁷Lu, ²²⁵Ac, ⁴⁴Sc, ⁶⁸Ga, ⁶⁴CU, ⁶²Zn, ⁸⁶Y, ¹³⁴Ce, ¹⁴⁹Tb, ¹⁵²Tb, ¹⁵⁵Tb, and ²⁰³Pb. In various embodiments, the radioisotope is an ion of ⁸⁹Zr. In various embodiments, the radioisotope is an ion of ¹⁷⁷Lu. In various embodiments, the radioisotope is an ion of ²²⁵Ac.

In exemplary embodiments, the imaging agent has the structure:

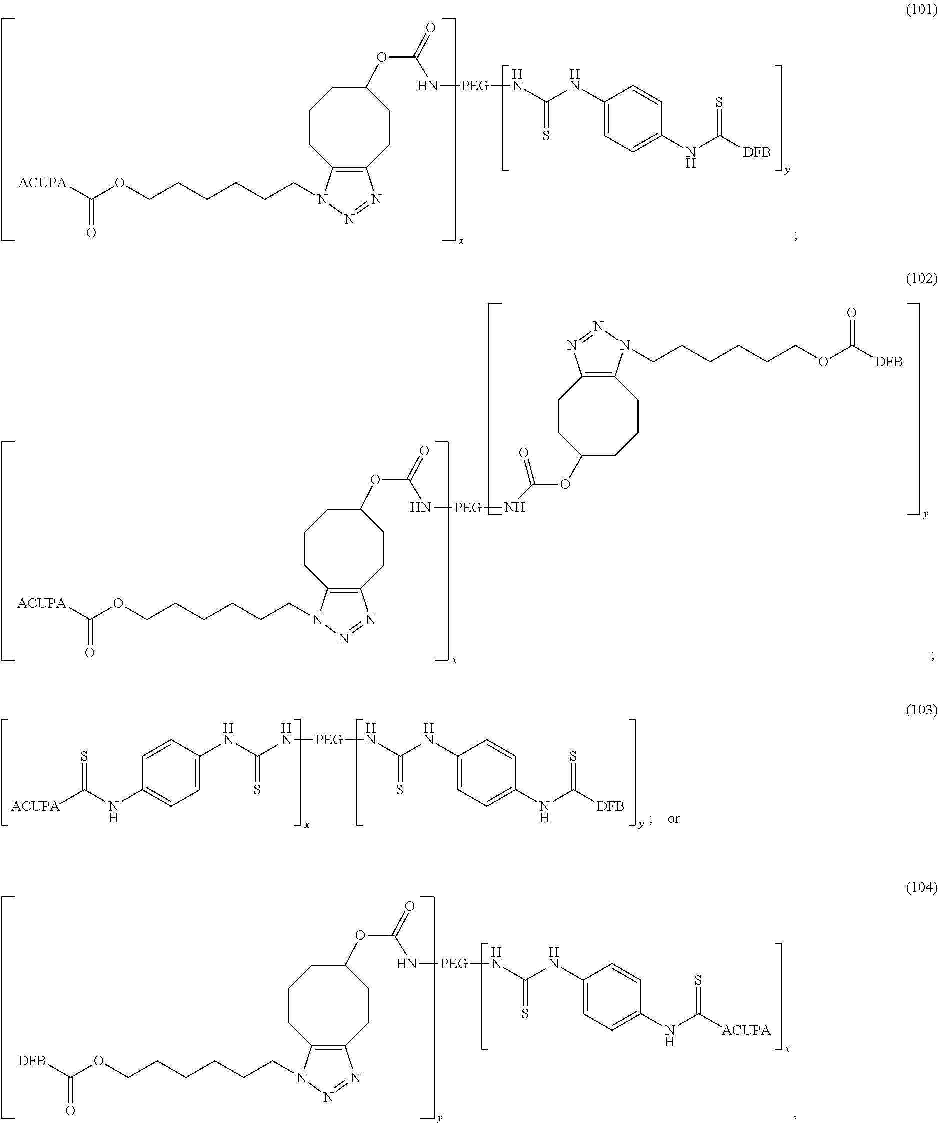

wherein PEG represents a branched polyethylene glycol; x is an integer of 1 to 8; y is an integer from 1 to 8, such that x+y is less than or equal to 8, wherein one desferrioxamine-B (DFB) moiety binds a radioisotope. In various embodiments, the radioisotope is a ⁸⁹Zr ion.

In exemplary embodiments, the imaging agent has a structure selected from the permutations below:

(101)

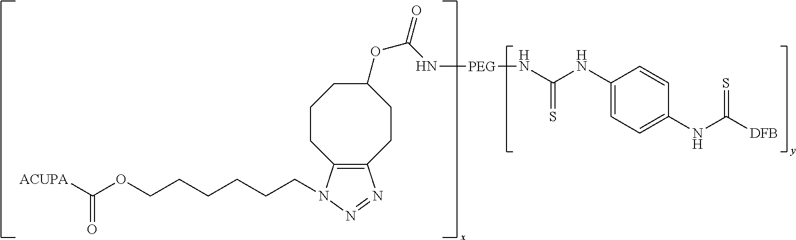

| Compound No. | x | y |
|---|---|---|
| 1001 | 1 | 1 |
| 1002 | 1 | 2 |
| 1003 | 1 | 3 |
| 1004 | 1 | 4 |
| 1005 | 1 | 5 |
| 1006 | 1 | 6 |
| 1007 | 1 | 7 |
| 1008 | 2 | 1 |
| 1009 | 2 | 2 |
| 1010 | 2 | 3 |
| 1011 | 2 | 4 |
| 1012 | 2 | 5 |
| 1013 | 2 | 6 |
| 1014 | 3 | 1 |
| 1015 | 3 | 2 |
| 1016 | 3 | 3 |
| 1017 | 3 | 4 |
| 1018 | 3 | 5 |
| 1019 | 4 | 1 |
| 1020 | 4 | 2 |
| 1021 | 4 | 3 |
| 1022 | 4 | 4 |
| 1023 | 5 | 1 |
| 1024 | 5 | 2 |
| 1025 | 5 | 3 |
| 1026 | 6 | 1 |
| 1027 | 6 | 2 |
| 1028 | 7 | 1 |

wherein one desferrioxamine-B (DFB) moiety binds metal ion, e.g., a ⁸⁹Zr ion; and/or

(102)

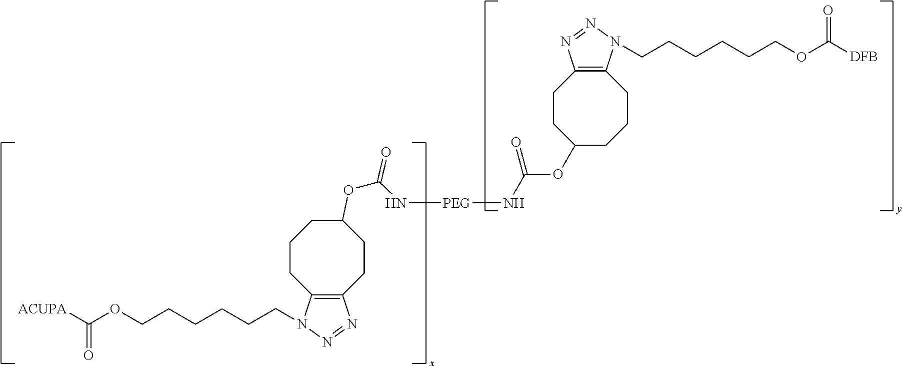

| Compound No. | x | y |
|---|---|---|
| 1029 | 1 | 1 |
| 1030 | 1 | 2 |
| 1031 | 1 | 3 |
| 1032 | 1 | 4 |
| 1033 | 1 | 5 |
| 1034 | 1 | 6 |
| 1035 | 1 | 7 |
| 1036 | 2 | 1 |
| 1037 | 2 | 2 |
| 1038 | 2 | 3 |
| 1039 | 2 | 4 |
| 1040 | 2 | 5 |
| 1041 | 2 | 6 |
| 1042 | 3 | 1 |
| 1043 | 3 | 2 |
| 1044 | 3 | 3 |
| 1045 | 3 | 4 |
| 1046 | 3 | 5 |
| 1047 | 4 | 1 |
| 1048 | 4 | 2 |
| 1049 | 4 | 3 |
| 1050 | 4 | 4 |
| 1051 | 5 | 1 |
| 1052 | 5 | 2 |
| 1053 | 5 | 3 |
| 1054 | 6 | 1 |
| 1055 | 6 | 2 |
| 1056 | 7 | 1 |

wherein one desferrioxamine-B (DFB) moiety binds a metal ion, e.g., a ⁸⁹Zr ion; and/or

(103)

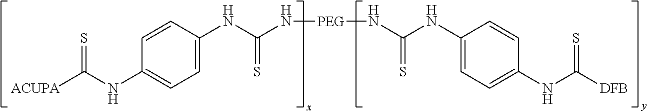

| Compound No. | x | y |
|---|---|---|
| 1057 | 1 | 1 |
| 1058 | 1 | 2 |
| 1059 | 1 | 3 |
| 1060 | 1 | 4 |
| 1061 | 1 | 5 |
| 1062 | 1 | 6 |
| 1063 | 1 | 7 |
| 1064 | 2 | 1 |
| 1065 | 2 | 2 |
| 1066 | 2 | 3 |
| 1067 | 2 | 4 |
| 1068 | 2 | 5 |
| 1069 | 2 | 6 |
| 1070 | 3 | 1 |
| 1071 | 3 | 2 |
| 1072 | 3 | 3 |
| 1073 | 3 | 4 |
| 1074 | 3 | 5 |
| 1075 | 4 | 1 |
| 1076 | 4 | 2 |
| 1077 | 4 | 3 |
| 1078 | 4 | 4 |
| 1079 | 5 | 1 |
| 1080 | 5 | 2 |
| 1081 | 5 | 3 |
| 1082 | 6 | 1 |
| 1083 | 6 | 2 |
| 1084 | 7 | 1 |

wherein one desferrioxamine-B (DFB) moiety binds a metal ion, e.g., ⁸⁹Zr ion; and/or

(104)

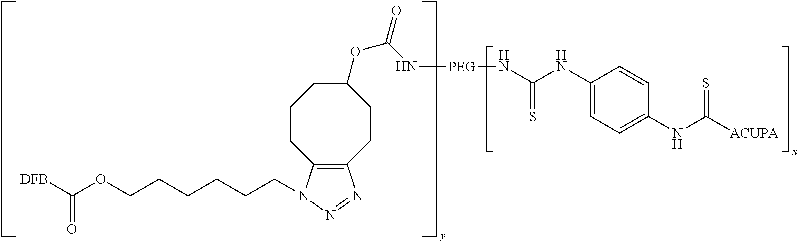

| Compound No. | x | y |
|---|---|---|
| 1085 | 1 | 1 |
| 1086 | 1 | 2 |
| 1087 | 1 | 3 |
| 1088 | 1 | 4 |
| 1089 | 1 | 5 |
| 1090 | 1 | 6 |
| 1091 | 1 | 7 |
| 1092 | 2 | 1 |
| 1093 | 2 | 2 |
| 1094 | 2 | 3 |
| 1095 | 2 | 4 |
| 1096 | 2 | 5 |
| 1097 | 2 | 6 |
| 1098 | 3 | 1 |
| 1099 | 3 | 2 |
| 1100 | 3 | 3 |
| 1101 | 3 | 4 |
| 1102 | 3 | 5 |
| 1103 | 4 | 1 |
| 1104 | 4 | 2 |
| 1105 | 4 | 3 |
| 1106 | 4 | 4 |
| 1107 | 5 | 1 |
| 1108 | 5 | 2 |
| 1109 | 5 | 3 |
| 1110 | 6 | 1 |
| 1111 | 6 | 2 |
| 1112 | 7 | 1 |

wherein one desferrioxamine-B (DFB) moiety binds a metal ion, e.g., a ⁸⁹Zr ion.

In various embodiments, the imaging agent has a structure of:

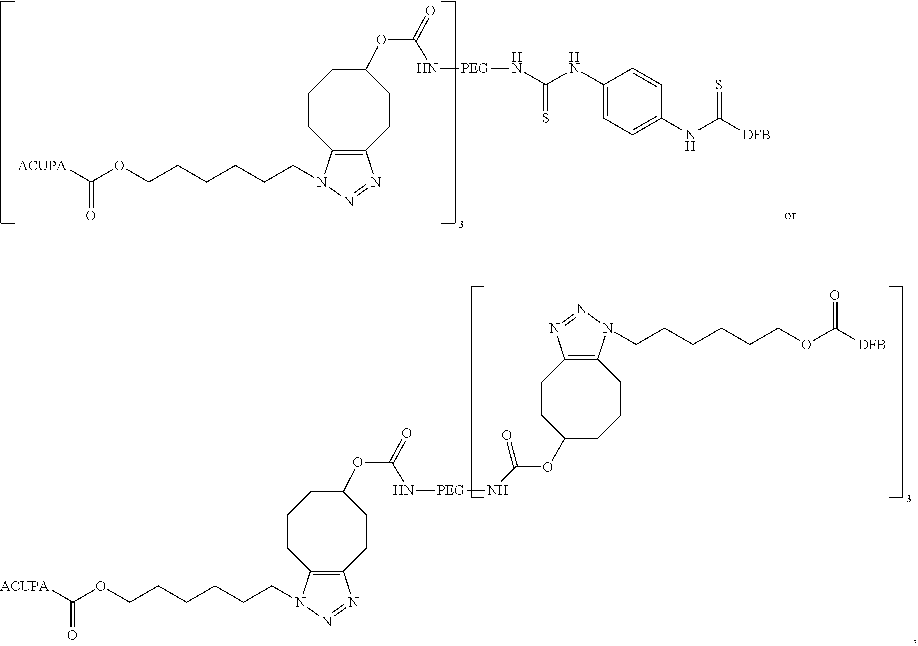

wherein one desferrioxamine-B (DFB) moiety binds a metal ion, e.g., a ⁸⁹Zr ion.

In exemplary embodiments, the imaging agent has the structure:

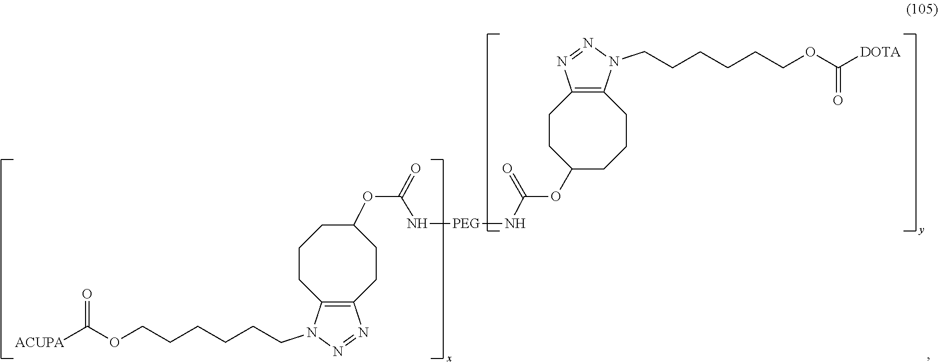

wherein PEG represents a branched polyethylene glycol; x is an integer from 1 to 8; y is an integer from 1 to 8, such that x+y is less than or equal to 8, wherein one 1,4,7,10-tetraazacyclododecane-1,4,7,10-tetraacetic acid (DOTA) moiety binds a radioisotope ion. In various embodiments, the radioisotope is a ¹⁷⁷Lu ion.

In exemplary embodiments, the imaging agent has the structure:

(105)

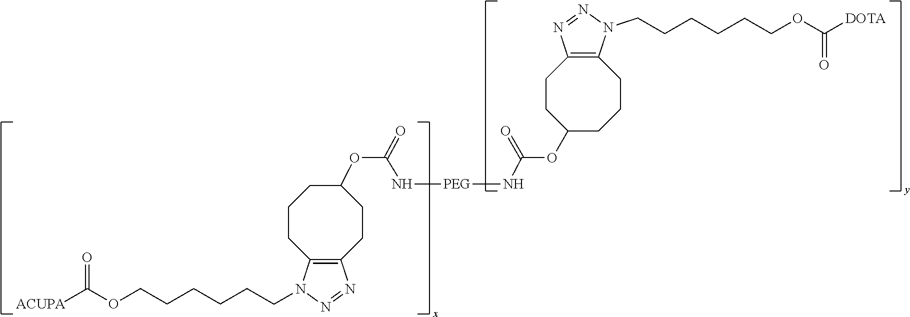

| Compound No. | x | y |
|---|---|---|
| 1113 | 1 | 1 |
| 1114 | 1 | 2 |
| 1115 | 1 | 3 |
| 1116 | 1 | 4 |
| 1117 | 1 | 5 |
| 1118 | 1 | 6 |
| 1119 | 1 | 7 |
| 1120 | 2 | 1 |
| 1121 | 2 | 2 |
| 1122 | 2 | 3 |
| 1123 | 2 | 4 |
| 1124 | 2 | 5 |
| 1125 | 2 | 6 |
| 1126 | 3 | 1 |
| 1127 | 3 | 2 |
| 1128 | 3 | 3 |
| 1129 | 3 | 4 |
| 1130 | 3 | 5 |
| 1131 | 4 | 1 |
| 1132 | 4 | 2 |
| 1133 | 4 | 3 |
| 1134 | 4 | 4 |
| 1135 | 5 | 1 |
| 1136 | 5 | 2 |
| 1137 | 5 | 3 |
| 1138 | 6 | 1 |
| 1139 | 6 | 2 |
| 1140 | 7 | 1 |

wherein one 1,4,7,10-tetraazacyclododecane-1,4,7,10-tetraacetic acid (DOTA) moiety binds a metal ion, e.g., a ¹⁷⁷Lu or ²²⁵Ac ion.

In various embodiments, the imaging agent has a structure of:

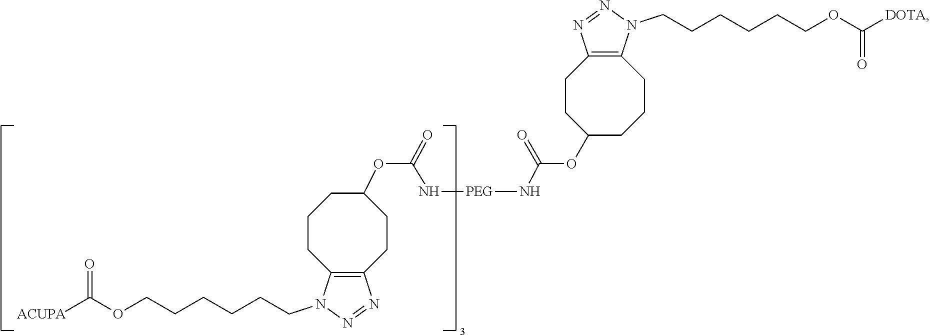

wherein the DOTA moiety binds a metal ion, e.g., a ¹⁷⁷Lu or ²²⁵Ac ion.

In exemplary embodiments, imaging agents of the present invention exhibit cell binding and internalization and/or enhanced permeability and retention (EPR) and/or targeted accumulation and/or deep penetration in prostate-specific membrane antigen (PSMA)-expressing cells and/or tumors relative to cells and/or tumors that do not express PSMA.

In exemplary embodiments, the imaging agent exhibits an enhanced dissociation constant (K_{d}). In some embodiments, the dissociation constant is from about 30 nM to about 800 nM, from about from about 30 nM to about 700 nM, from about 30 nM to about 600 nM, from about 30 nM to about 500 nM, from about 30 nM to about 400 nM, from about 30 nM to about 300 nM, from about 30 nM to about 200 nM, from about 30 nM to about 100 nM, from about 40 nM to about 100 nM, from about 40 nM to about 90 nM, from about 40 nM to about 80 nM, from about 40 nM to about 70 nM, from about 40 nM to about 60 nM, from about 50 nM to about 60 nM, from about 50 nM to about 70 nM, from about 50 nM to about 80 nM, from about 53 nM to about 67 nM, from about 52 nM to about 64 nM, or from about 52 nM to about 66 nM.

In some embodiments, the dissociation constant is about 30 nM, or about 40 nM, or about 50 nM, or about 60 nM, or about 70 nM, or about 80 nM, or about 90 nM, or about 100 nM, or about 110 nM, or about 120 nM, or about 130 nM, or about 140 nM, or about 150 nM, or about 160 nM, or about 170 nM, or about 180 nM, or about 190 nM, or about 200 nM, or about 210 nM, or about 220 nM, or about 230 nM, or about 240 nM, or about 250 nM, or about 260 nM, or about 270 nM, or about 280 nM, or about 290 nM, or about 300 nM, or about 310 nM, or about 320 nM, or about 330 nM, or about 340 nM, or about 350 nM, or about 360 nM, or about 370 nM, or about 380 nM, or about 390 nM, or about 400 nM, or about 410 nM, or about 420 nM, or about 430 nM, or about 440 nM, or about 450 nM, or about 460 nM, or about 470 nM, or about 480 nM, or about 490 nM, or about 500 nM, or about 510 nM, or about 520 nM, or about 530 nM, or about 540 nM, or about 550 nM, or about 560 nM, or about 570 nM, or about 580 nM, or about 590 nM, or about 600 nM, or about 610 nM, or about 620 nM, or about 630 nM, or about 640 nM, or about 650 nM, or about 660 nM, or about 670 nM, or about 680 nM, or about 690 nM, or about 700 nM, or about 710 nM, or about 720 nM, or about 730 nM, or about 740 nM, or about 750 nM, or about 760 nM, or about 770 nM, or about 780 nM, or about 790 nM, or about 800 nM.

In exemplary embodiments, the imaging agent exhibits reduced IC₅₀ values. In various embodiments, IC₅₀ value is from about 100 nM to about 2000 nM, from about 200 nM to about 2000 nM, from about 300 nM to about 2000 nM, from about 400 nM to about 2000 nM, from about 500 nM to about 2000 nM, from about 600 nM to about 2000 nM, from about 500 nM to about 1900 nM, from about 500 nM to about 1800 nM, from about 500 nM to about 1700 nM, from about 500 nM to about 1600 nM, from about 500 nM to about 1500 nM, from about 500 nM to about 1400 nM, from about 200 nM to about 1000 nM, from about 200 nM to about 900 nM, from about 200 nM to about 800 nM, from about 200 nM to about 700 nM, from about 200 nM to about 600 nM, from about 359 nM to about 706 nM, from about 687 nM to about 1380 nM, from about 459 nM to about 575 nM, from about 857 nM to about 1344 nM, from about 687 nM to about 1380 nM, or from about 493 nM to about 818 nM.

In some embodiments, IC₅₀ value is about 350 nM, or about 360 nM, or about 370 nM, or about 380 nM, or about 390 nM, or about 400 nM, or about 410 nM, or about 420 nM, or about 430 nM, or about 440 nM, or about 450 nM, or about 460 nM, or about 470 nM, or about 480 nM, or about 490 nM, or about 500 nM, or about 510 nM, or about 520 nM, or about 530 nM, or about 540 nM, or about 550 nM, or about 560 nM, or about 570 nM, or about 580 nM, or about 590 nM, or about 600 nM, or about 610 nM, or about 620 nM, or about 630 nM, or about 640 nM, or about 650 nM, or about 660 nM, or about 670 nM, or about 680 nM, or about 690 nM, or about 700 nM, or about 710 nM. In various embodiments, IC₅₀ value is no less than about 359 nM. In various embodiments, C₀o value is no more than about 706 nM. In various embodiments, IC₅₀ value is no less than about 459 nM. In various embodiments, IC₅₀ value is no more than about 575 nM.

In exemplary embodiments, the imaging agent is a prodrug. In various embodiments, the prodrug delivers a chemotherapeutic. In some embodiments, the chemotherapeutic delivered by the prodrug is selected from a vinca alkaloid, an anthracycline, an epidophyllotoxin, a taxane, an antimetabolite, an alkylating agent, an antibiotic, a Cox-2 inhibitor, an antimitotic, an antiangiogenic, and an apoptotic agent. In various embodiments, the prodrug delivers a therapeutic radionuclide. In some embodiments, the therapeutic radionuclide is a metal or metal ion described herein. In some embodiments, the therapeutic radionuclide is selected from actinium-225, astatine-211, bismuth-212, bismuth-213, bromine-77, copper-62, copper-64, copper-67, gold-199, holmium-166, indium-111, lead-212, lutetium-177, promethium-149, radium-223, rhenium-186, rhenium-188, rhodium-105, samarium-153, strontium-89, thorium-227, terbium-161, terbium-149, and yttrium-90. In some embodiments, the therapeutic radionuclide is selected from ¹⁷⁷Lu, ²¹²Bi, ²¹³Bi, ²¹¹At, ⁶⁴Cu, ⁶⁷Cu, ⁹⁰Y, ³²P, ³³P, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ²²⁷Th, ¹⁶¹Tb, ¹⁴⁹Tb, and ²¹¹Pb.

### Uses

The compounds, conjugates, and prodrugs disclosed herein can be used in a wide variety of therapeutic and diagnostic settings.

In an exemplary embodiment, the invention provides a method of diagnosing a disease in an animal comprising (a) administering an agent disclosed herein to the animal and (b) detecting the presence or absence of a signal emitted by the agent. In some embodiments, the detecting step comprises obtaining an image based on the signal.

In various embodiments, the invention provides a method of treating a disease in an animal comprising administering an agent disclosed herein to the animal, whereby the disease is ameliorated or eliminated.

In some embodiments, the disease is cancer.

### EXAMPLES

The compounds and complexes of the invention are synthesized by an appropriate combination of generally well-known synthetic methods. Techniques useful in synthesizing the compounds of the invention are both readily apparent and accessible to those of skill in the relevant art. The discussion below is offered to illustrate certain of the diverse methods available for use in assembling the compounds of the invention, but it is not intended to limit the scope of reactions or reaction sequences useful in preparing the compounds of the present invention.

### Example 1. Prostate-Specific Membrane Antigen Targeted Deep-Tumor Penetration of Polymer Nanodrugs

Enhanced permeability and retention (EPR) effect mediated passive uptake of large-size nanodrugs can induce high tumor uptake, but the results are variable. Depending on the tumor phenotype, EPR mediated tumor uptake can cause low tumor uptake with only peripheral accumulation. Active tumor targeting of nanodrugs has the potential to improve imaging contrast and therapeutic efficacy in vivo with their deep-tissue penetration ability. Two prostate cancer targeting nanodrugs, [⁸⁹Zr]PEG-(DFB)₃(ACUPA)₁ and [⁸⁹Zr]PEG-(DFB)₁(ACUPA)₃, with one or three prostate-specific membrane antigen (PSMA) targeting ACUPA ligands were designed and synthesized herein. The in vitro PSMA binding affinity and in vivo pharmacokinetics of the targeted nanodrugs were compared with a nontargeted nanodrug, [⁸⁹Zr]PEG-(DFB)₄, in PSMA+PC3-Pip and PSMA-PC3-Flu cells and xenografts.

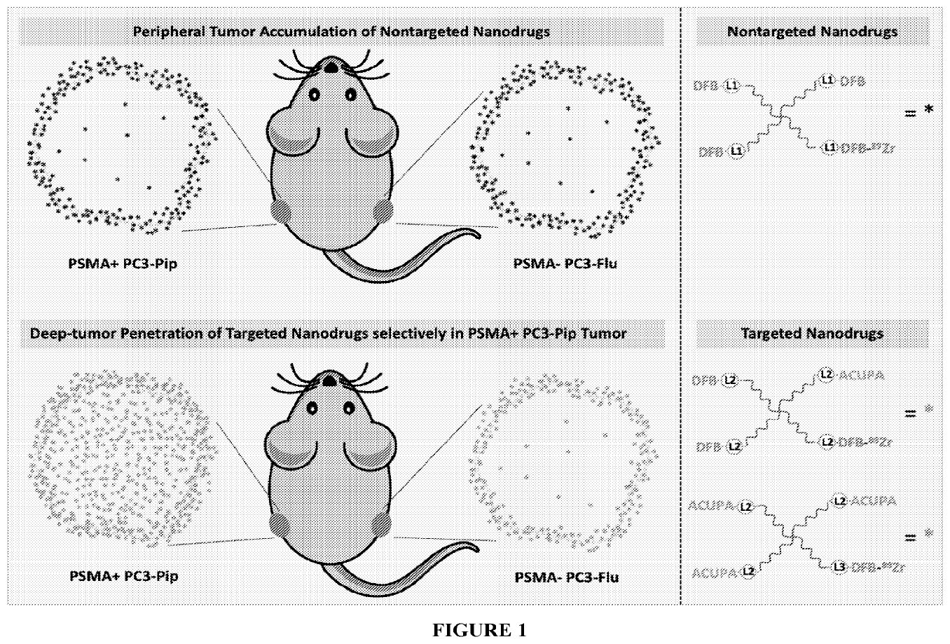

Previously, 4-armed Star-PEG nanodrugs of 40 kDa conjugated with ⁸⁹Zr chelator deferoxamine B (DFB) had been explored as nontargeted PET radiopharmaceuticals in MX-1 and HT-29 tumor models. These studies demonstrated EPR mediated deep tumor penetration with longer tumor and high retention (about 10% ID) even after 9 days postinjection. In contrast, PET imaging studies of macromolecules and nanoparticles in preclinical human prostate cancer tumor models like CWR22rv1, DU-145, and PC-3 have demonstrated an “EPR low” effect with poor tumor accumulation. Herein, three 4-armed Star-PEG_{40 kDa} nanodrugs with zero, one, or three copies of PSMA targeted ACUPA ligands were evaluated and their targeted uptake and deep-tumor penetrating ability were compared using PET imaging and biodistribution studies in PSMA+PC3-Pip and PSMA-PC3-Flu prostate cancer xenografts (FIG. 1).

### Materials and Instrumentation

Materials: 4-armed PEG_{40 kDa}-(NH₂)₄ (catalog no. GL4-400PA) was purchased from NOF America. ⁸⁹Zr oxalate was purchased from 3D Imaging, and DFB (catalog no. B-705) was purchased from Macrocyclics. Fetal bovine serum (FBS), penicillin-streptomycin (P/S) solutions, and RPMI-1640 medium were acquired from Life Technologies, Thermo Fisher Scientific (Waltham, MA). All other commercially available chemicals (building blocks, reagents, and solvents) were purchased as reagent grade from Sigma Aldrich, VWR, or Thermo Fisher Scientific and used as received. HPLC was performed using a 4.6×150 mm 5 um 300 Å Jupiter C18 reversed-phase column. ¹H NMR spectra were acquired utilizing a Bruker 400 MHz NMR. Chemical shifts were presented in parts per million (ppm, δ). HRMS was performed at QB3/Chemistry Mass Spectrometry Facility, University of California, Berkeley.

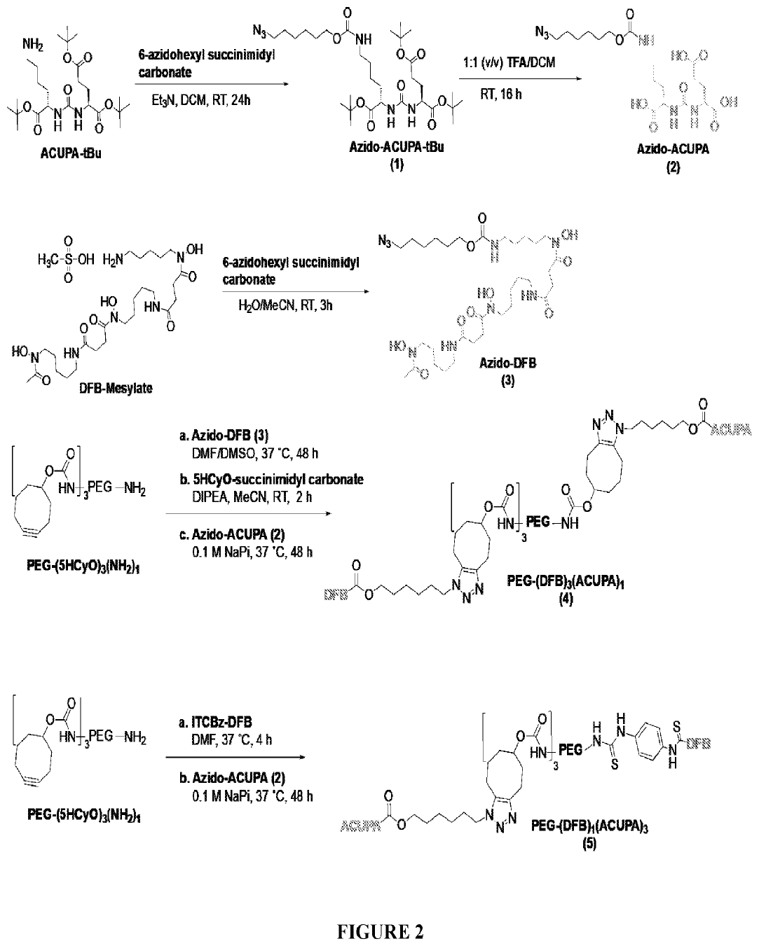

### Synthesis of StarPEG Conjugates (FIG. 2)

General: HPLC was performed using a 4.6×150 mm 5 μm 300 Å Phenomenex Jupiter C₁₈ reversed-phase column at a flow rate of 1.0 mL/min, with a 15-minute linear gradient of 0-100% acetonitrile/water/0.1% TFA beginning 2 min after injection unless otherwise specified.

Azido-ACUPA-tBu (1): A solution of 6-azidohexyl succinimidyl carbonate (150 mg, 0.52 mmol, 1 Eq) in 1 mL of DCM was added to ACUPA-tBu (283 mg, 0.58 mmol, 1.1 Eq) in 5 mL DCM containing 176 μL of DIPEA. The reaction was subjected to magnetic stirring at room temperature for 24 h under argon. After completing the reaction, the solvent was removed under reduced pressure. The mixture was purified by a column chromatography on silica gel eluting with 5% MeOH in DCM to afford Azido-ACUPA-tBu (1) (282 mg, 74% yield). ¹H NMR (400 MHz, CDCl₃): δ 5.17 (br, 2H), 4.96 (br, 1H), 4.33 (m, 2H), 4.04 (m, 2H), 3.26 (t, 2H, J=6.9 Hz), 3.14 (m, 2H), 2.30 (m, 2H), 2.07 (m, 1H), 1.85 (m, 1H), 1.74 (m, 2H), 1.60 (m, 6H), 1.45-1.39 (m, 33H). ¹³C NMR (100 MHz, CDCl₃): δ 172.56, 172.44, 157.04, 156.99, 82.22, 81.87, 80.66, 64, 74, 53.42, 53.12, 51.49, 40.64, 32.79, 31.72, 29.83, 29.53, 29.05, 28.89, 28.53, 28.21, 28.15, 26.54, 25.62, 22.35. HRMS for C₃₁H₅₆N₆O₉ [M+H]⁺ 657.4187 (Calculated: 656.4109).

Azido-ACUPA (2): A solution of Azido-ACUPA-tBu (1) (450 mg, 0.68 mmol) in 3 mL of anhydrous DCM was added to 3 mL of TFA, and the mixture was subjected to magnetic stirring at room temperature for 16 h. After completion of the reaction, the solvent was removed by a rotary evaporator. The residue was dissolved in H₂O and evaporated again to remove the remaining TFA. The residue was dissolved in CHCl₃ and evaporated to yield the product Azido-ACUPA (2) which was utilized without further purification. ¹H NMR (400 MHz, CDCl₃): δ 4.31 (m, 1H), 4.24 (m, 1H), 4.02 (m, 2H), 3.88 (t, 1H), 3.57 (s, 1H), 3.21 (q, 2H), 3.09 (t, 2H), 2.56 (m, 1H), 2.51 (s, 1H), 2.41 (m, 2H), 2.14 (m, 1H), 1.81 (m, 3H), 1.63 (m, 4H), 1.47-1.35 (m, 10H). ¹³C NMR (100 MHz, CDCl₃): δ 176.45, 176.40, 175.82, 160.14, 159.29, 65.66, 58.66, 55.84, 53.50, 52.36, 47.92, 33.20, 31.08, 30.49, 29.83, 28.94, 27.47, 23.84, 18.70. HRMS for C₁₉H₃₂N₆O₉ [M−H]⁺ 487.2162 (Calculated: 488.2231).

Azido-DFB (3): Desferrioxamine B mesylate (Sigma, 165 mg, 0.25 mmol, 1.0 Eq) was dissolved in 1 mL of water in a 15-mL Falcon tube and neutralized by addition of 0.5 mL of 1 M Na₂CO₃. A solution of 6-azidohexyl succinimidyl carbonate (75 mg, 0.26 mmol, 1.04 Eq) in 1 mL of acetonitrile was added, leading to slow formation of a precipitate. After 3 hours at ambient temperature, the precipitate was collected by centrifugation and washed successively with water and acetonitrile. Drying under vacuum provided the product (Yield 60%, 110 mg). HPLC: Rv=10.2 mL; purity 96% (ELSD detection). ¹H NMR (400 MHz, DMSO-d6): δ 9.63 (br, 1H), 9.58 (br, 2H), 7.76 (t, 2H, j=5.6 Hz), 7.02 (t, 1H, j=5.6 Hz), 3.91 (t, 2H, j=6.6 Hz), 3.45 (m, 6H), 3.31 (m, 4H), 2.99 (m, 4H), 2.94 (m, 2H), 2.57 (t, 4H, J=7.6 Hz), 2.27 (t, 4H, J=7.4 Hz), 1.50 (m, 10H), 1.41-1.30 (m, 11H), 1.21 (m, 6H). ¹³C NMR (100 MHz, DMSO-d6): δ 171.95, 171.26, 170.11, 156.30, 63.38, 50.53, 47.08, 46.77, 38.40, 29.89, 29.09, 28.80, 28.56, 28.13, 27.55, 26.02, 25.80, 24.92, 23.48, 23.33, 20.32. HRMS for C₃₂H₅₉N₉O₁₀ [M+H]⁺ 730.4465 (Calculated: 729.4385).

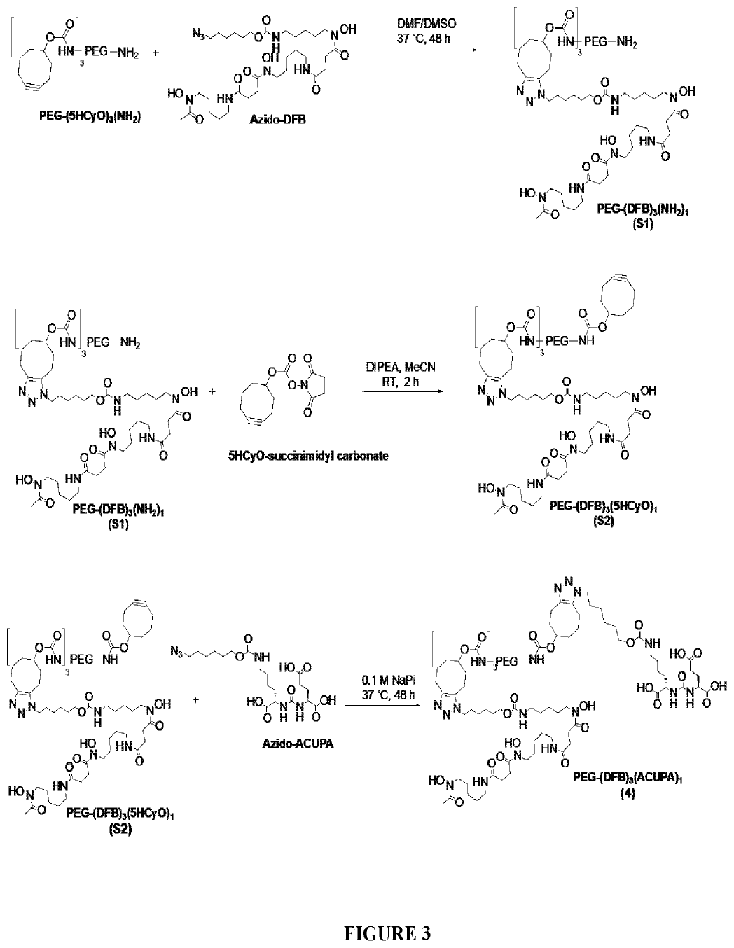

Synthesis of PEG-(DFB)₃(ACUPA)₁ (4) (FIG. 3): A 100 mg/mL solution of PEG-(5HcyO)₃(NH₂)₁ in DMF (1.0 mL, 100 mg, 2.5 mmol, 1.0 Eq) was mixed with a 45 mg/mL solution of Azido-DFB (3) in DMSO (9 mg, 12.3 mmol, 1.5 Eq) and kept at 37° C. for 48 h. The mixture was dialyzed against water (SpectraPor 2 membrane, 12-14 kDa cutoff) followed by methanol to removed unconjugated materials, then dried under reduced pressure to give PEG-(DFB)₃(NH₂)₁ (S1). The residue was dissolved in 1 mL of acetonitrile, filtered to remove insoluble material, and treated with a 10 mg/mL solution of 5-hydroxycyclooctyne (5HcyO) succinimidyl carbonate in acetonitrile (75 mL, 0.75 mg, 2.8 mmol, 1.5 Eq) and DIPEA (1 mL, 5 mmol, 2.8 Eq) for 30 min at ambient temperature. The mixture was added slowly to 10 mL of MTBE and the precipitated product was collected, washed with MTBE, and dried to give PEG-(DFB)₃(5HcyO)₁ (S2), which was advanced to next step without further characterization or purification.

PEG-(DFB)₃(5HcyO)₁ (S2) was dissolved in 1 mL of 0.1 M sodium phosphate, pH 7.4, and treated with a 25 mM solution of Azido-ACUPA (2) in 0.1 M sodium phosphate, pH 7.4, (200 μL, 5 μmol, 2.8 Eq) for 48 h at 37° C., then dialyzed (SpectraPor 2 membrane, 12-14 kDa cutoff) against water followed by methanol, and dried under reduced pressure to provide the product PEG-(DFB)₃(ACUPA)₁ (4) (Yield 45%, 48 mg).

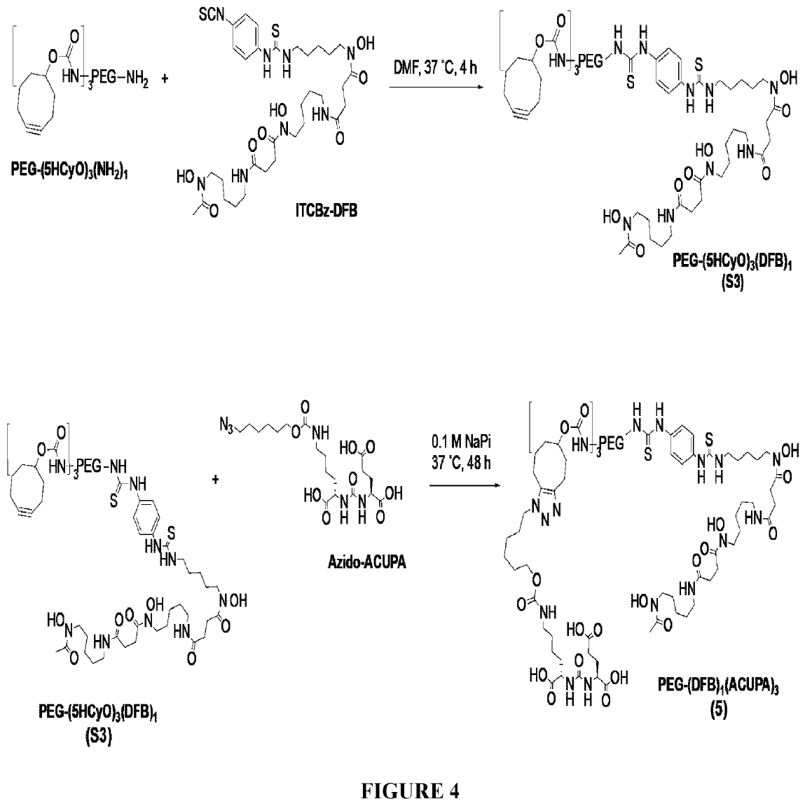

Synthesis of PEG-(DFB)₁(ACUPA)₃ (5) (FIG. 4): A 100 mg/mL solution of PEG-(5HcyO)₃(NH₂)₁ in DMF (1.0 mL, 100 mg, 2.5 μmol, 1.0 Eq) was mixed with a 10 mg/mL solution of p-isothiocyanatobenzyl-desferrioxamine B (ITCBz-DFB) in DMSO (Macrocyclics, 2.8 mg, 3.7 μmol, 1.5 Eq) and kept at ambient temperature for 16 h. The mixture was dialyzed against water (SpectraPor 2 membrane, 12-14 kDa cutoff) to removed unconjugated materials, then dried under reduced pressure. The residue was dissolved in 0.1 M sodium phosphate, pH 7.0, to provide a 75 mg/mL solution of PEG-(5HcyO)₃(DFB)₁ (S3), which was advanced to the next step without further characterization or purification.

A mixture of 75 mg/mL PEG-(5HcyO)₃(DFB)₁ (S3) in 0.1 M NaPᵢ, pH 7.0, (850 μL, 1.6 μmol, 4.8 μmol 5HcyO) and a 30 mM solution of Azido-ACUPA (2) in 0.1 M NaPᵢ, pH 7.4, (400 μL, 12 μmol, 2.5 Eq) was kept 48 h at 37° C., resulting in clean formation of a new product peak by HPLC. The mixture was dialyzed (SpectraPor 2 membrane, 12-14 kDa cutoff) against water followed by methanol, and dried under reduced pressure to provide the product PEG-(DFB)₁(ACUPA)₃ (5) (Yield 78%, 50 mg).

Cell Culture: The PSMA(−) PC3-Flu and PSMA(+) PC3-Pip cell lines (Dr. Martin Pomper, Johns Hopkins University) were cultured in RPMI1640 medium with 1% penicillin/streptomycin and 10% FBS and were maintained with 5% CO₂ at 37° C. According to experimental protocols, cells were detached from flasks by treating 0.25% trypsin solution for 1-2 minutes for further passage or transfer to suitable multi-well plates.

Competition Radioligand Binding Assay: The nanoparticles were utilized in a ⁶⁸Ga-PSMA-11 competition radioligand binding assay to obtain the IC₅₀ values, similar to our prior method. As previously reported, ⁶⁸Ga-PSMA-11 was using eluate from a ⁶⁸Ge/⁶⁸Ga generator, where 5 g of precursor yields around 370-1295 MBq. PC3-Pip cells were seeded in 96-well plates (˜20 k cells/wells) ˜24 h prior testing in RPMI1640 medium containing with 10% FBS and 1% penicillin/streptomycin. The growth medium was discarded, and the cells were washed with PBS two times. Different concentrations (0.01-100000 nM) of the nonradiolabeled nanodrugs were added to the wells in triplicate, and ˜0.185 MBq (2.5 ng) of ⁶⁸Ga-PSMA-11 were treated to each well in growth medium. The treated cells were incubated for 1 h at room temperature. Then, the radioactive medium was discarded, and the cells were washed twice with PBS. The cells in each well were then treated with NaOH (5N, 250 μL) and incubated for 30 min at 37° C. for lysis. The lysate from each well was collected in vials and analyzed in a Hidex gamma counter to quantify the bound activity. IC₅₀ was calculated by nonlinear regression analysis in Graph-Pad Prism software.

⁸⁹Zr Radiolabeling of StarPEGs: 9 μL of ⁸⁹Zr-oxalate (192.4 MBq) was neutralized with 9 μL of 1 M Na₂CO₃, followed by 400 μL of 1 M NH₄Oac. To this buffer solution, 4 mg of StarPEG in 100 μL DI water were added, and 1 h incubation of the reaction mixture was performed at 25° C. The radiolabeled product was purified by size-exclusion chromatography using a PD-10 desalting column (Fisher Scientific, Catalog No. 45-000-148) and saline solution as a mobile phase. After purification, the solution was subject to instant thin layer chromatography (iTLC) to confirm radiolabeling purity using glass microfiber chromatography paper impregnated with silica gel (Neta Scientific, Catalog No. SGI0001) and developed using 50 mM EDTA solution as a mobile phase. The isolated bound activities were 159.1-185 MBq. Multiple radiolabeling studies were carried out with different varying amount of nanodrugs and ⁸⁹Zr, and were summarized in Table 1. The radiolabeling yields were in the range of 26.27-46.25 MBq/mg.

In Vitro Saturation Binding Assay: PC3-pip cells were seeded in 12-well plates (˜100 k cells/well) 24 h prior testing in RPMI1640 medium containing 1% penicillin/streptomycin and 10% FBS. Cells were washed with PBS twice, and 1 mL of RPMI1640 media was added to each well with/without 10 μM PSMA-2 (a previously described PSMA inhibitor), and incubated at 37° C. for 1 h. Then different concentrations (1-1000 nM) of the ⁸⁹Zr radiolabeled Star-PEG nanodrugs were added to the cells and incubated for another 1 h at 37° C. The radioactive medium was discarded, and cells were washed twice with PBS. Each of the wells was then treated with 1 mL NaOH (5N, 250 μL) for 30 min at 37° C. for cell lysis. The lysate from each well was transferred to vials and analyzed in a Hidex gamma counter to quantify the bound activity. K_{d} value was calculated by nonlinear regression one site-specific binding using GraphPad Prism Software. These data were further used to show the PSMA binding affinity and blocking of the Star-PEG nanodrugs at 1 h.

In Vitro Binding and Blocking Assay: PC3-Flu and PC3-pip cells were seeded in 24-well plates (˜50 k cells/well) 24 h prior testing in RPMI1640 medium containing 1% penicillin/streptomycin and 10% FBS. Cells were washed with PBS twice, and 1 mL of RPMI1640 media was added to each well with/without 10 μM PSMA-2 (a well-explored PSMA inhibitor), and incubated at 37° C. for 1 h. Then different concentrations (10-100 nM) of the ⁸⁹Zr radiolabeled Star-PEG nanodrugs were added to the cells and incubated further at 37° C. The radioactive medium was discarded at 4 h or 24 h time points, and cells were washed twice with PBS. Each of the wells was then treated with 1 mL NaOH (5N, 250 μL) and was incubated for 30 min at 37° C. for cell lysis. The lysate from each well was transferred to small vials and analyzed in a Hidex gamma counter along with the standard treated activity to calculate the % bound activity.

In Vitro Membrane-Bound & Internalization Assay: Four sets of the PC3-flu and PC3-pip cells were plated in 24-well plates (˜50 k cells/well) 24 h before assay in the RPMI1640 growth medium containing 10% FBS and 1% penicillin/streptomycin. Each well was treated with 1 μM ⁸⁹Zr radiolabeled Star-PEG nanodrugs and incubated at 37° C. At each time point (1 h, 2 h, 4 h, and 24 h), one set of the PC3-flu and PC3-pip cells were washed with PBS twice. The cells were then treated with 0.5 mL ice-cold solution of 50 mM glycine and 150 mM NaCl and incubated at 4° C. for 5 min, and the acid buffer was transferred to vials, corresponding to the membrane-bound activity. Then the cells were treated with 500 μL 5N NaOH and incubated for 30 minutes at 37° C. to lyse them. The lysate was collected in different small vials corresponding to the internalized activity. All the activities were analyzed using a Hidex gamma counter along with the standard treated activity of the ⁸⁹Zr radiolabeled Star-PEG nanodrugs to calculate the % of membrane-bound and internalized activities.

Inoculation of Mice with Dual Xenograft: All in vivo animal experiments were conducted under a protocol approved by the UCSF Institutional Animal Care & Use Committee (IACUC). 5-6 weeks old male athymic mice (nu/nu, homozygous; Jackson Laboratories or Envigo-Harlan Laboratories, Livermore CA) kept under sterile environments were inoculated subcutaneously with tumor cells. Both the PC3-Flu and PC3-Pip cells were cultured using the mentioned general protocol. Around 2.5 million cells for PC3-Flu and 3 million cells for PC3-Pip in a 100 μL 1:1 mixture of matrigel (Fisher Scientific, Catalog No. 08774391) and RPMI-1640 medium were injected in the right (PC3-Flu) and left (PC3-Pip) flank of the animals, respectively. Tumor size of around 150-300 mm³ was observed after 1-2 weeks post-inoculation.

In Vivo PET Imaging and Biodistribution Studies: Nine days after inoculation, when the tumor size reached 150-300 mm³, the animals were anesthetized by isoflurane, and the respective ⁸⁹Zr radiolabeled Star-PEG nanodrugs were administered through tail vein injection (˜7.4 MBq/mouse in 100 μL of saline). The study population comprised two groups (for each group, n=4 mice). The animals were scanned at 24 h, 48 h, 168 h, and 216 h post radiopharmaceutical injection in a μPET/CT imaging system (Inveon, Siemens, Germany). PET imaging data were acquired with 20 min of acquisition for 24 h and 48 h scan, 30 min acquisition for 168 h scan, and 40 min acquisition for 216 h scan in list mode, and the manufacturer's iterative OSEM 2-D reconstruction algorithm were followed to reconstruct the data. The resulting image data were then normalized to the administered activity to parameterize images in terms of % ID/cc. Open-source AMIDE software (http://amide.sourceforge.net/) was used to process and vied the imaging data. The tumor-bearing mice were sacrificed at 216 h post-injection of the ⁸⁹Zr radiolabeled Star-PEG nanodrugs. 216 h post-injection, blood was collected by cardiac puncture, and the mice were sacrificed to harvest major organs (Liver, Kidney, Spleen, Heart, Pancreas, Lung, Brain, Femur, Muscle, Testis, and subcutaneous tumor). The major organs and blood were weighed and analyzed in an automated gamma counter (Hidex). The percent injected dose per gram of tissue (% ID/g) was estimated by comparing known radioactivity standards.

Autoradiography: After analyzing the dissected organ samples in the gamma counter (Hidex), the tumors were immediately collected and flash frozen in optimal cutting temperature (OCT) compound on dry ice. Tissues were sectioned on a microtome at a thickness of 20 μm and immediately mounted on iQID charged-particle digital autoradiography imaging systems (Qscint Imaging Solutions, LLC). The autoradiography images were processed using ImageJ software.

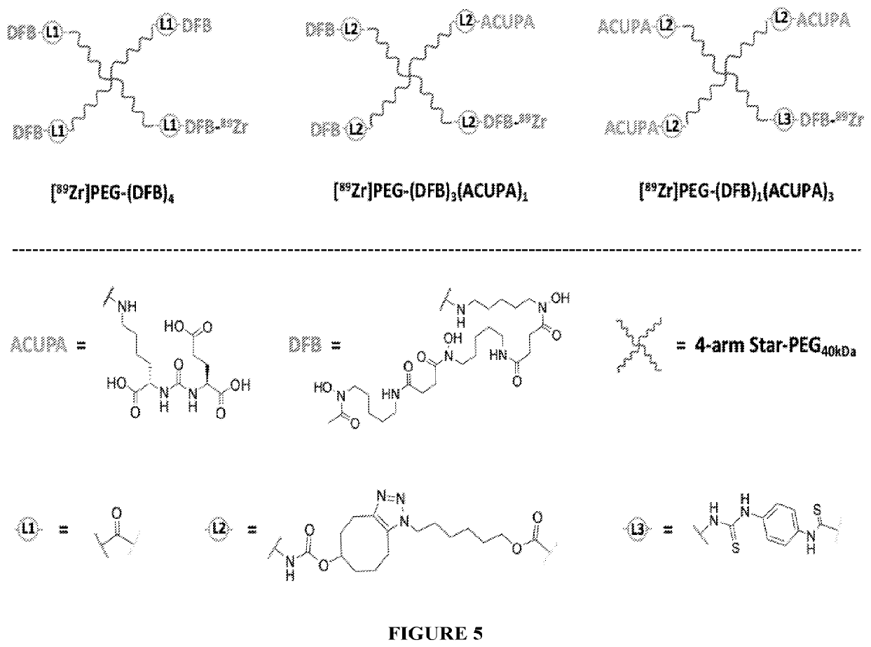

Design, Synthesis, and Radiolabeling of Star-PEG conjugates: It was hypothesized that PSMA targeting ACUPA ligands would increase tumor accumulation of the nanodrugs. To test this hypothesis, two Star-PEGs conjugated with one or three PSMA targeting ACUPA ligands were designed and synthesized, and their PSMA targeting ability with deep-tissue penetration was compared with a non-targeted congener without any ACUPA ligands (FIG. 5). All the Star-PEG conjugates were tethered with deferoxamine B (DFB) ligands, which is a robust ⁸⁹Zr chelator commonly used in the development of PET imaging agents. The PEG polymer of 40 kDa, a Food and Drug Administration (FDA) approved polymer for safe human use, was used to synthesize the nanodrugs.

PEG-DFB₄ was synthesized by the reaction of 4-armed PEG succinimidyl carbonate with DFB mesylate following our previously reported synthetic route (FIG. 2). The two Star-PEG conjugates, i.e., PEG-(DFB)₃(ACUPA)₁ (4) and PEG-(DFB)₁(ACUPA)₃ (5) were synthesized using second-generation azide click reaction with cyclooctyne counterpart (FIGS. 2, 3, and 4). The azide counterparts Azido-ACUPA (2) and Azido-DFB (3) were synthesized following reported procedures (FIG. 2).

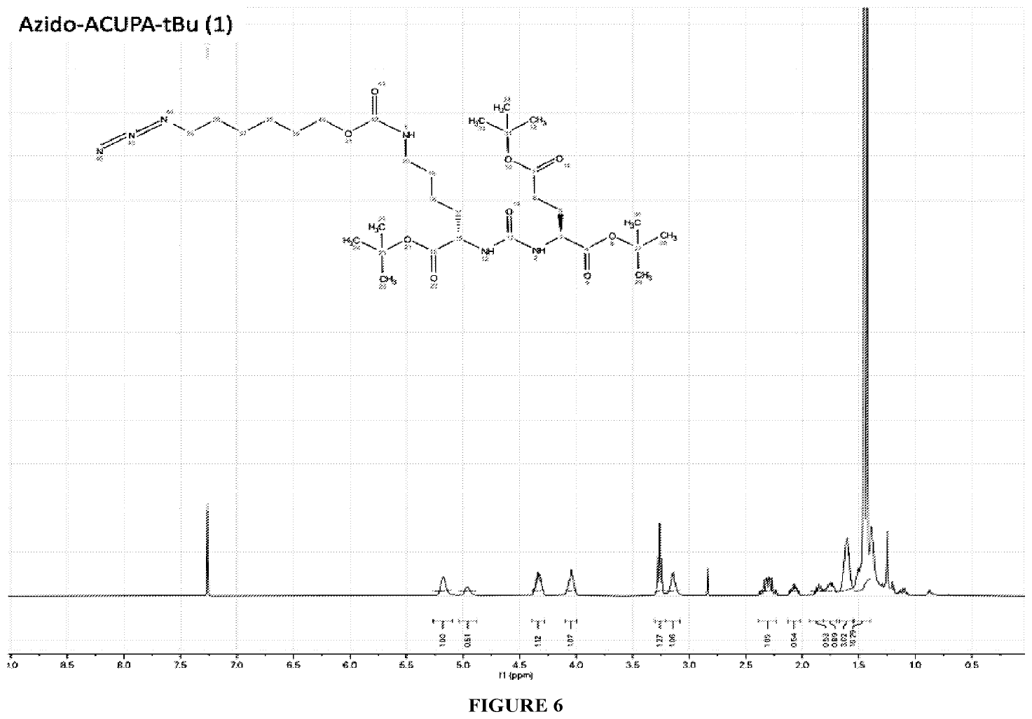

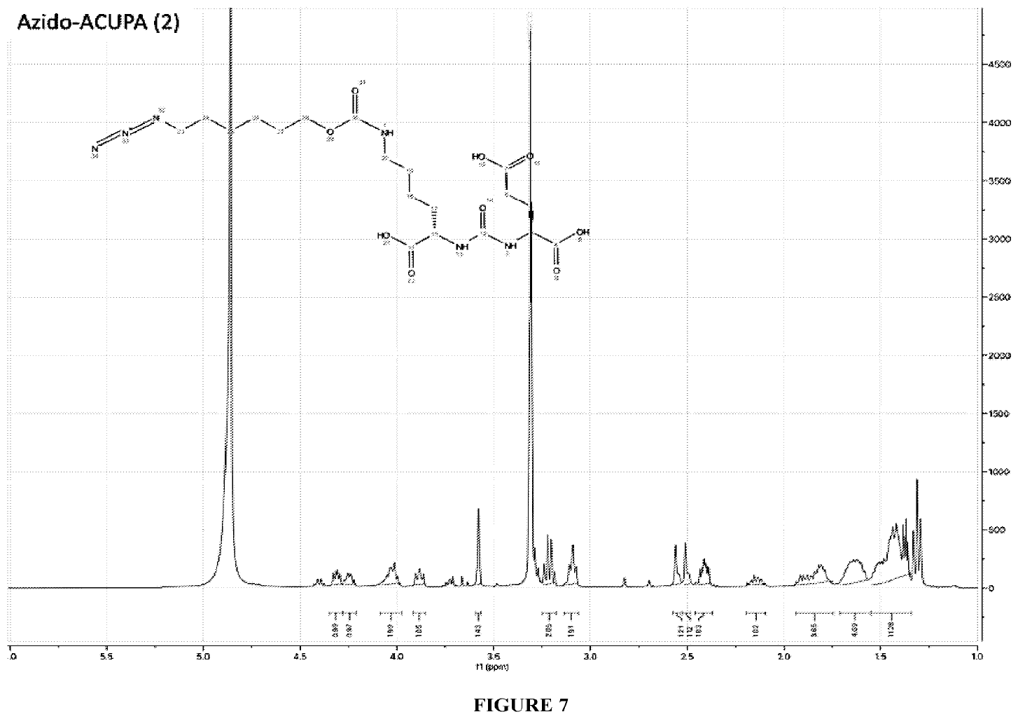

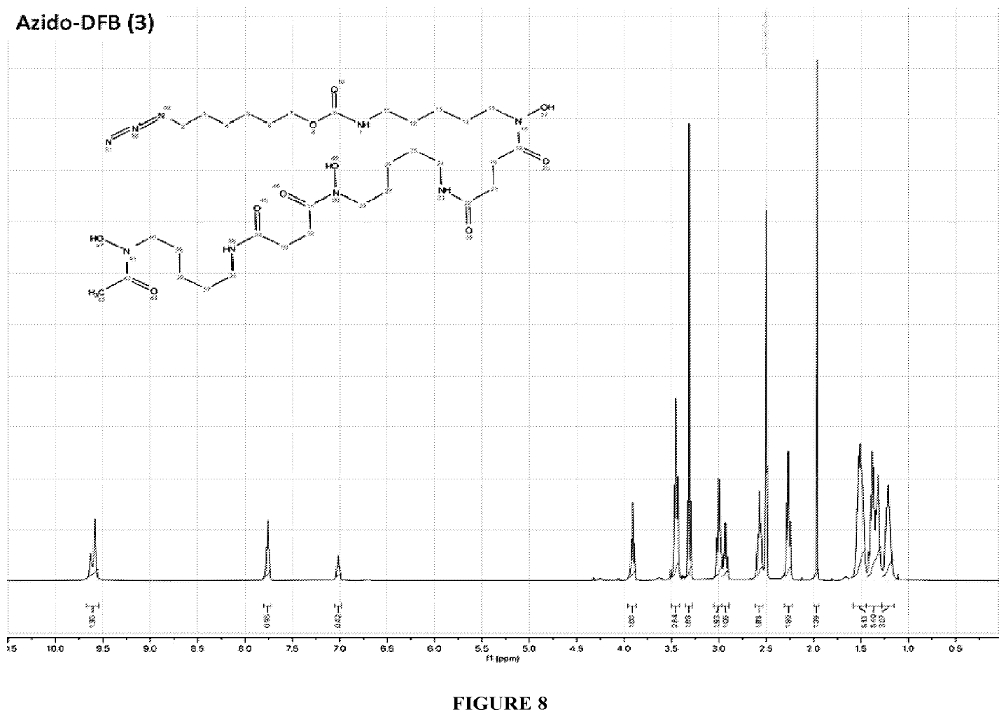

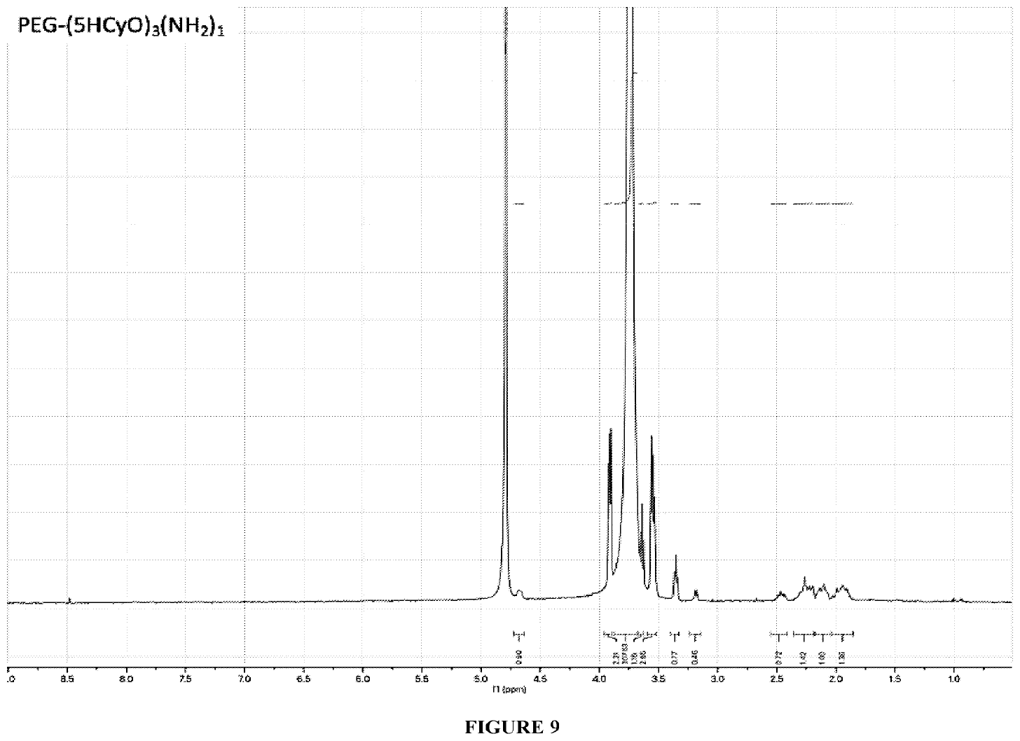

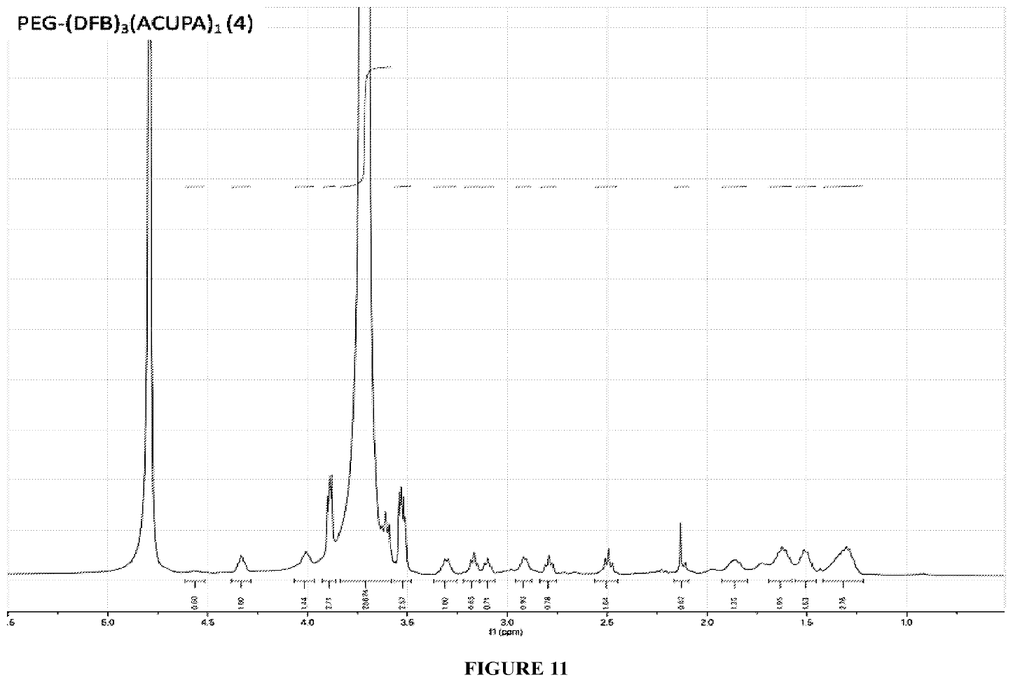

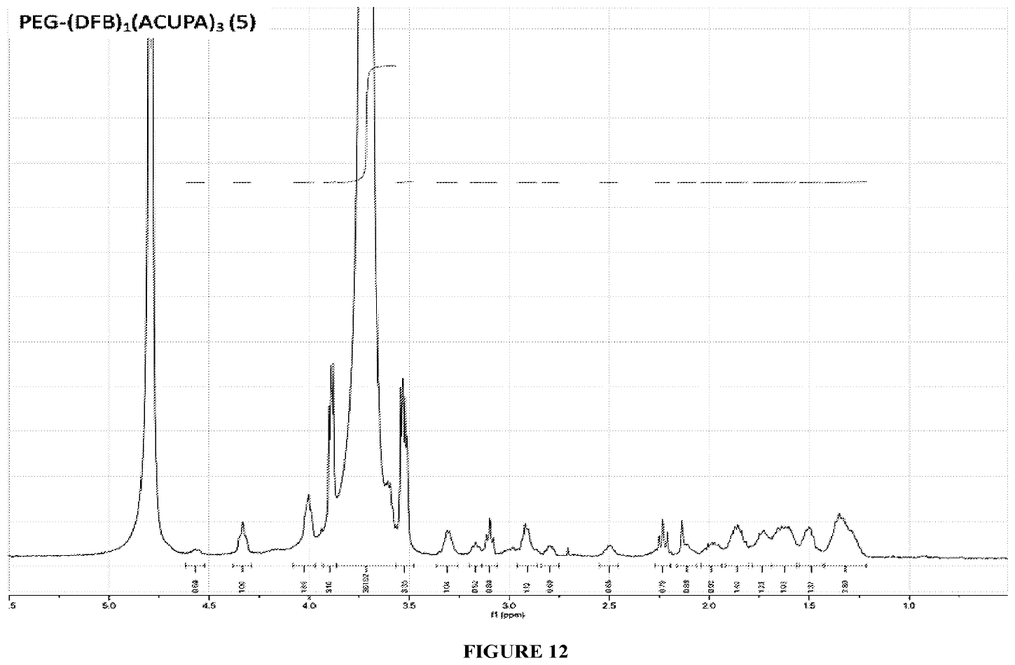

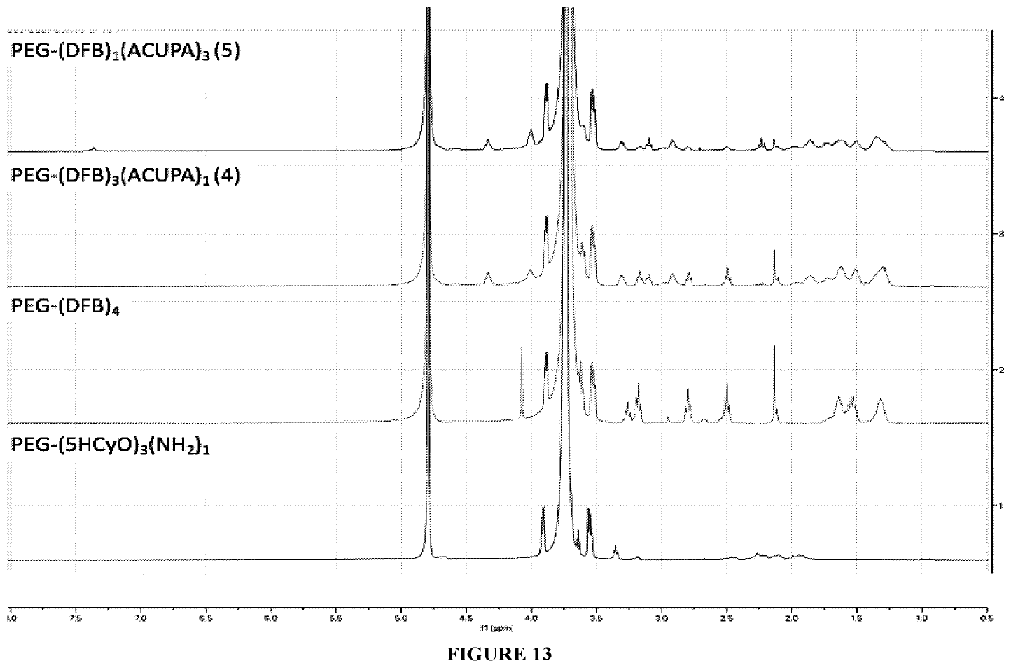

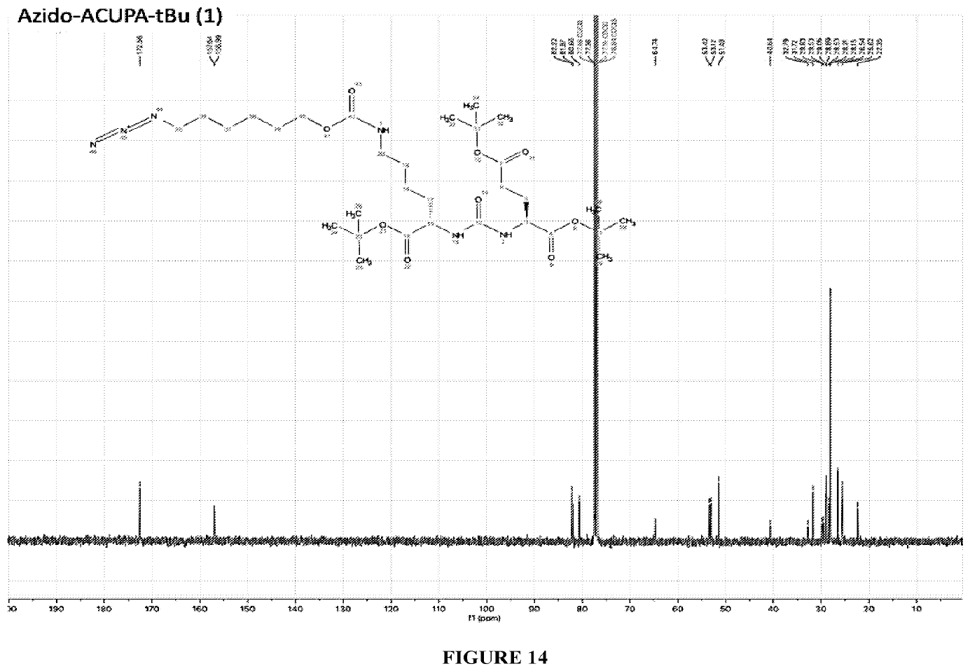

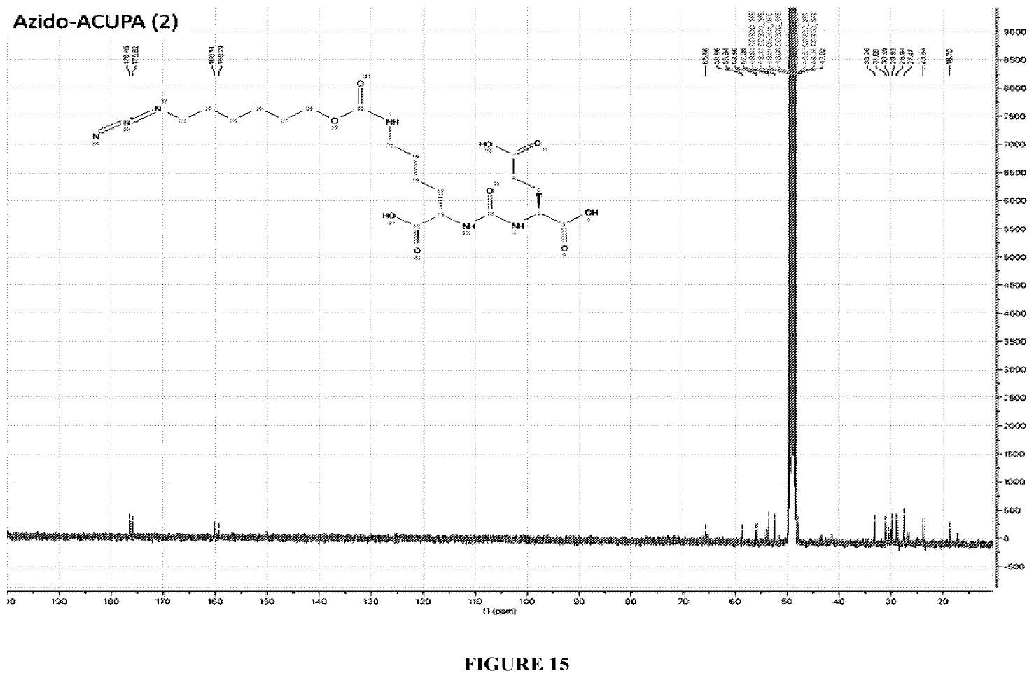

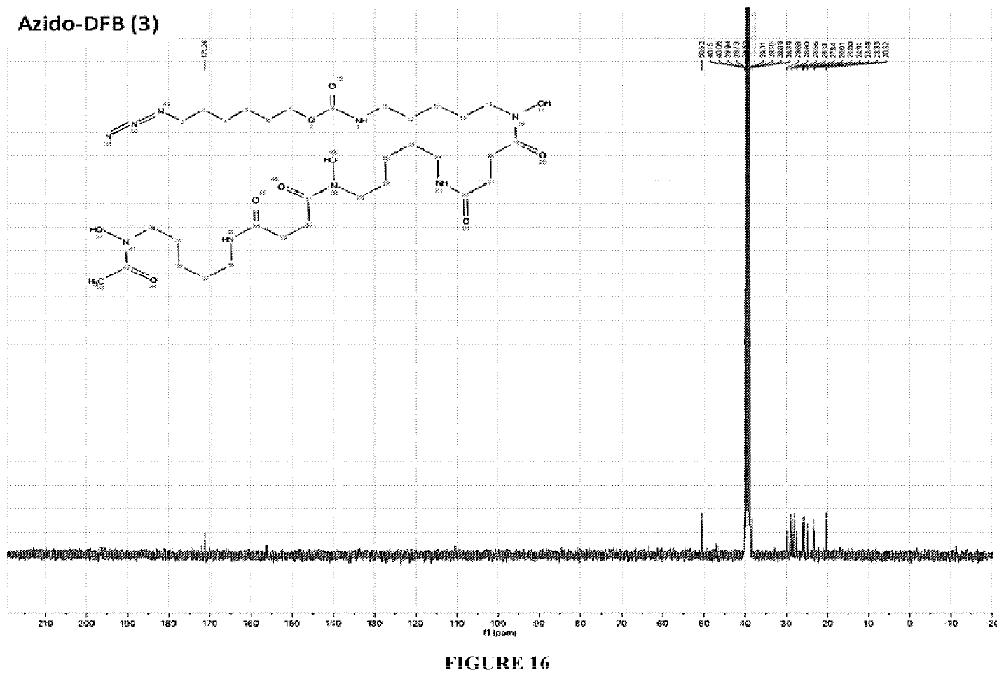

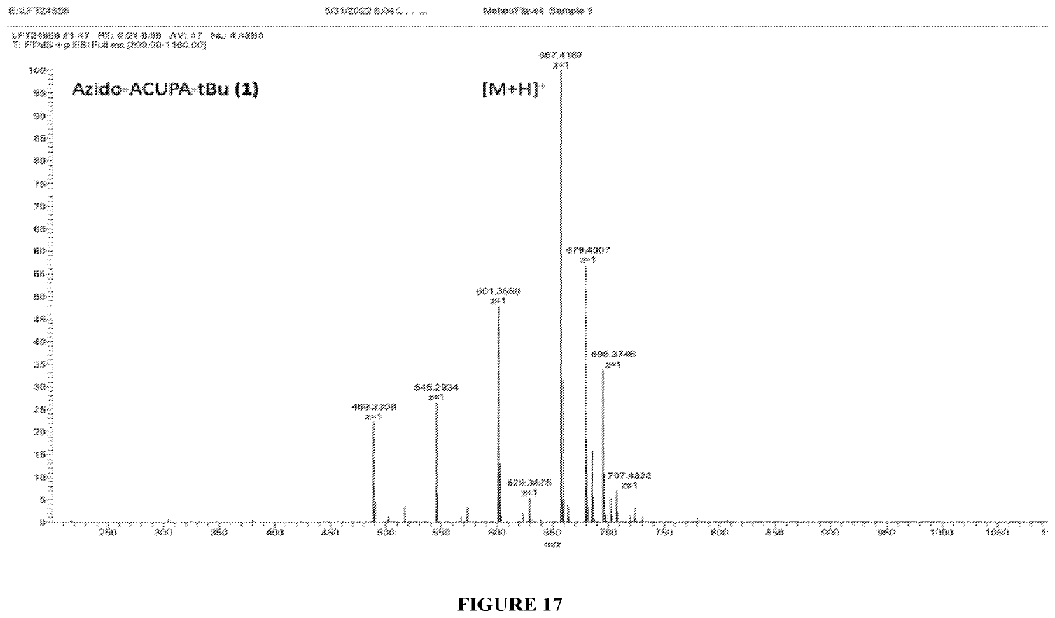

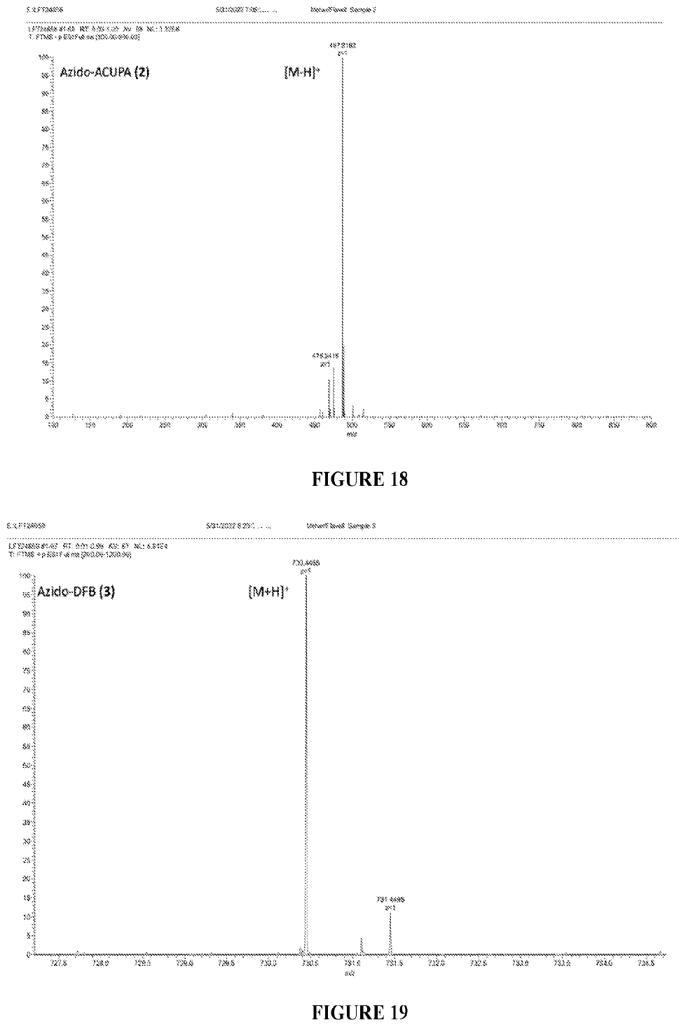

The previously synthesized and reported PEG-(5HcyO)₃(NH₂)₁ was used as the starting material for the synthesis of the targeted nanodrugs. PEG-(5HcyO)₃(NH₂)₁ conjugate with free amine was treated with Azido-DFB (3) to form PEG-(DFB)₃(NH₂)₁ (S1) and was subsequently treated with 5-hydroxycyclooctyne (5HcyO)-succinimidyl carbonate followed by Azido-ACUPA (2) to produce PEG-(DFB)₃(ACUPA)ᵢ(4). Whereas the PEG-(5HcyO)₃(NH₂)₁ was reacted with ITCBz-DFB to produce PEG-(5HcyO)₃(DFB)₁, (S3) which was further reacted with Azido-ACUPA (2) to yield PEG-(DFB)₁(ACUPA)₃ (5). ¹H and/or ¹³C NMR were recorded for the newly synthesized ligands and Star-PEG conjugates (FIGS. 6-16). Additionally, HRMS of the small molecule intermediates like Azido-ACUPA-tBu, Azido-ACUPA and Azido-DFB ligands were also recorded (FIGS. 17-19).

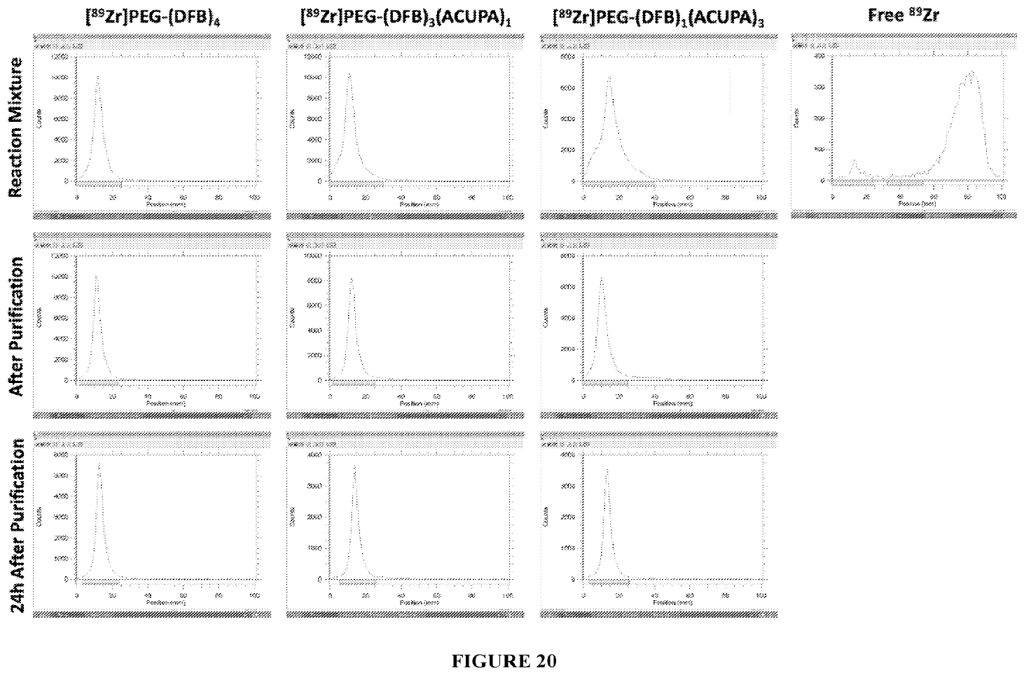

The radiolabeling was carried out by treating the respective Star-PEG conjugates with ⁸⁹Zr oxalate. The resulting complex was purified by size exclusion chromatography technique using a PD-10 desalting column and saline solution as mobile phase. The yield of the ⁸⁹Zr radiolabeling was 90-95% by iTLC analysis (FIG. 20). The isolated yields were 93-98% for PEG-(DFB)₄ (n=3) and PEG-(DFB)₃(ACUPA)₁ (n=2), whereas 81-82% (n=2) of radiolabeled PEG-(DFB)₁(ACUPA)₃ were isolated (Table 1). The specific activities were ranged from 31.07-44.77 MBq/mg for PEG-(DFB)₄, 31.81-46.25 MBq/mg for PEG-(DFB)₃(ACUPA)₁, and 26.27-39.77 MBq/mg for PEG-(DFB)₁(ACUPA)₃.

TABLE 1
Radiolabeling yield and molar activity ⁸⁹Zr labeled nanodrugs.

| | | Activity | Isolated | Specific |
|---|---|---|---|---|
| | Wt | Taken | Yield | Activity |
| PEG conjugates | (mg) | (MBq) | (MBq) | (MBq/mg) |
| PEG-(DFB)₄ | 2.58 | 91.02 | 88.43 | 34.27 |
| | 4.0 | 192.4 | 179.08 | 44.77 |
| | 4.93 | 155.77 | 153.18 | 31.07 |
| PEG-(DFB)₃(ACUPA)₁ | 2.71 | 91.02 | 86.21 | 31.81 |
| | 4.0 | 192.4 | 185.0 | 46.25 |
| PEG-(DFB)₁(ACUPA)₃ | 2.83 | 91.02 | 74.37 | 26.27 |
| | 4.0 | 192.4 | 159.1 | 39.77 |

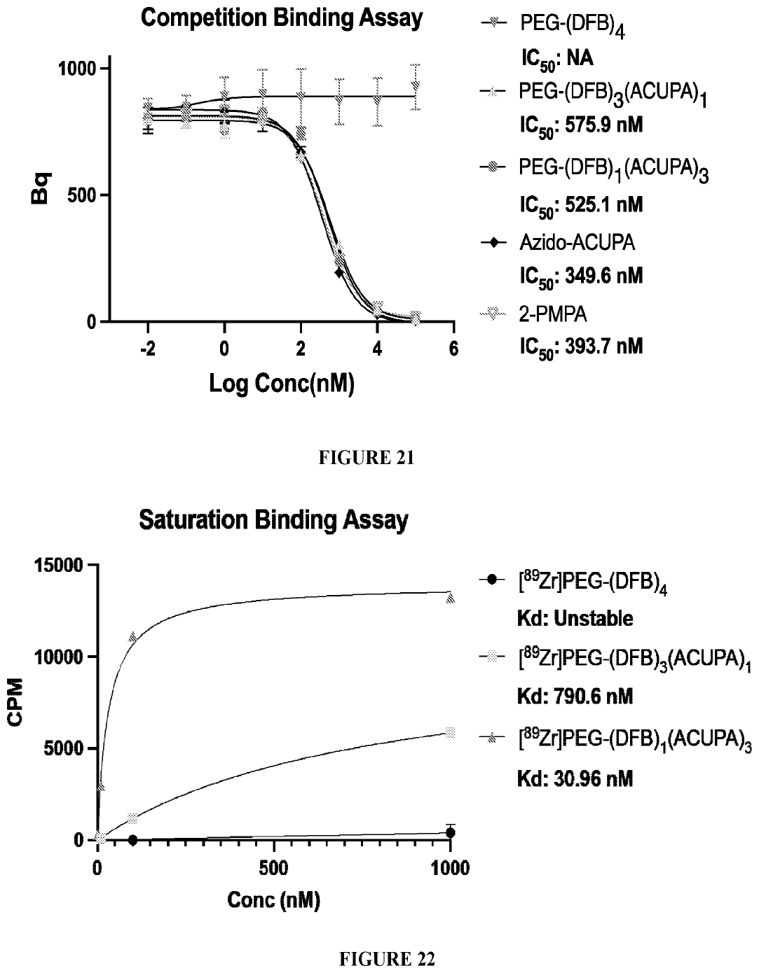

In Vitro Cell Binding Assay: The relative binding affinity of the nonradiolabeled Star-PEG nanodrugs was determined by using ⁶⁸Ga-PSMA-11 based competitive radioligand binding assay in PSMA+PC3-Pip cells (FIG. 21, Table 2). 2-PMPA and the intermediate Azido-ACUPA were used as positive controls. As expected, no evidence of specific binding was observed for the nontargeted nanodrug PEG-(DFB)₄. However, the IC₅₀ values for the targeted nanodrugs PEG-(DFB)₃(ACUPA)₁ and PEG-(DFB)₁(ACUPA)₃ were found to be 459-575 nM with 95% confidence interval in the range of 359-706 nM, demonstrating relatively similar competition binding affinity despite the presence of different number of PSMA targeting ACUPA ligands. A slightly lower IC₅₀ was obtained for Azido-ACUPA (349.6 nM) and 2-PMPA (393.7 nM) with 95% confidence interval in the range of 182-482 nM.

TABLE 2
Competition radioligand binding assay results of the unlabeled
nanodrugs in PSMA+ PC3-Pip cells using ⁶⁸Ga-PSMA-11.

| | Experiment 1 | | Experiment 2 | |
|---|---|---|---|---|
| | IC₅₀ | IC₅₀, 95% CI | IC₅₀ | IC₅₀, 95% CI |
| Nanodrugs | (nM) | (nM) | (nM) | (nM) |
| PEG-(DBF)₄ | NA | NA | NA | NA |
| PEG-(DBF)₃(ACUPA)₁ | 575.9 | 466.3-706.6 | 459.8 | 359.8-584.4 |
| PEG-(DBF)₁(ACUPA)₃ | 525.1 | 406.4-670.7 | 527.8 | 404.4-683.2 |
| Azido-ACUPA | 349.6 | 290.1-420.9 | — | — |
| 2-PMPA | 393.7 | 320.3-482.4 | 214.6 | 182.3-253.3 |

The binding affinity of the ⁸⁹Zr labeled nanodrugs was further evaluated in a saturation binding assay that demonstrated ˜25 fold lower dissociation constant for [⁸⁹Zr]PEG-(DFB)₁(ACUPA)₃ (K_{d}=30.96 nM) with three copies of ACUPA ligands compared to the [⁸⁹Zr]PEG-(DFB)₃(ACUPA)₁ (K_{d}=790.6 nM) with only one copy of ACUPA ligands (FIG. 22, Table 3). In contrast, no evidence of specific binding was observed for the nontargeted [⁸⁹Zr]PEG-(DFB)₄ nanodrug.

TABLE 3
Saturation binding assay results of the ⁸⁹Zr
labeled nanodrugs in PSMA+ PC3-Pip cells.

| | | K_{d}, 95% CI | Bₘₐₓ | Bₘₐₓ, 95% |
|---|---|---|---|---|
| Nanodrugs | K_{d} (nM) | (nM) | (nM) | CI (nM) |
| [⁸⁹Zr]PEG-(DBF)₄ | No | NA | NA | NA |
| | binding | | | |
| [⁸⁹Zr]PEG-(DBF)₃(ACUPA)₁ | 790.6 | 653.2-987.7 | 10464 | 9603-11669 |
| [⁸⁹Zr]PEG-(DBF)₁(ACUPA)₃ | 30.96 | 26.10-36.69 | 13934 | 13427-14455 |

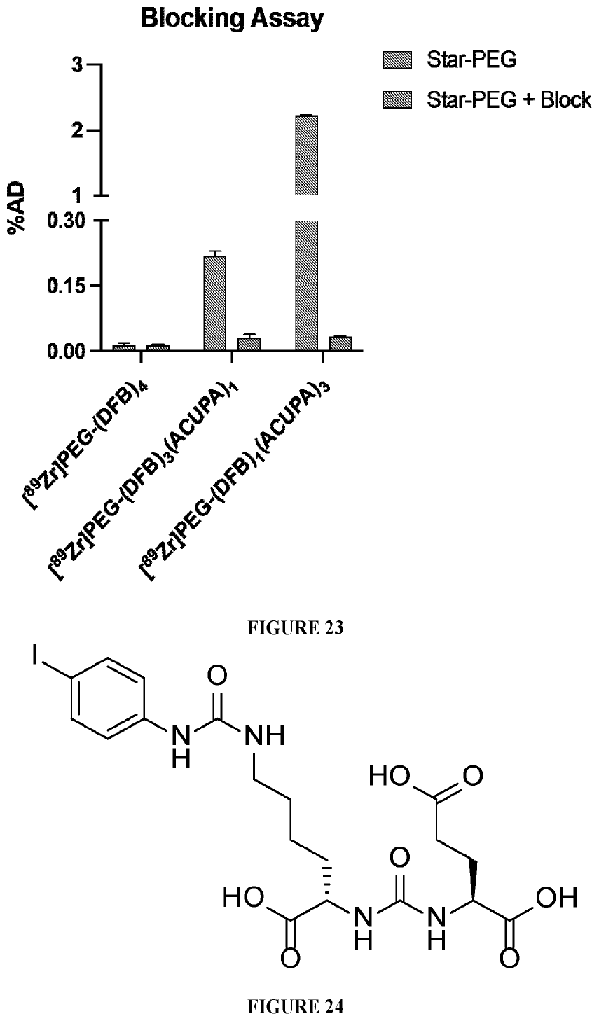

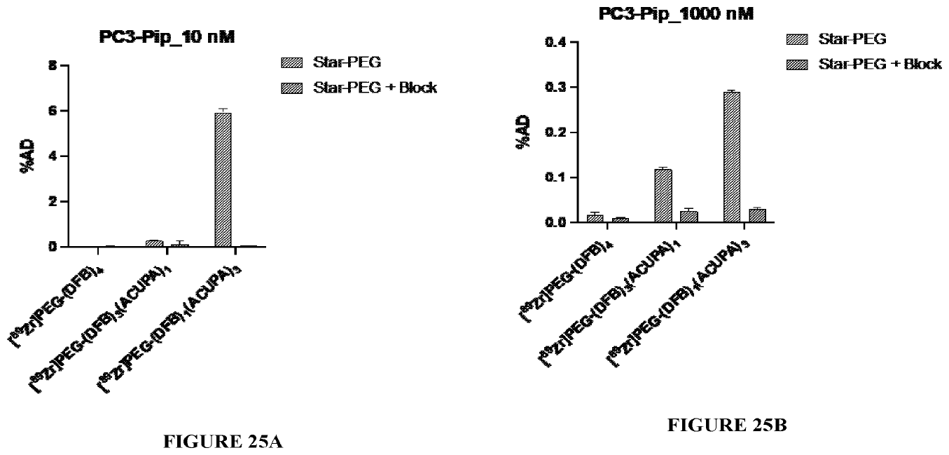

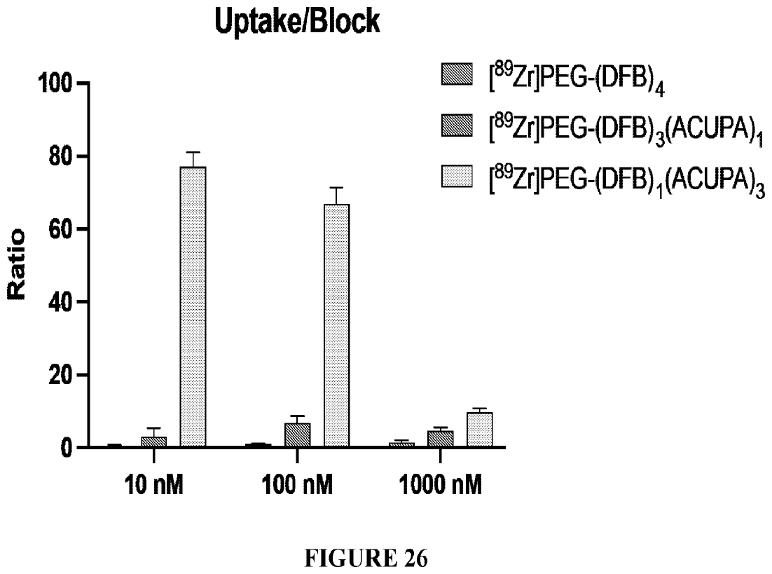

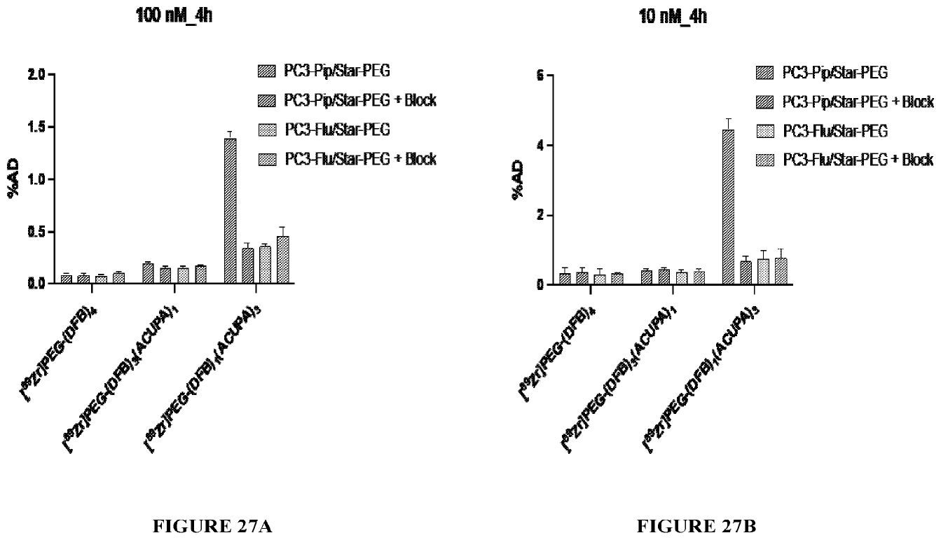

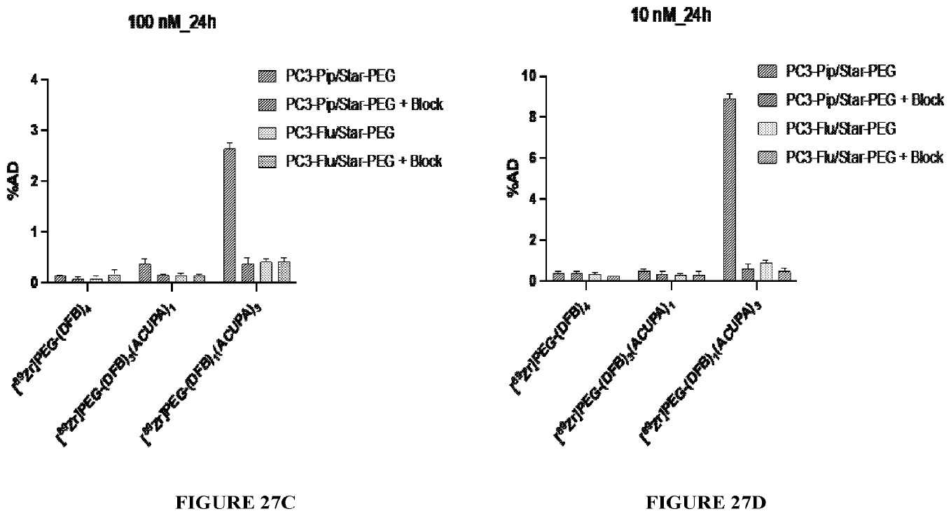

Further, a binding and blocking assay was performed using PSMA-2 as the blocking agent at 1 h, 4 h, and 24 h time points and different nanodrug concentrations (FIGS. 23-27). The targeted nanodrugs, [⁸⁹Zr]PEG-(DFB)₃(ACUPA)₁ and [⁸⁹Zr]PEG-(DFB)₁(ACUPA)₃, demonstrated higher uptake selectively in PSMA+PC3-Pip cells, and the uptake was significantly reduced in the presence of the known PSMA binder PSMA-2. In comparison, no specific uptake of the nanodrugs was observed in PSMA-PC3-Flu cells. Noticeably, at lower probe concentrations (10 nM and 100 nM), [⁸⁹Zr]PEG-(DFB)₁(ACUPA)₃ with three copies of ACUPA ligands demonstrated exceptionally high PSMA targeted cell uptake compared to [⁸⁹Zr]PEG-(DFB)₃(ACUPA)₁ with just one copy of ACUPA (FIGS. 23 and 25A). However, as the concentration of the nanodrugs was increased to 1000 nM, the ratio of uptake to block in PSMA+PC3-Pip cells decreased, demonstrating relatively higher non-specific cell uptake at higher concentrations, presumably due to saturation of the binding sites (FIGS. 25B and 26). Similarly, an increase in the non-specific cell uptake was also observed at higher timepoints (FIGS. 27A-27D).

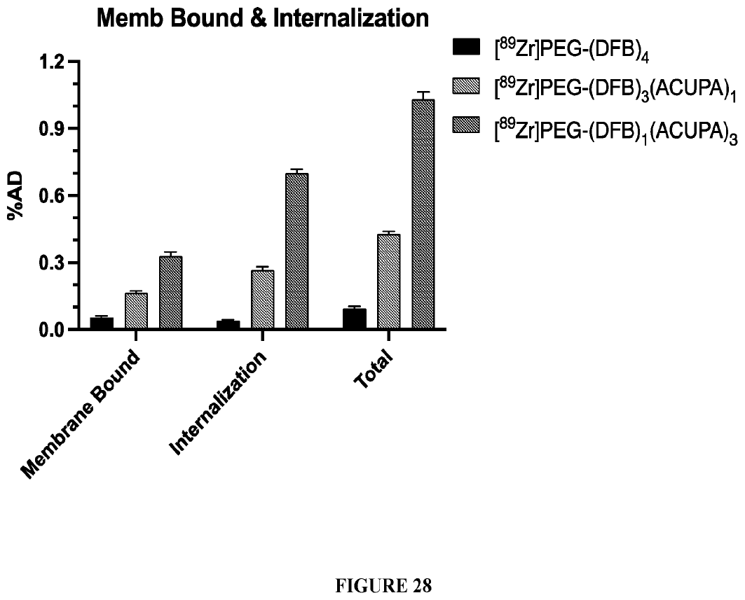

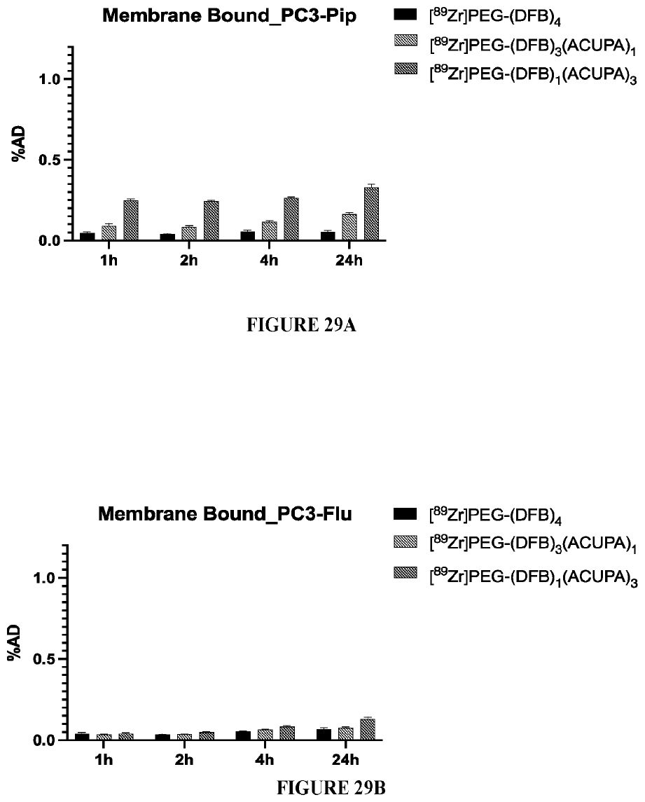

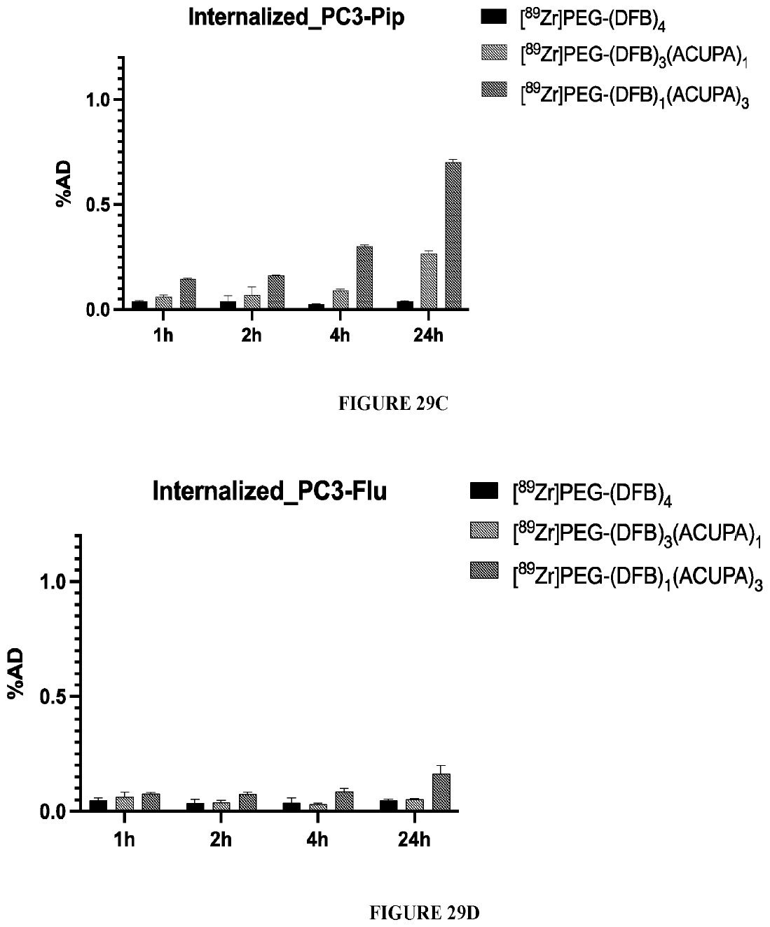

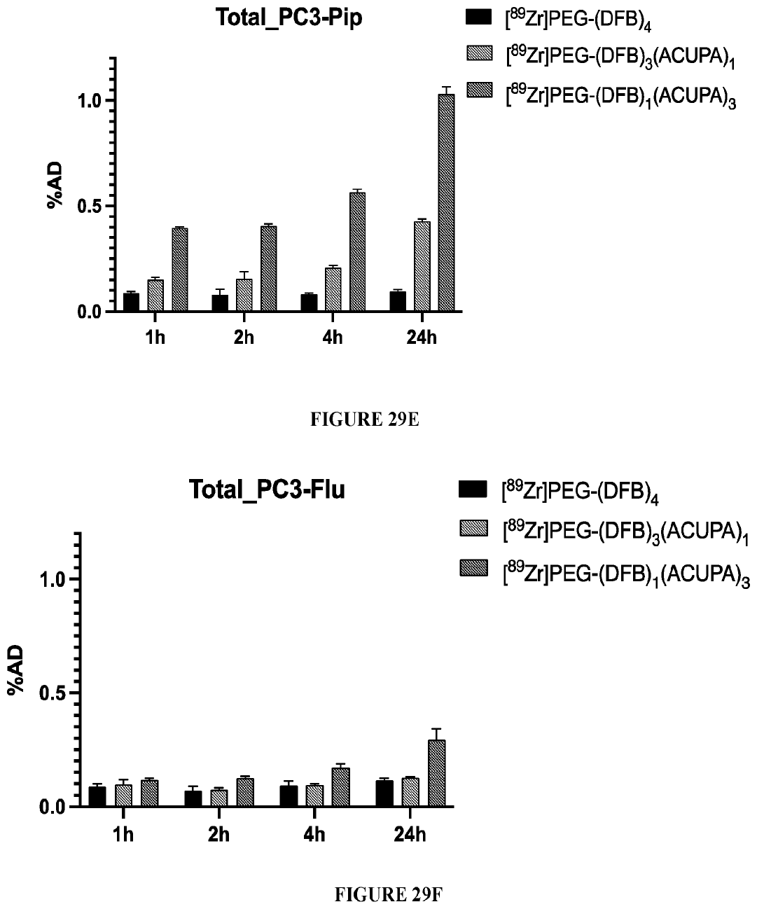

Next, we tested the degree of cellular uptake and internalization. The membrane-bound and internalized activities were isolated in both PSMA+PC3-Pip and PSMA-PC3-Flu cells by acid wash (ice-cold mixture of 50 mM glycine and 150 mM NaCl) at different time points (FIGS. 28-29F). Significantly higher membrane-bound activities were observed for the targeted nanodrugs in PSMA+PC3-Pip cells, which remained almost similar over time up to 24 h (FIG. 29A). The internalized activities for the targeted nanodrugs increased steadily from 1 h to 24 h (FIG. 29C). Overall, the nanodrug with three copies of PSMA targeting ACUPA ligands demonstrated higher membrane-bound and internalization than its counterpart with one ACUPA ligand. On the contrary, no evidence of PSMA targeted cell uptake was observed for the non-targeted nanodrug [⁸⁹Zr]PEG-(DFB)₄. Taken together, these results demonstrate efficient cell binding and internalization for the PSMA targeted nanodrugs, with relatively higher affinity and cellular uptake when comparing [⁸⁹Zr]PEG-(DFB)₁(ACUPA)₃ to [⁸⁹Zr]PEG-(DFB)₃(ACUPA)₁.

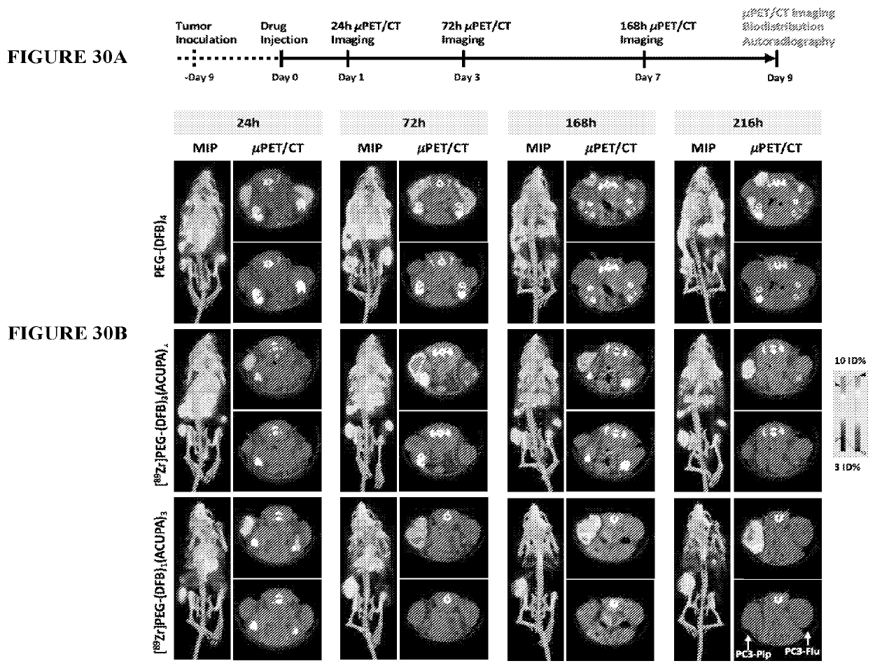

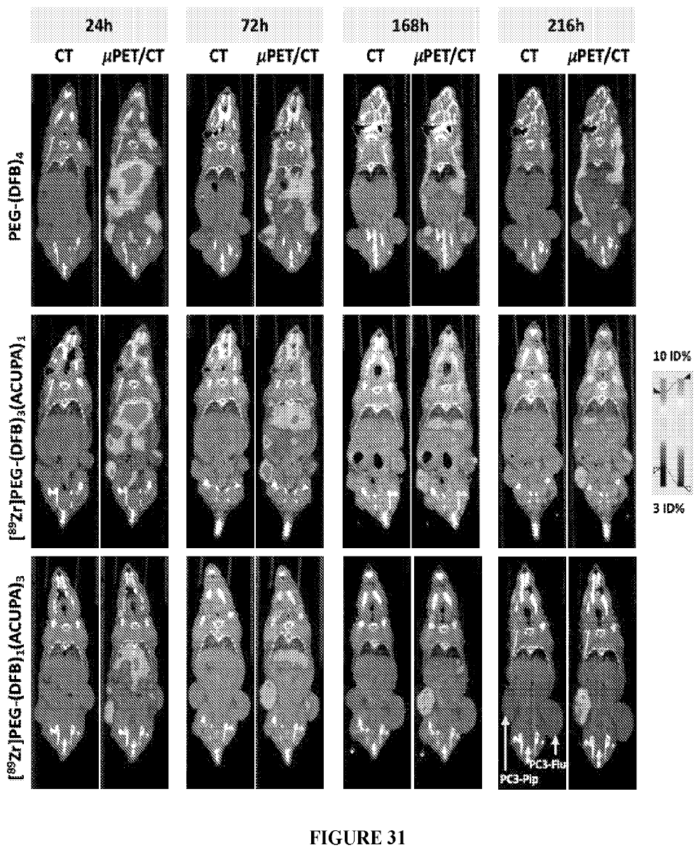

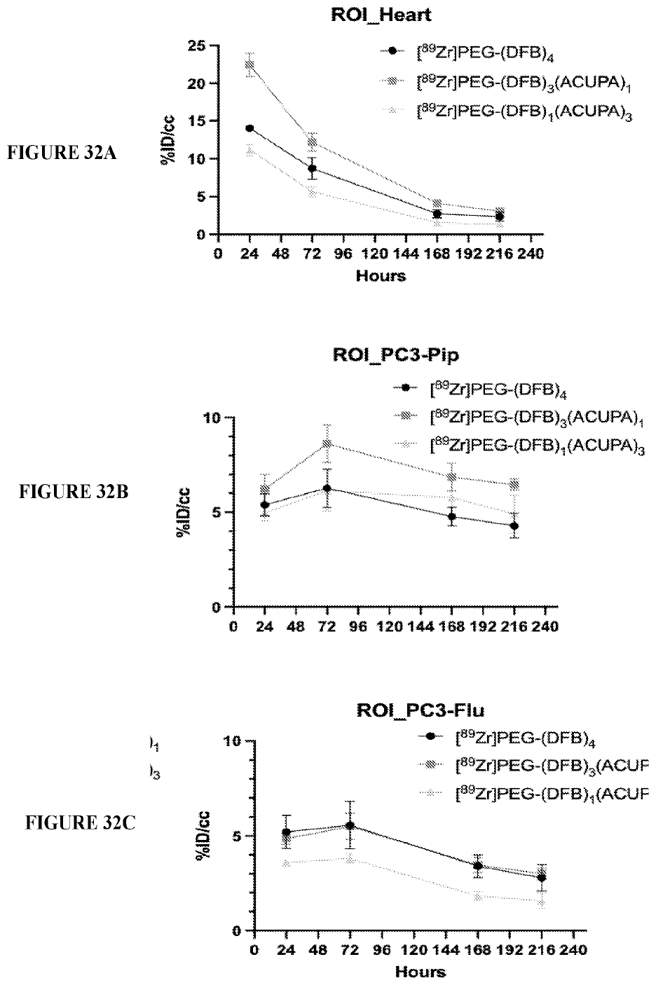

In Vivo μPET/CT imaging: In vivo μPET/CT imaging of the ⁸⁹Zr labeled nanodrugs was performed in the nu/nu athymic mice model with subcutaneous dual xenograft of PSMA+PC3-Pip (left flank) and PSMA-PC3-Flu (right flank). When the tumor size reached 100-200 mm³, the mice were administered the ⁸⁹Zr labeled nanodrugs via tail vein injection and were subjected to multiple time point μPET/CT imaging up to 216 h, as presented in FIG. 30A. The study population was composed of three groups, with one group for each nanodrug (n=4 mice). The maximum intensity projection (MIP), axial μPET/CT, and axial CT images of the nanodrugs are presented in FIG. 30B, and respective coronal images are presented in FIG. 31. All μPET/CT images were segmented into representative regions of interest (ROI) over heart and tumors (Table 4-6), and time-activity curves were plotted (FIGS. 32A-32C).

TABLE 4
Region of interest analysis data reported as % ID/g of the ⁸⁹Zr labeled
nanodrugs on heart at 24 h, 72 h, 168 h, and 216 h. (n = 4)

| Hours | [⁸⁹Zr]PEG-(DFB)₄ | | | | [⁸⁹Zr]PEG-(DFB)₃(ACUPA)₁ | | | | [⁸⁹Zr]PEG-(DFB)₁(ACUPA)₃ | | | |
|---|---|---|---|---|---|---|---|---|---|---|---|---|
| 24 | 14.17 | 13.74 | 14.17 | 14.00 | 22.10 | 20.79 | 22.24 | 24.52 | 11.67 | 11.94 | 10.75 | 10.35 |
| 72 | 7.11 | 7.90 | 9.93 | 9.95 | 12.29 | 11.24 | 11.43 | 13.8 | 6.63 | 5.25 | 5.42 | 5.28 |
| 168 | 2.25 | 2.34 | 3.38 | 2.94 | 3.59 | 4.04 | 4.14 | 4.67 | 1.68 | 1.54 | 1.42 | 1.53 |
| 216 | 1.80 | 1.86 | 2.94 | 2.77 | 2.82 | 3.01 | 3.06 | 3.36 | 1.46 | 1.37 | 1.25 | 1.35 |

TABLE 5
Region of interest analysis data reported as % ID/g of the ⁸⁹Zr labeled
nanodrugs on PC3-Pip tumors at 24 h, 72 h, 168 h, and 216 h. (n = 4)

| Hours | [⁸⁹Zr]PEG-(DFB)₄ | | | | [⁸⁹Zr]PEG-(DFB)₃(ACUPA)₁ | | | | [⁸⁹Zr]PEG-(DFB)₁(ACUPA)₃ | | | |
|---|---|---|---|---|---|---|---|---|---|---|---|---|
| 24 | 5.87 | 4.97 | 5.89 | 4.80 | 5.46 | 6.04 | 7.33 | 6.05 | 5.58 | 4.69 | 4.63 | 4.98 |
| 72 | 7.07 | 5.82 | 7.15 | 5.04 | 7.28 | 8.62 | 8.98 | 9.59 | 7.64 | 5.66 | 5.14 | 6.09 |
| 168 | 5.32 | 4.27 | 5.03 | 4.43 | 6.07 | 6.36 | 7.45 | 7.55 | 7.33 | 5.08 | 4.82 | 5.86 |
| 216 | 4.72 | 4.18 | 4.82 | 3.40 | 6.14 | 6.88 | 6.47 | 6.39 | 6.38 | 4.56 | 4.15 | 4.54 |

TABLE 6
Region of interest analysis data of the ⁸⁹Zr labeled nanodrugs reported
as % ID/g on PC3-Flu tumors at 24 h, 72 h, 168 h, and 216 h. (n = 4)

| Hours | [⁸⁹Zr]PEG-(DFB)₄ | | | | [⁸⁹Zr]PEG-(DFB)₃(ACUPA)₁ | | | | [⁸⁹Zr]PEG-(DFB)₁(ACUPA)₃ | | | |
|---|---|---|---|---|---|---|---|---|---|---|---|---|
| 24 | 4.841 | 5.97 | 5.91 | 4.13 | 4.81 | 4.47 | 4.93 | 5.22 | 3.84 | 3.47 | 3.50 | 3.58 |
| 72 | 4.24 | 6.89 | 6.37 | 4.77 | 4.73 | 5.12 | 6.28 | 5.88 | 3.46 | 3.75 | 3.93 | 4.04 |
| 168 | 3.29 | 4.24 | 3.24 | 2.80 | 3.34 | 3.69 | 3.81 | 2.95 | 1.87 | 1.59 | 1.73 | 2.12 |
| 216 | 1.97 | 3.62 | 2.96 | 2.54 | 2.72 | 3.34 | 2.79 | 3.12 | 1.55 | 1.11 | 2.10 | 1.50 |

Overall, starting from 24 h, the targeted nanodrugs [⁸⁹Zr]PEG-(DFB)₃(ACUPA)₁ and [⁸⁹Zr]PEG-(DFB)₁(ACUPA)₃ with one or three ACUPA ligands demonstrated significantly increased PSMA targeted accumulation in PSMA+PC3-Pip tumors compared to PSMA-PC3-Flu tumors (FIG. 30B). In comparison, the non-targeted nanodrug [⁸⁹Zr]PEG-(DFB)₄ did not show any difference in the tumor uptake irrespective of PSMA expression with very high background contrast even after 216 h. The ROI plot demonstrated an increase in tumor accumulation up to 72 h and a mild decrease afterward up to 216 h, visualized in the PET/CT images as well (FIGS. 31 and 32A-32C). Increased background clearance along with prominent deep-tumor retention was observed for the targeted nanodrugs in PSMA+PC3-Pip. However, at 216 h timepoint, [⁸⁹Zr]PEG-(DFB)₁(ACUPA)₃ showed central photomenia, which could be due to the development of central necrosis inside the large size tumor (FIG. 30B).

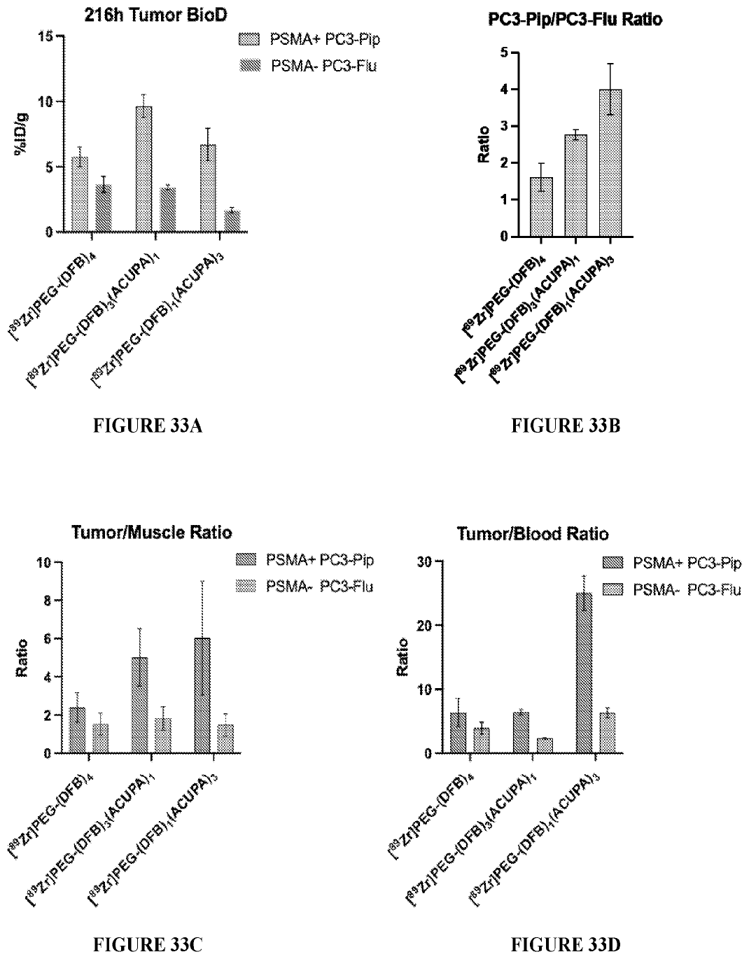

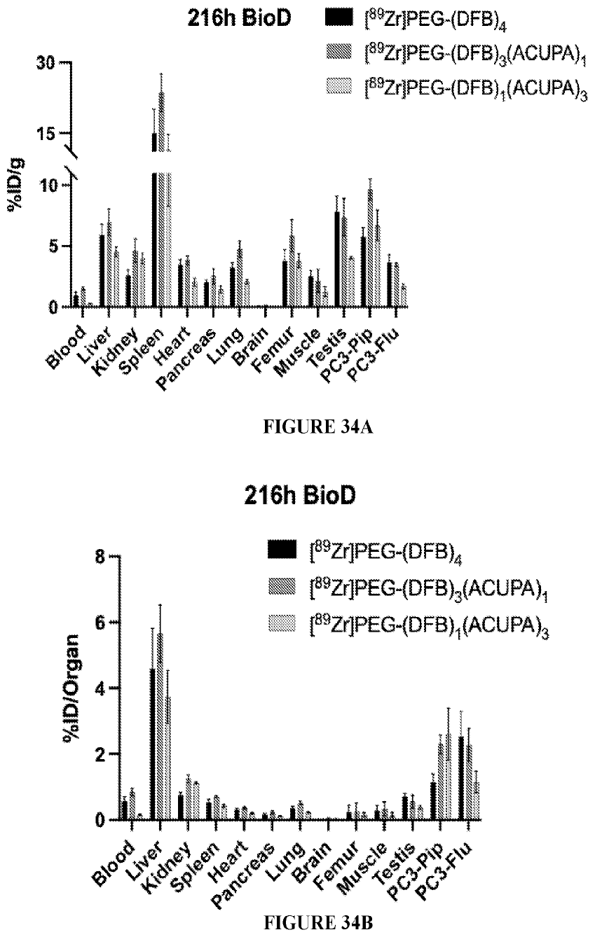

Ex Vivo Organ Biodistribution: The tumor-bearing mice were sacrificed after the 216 h time point μPET/CT imaging, and the major organs including tumors were collected to quantify the distribution of ⁸⁹Zr labeled nanodrugs. The ex vivo organ biodistribution results are presented in FIG. 33A-33D, FIG. 34A-34B, and Tables 7-8. Interestingly, 9.64±0.87% ID PSMA+PC3-Pip tumor uptake was observed for [⁸⁹Zr]PEG-(DFB)₃(ACUPA)₁, which was even higher than the uptake (6.69±1.24% ID) obtained for [⁸⁹Zr]PEG-(DFB)₁(ACUPA)₃. However, despite higher PC3-Pip tumor uptake of [⁸⁹Zr]PEG-(DFB)₃(ACUPA)₁, a superior PC3-Pip/PC3-Flu ratio was obtained for [⁸⁹Zr]PEG-(DFB)₁(ACUPA)₃, demonstrating potentially high PSMA targeted uptake of the later. On the other hand, the nontargeted [⁸⁹Zr]PEG-(DFB)₄ demonstrated 5.75±0.74% ID uptake in PC3-Pip tumors but failed to provide a higher PC3-Pip/PC3-Flu ratio, consistent with EPR based non-specific uptake. Although, PC3-Pip/muscle ratios of both the targeted nanodrugs were comparable at around 5-6, the PC3-Pip/blood ratio of [⁸⁹Zr]PEG-(DFB)₁(ACUPA)₃ was remarkably high at 25 compared to 6 obtained for its counterparts (FIGS. 33C-33D). Overall, while the PSMA+PC3-Pip tumor uptake of both the targeted nanodrugs are comparable, [⁸⁹Zr]PEG-(DFB)₁(ACUPA)₃ possessed highly improved PSMA+PC3-Pip to background contrast to that of other nanodrugs without or with one PSMA targeting ACUPA ligands.

TABLE 7
Organ biodistribution analysis data in ID %/g tissue for ⁸⁹Zr labeled nanodrugs at 216 h. (n = 4)

| Organs | [⁸⁹Zr]PEG-(DFB)₄ | | | | [⁸⁹Zr]PEG-(DFB)₃(ACUPA)₁ | | | | [⁸⁹Zr]PEG-(DFB)₁(ACUPA)₃ | | | |
|---|---|---|---|---|---|---|---|---|---|---|---|---|
| Blood | 0.69 | 0.8 | 1.16 | 1.22 | 1.41 | 1.67 | 1.45 | 1.46 | 0.32 | 0.23 | 0.27 | 0.24 |
| Liver | 4.9 | 5.87 | 5.87 | 7.07 | 6.22 | 6.51 | 6.549 | 8.55 | 5.04 | 4.4 | 4.07 | 4.58 |
| Kidney | 2.4 | 2.41 | 2.2 | 3.28 | 3.96 | 3.96 | 4.536 | 6.01 | 4.44 | 3.76 | 3.5 | 4.21 |
| Spleen | 9.9 | 11.96 | 16.88 | 21.18 | 28.51 | 23.14 | 18.745 | 24.05 | 16.16 | 9.85 | 9.39 | 10.46 |
| Heart | 3.06 | 3.22 | 3.99 | 3.63 | 3.83 | 3.47 | 3.732 | 4.33 | 2.27 | 1.84 | 1.73 | 2.37 |
| Pancreas | 1.75 | 2.08 | 2.19 | 2.09 | 2.22 | 1.96 | 2.54 | 3.37 | 1.73 | 1.66 | 1.16 | 1.24 |
| Lung | 2.74 | 3.01 | 3.64 | 3.53 | 4.19 | 4.4 | 4.708 | 5.7 | 2.33 | 1.99 | 1.91 | 2.08 |
| Brain | 0.07 | 0.08 | 0.09 | 0.08 | 0.1 | 0.1 | 0.124 | 0.1 | 0.07 | 0.05 | 0.06 | 0.05 |
| Femur | 4.27 | 3.07 | 4.83 | 3 | 6.85 | 4.58 | 4.878 | 7.13 | 4.61 | 3.43 | 3.42 | 3.72 |
| Muscle | 2.51 | 2.8 | 1.85 | 2.88 | 1.31 | 1.9 | 1.841 | 3.48 | 0.83 | 1.47 | 0.99 | 1.67 |
| Testis | 7.09 | 7.74 | 9.65 | 6.89 | 6.64 | 6.6 | 9.697 | 6.54 | 4.15 | 4.14 | 3.96 | 3.96 |
| PC3-Pip | 6.48 | 4.99 | 6.31 | 5.25 | 8.41 | 10.43 | 9.701 | 10.03 | 8.49 | 6.05 | 5.72 | 6.52 |
| PC3-Flu | 3.02 | 3.94 | 4.38 | 3.29 | 3.27 | 3.63 | 3.462 | 3.53 | 1.77 | 1.4 | 1.77 | 1.79 |

TABLE 8
Organ biodistribution analysis data in ID %/organ
tissue for ⁸⁹Zr labeled nanodrugs at 216 h. (n = 4)

| Organs | [⁸⁹Zr]PEG-(DFB)₄ | | | | [⁸⁹Zr]PEG-(DFB)₃(ACUPA)₁ | | | | [⁸⁹Zr]PEG-(DFB)₁(ACUPA)₃ | | | |
|---|---|---|---|---|---|---|---|---|---|---|---|---|
| Blood | 0.4 | 0.49 | 0.7 | 0.68 | 0.87 | 0.97 | 0.85 | 0.7 | 0.18 | 0.14 | 0.18 | 0.15 |
| Liver | 5.24 | 3.76 | 5.99 | 3.41 | 6.33 | 4.69 | 6.46 | 5.16 | 4.15 | 3.66 | 4.49 | 2.64 |
| Kidney | 0.69 | 0.77 | 0.7 | 0.86 | 1.1 | 1.38 | 1.24 | 1.29 | 1.12 | 1.1 | 1.17 | 1.13 |
| Spleen | 0.44 | 0.64 | 0.56 | 0.53 | 0.74 | 0.73 | 0.69 | 0.68 | 0.41 | 0.46 | 0.48 | 0.37 |
| Heart | 0.3 | 0.32 | 0.36 | 0.32 | 0.4 | 0.39 | 0.34 | 0.34 | 0.2 | 0.17 | 0.19 | 0.22 |
| Pancreas | 0.13 | 0.19 | 0.19 | 0.15 | 0.2 | 0.24 | 0.19 | 0.29 | 0.11 | 0.13 | 0.13 | 0.1 |
| Lung | 0.34 | 0.35 | 0.43 | 0.34 | 0.47 | 0.51 | 0.59 | 0.5 | 0.24 | 0.21 | 0.25 | 0.22 |
| Brain | 0.03 | 0.03 | 0.03 | 0.03 | 0.04 | 0.04 | 0.05 | 0.03 | 0.03 | 0.02 | 0.02 | 0.01 |
| Femur | 0.41 | 0.05 | 0.42 | 0.05 | 0.66 | 0.1 | 0.09 | 0.16 | 0.23 | 0.09 | 0.17 | 0.12 |
| Muscle | 0.11 | 0.42 | 0.18 | 0.4 | 0.12 | 0.56 | 0.2 | 0.47 | 0.07 | 0.19 | 0.11 | 0.24 |
| Testis | 0.64 | 0.69 | 0.84 | 0.68 | 0.58 | 0.69 | 0.7 | 0.28 | 0.43 | 0.32 | 0.42 | 0.33 |
| PC3-Pip | 0.84 | 1.43 | 1.12 | 1.21 | 2.65 | 1.98 | 2.38 | 2.19 | 3.73 | 2.51 | 1.92 | 2.26 |
| PC3-Flu | 2.34 | 1.71 | 3.53 | 2.57 | 2.07 | 1.69 | 2.51 | 2.83 | 1.47 | 1.34 | 1.05 | 0.74 |

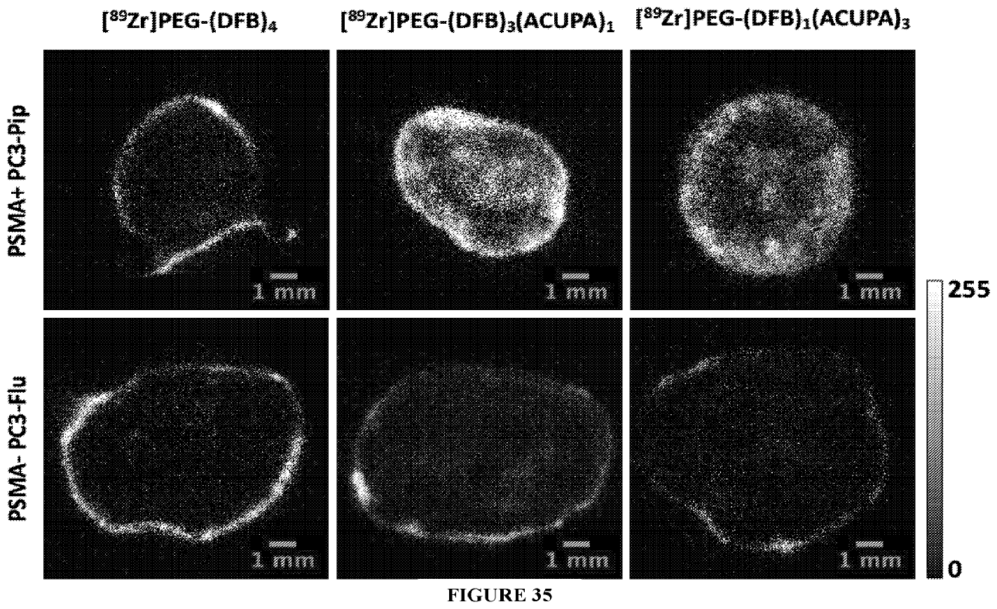

The tumors dissected at 216 h post-injection were subjected to autoradiography analysis to explore the distribution of the ⁸⁹Zr labeled nanodrugs inside the bulk tumor tissue (FIG. 35). Both the targeted nanodrugs with one or three PSMA targeted ACUPA ligands demonstrated excellent uptake with deep-tumor penetration in the PSMA+PC3-Pip tumors. In contrast, only peripheral accumulation of the nontargeted nanodrug [⁸⁹Zr]PEG-(DFB)₄ was witnessed in the PSMA+PC3-Pip tumor. Irrespective of presence of PSMA targeted ACUPA ligands, all the nanodrugs demonstrated only peripheral accumulation in the PSMA-PC3-Flu tumors. Moreover, the distribution of the targeted nanodrugs in PC3-Pip tumors was not homogenous, which could be due to the specific tumor vasculature, and/or central necrosis of the large size tumor. Taken together, Star-PEG nanodrugs demonstrated very good PSMA targeted imaging of prostate cancer with deep-tumor penetration.

### Discussion

Two newly designed ⁸⁹Zr labeled Star-PEG_{40 kDa} nanodrugs with one or three copies of ACUPA ligands were evaluated for their PSMA targeted imaging and deep-tumor penetrating ability in PSMA+PC3-Pip and PSMA-PC3-Flu prostate cancer xenografts (FIG. 5). The nanodrugs were tethered to ⁸⁹Zr chelator DFB ligands for PET imaging. The pharmacokinetics of both the ⁸⁹Zr labeled PSMA targeted nanodrugs were compared with the previously reported nontargeted nanodrug of 40 kDa without any ACUPA ligands, demonstrating very high passive accumulation (>10% ID 9 days post-injection) in MX-1 and HT-29 tumor models.

The 4-armed Star-PEG nanodrug of 40 kDa molecular weight (10 nm of size) provides an optimal size for high EPR based tumor accumulation with an extended half-life, whereas PEGs smaller or larger than 40 kDa eliminate more rapidly with low passive accumulation. However, it has been well established that, along with the nanodrug size, the pharmacokinetics of EPR mediated passive uptake is strongly influenced by tumor vasculature and macrophages. A nontargeted nanostar polymer has been demonstrated by Goos et al., where CT26 tumors with highly leaky vasculature showed high and homogeneous tumor accumulation (14.8% ID/g). However, the nanostar polymer was unable to penetrate deep into the poorly leaky BxPC3 tumor and mostly accumulated at the tumor periphery (>5.5% ID/g). Most of the prostate cancer tumor models like CWR22rv1, DU-145, and PC-3 human prostate xenografts have been evaluated to be EPR-low phenotype with poor deep-tumor penetration of those nanodrugs relying on the EPR mediated passive uptake. It was hypothesized that by conjugating the PSMA targeting ACUPA ligands to the 4-armed Star-PEG nanodrugs, the target-specific tumor accumulation with deep-tissue penetration of the nanodrugs could be improved in prostate cancer xenograft beyond passive uptake.

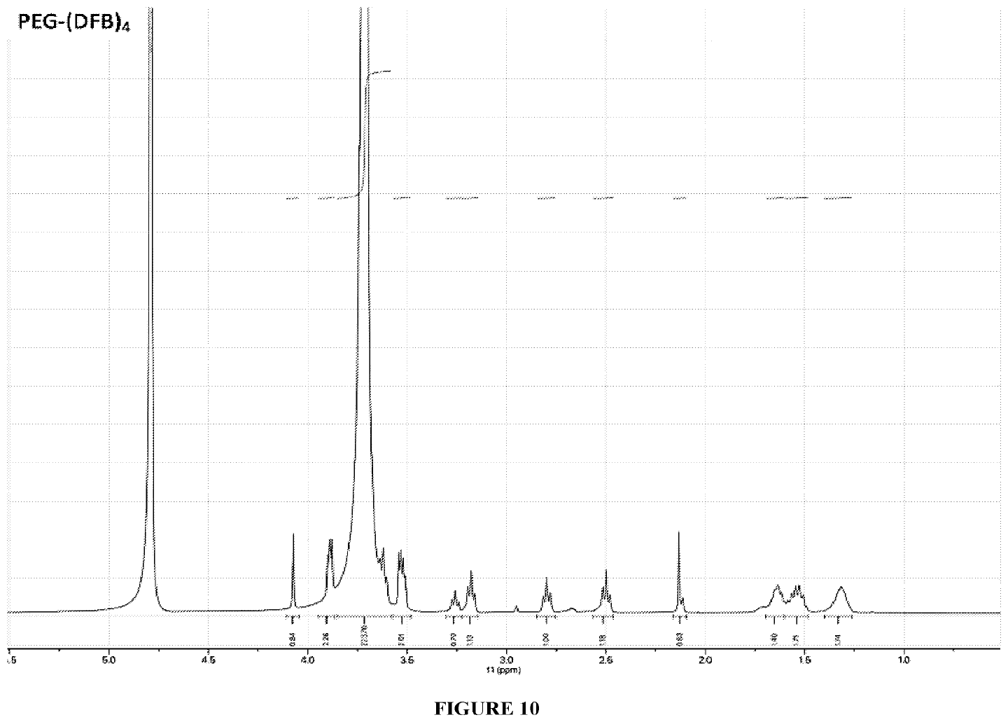

The targeted nanodrugs were developed by conjugating PSMA targeted ACUPA ligands to the 4-armed Star-PEG through cyclooctyne linker using azide-cyclooctyne based metal catalyst-free click reaction (FIG. 2). However, depending on the synthetic convenience, the ⁸⁹Zr chelator DFB was linked to the polymer core via amide linker in [⁸⁹Zr]PEG-(DFB)₄, cyclooctyne triazole linker in [⁸⁹Zr]PEG-(DFB)₃(ACUPA)₁, and p-phenylene diisothiocyanate linker in [⁸⁹Zr]PEG-(DFB)₁(ACUPA)₃ (FIG. 2). Prior reports demonstrated no marked difference in the pharmacokinetics of the PSMA targeted probes with distinct linkers. The Star-PEG nanodrugs were purified by dialysis (12-14 kDa cutoff), and the conjugation of the ligands was confirmed by ¹H NMR analysis (FIG. 10-13).

⁸⁹Zr radiolabeling of the nanodrugs was performed using the reported protocol yielding 26.27-46.25 MBq/mg molar activity with around 81-98% (isolated) radiolabeled yield (FIG. 20 and Table 1). The molar activity and isolated yield of PEG-(DFB)₄ and PEG-(DFB)₃(ACUPA)₁ were relatively higher than that of the PEG-(DFB)₁(ACUPA)₃, which could be rationalized with the number of DFB ligands conjugated to the nanodrug. Overall, three 4-armed PEG-based nanodrugs without or with different numbers of ACUPA ligands were synthesized and radiolabeled with better specific activity to that of the prior reported Star-PEG nanodrugs. The PSMA targeted in vitro cell binding and in vivo pharmacokinetics of those polymer nanodrugs were evaluated in PSMA+PC3-Pip and PSMA-PC3-Flu cells/tumors.

Various cell-binding assays, including competition radioligand binding, saturation binding, blocking, and internalization assays were performed to evaluate the PSMA targeted in vitro binding affinity of the nanodrugs. Around 25.5 fold lower dissociation constant (K_{d}) of 30.9 nM was obtained for [⁸⁹Zr]PEG-(DFB)₁(ACUPA)₃ with three PSMA targeted ACUPA ligands to that of [⁸⁹Zr]PEG-(DFB)₃(ACUPA)₁ (FIG. 22, Table 3). The blocking, membrane-bound, and internalization assay too demonstrated notably higher PSMA targeted cell uptake of [⁸⁹Zr]PEG-(DFB)₁(ACUPA)₃ with three copies of ACUPA ligands (FIGS. 23, 25A-25B, 26, 27A-27D, 28, and 29A-29F). The blocking assay performed at different nanodrug concentrations demonstrated significantly enhanced binding affinity of [⁸⁹Zr]PEG-(DFB)₁(ACUPA)₃ at a lower concentration, compared to when the assay was performed at a higher probe concentration (FIG. 26). These results demonstrated a crucial role of concentration in the binding affinity of the targeted nanodrugs and could be due to a saturation of PSMA binding sites and an increase in the non-specific cell uptake at higher concentrations. Surprisingly, the competition radioligand binding assay against ⁶⁸Ga-PSMA-11 in PSMA+PC3-Pip cells demonstrated highly comparable IC₅₀ (459-575 nM obtained in two independent experiments, Table 2) for both the targeted nanodrugs with one or three ACUPA ligands, respectively. It should be noted that the non-radiolabeled nanodrugs with free DFB ligands were evaluated in the competition radioligand binding assay to determine the IC₅₀ values. It is thought that the complexation of metal in the chelator could alter the overall charge and hydrophilicity of the nanodrugs and thereby could alter the targeted binding affinity of the nanodrugs. Additionally, few other prior studies on PSMA targeted probes demonstrated similar inconsistence correlation of the IC₅₀ to other in vitro binding assay and in vivo pharmacokinetics. No evidence of PSMA targeted binding affinity was observed for [⁸⁹Zr]PEG-(DFB)₄ in any of those in vitro cell binding assays. Overall, it was observed that the conjugation of ACUPA ligands to the polymer nanodrugs has a strong influence on their PSMA targeted in vitro cell binding affinity and internalization. Moreover, the use of the multivalent PSMA binder [⁸⁹Zr]PEG-(DFB)₁(ACUPA)₃ demonstrated increased affinity compared against the single ACUPA containing version. These findings are consistent with other reports utilizing bivalent or multivalent PSMA binders. overall, the in vitro findings support the use of multivalent PSMA binding to maximize cell binding affinity and uptake.

All the nanodrugs were subjected to in vivo μPET/CT imaging (24 h, 72 h, 168 h, and 216 h) and organ biodistribution post 216 h imaging in nu/nu athymic mice model implanted with subcutaneous PSMA positive PC3-Pip (left flank) and PSMA negative PC3-Flu (right flank) tumors (FIG. 30A). Both the targeted nanodrugs [⁸⁹Zr]PEG-(DFB)₃(ACUPA)₁ and [⁸⁹Zr]PEG-(DFB)₁(ACUPA)₃ demonstrated remarkably high PSMA targeted uptake in PC3-Pip tumors as compared to the non-targeted [⁸⁹Zr]PEG-(DFB)₄, showing only peripheral accumulation irrespective of the tumor type (FIG. 30B, 31). The organ biodistribution demonstrated that, as the number of PSMA targeted ACUPA ligands conjugated to the nanodrugs increased, the ratio of PC3-Pip/PC3-Flu, PC3-Pip/muscle, and PC3-Pip/blood increased significantly (FIG. 33B-33D). Overall, the background clearance was improved with a targeted accumulation of the nanodrugs in PSMA+PC3-Pip tumors as the number of ACUPA ligands increased (FIGS. 30B, 31, and 33A-33D). It should be noted that, although [⁸⁹Zr]PEG-(DFB)₁(ACUPA)₃ demonstrated the highest background clearance and PC3-Pip/blood ratio, [⁸⁹Zr]PEG-(DFB)₃(ACUPA)₁ demonstrated relatively higher PC3-Pip uptake (9.64±0.87% ID) (FIG. 33A-33D, Table 7). Interestingly, despite the presence of only one ACUPA ligand, [⁸⁹Zr]PEG-(DFB)₃(ACUPA)₁ demonstrated highly comparable or even better deep-tissue penetration to that of the [⁸⁹Zr]PEG-(DFB)₁(ACUPA)₃ with three ACUPA ligands (FIG. 35). Such peculiar in vivo pharmacokinetics of those PSMA targeted polymer nanodrugs could be explained by the binding site barrier (BSB) effect, where large size macromolecules with higher target binding affinity could bind to cells around the periphery of the blood vessels and restrict their further smooth diffusion into the bulk tumors. As demonstrated by Simanek and coworkers, despite increasing the PSMA targeting motifs from four to sixty four copies, the targeted tumor uptake of the large size nanodrugs could get restricted by BSB effect leading to poor deep tumor penetration. Several other reports also clearly demonstrate that nanodrugs with high target binding affinity might not be very effective for uniform tumor penetration, despite their very high in vitro cell binding affinity. In this present study, it was evident from the in vitro cell binding assay that the increasing number of ACUPA ligands effectively enhanced the PSMA binding affinity of the [⁸⁹Zr]PEG-(DFB)₁(ACUPA)₃ to that of its counterpart [⁸⁹Zr]PEG-(DFB)₃(ACUPA)₁, and could experience relatively higher BSB effect. Being a relatively weak PSMA binder, [⁸⁹Zr]PEG-(DFB)₃(ACUPA)₁ experienced comparatively low BSB effect to that of [⁸⁹Zr]PEG-(DFB)₁(ACUPA)₃ and thus demonstrated higher PSMA targeted tumor accumulation with deep-tissue penetration.

However, considering the exceptionally higher background clearance and PC3-Pip/blood ratio, [⁸⁹Zr]PEG-(DFB)₁(ACUPA)₃ is more efficient for chemotherapeutics and/or therapeutic radionuclide delivery. The autoradiography images clearly demonstrated that the nontargeted [⁸⁹Zr]PEG-(DFB)₄ in PC3-Pip and all the nanodrugs in PC3-Flu were unable to penetrate the bulk prostate cancer tumors (FIG. 35). Similar to other prostate cancer xenografts like CWR22rv1, DU-145, and PC-3, the poorly leaky vasculature and macrophages of PC3-Pip and PC3-Flu could be the primary reason for the EPR mediated very low tumor uptake and deep-tissue penetration. Thus, as evaluated in our prior study, the nontargeted [⁸⁹Zr]PEG-(DFB)₄ demonstrated very high EPR mediated tumor uptake and deep-tissue penetration in highly leaky MX-1 and HT-29 tumor models but failed in the poorly leaky prostate cancer xenograft evaluated in this study. Considering the EPR-driven low tumor uptake of those large size nanodrugs, active targeting of prostate cancer is highly essential to facilitate high targeted tumor uptake with deep-tumor penetration for improved therapeutic efficacy.

### Conclusions

In conclusion, three 4-armed Star-PEG based nanodrugs without or with different numbers of ACUPA ligands were synthesized and radiolabeled with good yields. The PSMA targeted in vitro cell binding and in vivo pharmacokinetics of the nanodrugs were evaluated in PSMA+PC3-Pip and PSMA-PC3-Flu cells/tumors, demonstrating the potential influence of the number of PSMA targeting ACUPA motifs attached to the nanodrugs. Although the targeted nanodrugs [⁸⁹Zr]PEG-(DFB)₃(ACUPA)₁ with one ACUPA ligand demonstrated relatively higher uptake in PSMA+PC3-Pip tumor, the nanodrug [⁸⁹Zr]PEG-(DFB)₁(ACUPA)₃ with three ACUPA ligands showed remarkably higher PC3-Pip/blood ratio and background clearance. Besides, unlike the EPR mediated low peripheral accumulation of nontargeted [⁸⁹Zr]PEG-(DFB)₄, the targeted nanodrugs with one or three copies of ACUPA ligands offered deep-tissue penetration in PSMA+PC3-Pip tumors.

### Example 2. PSMA Targeted Star-PEG Nanodrugs for SPECT Imaging and Radioligand Therapy

Background: Depending on the macrophages and leakiness, passive uptake of large nanocarriers may be dominant in bulk tumors due to the enhanced permeability and retention (EPR) effect, leading to low peripheral accumulation. Our prior results on 4-arm StarPEG polymers of 40 kDa have demonstrated that active tumor targeting could significantly improve imaging contrast and deep-tumor penetration of large nanocarriers, which is desirable for better therapeutic efficacy.

Methods: To evaluate the therapeutic efficacy of PSMA-targeted StarPEG nanocarriers, we designed and synthesized StarPEG nanodrugs with or without three copies of PSMA-targeting ACUPA ligands. One copy of the radiometal chelator DOTA was conjugated to each nanocarrier to radiolabel the b-emitting ¹⁷⁷Lu for simultaneous SPECT imaging and therapy. The radiolabeled nanodrugs were evaluated in vitro and in vivo using PSMA+PC3-Pip and PSMA-PC3-Flu cell lines, subcutaneous xenografts, and metastatic models.

Results: The nanocarriers [⁷⁷Lu]PEG-DOTA₁ and [¹⁷⁷Lu]PEG-DOTA₁-ACUPA₃ were efficiently radiolabeled with ¹⁷⁷Lu (Molar activities of 294.4-312.8 and 395.2-427.1 uCi/nmol, respectively), and the targeted StarPEG showed significantly higher in vitro PSMA binding affinity with a dissociation constant (k_{D}) of 51.7 nM in PC3-Pip cells. The in vivo SPECT images and ex vivo organ biodistribution of ACUPA conjugated StarPEG demonstrated high imaging contrast with 21.3% ID/g uptake of PSMA+PC3-Pip tumors at 192 h. A single dose of 500 uCi [¹⁷⁷Lu]PEG-DOTA₁-ACUPA₃ demonstrated potential suppression of subcutaneous PC3-Pip tumors monitored for up to 138 days, while 250 μCi dose was witnessed to restrict the growth of PC3-Pip metastatic tumors, but showed reasonable lungs and kidneys toxicity.

Conclusions and Future Directions: The StarPEG nanocarriers demonstrated high PSMA-targeted delivery of the therapeutic isotope ¹⁷⁷Lu with excellent imaging contrast. The targeted nanocarrier efficiently suppressed subcutaneous and metastatic PC3-Pip tumor growth. Overall, these preclinical results demonstrated very high treatment efficacy of the PSMA-targeted nanocarrier, [¹⁷⁷Lu]PEG-DOTA₁-ACUPA₃, for prostate cancer with resilient clinical translation potential.

### Materials.

Materials and Instrumentations: The 4-armed PEG_{40 kDa}-(NH₂)₄ was purchased from SINOPEG (Fujian, China). ¹⁷⁷LuCl₄ was procured from MU Research Reactor (MURR) Shi University of Missouri (Columbia, MO), and p-SCN-Bn-DOTA from Macrocyclics (Plano, TX). RPMI-1640 media, penicillin-streptomycin (P/S) solutions, and Fetal bovine serum (FBS), were purchased from Life Technologies (Carlsbad, CA), Thermo Fisher Scientific (Waltham, MA). Other chemicals (solvents, reagents, and building blocks) were bought from Thermo Fisher Scientific, VWR, or Sigma Aldrich, and used without further processing. ¹H NMR spectra were recorded in a Bruker 400 MHz NMR, respectively. Chemical shifts were shown in parts per million (ppm, δ).

### Synthesis of StarPEG Conjugates.

General: HPLC was performed using a 4.6×150 mm 5 um 300 A Phenomenex Jupiter C18 reversed-phase column with a 15-minute linear gradient of 0-100% acetonitrile/water/0.1% TFA (1.0 mL/min) beginning 2 min after injection unless otherwise mentioned. The starting materials and linkers like PEG-(5HcyO)₃(NH₂)₁, Azido-PEG₇, and Azido-ACUPA were synthesized following the previously reported procedure.

Synthesis of PEG-(PEG₇)₃(NH₂) and PEG-(ACUPA)₃(NH₂): PEG-(5HcyO)₃(NH₂) (200 mg, 5 μmol) was dissolved in 1 mL of 0.1 M sodium phosphate (pH 7.4), and treated with either a 25 mM solution of Azido-ACUPA (1.0 mL, 25 μmol, 1.7 Eq) or Azido-PEG₇ (9 mg, 10 μL, 22.5 μmol, 1.5 Eq) in 0.1 M sodium phosphate, pH 7.4, for 48 h at 37° C., then dialyzed (SpectraPor 2 membrane, 12-14 kDa cutoff) against water followed by methanol, and dried under reduced pressure to afford the respective intermediates, PEG-(PEG₇)₃(NH₂) (Yield 75%, 150 mg) and PEG-(ACUPA)₃(NH₂) (Yield 88%, 175 mg).

Synthesis of PEG-(DOTA)₁ and PEG-(DOTA)₁(ACUPA)₃: A solution of the respective intermediates, PEG-(PEG₇)₃(NH₂) (97 mg, 2.4 μmol) and PEG-(ACUPA)₃(NH₂) (164 mg, 4.1 μmol), in DMF (1.0 mL) were mixed with a 10 mg/mL solution of p-isothiocyanatobenzyl-DOTA (p-SCN-Bn-DOTA) in DMSO (Macrocyclics, 1.5 Eq) and N,N-diisopropylethylamine (10 Eq) in separate reaction vials and kept at 37° C. for 24 h. The mixtures were dialyzed against water (SpectraPor 2 membrane, 12-14 kDa cutoff) to remove unconjugated materials, followed by methanol and dried under reduced pressure to provide the product PEG-(DOTA)₁ (Yield 93%, 90 mg) and PEG-(DOTA)₁(ACUPA)₃ (Yield 91%, 150 mg).

Cell Culture: Unless and otherwise specified, the PSMA-PC3-Flu and PSMA+PC3-Pip cell lines were cultured in RPMI1640 medium containing 10% FBS and 1% penicillin/streptomycin (P/S) at 37° C. with 5% CO₂. The cells were obtained from Dr. Martin Pomper's lab, Johns Hopkins University. According to experimental protocols, cells were trypsinized (0.25%) for 2-3 minutes to detach from the culture flasks for further passage or to seed the cells in suitable multi-well plates to perform cell binding assays.

Competition Radioligand Binding Assay: Similar to our prior reported protocol, ⁶⁸Ga-PSMA-11 was produced in a ⁶⁸Ge/⁶⁸Ga generator, and used in a competition radioligand binding assay to acquire the IC₅₀ values for the nanocarriers. Briefly, around ˜0.185 MBq (2.5 ng) of ⁶⁸Ga-PSMA-11 along with different concentrations (0.01-100000 nM) of the nonradiolabeled nanocarriers was treated to each well of 96-well plates containing PSMA+PC3-Pip cells (˜20 k cells/wells). After 1 h incubated at room temperature, the radioactive medium was removed, and the cells were washed with PBS twice. The cells were lyzed with sodium hydroxide and the radioactivity of the lysate in each well was counted in a Hidex gamma counter. IC₅₀ was determined by nonlinear regression analysis in Prism software (Graph-Pad).

¹⁷⁷Lu Radiolabeling of StarPEGs: StarPEG nanocarrier in water was added to a solution of 10 mM NH₄Oac containing 10 vol % of ascorbic acid (20 mg/ml), and ¹⁷⁷LuCL₄ in a ratio of 10 μCi/μg of nanocarriers were mixed to it. The mixture was incubated for 1 h at 55° C. The radiolabeled product was purified using PD-10 size-exclusion desalting column (Fisher Scientific, Hampton, NH) and eluted with saline solution containing 0.2 mg of L-ascorbic acid per mL. Instant thin layer chromatography (iTLC) was performed using silica gel-impregnated glass microfiber chromatography paper (Neta Scientific, Hainesport, NJ) and developed with 20 mM citric acid solution to confirm radiolabeling purity. Multiple radiolabeling studies were carried out with different amounts of nanocarriers and ¹⁷⁷Lu, and were summarized in Table 9.

TABLE 9
Radiolabeling condition, yields and molar activity of ¹⁷⁷Lu labeled nanocarriers.

| Parameters | PEG-(DOTA)₁ | PEG-(DOTA)₁(ACUPA)₃ |
|---|---|---|
| No of Reactions | 4 | 9 |
| ¹⁷⁷LuCl₄/StarPEGs | 10 μCi/μg | 10 μCi/μg |
| Reaction Buffer | 900 uL 0.01 M NH₄Oac | 900 uL 0.01 M NH4Oac |
| Additive | 100 uL L-Ascorbic Acid | 100 uL L-Ascorbic Acid |
| | (20 mg/mL) | (20 mg/mL) |
| pH | 4.5 | 4.5 |
| Temp (° C.) | 55 | 55 |
| % Yield (iTLC) | 74-79 | 97-100 |
| % Yield (Isolated) | 70.9-76.6 | 91.8-99.2 |
| Molar Activity | 294.4-312.8 | 395.2-427.1 |
| (uCi/nmol) | | |

In Vitro Saturation Binding Assay: PC3-pip cells were seeded in 12-well plates (˜100 k cells/well) 24 h before testing. Cells were washed with PBS twice, and each well was treated with 1 mL of growth media without/with 10 μM PSMA-2 (a previously described PSMA inhibitor),³⁷⁻³⁹ and incubated at 37° C. for 1 h. Then different concentrations (0.015-150 nM) of the ¹⁷⁷Lu radiolabeled StarPEG nanocarriers were treated to the cells and incubated at 37° C. After 1 h of incubation, the radioactive medium was removed, and cells were washed with PBS. The cells were lyzed with sodium hydroxide, and each well's radioactivity was analyzed in a Hidex gamma counter. The respective non-specific bound activities were subtracted and the dissociation constant (K_{d}) was calculated by nonlinear regression one site-specific binding in Prism Software (GraphPad). These data were further used to show the PSMA binding affinity and blocking of the StarPEG nanocarriers at 1 h.

In Vitro Binding and Blocking Assay: Multiple PC3-Flu and PC3-pip cells were seeded in 24-well plates (˜50 k cells/well) 24 h before testing. Cells were washed with PBS twice, and each well was treated with 0.5 mL of growth media with/without 10 μM PSMA-2 (a previously described PSMA inhibitor),³⁷⁻³⁹ and incubated for 1 h at 37° C. Then 5 nM of the ¹⁷⁷Lu radiolabeled StarPEG nanocarriers were treated to the cells and incubated further at 37° C. The radioactive medium was discarded at 1 h, 4 h or 24 h time points, and cells were lyzed with NaOH (5N, 250 μL) after washing with PBS. The lysate was analyzed in a Hidex gamma counter (along with the standard treated activity to calculate the % bound activity.

In Vitro Membrane-Bound & Internalization Assay: Multiple PC3-pip and PC3-flu cells were seeded in 24-well plates (˜50 k cells/well) 24 h before assay. Each well was treated with 5 nM ¹⁷⁷Lu radiolabeled StarPEG nanocarriers and incubated at 37° C. At each time point (1 h, 4 h, and 24 h), one set of the PC3-flu and PC3-pip cells were washed with PBS twice, and incubated with a mixture of 0.5 mL ice-cold glycine (50 mM) and NaCl (150 mM) for 5 min at 4° C. The acid buffer corresponding to the membrane-bound activity was collected. Then the cells were lyzed with sodium hydroxide (5N), and the lysate corresponding to the internalized activity was collected. The respective radioactivities were analyzed in a Hidex gamma counter (Turku, Finland) along with the standard treated activity of the ¹⁷⁷Lu radiolabeled StarPEG nanocarriers to calculate the % of membrane-bound and internalized activities.

Inoculation of Mice with Dual Xenograft and Metastatic Tumors: The in vivo animal studies were performed under a protocol approved by the UCSF Institutional Animal Care & Use Committee (IACUC). Using precisely similar protocol from our prior report, homozygous (nu/nu) athymic male mice of 4-5 weeks old (Jackson Laboratories or Envigo-Harlan Laboratories, Livermore CA) inoculated with PC3-Pip (left flank, 1 million cells) and PC3-Flu (right flank, 1 million cells) dual xenografts.² Around 100-200 mm³ tumor size was perceived after 1-2 weeks post-inoculation.

The metastatic models were obtained by injecting 1 million of PC3-Pip cells by intracardiac injection (in 100 μL PBS) into the left ventricle of the heart of homozygous (nu/nu; strain #: 002019) athymic male mice of 6-8 weeks old. The UCSF Preclinical Therapeutic Core performed the intracardiac injections. The tumor growth was monitored by [⁶⁸Ga]PSMA-11 μPET/CT Imaging and biodistribution at multiple time points.

In Vivo μSPECT/CT Imaging and Biodistribution Studies: One week post-inoculation, when the tumor size reached 100-200 mm³, the animals were anesthetized using 2% isoflurane, and the respective ¹⁷⁷Lu radiolabeled StarPEG nanocarriers were administered via tail vein. Around 37 MBq in 100 μL of saline per mouse were injected for p SPECT/CT imaging (4*2), whereas around 9-10 MBq in 100 μL of saline per mouse were injected for ex vivo biodistribution (6*2)). The mice were scanned at 24 h, 72 h, 144 h, and 192 h post radiopharmaceutical injection in a μSPECT/CT imaging system (VECT or 4CT, MILabs, BV). Image reconstruction was performed using a vendor-provided similarity-regulated ordered subsets expectation maximization algorithm. CT-based attenuation correction and energy-window-based scatter correction were performed, and quantification calibration to convert the raw reconstructed pixel value to a physical unit of Bq/mL was performed using a pre-calibrated conversion factor. The imaging data was processed and viewed in an open-source AMIDE software (http://amide.sourceforge.net/). The tumor-bearing mice injected with 9-10 MBq of ¹⁷⁷Lu radiolabeled StarPEG nanocarriers were sacrificed at 72 h (n=3) and 192 h (n=3). Blood was collected through a cardiac puncture, and major organs (liver, kidney, spleen, heart, pancreas, lung, brain, femur, muscle, testis, and subcutaneous tumor) were harvested. Blood and major organs were weighed and analyzed in an automated gamma counter (Hidex, Turku, Finland). The percent injected dose per gram of tissue (% ID/g) was determined by comparing standard radioactivity.

Tumor Dose Estimation: Spherical volumes of interest (VOIs) were drawn on coregistered CT images for tumors at all time points. All VOIs were spheres (2 mm diameter) and placed well within the anatomical boundaries to minimize spill-over or spill-in of radioactivity. The percent of injected activity (% IA) for a normalized volume (0.5 ml) at each time point was computed for curve-fitting to derive TIACs, and the dose (mGy/MBq) was calculated for a sphere model.

Normal Tissue Radiation Dose Estimation: Volumes of interest (VOIs) were drawn on coregistered CT images for the brain, lungs, heart, liver, kidneys, and urinary bladder. All VOIs were either elliptical cylinders (5 mm long axis, 3 mm short axis, and 5 mm height for the brain), cylinders (3 mm diameter and 3 mm height for kidneys), or spheres (3 mm diameter for lungs, liver, and urinary bladder, and 4 mm diameter for heart), and they were placed well within the anatomical boundaries to minimize spill-over or spill-in of radioactivity. The mean values (in Bq/mL) in these VOIs were multiplied by standard mouse organ volumes (in mL) to estimate total activity (in Bq) within these organs. The total activity within the entire animal subtracted by all organ activities was used as activity in the remainder of the body. The percent of injected activity within the defined organs (% IA) was extrapolated to human-equivalent values using ratios of standard human organ weights to mouse organ weights.

These input % IA data for each organ and the remainder of the body were curve-fitted to derive time-integrated activity coefficients (TIACs, also known as residence times) (in Bq-hr/Bq) and organ and whole-body effective doses for human equivalents were estimated using each mouse data. The data from the animals were averaged to derive absorbed and equivalent organ doses (in mGy/MBq or mSv/MBq), and whole-body effective dose (in mSv/MBq). Organ and effective dose estimations were performed using OLINDA version 1.1 using ICRP Publication 60 tissue weighting factors and OLINDA version 2.0 using ICRP Publication 103 tissue weighting factors.

Autoradiography: After analyzing the dissected organ samples in the gamma counter (Hidex), the tumors were embedded in optimal cutting temperature (OCT) compound and flash frozen on dry ice. Using a microtome, the frozen tumor tissues were sectioned at 20 μm thickness and mounted on iQID charged-particle digital autoradiography imaging systems (Qscint Imaging Solutions, LLC, Tucson, AZ). The raw autoradiography data were processed in ImageJ software.

Treatment Studies: For the treatment studies, each mouse's body weights and tumor measurements were performed until the mice reached a humane endpoint including body condition score below 2, weight loss by 20%, or tumor volume of 2,000 mm³. The tumor measurements were carried out twice a week, and tumor volumes were calculated by formula V=[length×(width)²]/2.

Animals were randomized in groups, and [¹⁷⁷Lu]PEG-(DOTA)₁(ACUPA)₃ injections were performed when the tumor reached 100 mm³. The PC3-Pip xenograft-bearing mice were injected with a single dose of vehicle, 125 μCi, 250 μCi or 500 μCi of [¹⁷⁷Lu]PEG-(DOTA)₁(ACUPA)₃. In addition, a separate group received a 250 μCi dose of [¹⁷⁷Lu]PSMA-617.

For PC3-Pip metastatic models, the mice were either treated with a single dose of vehicle or 250 μCi of [¹⁷⁷Lu]PEG-(DOTA)₁(ACUPA)₃. The body weights for each mouse were measured until the mice reached a humane endpoint including body condition score below 2, weight loss by 20%, and were imaged with ⁶⁸Ga-PSMA-11.

Chronic Toxicity study: The nude mice bearing PC3-Pip subcutaneous tumors and treated with different doses of [¹⁷⁷Lu]PEG-(DOTA)₁(ACUPA)₃ survived in the treatment group and were subjected to chronic toxicity analysis on day 138 post-treatment. A complete blood count and laboratory liver and kidney function tests were conducted to study chronic toxicity. Blood was collected by cardiac puncture in EDTA-coated tubes to study blood cell counts. Besides, small fractions of blood were allowed to sit at 4° C. for 30 min in vials and the clotted blood samples were centrifuged (10,000×g, 10 min, at 4° C.) to separate the serum. Blood and serum samples were sent to the Comparative Pathology Laboratory, UC Davis School of Veterinary Medicine for blood cell counts and organ function tests.

Histology of the tissue sections: Following organ biodistribution, the tissue sections of thickness 10 μm were collected from OCT-embedded frozen organ samples on glass slides. Routine histologic analysis was performed to study microscopic features of the tumor samples. The tissue sections were shocked in acetone at −20° C. for 20 minutes followed by in methanol at 4° C. for 10 minutes. The tissue sections were rehydrated with water and treated with hematoxylin (5 minutes), bluing reagent (10 sec) and eosin (30 sec) followed by washing with water after each treatment. The stained tissue was washed with ethanol gradient (50%, 70%, and 95%) for 2 minutes each, followed by xylene treatment for 5 minutes, and the mounting medium was applied.

### Results

### Chemistry of StarPEG Conjugates

### Design of StarPEG Conjugates

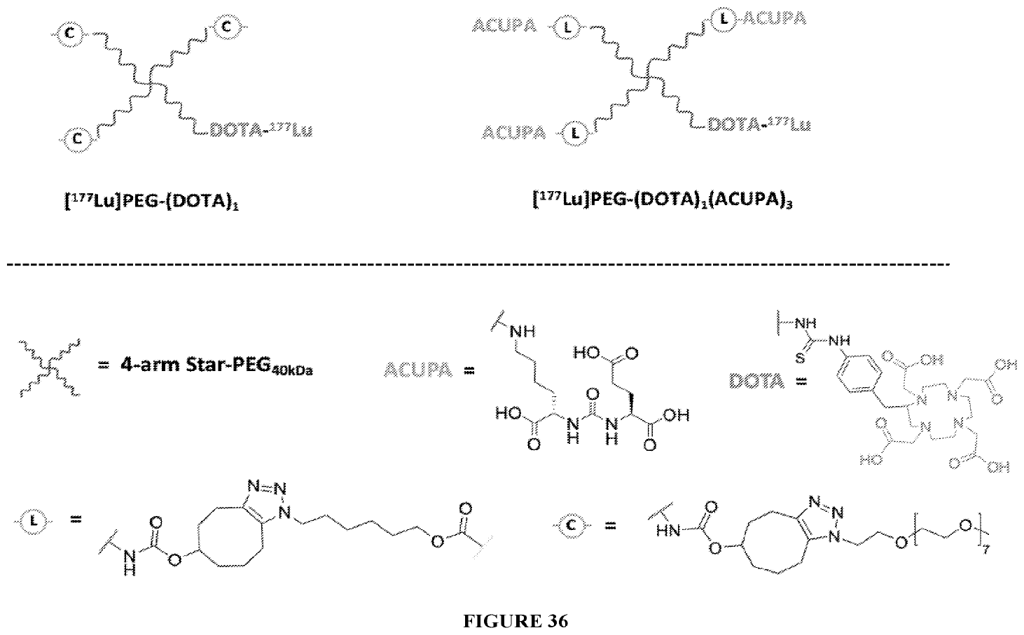

Reports suggest that large-size nanocarriers undergo EPR-mediated heterogeneous and peripheral passive accumulation in most prostate cancer tumor models due to poor vascular development. Recently, we demonstrated a series of 40 kDa, 15 nm, diagnostic StarPEG nanocarriers that demonstrated selectively improved tumor accumulation and penetration in PSMA+PC3-Pip tumors by conjugating PSMA-targeting ACUPA ligands. Herein, we hypothesized to develop the therapeutic version of the StarPEG nanocarriers by replacing the diagnostic radiometal-chelator pair of ⁸⁹Zr-DFB with the theranostic pair ¹⁷⁷Lu-DOTA. To test this hypothesis, two StarPEG conjugates, PEG-(DOTA)₁ and PEG-(DOTA)₁(ACUPA)₃, were designed with or without three copies of PSMA targeting ACUPA ligands (FIG. 36), in which both the nanocarriers were conjugated with one copies of DOTA to chelate the beta-emitting theranostic radiometal ¹⁷⁷Lu. The primary goal of this design strategy is to compare the in vitro as well as in vivo targeting efficacy of the PSMA-targeting nanocarriers with its nontargeting counterpart, and to evaluate their in vivo treatment efficacy in both PSMA+ subcutaneous and metastatic tumor models.

### Synthesis of StarPEG Conjugates

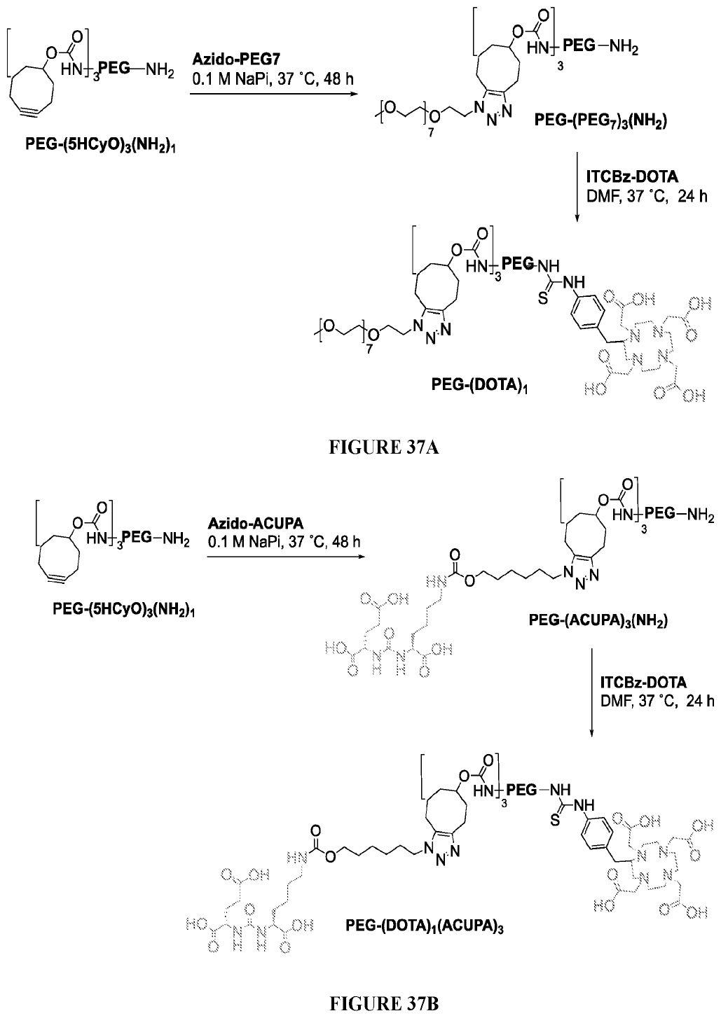

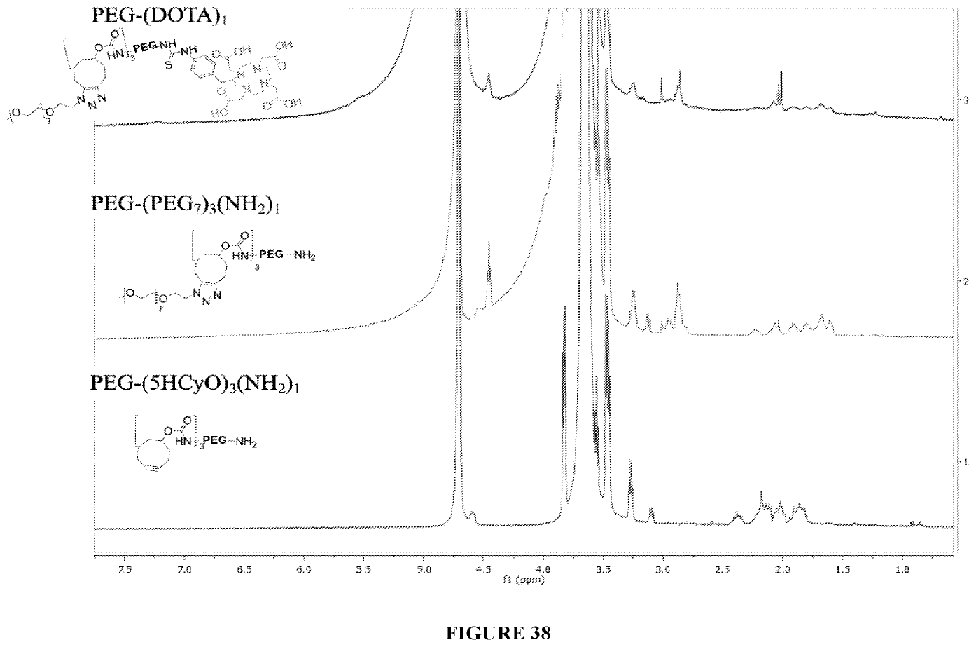

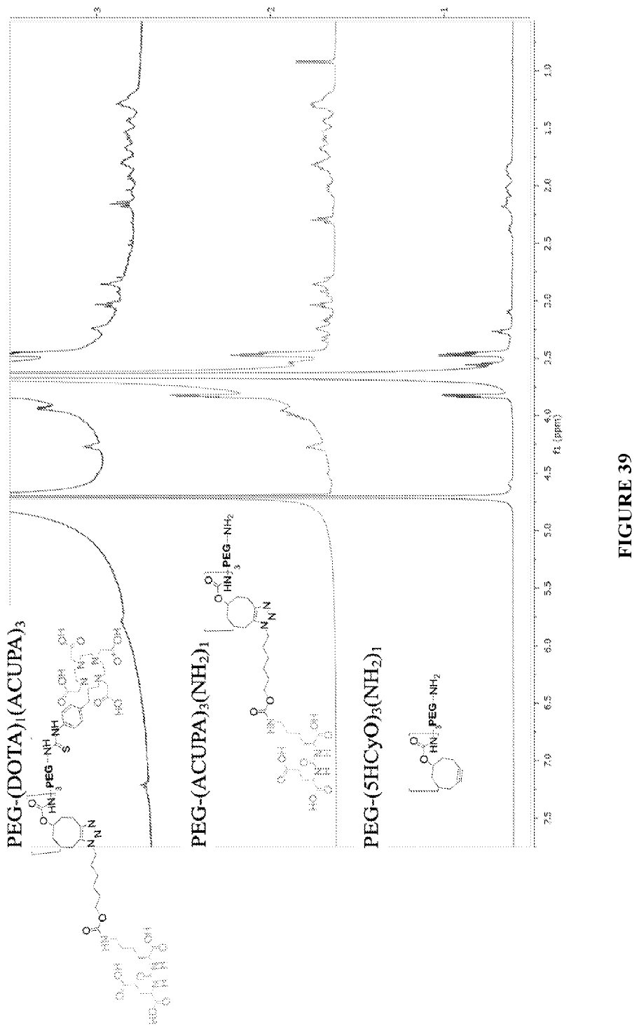

For the synthesis of the designed StarPEG nanocarriers, the previously synthesized and reported PEG-(5HcyO)₃(NH₂)₁ was employed as starting material. As summarized in FIG. 37, both the nontargeted nanocarrier [PEG-(DOTA)₁] and the targeted nanocarrier [PEG-(DOTA)₁(ACUPA)₃] were synthesized following similar synthetic routes. PEG-(5HcyO)₃(NH₂)₁ with three cyclooctyne counterparts were subjected to second-generation azide click reactions with either Azido-ACUPA or Azido-PEG7 to yield PEG-(ACUPA)₃(NH₂) or PEG-(PEG₇)₃(NH₂), respectively. The intermediates were purified using 12-14 kDa cutoff dialysis membrane and were further reacted with isothiocyanatobenzyl-DOTA (ITCBz-DOTA) to produce the polymer conjugates of interest, i.e. either PEG-(DOTA)₁ or PEG-(DOTA)₁(ACUPA)₃. Post DOTA conjugation, the polymer conjugates were again purified using 12-14 kDa cutoff dialysis membrane and dried under reduced pressure. The conjugation of ACUPA and DOTA ligands to the polymer nanocarriers was confirmed by comparing their proton NMR (FIGS. 38 and 39). Prominent peaks at around 4.45 ppm and 4.26 ppm were observed in both the intermediates and final polymer conjugates after click conjugation of azido-PEG7 or Azido-ACUPA, respectively, that correspond to the CH₂ protons close to triazole. Whereas no such peaks corresponding to CH₂ protons in the 4-4.5 ppm range were observed in the starting material PEG-(5HcyO)₃(NH2)₁. Furthermore, a noticeable aromatic peak at 7.21 ppm was observed after the conjugation of ITCBz-DOTA, which corresponds to the phenyl protons and confirms the conjugation of DOTA to the StarPEG nanocarriers.

### Radiolabeling of StarPEG Conjugates.

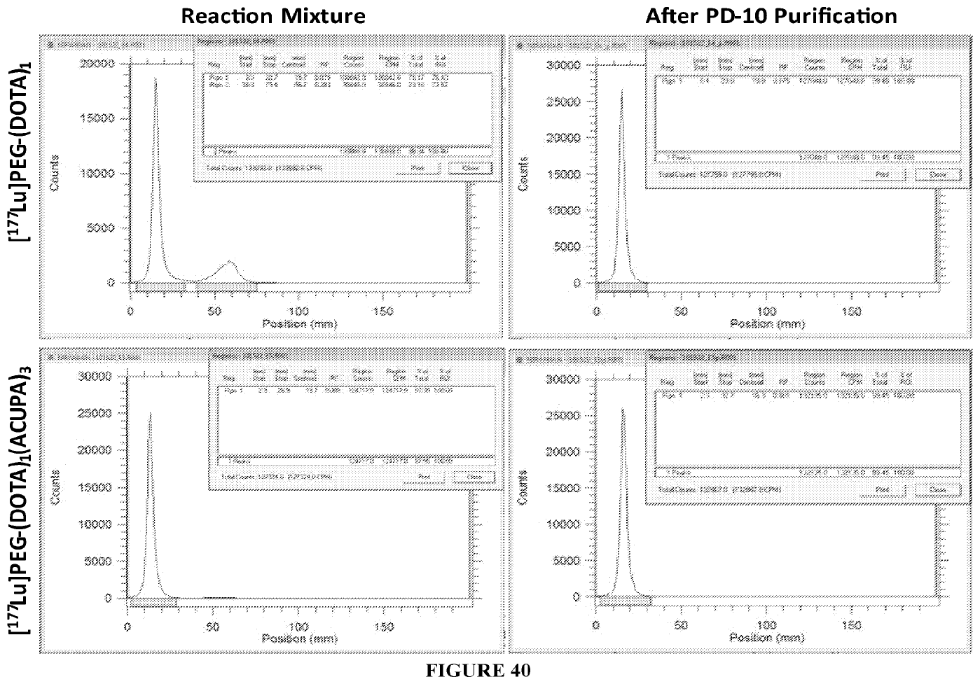

The radiolabeling of ¹⁷⁷Lu to the DOTA conjugated StarPEG nanocarriers were performed in NH₄Oac buffer containing ascorbic acid as stabilized. However, it was interesting to observe that despite efficient radiolabeling as found in iTLC, we were unable isolate the radiolabeled nanocarriers with good yield, especially in scale-up radiosynthesis (Table 9, FIG. 40). The isolated yield was below 10%, and a major percentage of radioactivity got stuck in the stationary phase of PD-10 size-exclusion desalting column, possibly due to radiolytic degradation of the StarPEG nanocarriers in the side PD-10 column. The purification protocols were further modified by adding ascorbic acid in saline while equilibrating and eluting PD-10 column to protect the polymer nanocarriers from free radical damage. Briefly, ¹⁷⁷LuCl₄ was treated with StarPEG nanocarriers at 10 μCi/μg in 10 mM NH₄Oac containing 0.2 mg/mL ascorbic acid, and the solutions were incubated for 1 h at 55° C. The resulting mixtures were purified using PD-10 size-exclusion desalting columns by eluting with saline solution containing 0.2 mg of L-ascorbic acid per mL. As summarized in Table S2, the isolated yield and molar activities were 70.9-76.6% and 294.4-312.8 μCi/nmol for [¹⁷⁷Lu]PEG-(DOTA)₁, respectively. However, relatively better isolated yield (91.8-99.2%) and molar activity were obtained for [¹⁷⁷Lu]PEG-(DOTA)₁(ACUPA)₃ (395.2-427.1 μCi/nmol), respectively.

### In Vitro Cell Binding Assay

The designed and synthesized StarPEG nanocarriers were first evaluated in vitro using PSMA+PC3-Pip and PSMA-PC3-Flu cell lines, and the PSMA targeting affinity of the targeted nanocarrier, PEG-(DOTA)₁(ACUPA)₃ was compared with previously reported conjugates as positive control.

### Competition Radioligand Binding Assay

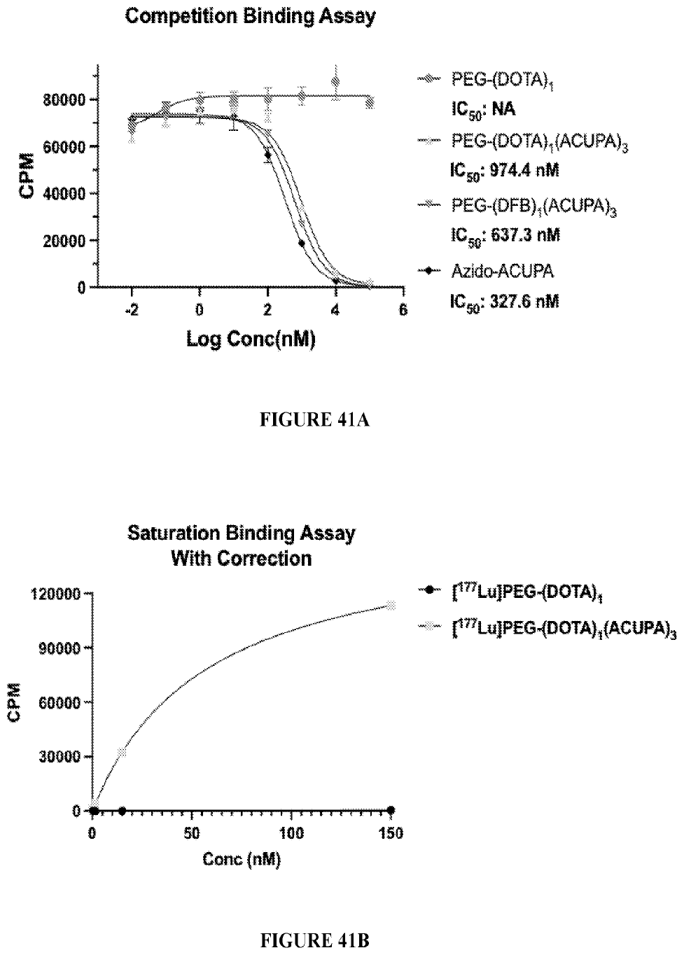

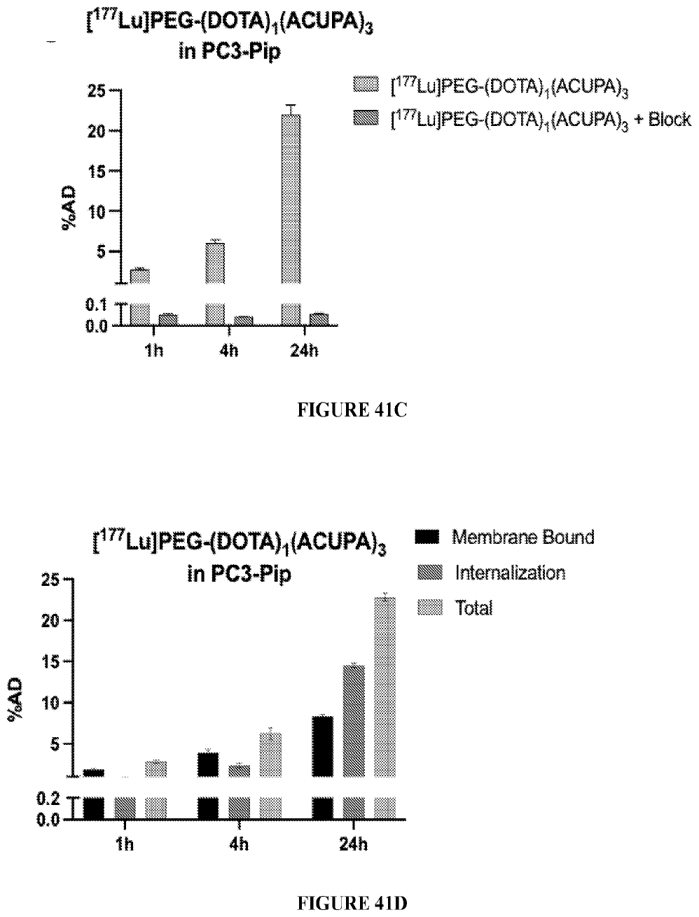

The nonradiolabeled StarPEG nanocarriers were subjected to ⁶⁸Ga-PSMA-11 based competitive radioligand binding assay to evaluate their relative binding affinity following previously reported protocol (FIG. 41A and Table 10). The PSMA-2, Azido-ACUPA along with previously PET imaging analog were also evaluated as positive controls and were found to be very consistent to published reports. From two independent experiments, the IC50 for the targeted nanocarrier PEG-(DOTA)₁(ACUPA)₃ was 1024±50 nM with 95% confidence interval in the range of 687-1344 nM, which demonstrated relatively lower binding affinity as compared to the structurally very close PET imaging analog PEG-(DFB)₁(ACUPA)₃ (637.3 nM) in PSMA+PC3-Pip cells. It was observed that, replacing DFB with DOTA radiometal chelator, the binding affinity of the nanocarrier reduced relatively. However, as expected, no sign of any specific binding was observed in the nontargeted nanocarrier PEG-(DOTA)₁.

TABLE 10
Competition radioligand binding assay results of the unlabeled
nanocarriers in PSMA+ PC3-Pip cells using 68Ga-PSMA-11.

| | Experiment 1 | | Experiment 2 | |
|---|---|---|---|---|
| Nanodrugs | IC₅₀ (nM) | IC₅₀, 95% CI (nM) | IC₅₀ (nM) | IC₅₀, 95% CI (nM) |
| PEG-(DOTA)₁ | NA | NA | NA | NA |
| PEG-(DOTA)₁(ACUPA)₃ | 1074 | 857.3-1344 | 974.4 | 687.8-1380 |
| PEG-(DBF)₁(ACUPA)₃ | — | — | 637.3 | 493.7-817.9 |
| Azido-ACUPA | — | — | 327.6 | 251.3-426.7 |
| PSMA-2 | 25.50 | 19.19-34.02 | NA | NA |

### Saturation Binding Assay

The dissociation constants (K_{d}) of the ¹⁷⁷Lu-labeled nanocarriers were calculated in PSMA+PC3-Pip cells by saturation binding assay (FIG. 41B). As summarized in Table 11, K_{d} of 57.5 nM and Bₘₐₓ of 156674 were obtained for the targeted nanocarrier [¹⁷⁷Lu]PEG-(DOTA)₁(ACUPA)₃ with 95% confidence interval in the range of 52.5-63.2 nM. Similar to the binding affinity observed in competition binding assay, the saturation binding assay demonstrated a relatively lower binding affinity of the theranostic nanocarrier [¹⁷⁷Lu]PEG-(DOTA)₁(ACUPA)₃ as compared to its previously reported PET imaging analog [⁸⁹Zr]PEG-(DFB)₁(ACUPA)₃ (K_{d}=30.96 nm) in PSMA+PC3-Pip cells. However, the nontargeted nanocarrier [¹⁷⁷Lu]PEG-(DOTA)₁ did not show any sign of specific binding in the PSMA+PC3-Pip cells (Table 11).

TABLE 11
Saturation binding assay results of the 177Lu
labeled nanocarriers in PSMA+ PC3-Pip cells.

| | Without Correction | | With Correction | |
|---|---|---|---|---|
| | [¹⁷⁷Lu]PEG- | [¹⁷⁷Lu]PEG- | [¹⁷⁷Lu]PEG- | [¹⁷⁷Lu]PEG- |
| Parameters | (DOTA)₁ | (DOTA)₁(ACUPA)₃ | (DOTA)₁ | (DOTA)₁(ACUPA)₃ |
| Kd (nM) | NA | 59.56 | NA | 57.50 |
| 95% CI of Kd | NA | 53.89-66.21 | NA | 52.50-63.26 |
| Bmax | Unstable | 163909 | Unstable | 156674 |
| Bmax (95% CI) | Very wide | 158323 to 170171 | Very wide | 151865 to 161990 |

### Blocking Assay

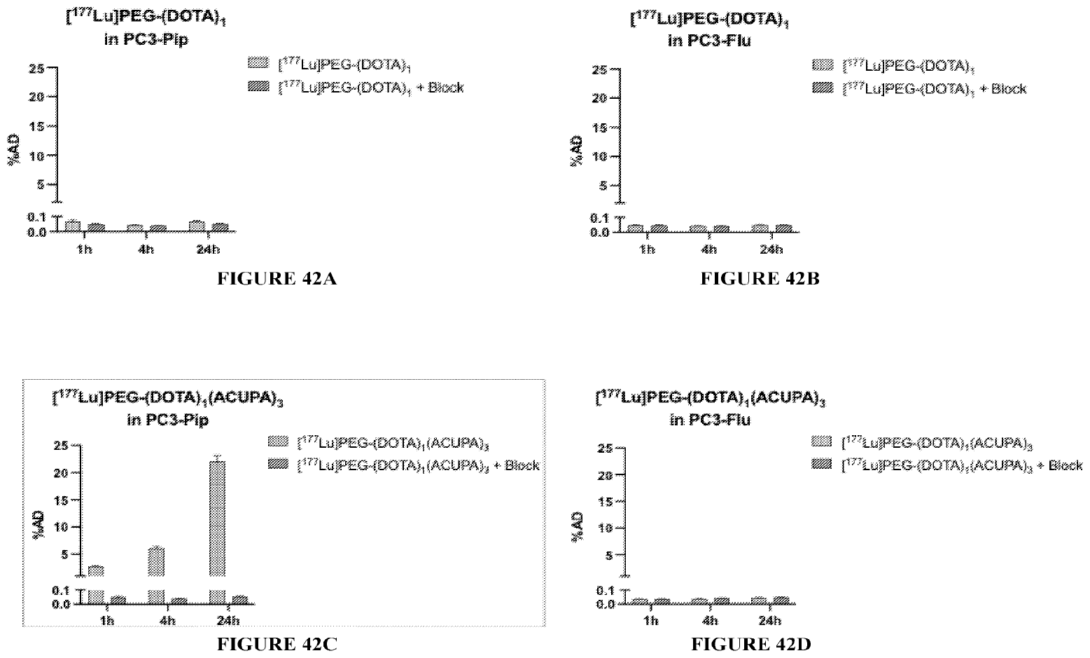

Further, the binding efficacy of both the targeted and nontargeted nanocarriers was tested in a blocking assay using PSMA-2 (PSMA-2, Ki=0.24 nM, and IC50=10 nM) as the blocking agent in both PSMA+PC3-Pip and PSMA-PC3-Flu cells (FIG. 41C and FIGS. 42A-D). Starting from one hour time point, the targeted nanocarrier [¹⁷⁷Lu]PEG-(DOTA)₁(ACUPA)₃ demonstrated significantly high accumulation (2.77±0.12% AD) in PSMA+PC3-Pip cells, which went on increasing up to 22.0±1.16% AD at 24 h time point. The targeted uptake of the nanocarrier was efficiently blocked to as low as 0.04% AD in the presence of a known PSMA binder PSMA-2.47 In comparison, the targeted nanocarrier in PSMA-PC3-Flu cells, along with the nontargeted nanocarrier [¹⁷⁷Lu]PEG-(DOTA)₁ in both PSMA+PC3-Pip and PSMA-PC3-Flu demonstrated no sign of specific binding, and the cell accumulations remained in the range of 0.04-0.06% AD.

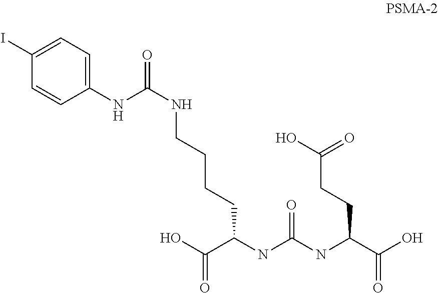 [SMILES: O=C(O)CC[C@H](NC(=O)N[C@@H](CCCCNC(=O)Nc1ccc(I)cc1)C(=O)O)C(=O)O]

### Membrane-Bound and Internalization Assay

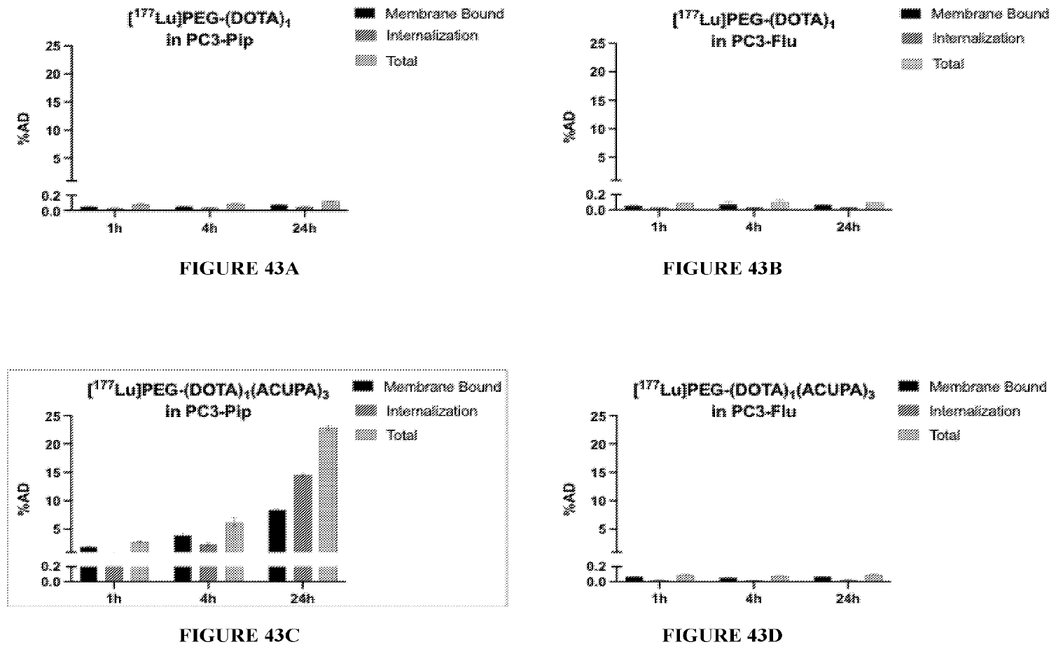

The degree of internalization of the nanocarriers was also evaluated in membrane-bound and internalization assay in both PSMA-PC3-Flu and PSMA+PC3-Pip cells (FIGS. 43A-D and FIG. 41D). The membrane-bound activities were isolated from the internalized activities by ice-cold acid wash containing a mixture of 50 mM glycine and 150 mM sodium chloride at different time points. A comparatively very high membrane-bound and internalized activities were observed for the targeted nanocarrier [¹⁷⁷Lu]PEG-(DOTA)₁(ACUPA)₃ in PSMA+PC3-Pip cells. At 1 h time point, the membrane-bound activities (1.89±0.11% AD) were relatively high as compared to the internalized activities (0.95±0.06% AD). However, at 24 h longer incubation time, the internalized activities increased significantly to 14.49±0.26% AD, which was 1.73 fold higher than the membrane-bound activity (8.33±0.25% AD). The total activities were 2.84±0.18, 6.24±0.73, 22.82±0.49% AD at 1 h, 4 h, and 24 h, respectively, and were very close to that of the bound activities obtained in blocking assay, i.e. 2.77±0.12, 6.08±0.37, 22.0±1.16% AD at 1 h, 4 h, and 24 h, respectively (FIG. 41C-D). As expected, no evidence of specific internalization or membrane binding was seen in PSMA-PC3-Flu cells and with the nontargeted nanocarrier.

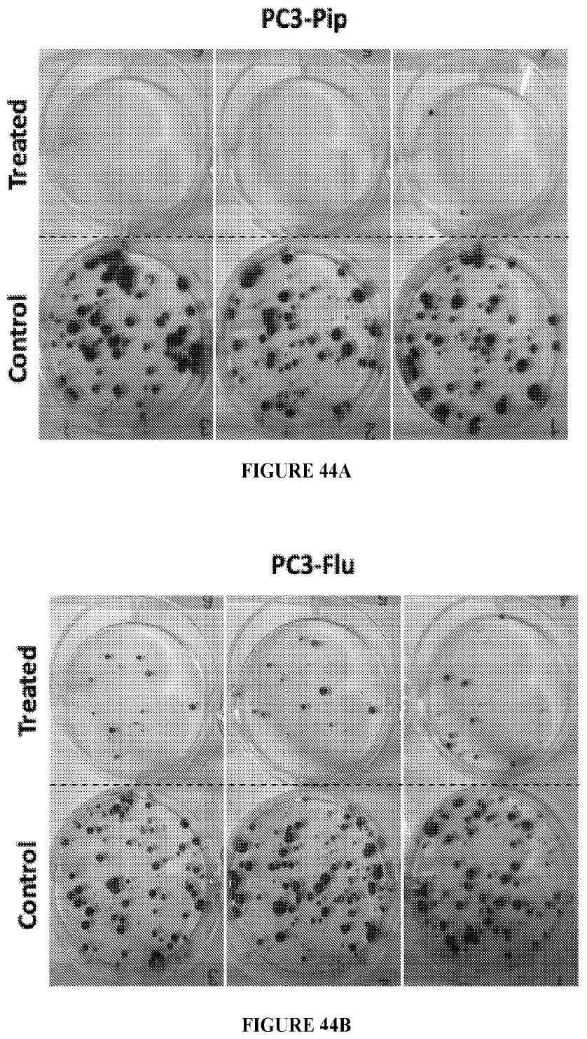

FIGS. 44A-B shows colony formation assay of PSMA+PC3-Pip (FIG. 44A) and PSMA-PC3-Flu (FIG. 44B) cells treated with 41 uCi (57 nM) of [¹⁷⁷Lu]PEG-(DOTA)₁(ACUPA)₃ for for 4 h.

Overall, the in vitro cell binding results demonstrated PSMA targeted efficient cell binding and internalization of the targeted nanocarrier [¹⁷⁷Lu]PEG-(DOTA)₁(ACUPA)₃ in PSMA+PC3-Pip cells. Although, replacing the DFB radiometal chelator with DOTA in PEG-(DFB)₁(ACUPA)₃ reduced the cell binding affinity slightly, PEG-(DOTA)₁(ACUPA)₃ still demonstrated excellent binding affinity worthy of further in vivo evaluation.

### In Vivo Evaluation of PC3-Pip and PC3-Flu Subcutaneous Dual Xenograft Models

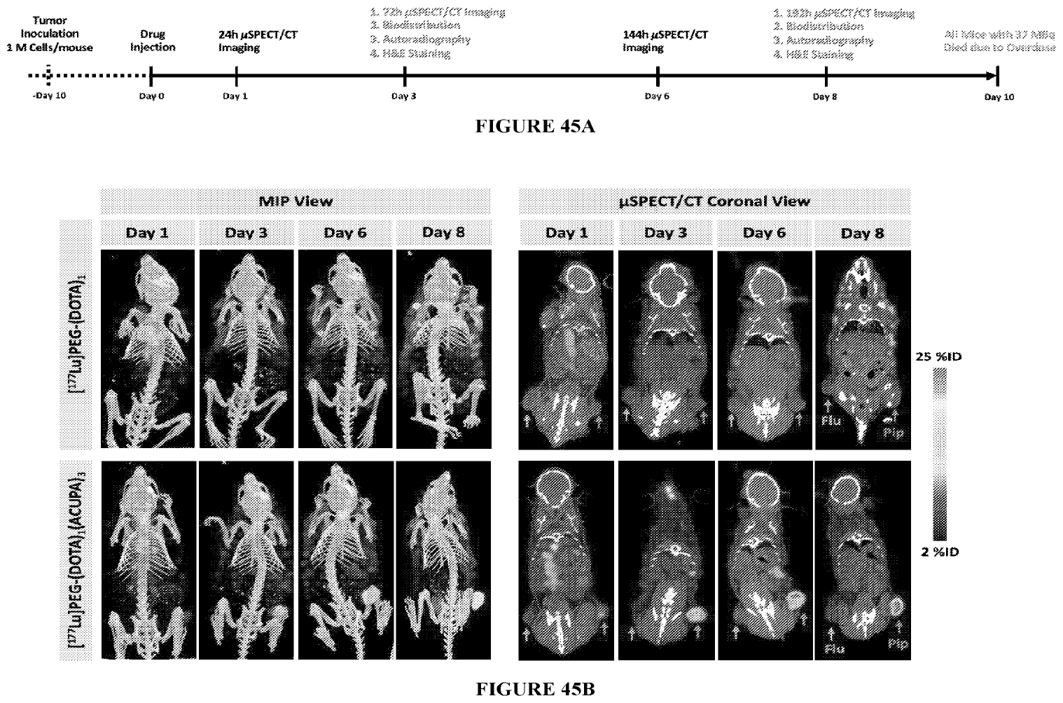

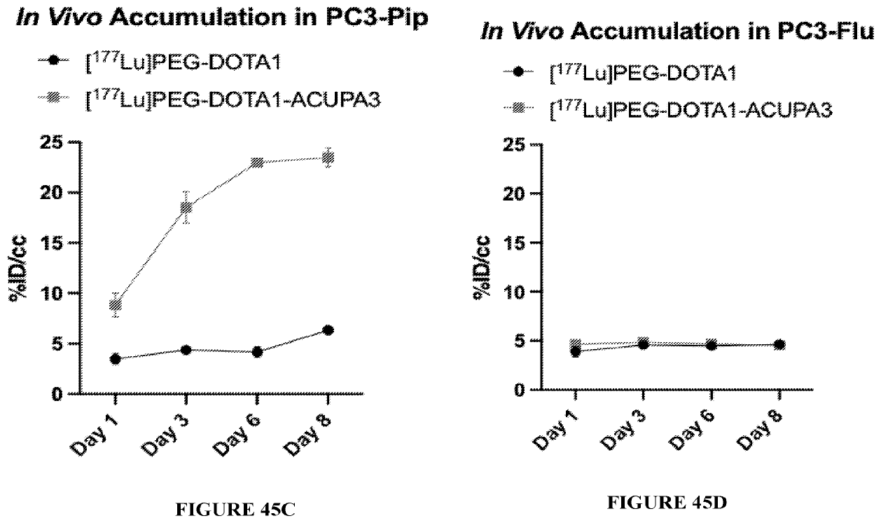

PSMA targeted tumor accumulation of both the ¹⁷⁷Lu radiolabeled nanocarriers, [¹⁷⁷Lu]PEG-(DOTA)₁ and [¹⁷⁷Lu]PEG-(DOTA)₁(ACUPA)₃, were evaluated in the nu/nu athymic mice model with both PSMA+PC3-Pip and PSMA-PC3-Flu tumors, which were subcutaneously xenografted at left and right flank, respectively. When the tumor volume reached 100-200 mm³, as schematically presented in FIG. 45A, the nanocarriers were administered to the mice via tail vein injection, and multiple time point SPECT/CT images were recorded, followed by ex vivo organ biodistribution up to 192 h. Primarily, the study population involved two groups (n=10 mice) for each nanocarrier. However, four mice from each group were injected with 37 MBq of the ¹⁷⁷Lu-labeled drug to acquire good quality μSPECT images, and rest six mice of each group were injected with 9-10 MBq for multiple timepoint ex vivo organ biodistribution, autoradiography, and H&E staining.

### μSPECT/CT Imaging

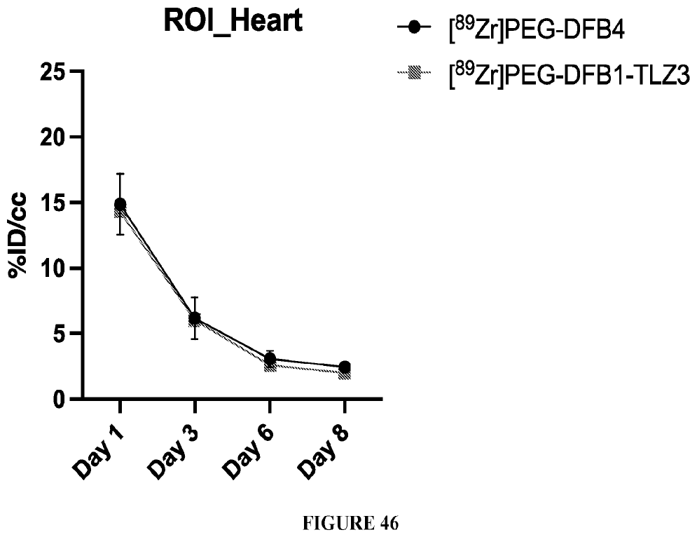

FIG. 45A shows representation of experimental design for in vivo evaluation of the 177Lu-labeled StarPEGs in mice bearing dual xenografts of PSMA+PC3-Pip and PSMA-PC3-Flu. FIG. 45B summarizes the maximum intensity projection (MTP) and coronal view of μSPECT/CT images captured at 24 h, 72 h, 168 h, and 192 h post-injection of the nanocarriers. Starting from 24 h post-injection, the targeted nanocarrier, [¹⁷⁷Lu]PEG-(DOTA)₁(ACUPA)₃, demonstrated noticeable PSMA targeted accumulation in PSMA+PC3-Pip tumors. In contrast, the accumulation increased significantly with very good background clearance at 72 h onwards. Respective regions of interest (ROI) were calculated from the μSPECT images to draw time-activity curves and were in line with the observed excellent accumulation in SPECT/CT images (FIGS. 45C-D, Table 12). At 24 h post-injection, the in vivo accumulation in PC3-Pip tumors was 8.85±1.17% ID/cc, which increased steadily to 22.96±0.19% ID/cc at 168 h. Interestingly, previously reported PET analogs of the StarPEG nanocarriers showed maximum PC3-Pip accumulation at 72 h post-injection, and afterward, the tumor accumulation decreased steadily. However, the theranostic version of the StarPEG nanocarrier tagged with 177Lu-DOTA pair demonstrated very stable and long time tumor retention at 23.47±0.94% ID/cc after 192 h post-injection, which is highly desirable for better treatment. Besides, the heart activity decreased drastically from 14.32±0.24% ID at 24 h to 1.99±0.08% ID at 192 h post-injection (FIG. 46). In comparison, the targeted nanocarrier in PSMA-PC3-Flu tumors and the nontargeted nanocarrier in bother PC3-Pip and PC3-Flu tumors demonstrated around 5% ID/cc accumulation, without any noticeable SPECT signal in the tumors.

TABLE 12
Region of interest analysis data reported as % ID/cc of the ¹⁷⁷Lu labeled nanocarriers
on heart, PC3-Pip, and PC3-Flu at 24 h, 72 h, 168 h, and 192 h (n = 2).

| | [¹⁷⁷Lu]PEG-(DOTA)₁ | | | | | | [¹⁷⁷Lu]PEG-(DOTA)₁(ACUPA)₃ | | | | | |
|---|---|---|---|---|---|---|---|---|---|---|---|---|
| Hours | Heart | | PC3-Pip | | PC3-Flu | | Heart | | PC3-Pip | | PC3-Flu | |
| 24 | 16.52 | 13.21 | 3.85 | 3.12 | 4.25 | 3.5 | 14.5 | 14.15 | 8.02 | 9.68 | 4.77 | 4.48 |
| 72 | 7.32 | 5.04 | 4.65 | 4.11 | 4.69 | 4.44 | 6.21 | 5.85 | 17.41 | 19.64 | 4.8 | 4.91 |
| 168 | 3.52 | 2.63 | 4.54 | 3.8 | 4.38 | 4.56 | 2.82 | 2.37 | 23.1 | 22.82 | 4.67 | 4.68 |
| 192 | 2.36 | 2.55 | 6.27 | 6.42 | 4.8 | 4.49 | 1.93 | 2.05 | 22.8 | 24.14 | 4.26 | 4.79 |

### Ex Vivo Organ Biodistribution

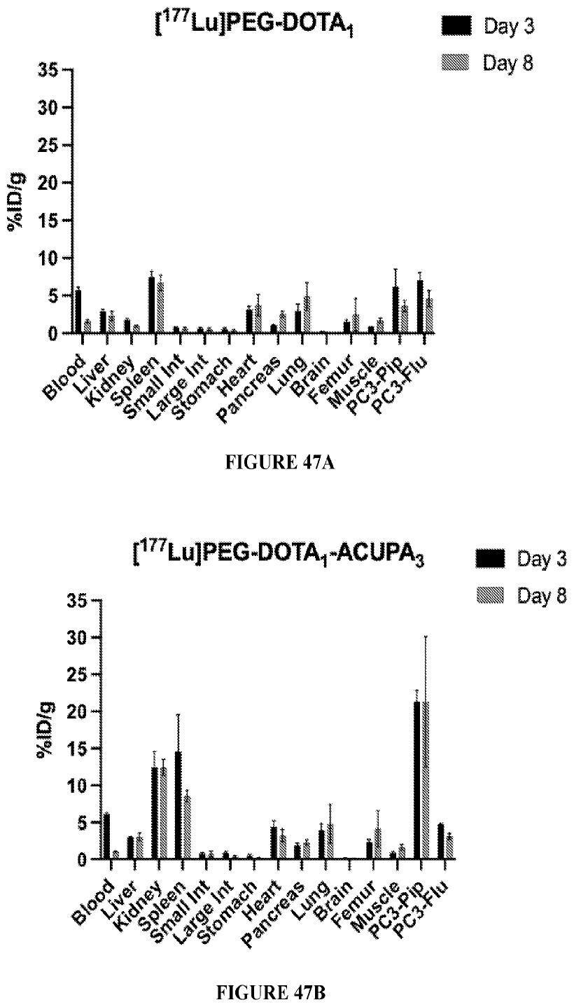

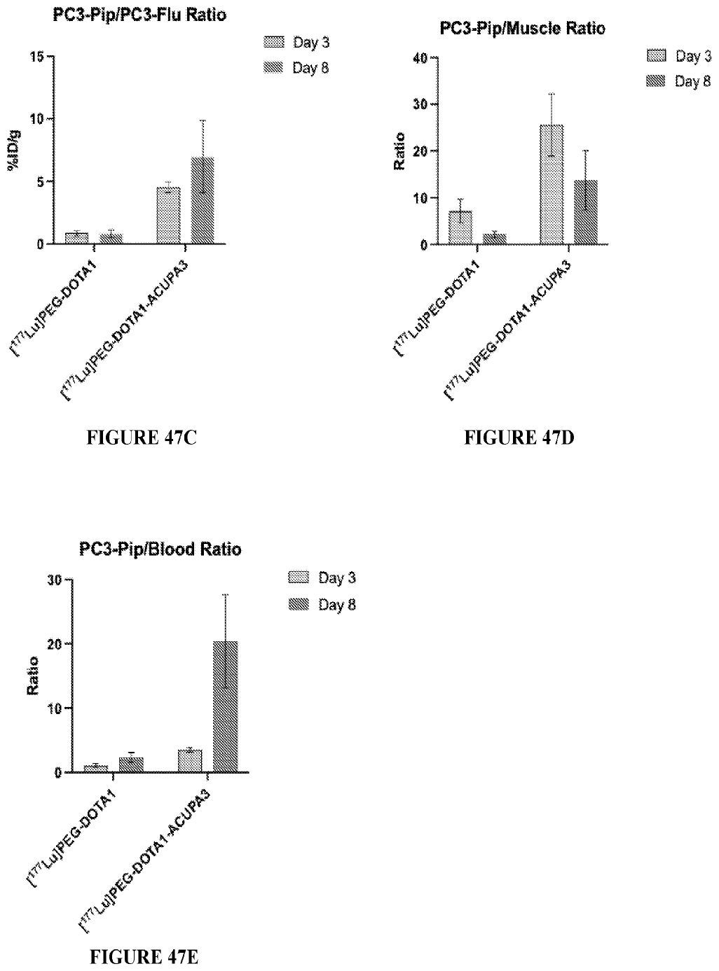

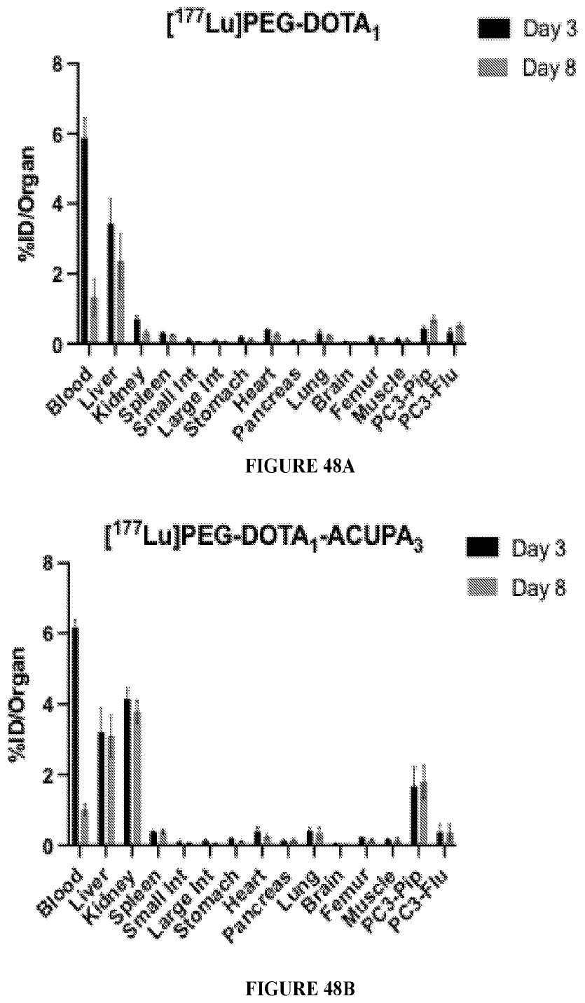

As schematically demonstrated in FIG. 45A, three mice injected with 9-10 MBq from each group were sacrificed at 72 h and 192 h post-injection to collect the major organs, and the distribution of the 177Lu-labeled nanocarriers was quantified in a gamma counter. All the ex vivo organ biodistribution results are summarized in FIGS. 47A-E, FIGS. 48A-B, and Tables 13-16. The targeted nanocarrier [¹⁷⁷Lu]PEG-(DOTA)₁(ACUPA)₃ showed excellent specific accumulation of 21.3±1.57 and 21.3±8.8% ID/g in PSMA+PC3-Pip tumors at 72 h and 192 h, respectively. In comparison, the targeted nanocarrier demonstrated only 4.7±0.11% ID/g accumulation in PSMA-PC3-Flu tumors, and the non-specific accumulation decreased further to 3.1±0.39% ID/g at 192 h post-injection. Similarly, the nontargeted nanocarrier [¹⁷⁷Lu]PEG-(DOTA)₁ demonstrated 6.2±2.26 and 7.0±1.02% ID/g non-specific accumulation in PC3-Pip and PC3-Flu tumors at 72 h, and decreased further to 3.6±0.73 and 4.6±1.06% ID/g at 192 h post-injection, respectively. Overall, the nanocarrier with three copies of ACUPA ligands demonstrated very high PSMA targeted accumulation in PSMA+PC3-Pip xenografts with excellent PC3-Pip to muscle ratio (25.5 at 72 h, and 13.7 at 192 h) and PC3-Pip to blood ratio (3.5 at 72 h, and 20.4 at 192 h). Besides, the PSMA-targeted nanocarrier showed around 12.4% ID/g accumulation in the kidney at 72 h, and remained almost stable at 192 h post-injection, whereas the non-specific nanocarrier demonstrated hardly 1.78 and 0.97% ID/g accumulation in the kidney at 72 h. Because mouse kidneys also well known to bear elevated PSMA expression, the above results further confirm high specificity of the developed StarPEG nanocarrier.

TABLE 13
Organ biodistribution analysis data in % ID/g tissue
for [¹⁷⁷Lu]PEG-(DOTA)₁ (n = 3).

| Organs | 72 h | | | 192 h | | |
|---|---|---|---|---|---|---|
| Blood | 5.433 | 5.455 | 6.25 | 1.82 | 1.381942 | 1.64 |
| Liver | 2.8 | 3.196 | 2.837 | 3.039 | 1.928413 | 2.01 |
| Kidney | 1.875 | 1.587 | 1.901 | 0.913 | 0.912162 | 1.11 |
| Spleen | 6.76 | 8.265 | 7.521 | 5.79 | 6.556393 | 7.75 |
| Small Int | 0.766 | 0.858 | 0.719 | 0.473 | 0.687284 | 0.8 |
| Large Int | 0.644 | 0.532 | 0.791 | 0.745 | 0.452388 | 0.37 |
| Stomach | 0.48 | 0.466 | 0.762 | 0.516 | 0.23626 | 0.34 |
| Heart | 2.891 | 2.966 | 3.693 | 3.362 | 5.316568 | 2.55 |
| Pancreas | 1.021 | 1.058 | 1.21 | 2.241 | 2.354366 | 3.01 |
| Lung | 2.746 | 2.189 | 3.961 | 3.705 | 7.025954 | 3.96 |
| Brain | 0.138 | 0.134 | 0.251 | 0.071 | 0.06236 | 0.07 |
| Femur | 1.205 | 1.772 | 1.618 | 4.961 | 1.130126 | 1.32 |
| Muscle | 0.831 | 0.888 | 0.881 | 1.979 | 1.804096 | 1.37 |
| PC3-Pip | 5.165 | 4.662 | 8.807 | 2.866 | 4.32525 | 3.712475 |
| PC3-Flu | 6.242 | 6.783 | 8.217 | 5.853 | 3.98762 | 4.023121 |

TABLE 14
Organ biodistribution analysis data in % ID/g tissue
for [¹⁷⁷Lu]PEG-(DOTA)₁(ACUPA)₃ (n = 3).

| Organs | 72 h | | | 192 h | | |
|---|---|---|---|---|---|---|
| Blood | 6.238 | 6.151 | 5.892 | 0.975 | 0.992767 | 1.12 |
| Liver | 3.058 | 2.96 | 2.852 | 3.246 | 2.457305 | 3.386589 |
| Kidney | 11.981 | 14.815 | 10.565 | 12.724 | 13.29727 | 11.2442 |
| Spleen | 16.146 | 9.015 | 18.595 | 8.372697 | 7.919506 | 9.437514 |
| Small Int | 0.878 | 0.765 | 0.636 | 1.101672 | 0.361912 | 0.647341 |
| Large Int | 0.944 | 0.679 | 0.97 | 0.54 | 0.292925 | 0.302282 |
| Stomach | 0.666 | 0.327 | 0.447 | 0.26043 | 0.137712 | 0.20241 |
| Heart | 5.19 | 4.332 | 3.578 | 2.295563 | 3.426993 | 3.931564 |
| Pancreas | 2.26 | 1.688 | 1.703 | 2.625753 | 1.944543 | 2.275291 |
| Lung | 4.268 | 4.632 | 3.024 | 7.669443 | 2.636102 | 4.092132 |
| Brain | 0.19 | 0.174 | 0.233 | 0.071268 | 0.077577 | 0.123464 |
| Femur | 2.623 | 2.388 | 2.007 | 2.885431 | 6.898916 | 2.561996 |
| Muscle | 0.972 | 0.906 | 0.698 | 1.854817 | 1.200526 | 1.841436 |
| PC3-Pip | 20.402 | 20.435 | 23.144 | 12.05354 | 22.29525 | 29.58977 |
| PC3-Flu | 4.746 | 4.83 | 4.604 | 3.320395 | 2.65879 | 3.346387 |

TABLE 15
Organ biodistribution analysis data in % ID/Organ tissue
for [¹⁷⁷Lu]PEG-(DOTA)₁ (n = 3).

| Organs | 72 h | | | 192 h | | |
|---|---|---|---|---|---|---|
| Blood | 5.58 | 5.541808 | 6.521061 | 0.74 | 1.434594 | 1.783977 |
| Liver | 3.76 | 3.935174 | 2.60438 | 1.54 | 2.380818 | 3.131179 |
| Kidney | 0.71 | 0.618888 | 0.799724 | 0.39 | 0.332301 | 0.264551 |
| Spleen | 0.31 | 0.287623 | 0.339959 | 0.282263 | 0.257011 | 0.242593 |
| Small Int | 0.09 | 0.158241 | 0.132506 | 0.058309 | 0.049416 | 0.034558 |
| Large Int | 0.12 | 0.130041 | 0.101854 | 0.088337 | 0.040082 | 0.049395 |
| Stomach | 0.22 | 0.181205 | 0.201278 | 0.160682 | 0.100411 | 0.113846 |
| Heart | 0.41 | 0.390874 | 0.430192 | 0.241442 | 0.250942 | 0.326097 |
| Pancreas | 0.09 | 0.121239 | 0.124349 | 0.098572 | 0.106182 | 0.11363 |
| Lung | 0.37 | 0.236168 | 0.366391 | 0.270965 | 0.175649 | 0.227464 |
| Brain | 0.05 | 0.035925 | 0.081975 | 0.021605 | 0.015559 | 0.01764 |
| Femur | 0.23 | 0.190158 | 0.205176 | 0.155133 | 0.151437 | 0.152305 |
| Muscle | 0.16 | 0.115307 | 0.157148 | 0.15935 | 0.081365 | 0.102732 |
| PC3-Pip | 0.53 | 0.385509 | 0.379603 | 0.670102 | 0.605535 | 0.818926 |
| PC3-Flu | 0.27 | 0.452445 | 0.289234 | 0.523006 | 0.477318 | 0.581829 |

TABLE 16
Organ biodistribution analysis data in % ID/Organ tissue
for [¹⁷⁷Lu]PEG-(DOTA)₁(ACUPA)₃ (n = 3).

| Organs | 72 h | | | 192 h | | |
|---|---|---|---|---|---|---|
| Blood | 6.34 | 6.24702 | 5.94473 | 0.837437 | 1.016594 | 1.150573 |
| Liver | 3.7 | 2.415584 | 3.487382 | 2.946966 | 2.606955 | 3.725248 |
| Kidney | 4.24 | 4.388248 | 3.837212 | 3.435414 | 3.877484 | 4.036668 |
| Spleen | 0.36 | 0.39126 | 0.403521 | 0.38933 | 0.322324 | 0.436957 |
| Small Int | 0.09 | 0.129133 | 0.111259 | 0.033381 | 0.057653 | 0.066806 |
| Large Int | 0.16 | 0.086285 | 0.136331 | 0.057571 | 0.062774 | 0.04767 |
| Stomach | 0.16 | 0.196473 | 0.217365 | 0.093989 | 0.10955 | 0.112682 |
| Heart | 0.52 | 0.287204 | 0.37356 | 0.193746 | 0.307401 | 0.246902 |
| Pancreas | 0.13 | 0.125756 | 0.141989 | 0.099516 | 0.182398 | 0.153355 |
| Lung | 0.44 | 0.459995 | 0.319921 | 0.526124 | 0.22196 | 0.268444 |
| Brain | 0.05 | 0.054263 | 0.06296 | 0.013605 | 0.020752 | 0.021791 |
| Femur | 0.23 | 0.223786 | 0.217923 | 0.115417 | 0.164884 | 0.162431 |
| Muscle | 0.18 | 0.147032 | 0.148009 | 0.066217 | 0.190163 | 0.187274 |
| PC3-Pip | 1.34 | 2.286641 | 1.390971 | 1.451246 | 2.332083 | 1.583053 |
| PC3-Flu | 0.25 | 0.625992 | 0.261021 | 0.611949 | 0.29805 | 0.166985 |

### Autoradiography and Histology Analysis

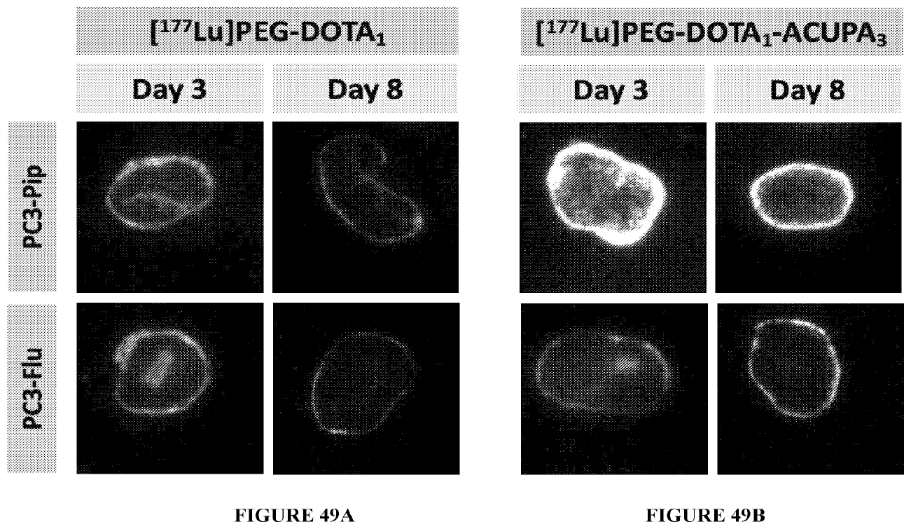

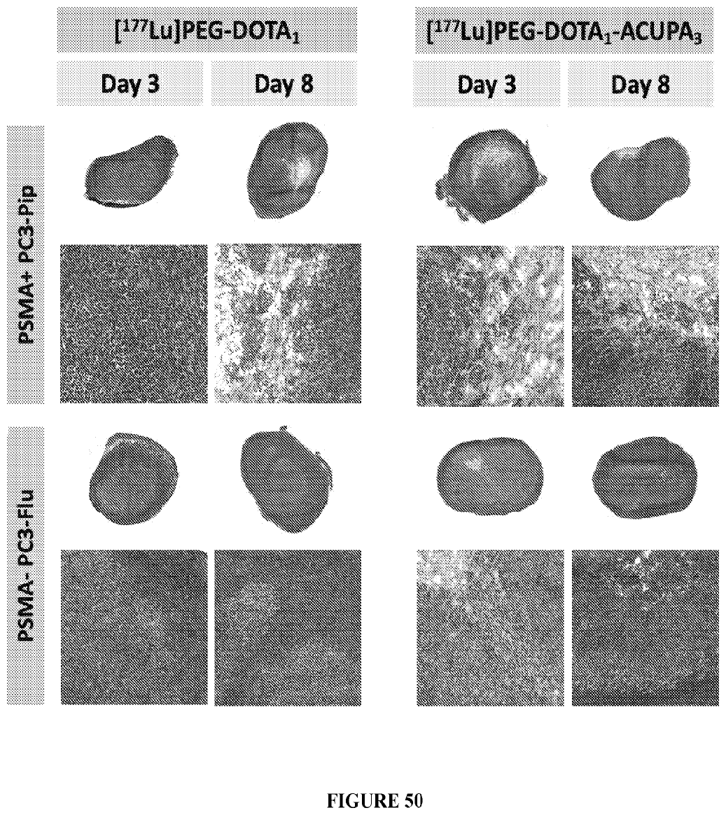

Post gamma counting of the collected major organs at 72 h and 192 h post-injection, the tumors were sectioned at 10 μM thickness and subjected to autoradiography and histology analysis to explore the drug distribution. FIGS. 49A-B demonstrated a highly elevated intensity of β-particles accumulated in PSMA+PC3-Pip tumors treated with the targeted nanocarrier [¹⁷⁷Lu]PEG-(DOTA)₁(ACUPA)₃. Whereas the targeted nanocarriers in PSMA-PC3-Flu xenografts and the nontargeted nanocarrier in both the PC3-Pip and PC3-Flu tumors showed very faint peripheral intensity. Despite very high PC3-Pip tumor accumulation of the targeted nanocarrier in PC3-Pip tumors compared to PC3-Flu, mostly bright signals of accumulations were observed around the tumor periphery. For further analysis of the homogenous peripheral accumulation, the tumor tissues collected at 72 h and 192 h post-injection were stained with hematoxylin and eosin (H&E) (FIG. 50). The H&E staining demonstrated the development of central necrosis in almost all the tumor sections collected for both the PC3-Pip and PC3-Flu tumors, and thereby demonstrated reduced tissue penetration despite efficient PSMA binding affinity of [¹⁷⁷Lu]PEG-(DOTA)₁(ACUPA)₃.

### Absorbed Dose

Both tumor and normal tissue dosimetry were performed by drawing volumes of interest (VOIs) on coregistered CT images in SPECT/CT data of the subcutaneous tumor-bearing mice model. It should be noted that the mice were injected with 1 mCi ¹⁷⁷Lu-labeled nanocarriers for better μSPECT/CT imaging, but all the mice died on day 10 post-injection due to high activity. Because of high dose injection, all the tumors received a good amount of therapeutic absorbed dose (above 20-30 Gy/mCi) irrespective of nanocarriers and tumor model (Table 17). However, the targeted nanocarrier [¹⁷⁷Lu]PEG-(DOTA)₁(ACUPA)₃ demonstrated more than 5-fold higher absorbed dose in PSMA+PC3-Pip tumors as compared to PC3-Flu suggesting highly potent targeted antitumor response of the nanocarrier. The tumor dosimetry analysis suggested that the PSMA-targeted nanocarrier could have a considerable therapeutic efficacy at as low as 100-200 μCi dose in PSMA+Pc3-Pip tumors. Besides, the percent of injected activity (% IA) within the defined organs was extrapolated to human-equivalent organ and whole-body effective doses using ratios of standard human organ weights to mouse organ weights. Tables 18-21 summarized the organ and effective dose estimated using OLINDA version 1.1 with ICRP Publication 60 tissue weighting factors and OLINDA version 2.0 with ICRP Publication 103 tissue weighting factors. The normal tissue dose estimation demonstrated noticeable lungs and liver retention but washed out well over time. Besides, skin uptake at later time points was observed for both the nanocarriers. The normal tissue dosimetry suggested that lungs are at the highest risk for radiation exposure among all major organs.

TABLE 17
Absorbed tumor radiation dose in mice models bearing
PC3-Pip and PC3-Flu dual xenografts and injected with
1 mCi of the ¹⁷⁷Lu labeled nanocarriers.

| Absorbed Tumor Dose (mSv/MBq) | | PC3-Pip | PC3-Flu |
|---|---|---|---|
| [¹⁷⁷Lu]PEG-(DOTA)₁ | Mouse 1 | 979 | 849 |
| | Mouse 2 | 571 | 603 |
| [¹⁷⁷Lu]PEG-(DOTA)₁(ACUPA)₃ | Mouse 1 | 4007 | 853 |
| | Mouse 2 | 4303 | 908 |

TABLE 18
Human-equivalent normal tissue radiation dose estimation of
[¹⁷⁷Lu]PEG-(DOTA)₁ treated in nude mice using using OLINDA
version 1.1 and ICRP Publication 60 tissue weighting factors.

| Adult male Organs (73 kg) | Absorbed Dose (mSv/MBq) |
|---|---|
| Adrenals | 0.1420 ± 0.0042 |
| Brain | 0.0576 ± 0.0175 |
| Breasts | 0.1330 ± 0.0042 |
| Gallbladder Wall | 0.1425 ± 0.0035 |
| LLI Wall | 0.1380 ± 0.0057 |
| Small Intestine | 0.1390 ± 0.0057 |
| Stomach Wall | 0.1385 ± 0.0049 |
| ULI Wall | 0.1385 ± 0.0049 |
| Heart Wall | 0.3245 ± 0.0276 |
| Kidneys | 0.1525 ± 0.0332 |
| Liver | 0.3240 ± 0.0849 |
| Lungs | 1.0950 ± 0.1344 |
| Muscle | 0.1345 ± 0.0049 |
| Pancreas | 0.1420 ± 0.0042 |
| Red Marrow | 0.1045 ± 0.0035 |
| Osteogenic Cells | 0.4175 ± 0.0163 |
| Skin | 0.1295 ± 0.0049 |
| Spleen | 0.1385 ± 0.0049 |
| Testes | 0.1330 ± 0.0057 |
| Thymus | 0.1390 ± 0.0042 |
| Thyroid | 0.1355 ± 0.0049 |
| Urinary Bladder Wall | 0.1905 ± 0.0106 |
| Total Body | 0.1605 ± 0.0007 |
| Effective Dose (mSv/MBq) | 0.2560 ± 0.0170 |

TABLE 19
Human-equivalent normal tissue radiation dose estimation
of [¹⁷⁷Lu]PEG-(DOTA)₁(ACUPA)₃ treated in nude
mice using using OLINDA version 1.1 and ICRP Publication
60 tissue weighting factors.

| Adult male Organs (73 kg) | Absorbed Dose (mSv/MBq) |
|---|---|
| Adrenals | 0.1425 ± 0.0007 |
| Brain | 0.0576 ± 0.0066 |
| Breasts | 0.1335 ± 0.0007 |
| Gallbladder Wall | 0.1435 ± 0.0007 |
| LLI Wall | 0.1385 ± 0.0007 |
| Small Intestine | 0.1395 ± 0.0007 |
| Stomach Wall | 0.1385 ± 0.0007 |
| ULI Wall | 0.1395 ± 0.0007 |
| Heart Wall | 0.3105 ± 0.0035 |
| Kidneys | 0.2435 ± 0.0219 |
| Liver | 0.3415 ± 0.0233 |
| Lungs | 0.9390 ± 0.1146 |
| Muscle | 0.1355 ± 0.0007 |
| Pancreas | 0.1425 ± 0.0007 |
| Red Marrow | 0.1045 ± 0.0007 |
| Osteogenic Cells | 0.4190 ± 0.0028 |
| Skin | 0.1300 ± 0.0014 |
| Spleen | 0.1385 ± 0.0007 |
| Testes | 0.1335 ± 0.0007 |
| Thymus | 0.1390 ± 0.0000 |
| Thyroid | 0.1365 ± 0.0007 |
| Urinary Bladder Wall | 0.1870 ± 0.0184 |
| Total Body | 0.1595 ± 0.0007 |
| Effective Dose (mSv/MBq) | 0.2390 ± 0.0127 |

TABLE 20
Human-equivalent normal tissue radiation dose estimation of
[¹⁷⁷Lu]PEG-(DOTA)₁ treated in nude mice using using OLINDA
version 2.0 and ICRP Publication 103 tissue weighting factors.

| Adult male Organs (73 kg) | Absorbed Dose (mSv/MBq) |
|---|---|
| Adrenals | 0.1835 ± 0.0502 |
| Brain | 0.0568 ± 0.0173 |
| Esophagus | 0.1585 ± 0.0148 |
| Eyes | 0.1335 ± 0.0049 |
| Gallbladder Wall | 0.2030 ± 0.0750 |
| Left colon | 0.1460 ± 0.0014 |
| Small Intestine | 0.1460 ± 0.0014 |
| Stomach Wall | 0.1555 ± 0.0106 |
| Right colon | 0.1545 ± 0.0134 |
| Rectum | 0.1405 ± 0.0049 |
| Heart Wall | 0.3255 ± 0.0460 |
| Kidneys | 0.1640 ± 0.0537 |
| Liver | 2.1355 ± 2.6227 |
| Lungs | 0.9270 ± 0.1315 |
| Pancreas | 0.1600 ± 0.0170 |
| Prostate | 0.1405 ± 0.0035 |
| Salivary Glands | 0.1365 ± 0.0049 |
| Red Marrow | 0.1125 ± 0.0021 |
| Osteogenic Cells | 0.0832 ± 0.0047 |
| Spleen | 0.1445 ± 0.0007 |
| Testes | 0.1350 ± 0.0057 |
| Thymus | 0.1495 ± 0.0035 |
| Thyroid | 0.1435 ± 0.0021 |
| Urinary Bladder Wall | 0.1930 ± 0.0113 |
| Total Body | 0.2080 ± 0.0622 |
| Effective Dose (mSv/MBq) | 0.2888 ± 0.1262 |

TABLE 21
Human-equivalent normal tissue radiation dose estimation
of [¹⁷⁷Lu]PEG-(DOTA)1(ACUPA)3 treated in nude
mice using using OLINDA version 2.0 and ICRP Publication
103 tissue weighting factors.

| Adult male Organs (73 kg) | Absorbed Dose (mSv/MBq) |
|---|---|
| Adrenals | 0.1480 ± 0.0014 |
| Brain | 0.0568 ± 0.0064 |
| Esophagus | 0.1455 ± 0.0007 |
| Eyes | 0.1340 ± 0.0014 |
| Gallbladder Wall | 0.1490 ± 0.0014 |
| Left colon | 0.1420 ± 0.0014 |
| Small Intestine | 0.1420 ± 0.0014 |
| Stomach Wall | 0.1450 ± 0.0000 |
| Right colon | 0.1425 ± 0.0007 |
| Rectum | 0.1405 ± 0.0007 |
| Heart Wall | 0.2985 ± 0.0021 |
| Kidneys | 0.2365 ± 0.0205 |
| Liver | 0.3640 ± 0.0240 |
| Lungs | 0.7825 ± 0.0940 |
| Pancreas | 0.1455 ± 0.0007 |
| Prostate | 0.1395 ± 0.0007 |
| Salivary Glands | 0.1370 ± 0.0014 |
| Red Marrow | 0.1085 ± 0.0007 |
| Osteogenic Cells | 0.0784 ± 0.0004 |
| Spleen | 0.1405 ± 0.0007 |
| Testes | 0.1355 ± 0.0007 |
| Thymus | 0.1440 ± 0.0000 |
| Thyroid | 0.1415 ± 0.0007 |
| Urinary Bladder Wall | 0.1895 ± 0.0191 |
| Total Body | 0.1625 ± 0.0007 |
| Effective Dose (mSv/MBq) | 0.1967 ± 0.0109 |

Treatment Studies with [¹⁷⁷Lu]PEG-(DOTA)₁(ACUPA)₃

As observed in the in vivo evaluation in the mice models bearing subcutaneous dual xenografts, the PSMA targeted nanocarrier with three ACUPA ligands demonstrated very high specificity towards PSMA+PC3-Pip tumors with around 5-fold higher absorbed dose as compared to the nontargeted nanocarrier or in the PSMA-PC3-Pip tumors. This encouraged us to further evaluate the treatment efficacy of the 177Lu labeled targeted nanocarrier, [¹⁷⁷Lu]PEG-(DOTA)₁(ACUPA)₃, in subcutaneous as well as metastatic tumors bearing mice models. As observed in the dosimetry analysis, 1 mCi dose demonstrated high radiation exposure and toxicity to the normal tissue. Thus, the mice were treated with 500 μCi or less for the therapy study.

### Treatment Studies in PSMA+PC3-Pip Subcutaneous Models

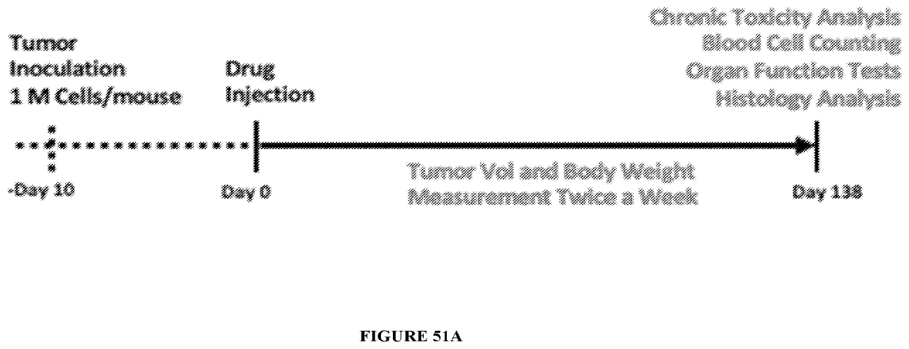

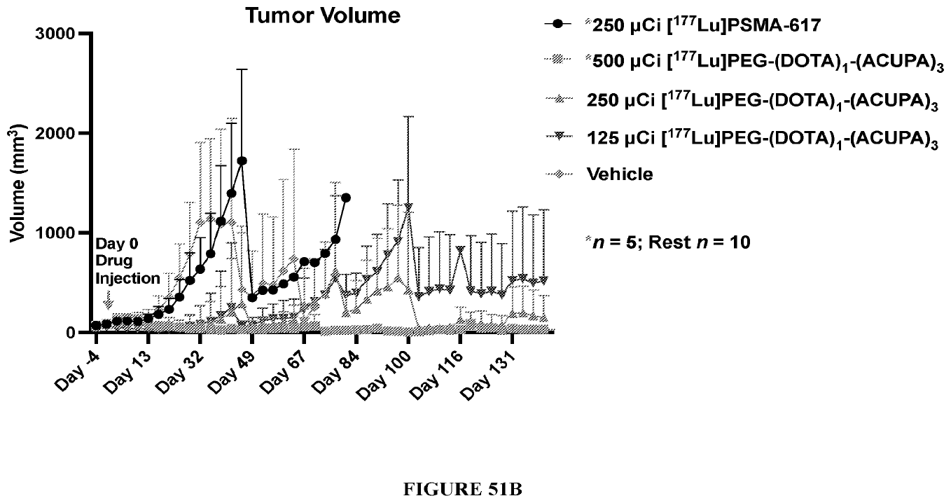

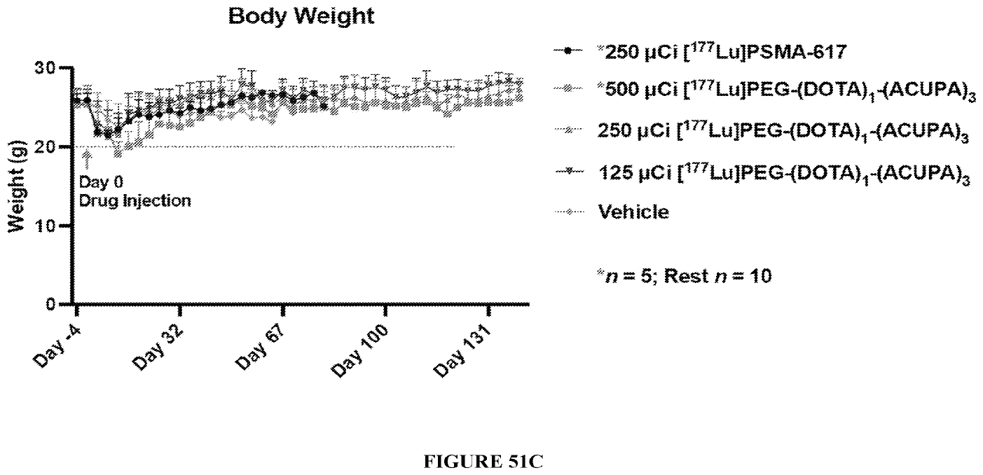

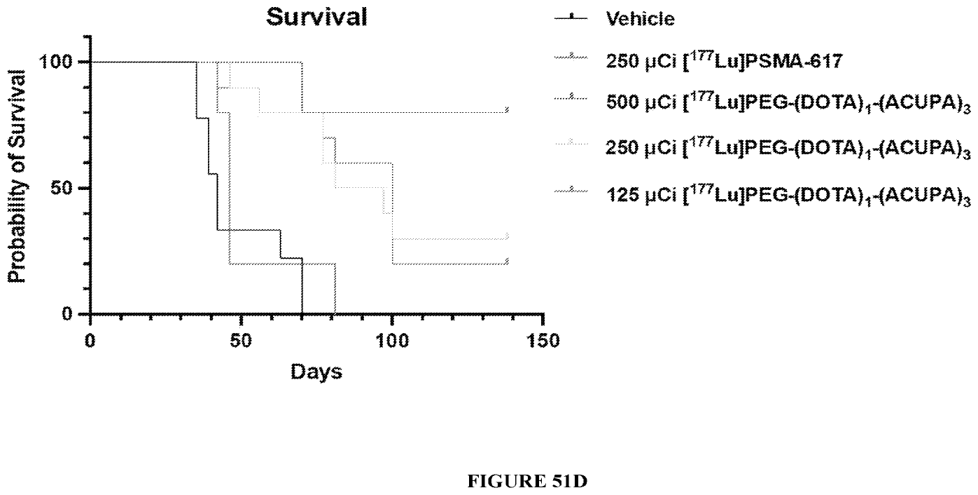

As demonstrated in FIG. 51A, after 10 days of cell inoculation, the mice with PC3-Pip subcutaneous tumors of 100-200 mm³ were treated with the targeted nanocarrier [¹⁷⁷Lu]PEG-(DOTA)₁(ACUPA)₃, and the tumor volume and bodyweight of the mice were monitored. Along with the injection of different doses (500, 250, and 125 μCi) of the targeted nanocarrier, one cohort of mice (n=5) was treated with 250 μCi of [¹⁷⁷Lu]PSMA-617 as a positive control. FIG. 51B-D show the tumor volume, body weight, and survival of the mice up to 138 days post-treatment of the drugs. 500 μCi single dose of [¹⁷⁷Lu]PEG-(DOTA)₁(ACUPA)₃ demonstrated highly efficient suppression of the tumor volume without any regrowth of tumors with 80% survival at 138 days post drug injection. Both 250 and 125 μCi doses of [¹⁷⁷Lu]PEG-(DOTA)₁(ACUPA)₃ significantly delayed the tumor growth compared to the vehicle and 250 μCi single dose of [¹⁷⁷Lu]PSMA-617. The median survival of 250 and 125 μCi doses of [¹⁷⁷Lu]PEG-(DOTA)₁(ACUPA)₃ were 89 and 100 days, respectively. However, the tumor growth pattern in mice injected with a vehicle and 250 μCi single doses of [¹⁷⁷Lu]PSMA-617 were almost similar, with 42 and 46 days of median survival, respectively.

### Treatment Studies in PC3-Pip Metastatic Models Monitored by [⁶⁸Ga]PSMA-II μPET CT Imaging

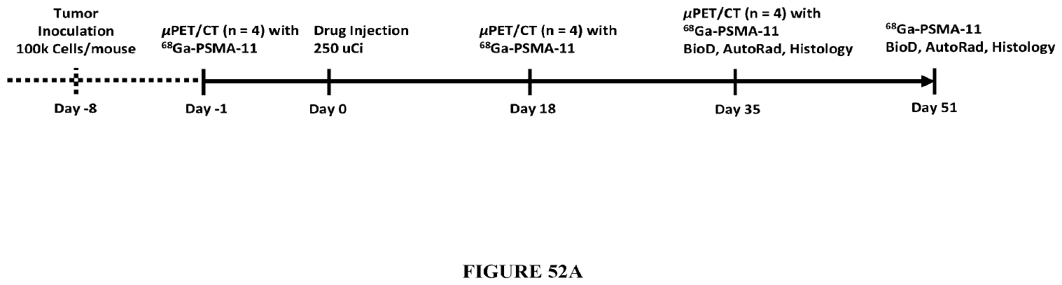

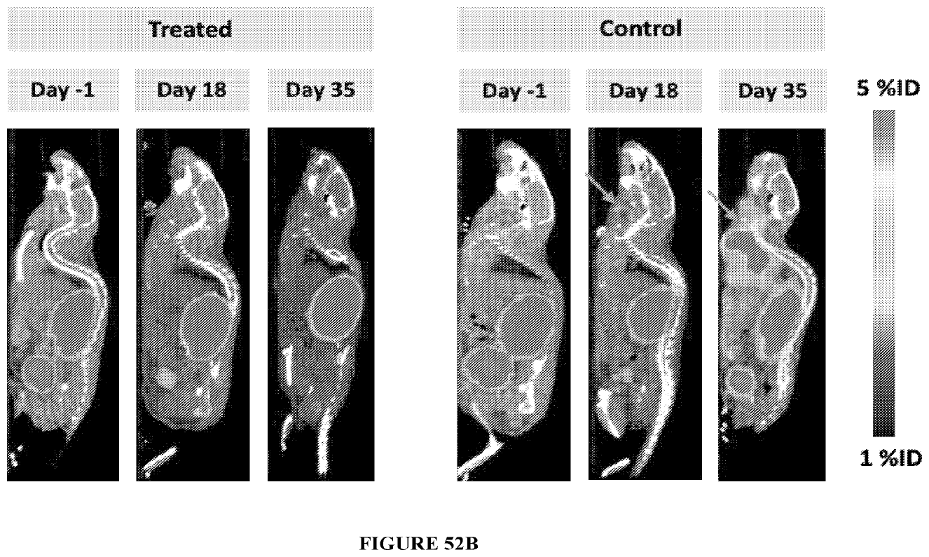

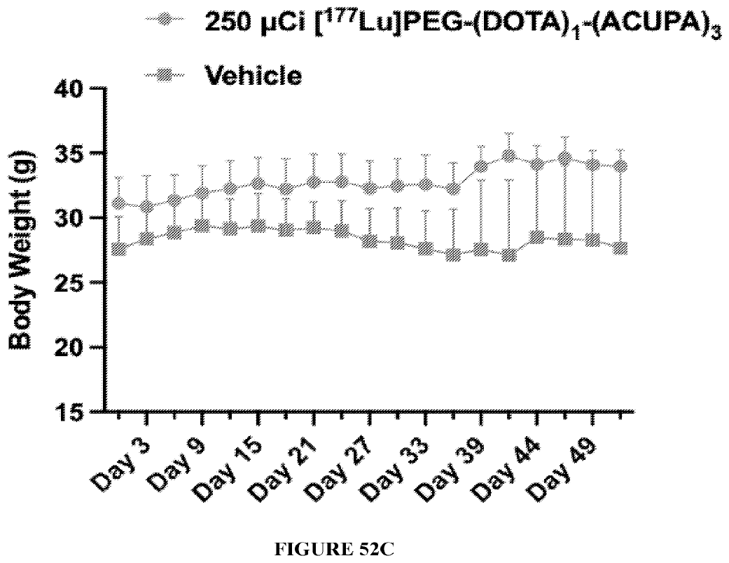

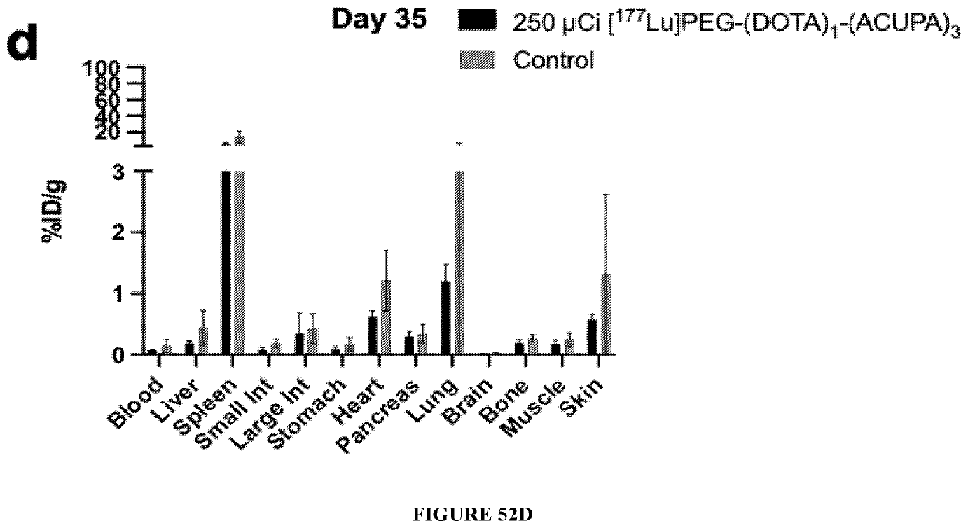

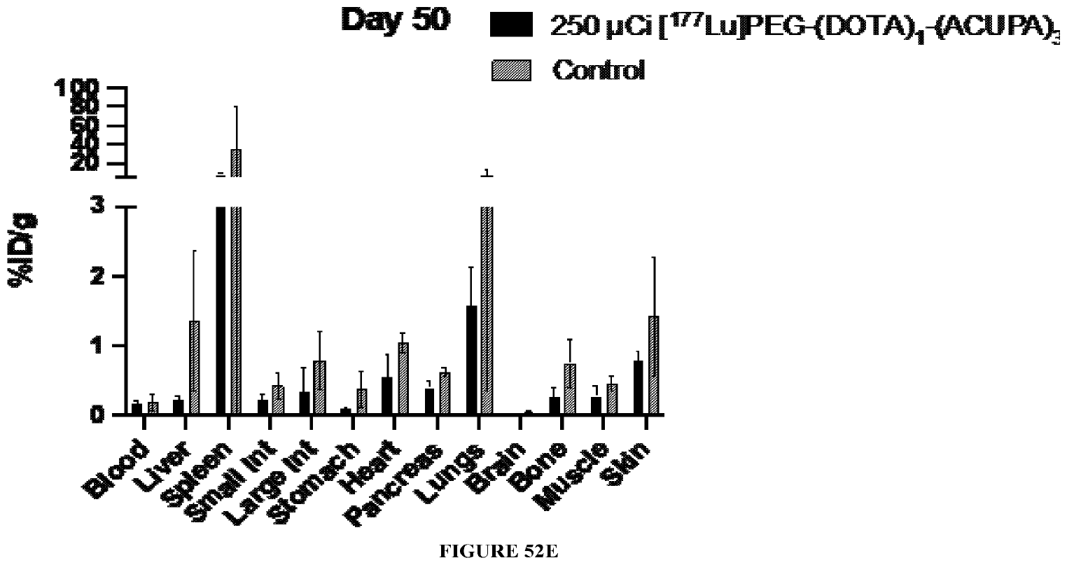

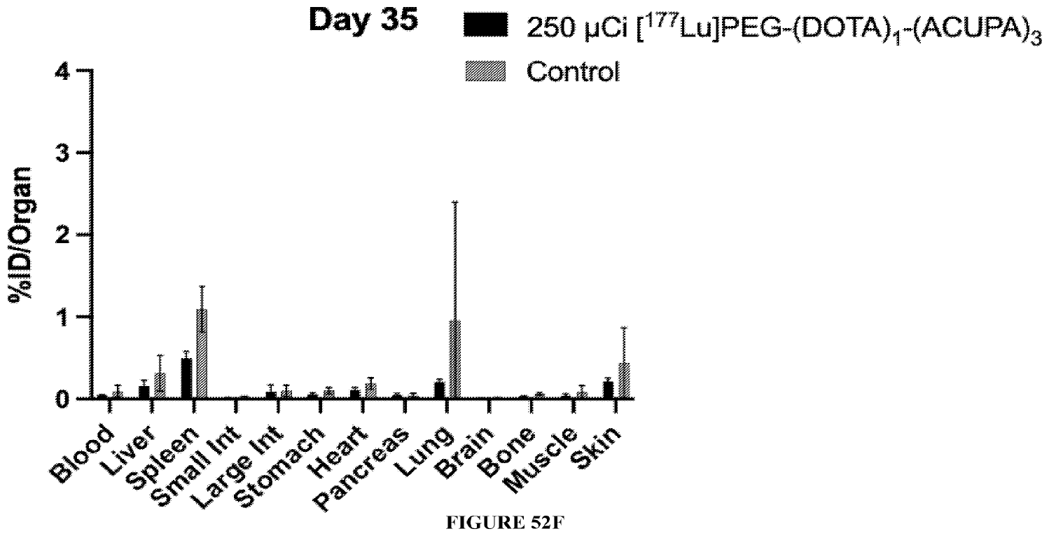

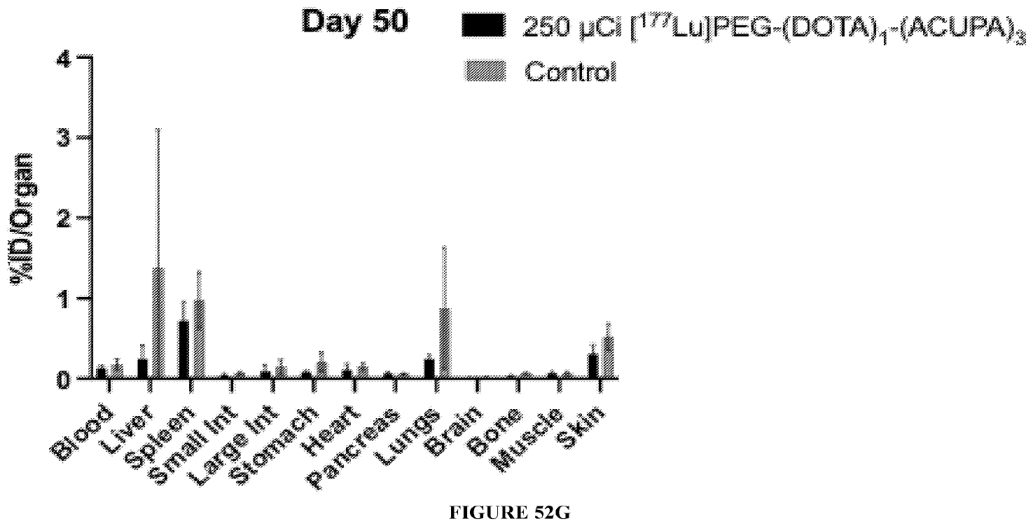

The treatment efficacy of the targeted nanocarrier [¹⁷⁷Lu]PEG-(DOTA)ᵢ(ACUPA)₃ was further evaluated in mice bearing PC3-Pip metastatic tumors developed by the inoculation of PC3-Pip cells in the left ventricle of the heart by intracardiac injection. As depicted in the experimental protocol (FIG. 52A), two cohorts of mice (n=10) were injected with either vehicle or 250 μCi of [¹⁷⁷Lu]PEG-(DOTA)₁(ACUPA)₃ after 10 days of cell injection. The treatment efficacy of the nanocarrier was monitored by [⁶⁸Ga]PSMA-11 based μPET/CT imaging, body weight measurement, and organ biodistribution. The mice were subjected to [⁶⁸Ga]PSMA-11 PET/CT imaging one day before, on day 18, and day 35 of drug treatment (¹⁷⁷Lu labeled nanocarrier) (FIG. 52B). All mice from both groups demonstrated drug accumulation in the kidneys and bladder. The control mice without drug treatment developed metastatic tumors in the neck region, including at axillary lymph nodes and salivary glands, and demonstrated strong accumulation of [⁶⁸Ga]PSMA-11. However, no such metastatic tumors were observed in the treated group indicating efficient suppression of metastatic tumor growth. Besides, no significant body weight loss was observed in the treated and control groups up to 51 days post drug treatment (FIG. 52C). A small cohort of three mice from each group were subjected to organ biodistribution on day 35 and day 51 post drug injection. As shown in FIGS. 52D-G and Table 22-25, a relatively high accumulation of [⁶⁸Ga]PSMA-11 was observed in most of the major organs, including the liver, lungs, and spleen of the control group as compared to the drug-treated group indicating the burden of metastatic tumor in those organs.

TABLE 22
Ex vivo organ biodistribution (% ID/g) of [⁶⁸Ga]PSMA-11 in mice model bearing PC3-
Pip metastatic tumors and 35 days post treatment of 250 μCi [¹⁷⁷Lu]PEG-(DOTA)₁(ACUPA)₃.

| Organs | Treated | | | | Control | | | |
|---|---|---|---|---|---|---|---|---|
| Blood | 0.083 | 0.081156 | 0.08 | 0.07 | 0.114 | 0.299866 | 0.08 | 0.1 |
| Liver | 0.23 | 0.219532 | 0.15 | 0.15 | 0.855 | 0.405352 | 0.231959 | 0.289 |
| Splcon | 5.652 | 5.362448 | 4.45 | 7.27 | 22.586 | 8.850674 | 8.108291 | 16.37588 |
| Small Int | 0.151 | 0.069608 | 0.05 | 0.06 | 0.29 | 0.139685 | 0.166323 | 0.14785 |
| Large Int | 0.767 | 0.052214 | 0.49 | 0.12 | 0.663 | 0.333776 | 0.129225 | 0.59202 |
| Stomach | 0.14 | 0.116191 | 0.07 | 0.05 | 0.171 | 0.32708 | 0.115715 | 0.094409 |
| Heart | 0.52 | 0.701134 | 0.71 | 0.56 | 1.938 | 1.092544 | 0.884232 | 0.937111 |
| Pancreas | 0.359 | 0.318706 | 0.35 | 0.19 | 0.464 | 0.206568 | 0.229679 | 0.500656 |
| Lung | 1.015 | 1.364313 | 1.5 | 0.95 | 8.218 | 1.814282 | 1.285 | 1.336 |
| Brain | 0.016 | 0.015377 | 0.03 | 0.01 | 0.046 | 0.013714 | 0.024038 | 0.034044 |
| Bone | 0.275 | 0.168998 | 0.17 | 0.16 | 0.335 | 0.215027 | 0.226854 | 0.303255 |
| Muscle | 0.177 | 0.12963 | 0.14 | 0.27 | 0.414 | 0.204413 | 0.219211 | 0.162355 |
| Skin | 0.487 | 0.656398 | 0.541385 | 0.644673 | 3.25 | 0.586015 | 0.981736 | 0.443305 |

TABLE 23
Ex vivo organ biodistribution (% ID/g) of [⁶⁸Ga]PSMA-11 in mice model bearing PC3-
Pip metastatic tumors and 50 days post treatment of 250 μCi [¹⁷⁷Lu]PEG-(DOTA)₁(ACUPA)₃.

| Organs | Treated | | | | | | | | Control | | |
|---|---|---|---|---|---|---|---|---|---|---|---|
| Blood | 0.173 | 0.166 | 0.223 | 0.13 | 0.201 | 0.178 | 0.169 | 0.207 | 0.07 | 0.212 | 0.29 |
| Liver | 0.184 | 0.213 | 0.331 | 0.12 | 0.194 | 0.165 | 0.308 | 0.215 | 0.336 | 2.351 | 1.41 |
| Spleen | 6.622 | 7.974 | 10.736 | 5.426 | 6.824 | 5.625 | 12.352 | 5.37 | 8.812 | 10.073 | 86.889 |
| Small Int | 0.217 | 0.303 | 0.352 | 0.186 | 0.125 | 0.095 | 0.212 | 0.23 | 0.289 | 0.365 | 0.631 |
| Large Int | 0.228 | 0.175 | 0.457 | 0.186 | 0.424 | 0.156 | 0.065 | 1.093 | 1.205 | 0.766 | 0.402 |
| Stomach | 0.126 | 0.063 | 0.105 | 0.078 | 0.093 | 0.081 | 0.079 | 0.154 | 0.107 | 0.605 | 0.424 |
| Heart | 0.575 | 0.553 | 1.305 | 0.241 | 0.236 | 0.518 | 0.345 | 0.615 | 0.906 | 1.037 | 1.182 |
| Pancreas | 0.479 | 0.34 | 0.581 | 0.389 | 0.291 | 0.338 | 0.314 | 0.447 | 0.649 | 0.568 | 0.668 |
| Lungs | 1.274 | 1.331 | 2.901 | 1.202 | 1.263 | 1.613 | 1.567 | 1.403 | 2.187 | 4.185 | 14.472 |
| Brain | 0.018 | 0.019 | 0.023 | 0.015 | 0.033 | 0.021 | 0.024 | 0.024 | 0.025 | 0.037 | 0.073 |
| Bone | 0.228 | 0.161 | 0.273 | 0.188 | 0.18 | 0.559 | 0.331 | 0.335 | 0.768 | 0.398 | 1.082 |
| Muscle | 0.265 | 0.247 | 0.562 | 0.204 | 0.13 | 0.217 | 0.259 | 0.405 | 0.533 | 0.335 | 0.498 |
| Skin | 0.707 | 0.551 | 0.981 | 0.761 | 0.675 | 0.799 | 0.91 | 0.883 | 0.807 | 1.068 | 2.397 |

TABLE 24
Ex vivo organ biodistribution (% ID/Organ) of [⁶⁸Ga]PSMA-
11 in mice model bearing PC3-Pip metastatic tumors and 35 days post treatment
of 250 μCi [¹⁷⁷Lu]PEG-(DOTA)₁(ACUPA)₃.

| Organs | Treated | | | | Control | | | |
|---|---|---|---|---|---|---|---|---|
| Blood | 0.05 | 0.051834 | 0.039364 | 0.049169 | 0.06 | 0.20289 | 0.05813 | 0.053237 |
| Liver | 0.2 | 0.236853 | 0.104505 | 0.103847 | 0.62 | 0.298217 | 0.109902 | 0.224584 |
| Spleen | 0.45 | 0.507824 | 0.405544 | 0.605303 | 1.34 | 1.068276 | 0.716773 | 1.252755 |
| Small Int | 0.02 | 0.011639 | 0.006164 | 0.012019 | 0.03 | 0.023467 | 0.031418 | 0.027559 |
| Large Int | 0.18 | 0.011268 | 0.130802 | 0.03401 | 0.09 | 0.08014 | 0.030717 | 0.191874 |
| Stomach | 0.08 | 0.051926 | 0.059764 | 0.046133 | 0.16 | 0.100185 | 0.070621 | 0.08055 |
| Heart | 0.11 | 0.15446 | 0.097659 | 0.069446 | 0.29 | 0.157654 | 0.154829 | 0.147595 |
| Pancreas | 0.07 | 0.054594 | 0.046916 | 0.028915 | 0.04 | 0.024478 | 0.032063 | 0.077952 |
| Lung | 0.23 | 0.224975 | 0.220864 | 0.149256 | 3.12 | 0.276678 | 0.186915 | 0.239265 |
| Brain | 0.01 | 0.005417 | 0.00976 | 0.003105 | 0.02 | 0.004254 | 0.009255 | 0.012654 |
| Bone | 0.03 | 0.027428 | 0.0414 | 0.03033 | 0.08 | 0.054187 | 0.044395 | 0.068445 |
| Muscle | 0.06 | 0.022011 | 0.033675 | 0.058336 | 0.2 | 0.056438 | 0.04564 | 0.041725 |
| Skin | 0.25 | 0.215167 | 0.234799 | 0.15917 | 1.08 | 0.266695 | 0.195955 | 0.218683 |

TABLE 25
Ex vivo organ biodistribution (% ID/Organ) of [⁶⁸Ga]PSMA-
11 in mice model bearing PC3-Pip metastatic tumors and 50 days post treatment of
250 μCi [¹⁷⁷Lu]PEG-(DOTA)₁(ACUPA)₃. (n = 4)

| Organs | | | | Treated | | | | Control | | | |
|---|---|---|---|---|---|---|---|---|---|---|---|
| Blood | 0.13 | 0.15 | 0.17 | 0.15 | 0.07 | 0.13 | 0.12 | 0.14 | 0.14 | 0.13 | 0.26 |
| Liver | 0.2 | 0.12 | 0.54 | 0.1 | 0.15 | 0.1 | 0.45 | 0.3 | 0.41 | 3.36 | 0.35 |
| Spleen | 0.59 | 0.74 | 0.97 | 0.43 | 0.65 | 0.44 | 1.02 | 0.94 | 1.1 | 0.58 | 1.25 |
| Small Int | 0.06 | 0.04 | 0.07 | 0.04 | 0.03 | 0.02 | 0.05 | 0.06 | 0.07 | 0.05 | 0.09 |
| Large Int | 0.03 | 0.03 | 0.11 | 0.04 | 0.19 | 0.05 | 0.02 | 0.24 | 0.25 | 0.12 | 0.06 |
| Stomach | 0.09 | 0.06 | 0.08 | 0.07 | 0.06 | 0.09 | 0.05 | 0.12 | 0.11 | 0.34 | 0.17 |
| Heart | 0.1 | 0.12 | 0.27 | 0.05 | 0.04 | 0.09 | 0.05 | 0.14 | 0.18 | 0.16 | 0.09 |
| Pancreas | 0.06 | 0.06 | 0.09 | 0.07 | 0.05 | 0.04 | 0.06 | 0.07 | 0.06 | 0.05 | 0.07 |
| Lungs | 0.21 | 0.21 | 0.28 | 0.24 | 0.28 | 0.23 | 0.34 | 0.19 | 0.34 | 0.55 | 1.74 |
| Brain | 0.01 | 0 | 0.01 | 0.01 | 0.01 | 0.01 | 0.01 | 0.01 | 0.01 | 0.01 | 0.02 |
| Bone | 0.02 | 0 | 0.03 | 0.02 | 0.04 | 0.03 | 0.03 | 0.05 | 0.07 | 0.06 | 0.07 |
| Muscle | 0.07 | 0.07 | 0.09 | 0.05 | 0.02 | 0.05 | 0.06 | 0.11 | 0.09 | 0.04 | 0.06 |
| Skin | 0.43 | 0.19 | 0.37 | 0.42 | 0.2 | 0.16 | 0.41 | 0.31 | 0.33 | 0.62 | 0.6 |

### Chronic Toxicity Studies in PC3-Pip Subcutaneous Models

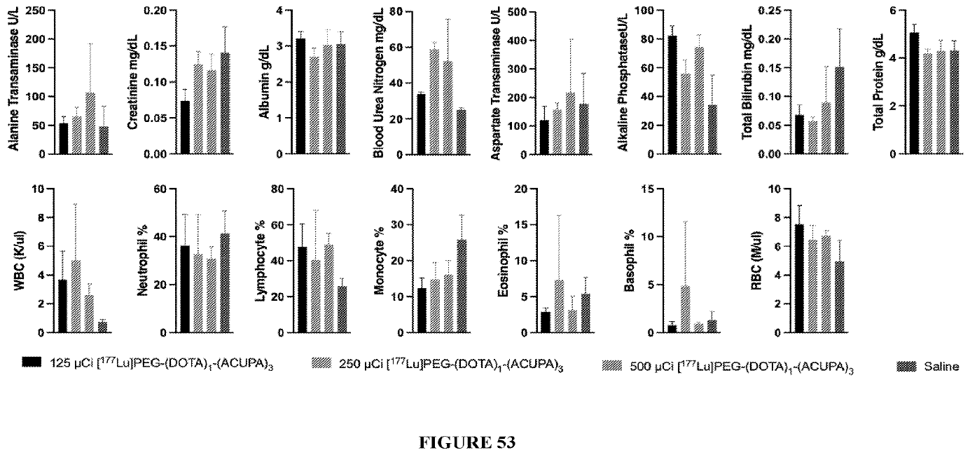

As schematically presented in the FIG. 51A, the therapy experiment ended 138 days post drug injection, and the mice that survived were subjected to chronic toxicity analysis, which includes 4 mice of 500 μCi dose, 2 mice of 250 μCi dose, and 3 mice of 125 μCi dose. Besides, 3 untreated healthy mice were included in the toxicity analysis as control. Laboratory analyses were performed for liver and kidney function tests and blood count measurements demonstrating no significant alteration in the albumin and total protein level (FIG. 53, Tables 26-27). Creatinine and bilirubin levels were found to be almost similar, whereas elevated levels of alanine transaminase, alkaline transaminase, and blood urea nitrogen levels were seen in the treated group as compared to the vehicle group, which correspond to poor kidney and liver function with possible damage in liver and bones. Blood counts demonstrated leukocytosis with elevated lymphocytes indicating an inflammatory condition in mice.

Histology analyses were performed in the organs collected in this chronic toxicity experiment on day 138 post-treatment of [¹⁷⁷Lu]PEG-(DOTA)₁(ACUPA)₃.

TABLE 26
Liver and kidney function tests results for chronic toxicity in
mice model bearing PC3-Pip subcutaneous tumor and injected with
different dose of 177Lu labeled targeted nanocarrier. (Mean ± SD)

| | | [¹⁷⁷Lu]PEG-(DOTA)₁(ACUPA)₃ | | |
|---|---|---|---|---|
| | Vehicle | 125 μCi | 250 μCi | 500 μCi |
| | (n = 3) | (n = 3) | (n = 2) | (n = 4) |
| Alanine transaminase U/L | 48.23 ± 34.8 | 53.63 ± 11.6 | 65.2 ± 15.6 | 107.12 ± 84.4 |
| Aspartate transaminase U/L | 178.23 ± 106.6 | 120.16 ± 48.1 | 157.55 ± 24.8 | 217.8 ± 186.1 |
| Albumin g/dL | 3.04 ± 0.3 | 3.21 ± 0.1 | 2.69 ± 0.2 | 3.02 ± 0.4 |
| Alkaline Phosphatase U/L | 34.36 ± 20.4 | 82.33 ± 6.6 | 55.8 ± 9.6 | 74.45 ± 8.3 |
| Blood Urea Nitrogen mg/dL | 25.16 ± 1.0 | 34.0 ± 1.0 | 58.8 ± 3.9 | 52.32 ± 23.2 |
| Creatinine mg/dL | 0.14 ± 0.03 | 0.074 ± 0.01 | 0.12 ± 0.01 | 0.11 ± 0.02 |
| Total Bilirubin mg/dL | 0.15 ± 0.06 | 0.068 ± 0.01 | 0.057 ± 0.007 | 0.089 ± 0.06 |
| Total Protein g/dL | 4.32 ± 0.3 | 5.06 ± 0.3 | 4.185 ± 0.1 | 4.28 ± 0.4 |

TABLE 27
Blood parameters for the chronic toxicity study in mice model bearing PC3-Pip subcutaneous
tumor and injected with different dose of 177Lu labeled targeted nanocarrier (MCV: mean
corpuscular volume, MCH: mean corpuscular hemoglobin, MCHC: mean corpuscular hemoglobin
concentration, RDW: red cell distribution width, MPV: mean platelet volume). (Mean ± SD)

| | | [¹⁷⁷Lu]PEG-(DOTA)₁(ACUPA)₃ | | |
|---|---|---|---|---|
| | Vehicle | 125 μCi | 250 μCi | 500 μCi |
| | (n = 3) | (n = 3) | (n = 2) | (n = 4) |
| WBC (K/ul) | 0.74 ± 0.1 | 3.66 ± 1.9 | 5.0 ± 3.9 | 2.60 ± 0.7 |
| Absolute Neutrophil cells (K/ul) | 0.3 ± 0.03 | 1.37 ± 0.8 | 1.96 ± 2.1 | 0.78 ± 0.1 |
| Absolute Lymphocyte cells (K/ul) | 0.19 ± 0.05 | 1.75 ± 1.2 | 1.48 ± 0.2 | 1.29 ± 0.4 |
| Absolute Monocyte cells (K/ul) | 0.2 ± 0.09 | 0.41 ± 0.1 | 0.64 ± 0.3 | 0.42 ± 0.1 |
| Absolute Eosinophil cells (K/ul) | 0.04 ± 0.02 | 0.1 ± 0.04 | 0.54 ± 0.7 | 0.09 ± 0.08 |
| Absolute Basophil cells (K/ul) | 0.01 ± 0.01 | 0.023 ± 0.006 | 0.37 ± 0.5 | 0.022 ± 0.01 |
| RBC (M/ul) | 41.45 ± 9.2 | 36.34 ± 12.9 | 32.65 ± 16.6 | 30.74 ± 5.0 |
| Hemoglobin (g/dL) | 25.95 ± 4.2 | 47.62 ± 12.8 | 40.47 ± 27.5 | 49.07 ± 6.2 |
| Hematocrit % | 25.84 ± 6.8 | 12.36 ± 2.8 | 14.71 ± 4.7 | 16.13 ± 3.8 |
| Neutrophil % | 5.41 ± 2.2 | 2.89 ± 0.5 | 7.3 ± 9.0 | 3.14 ± 1.9 |
| Lymphocyte % | 1.32 ± 0.9 | 0.76 ± 0.4 | 4.87 ± 6.6 | 0.90 ± 0.2 |
| Monocyte % | 4.94 ± 1.4 | 7.53 ± 1.2 | 6.46 ± 0.9 | 6.71 ± 0.3 |
| Eosinophil % | 7.36 ± 2.3 | 11.23 ± 1.8 | 9.85 ± 1.4 | 10.35 ± 0.5 |
| Basophil % | 23.76 ± 7.5 | 33.43 ± 5.8 | 29.5 ± 4.3 | 32.45 ± 2.0 |
| MCV (fL) | 47.93 ± 0.9 | 44.4 ± 1.1 | 45.7 ± 0.2 | 48.27 ± 0.7 |
| MCH (pg) | 14.86 ± 0.4 | 14.93 ± 0.8 | 15.25 ± 0.07 | 15.4 ± 0.2 |
| MCHC (g/dL) | 31 ± 0.4 | 33.63 ± 0.9 | 33.35 ± 0.07 | 31.9 ± 0.7 |
| RDW % | 18.23 ± 0.3 | 20.3 ± 0.4 | 19.4 ± 1.4 | 20.27 ± 0.6 |
| Platelets (K/uL) | 131.66 ± 23.2 | 569.3 ± 347.1 | 488.0 ± 517.6 | 671 ± 291.6 |
| MPV (fL) | 5.26 ± 0.1 | 5.33 ± 0.05 | 5.65 ± 0.35 | 5.55 ± 5.5 |

### Discussion

Herein, the PSMA targeted in vitro binding efficacy along with in vivo SPECT imaging and therapeutic efficacy of newly synthesized 4-armed StarPEG nanocarriers (40 kDa MW) conjugated with three copies of PSMA targeting ACUPA ligands was evaluated. This current study is the theranostic extension of our prior findings, where the nontargeted StarPEG nanocarriers, [⁸⁹Zr]PEG-(DOTA)₄, labeled with PET imaging isotope ⁸⁹Zr demonstrated significantly high passive accumulation in MX-1 and HT-29 tumor models, but unable to penetrate deep into the prostate cancer tumor models like PC3-Pip and PC3-Flu. Although, the StarPEG nanocarrier of 40 kDa molecular weight (15 nm hydrodynamic diameter) offers an ideal size for EPR-based passive tumor uptake, tumor vasculature, and macrophages strongly influence the EPR-mediated passive accumulation of the nanocarriers. As observed in a nontargeted polymer nanostar, the CT26 with leaky vasculature possessed good homogeneous tumor accumulation of 14.8% ID/g, whereas below 5.5% ID/g of heterogeneous peripheral accumulation of the polymer nanostar was observed in the poorly leaky BxPC3 tumor. In fact most of the prostate cancer tumor models evaluated with large size nanocarriers have been reported to be EPR low phenotype and demonstrate heterogenous low tumor uptake and tissue penetration of nanocarriers due to their poor vascular development. However, our prior finding demonstrated significant improvement in the tissue penetration of StarPEG nanocarriers in PSMA+PC3-Pip tumors with long time tumor retention after conjugating PSMA-targeting ACUPA ligands to the, which is highly desirable for better therapeutic efficacy. Therefore, the therapeutic version of the PET imaging scaffold was designed and synthesized by replacing the diagnostic radiometal-chelator pair of ⁸⁹Zr-DFB with the theranostic pair of ¹⁷⁷Lu-DOTA. Two StarPEG nanocarriers were synthesized, viz PSMA targeted PEG-(DOTA)₁(ACUPA)₃ and nontargeted PEG-(DOTA)₁, for comparative evaluation of PSMA targeted theranostic efficacy of the nanocarriers.

The designed StarPEG conjugates were successfully synthesized and purified in good yields by using PEG-(NH₂)₁(5HCyO)₃ as starting material and following previously reported synthetic protocols. The PSMA-targeting ACUPA ligands were conjugated to the StarPEG via click coupling of respective azide and cyclooctyne counterparts. However, in the case of the nontargeted StarPEG nanocarrier, the cyclooctyne counterparts in the StarPEG were capped with azido-PEG7 for better comparison. For both the targeted as well as nontargeted nanocarriers, DOTA was conjugated to the free amine group via p-phenylene thiourea linker, and the number of DOTA units in both the nanocarriers was kept same to evenly compare the PSMA targeted therapeutic efficacy of the nanocarriers.

DOTA conjugated StarPEG nanocarriers were radiolabeled with the β-emitting theranostic radioisotope ¹⁷⁷Lu in NH₄OAc buffer containing ascorbic acid as a radiolytic protectant. However, it was observed that despite efficient radiolabeling as found in iTLC, the isolated yield was below 10%, especially in scale-up radiosynthesis. Surprisingly, a major fraction of the activity got stuck in the stationary phase of PD-10 size-exclusion desalting column, indicating radiolytic degradation of the StarPEG nanocarriers. To restrict the radiolytic damage of the radiolabeled nanocarriers inside the PD-10 column, the antioxidant ascorbic acid (0.2 mg of L-ascorbic acid per mL) containing saline solution was used while equilibrating and eluting the polymer nanocarriers to restrict any free radical damage. This minor perturbation in the purification protocol resulted in excellent radiochemical yield. The molar activities for [¹⁷⁷Lu]PEG-(DOTA)₁ was 294.4-312.8 μCi/nmol, whereas relatively better molar activity of 395.2-427.1 μCi/nmol were obtained for [¹⁷⁷Lu]PEG-(DOTA)₁(ACUPA)₃, which could be due to relatively lower number of DOTA conjugated to PEG-(DOTA)₁. Overall, two StarPEG nanocarriers without or with three copies of PSMA targeting ACUPA ligands were synthesized and radiolabeled with ¹⁷⁷Lu with good molar activities.

In vitro PSMA binding affinity of the synthesized nanocarriers were evaluated in PSMA+PC3-Pip and PSMA-PC3-Flu prostate cancer cells via multiple cell binding assays such as competition radioligand binding, saturation binding, blocking, and membrane-bound & internalization assays. As expected, the three ACUPA ligands conjugated nanocarrier PEG-(DOTA)₁(ACUPA)₃ demonstrated excellent PSMA binding affinity in PSMA+PC3-Pip cells with 57.5 nM dissociation constant (K_{d}) and 1024 nM of IC₅₀ value. However, a relatively lower binding affinity of the theranostic nanocarrier PEG-(DOTA)₁(ACUPA)₃ was observed as compared to that of the previously reported diagnostic nanocarrier PEG-(DFB)₁(ACUPA)₃. The plausible explanation of such relatively lower PSMA binding affinity of the theranostic StarPEG nanocarrier could be due to the replacement of DFB with DOTA radiometal chelator. The blocking and membrane-bound and internalization assay demonstrated a steady increase of bound activity with increasing incubation time in PSMA+PC3-Pip cells (>20% AD). At 1 h, the internalized activity was lower than membrane-bound activity, which increased rapidly with time and became higher at 24 h time point, which suggests PSMA targeted cell internalization of the nanocarrier. However, no specific binding of the targeted nanocarrier was observed in PSMA-PC3-Flu cells with below 0.1% AD bound activity at all time points. As expected, the nanocarrier [¹⁷⁷Lu]PEG-(DOTA)₁ without any PSMA targeting ACUPA ligands showed no sign of specific binding in any of the prostate cancer cell lines. Overall, the in vitro cell binding results demonstrated ACUPA ligand-directed efficient PSMA binding affinity of [¹⁷⁷Lu]PEG-(DOTA)₁(ACUPA)₃ in PSMA+PC3-Pip cells. Although, replacing the DFB radiometal chelator of the nanocarrier with DOTA relatively reduced the cell binding affinity, PEG-(DOTA)₁(ACUPA)₃ still demonstrated excellent binding affinity worthy of further in vivo theranostic evaluation.

Multiple time points in vivo SPECT/CT imaging and ex vivo organ biodistribution were performed in nude mice bearing dual xenografts of PC3-Pip and PC3-Flu up to 192 h post-injection of the nanocarriers, [¹⁷⁷Lu]PEG-(DOTA)₁(ACUPA)₃ and [¹⁷⁷Lu]PEG-(DOTA)₁. Starting from 24 h post-injection, the targeted nanocarrier, [¹⁷⁷Lu]PEG-(DOTA)₁(ACUPA)₃, demonstrated noticeable PSMA targeted accumulation (8.85±1.17% ID/cc) in PSMA+PC3-Pip tumors, and increased steadily up to 23.47±0.94% ID/cc at 192 h post-injection with strong signal in SPECT imaging. The interesting observation was, unlike the previously reported diagnostic StarPEG nanocarrier [⁸⁹Zr]PEG-(DFB)₁(ACUPA)₃,¹ no partial tumor clearance of the theranostic nanocarrier was observed after 72 h post injection. In fact, the highest PC3-Pip to blood ratio of 20.4 was witnessed at 192 h, which was highly desirable and could potentially enhance the therapeutic efficacy of the nanocarrier. Besides, the PSMA-targeted excellent PC3-Pip tumor accumulation was also observed in the ex vivo organ biodistribution (21.3% ID/g uptake at both 72 h and 192 h, respectively). In comparison, the targeted nanocarrier in PSMA-PC3-Flu tumors and the nontargeted nanocarrier in both PC3-Pip and PC3-Flu tumors demonstrated around 5% TD/cc accumulation without any noticeable SPECT signal in the tumors. Such low nontargeted tumor accumulation could be the EPR-mediated passive uptake of the nanocarriers and are in line with previously reported nontargeted StarPEG nanocarriers. Besides, the PSMA-targeted nanocarrier showed a significantly high accumulation in the kidney compared to that of the nontargeted nanocarrier, which further confirmed very specificity of the developed StarPEG nanocarrier considering the fact that mouse kidney is also well known to bear elevated PSMA expression. Post gamma counting, the tumor tissues' sections were subjected to high-resolution autoradiography imaging that demonstrated the highly elevated intensity of β-particles accumulated in PSMA+PC3-Pip tumors treated with the targeted nanocarrier [¹⁷⁷Lu]PEG-(DOTA)₁(ACUPA)₃. However, bright signals of accumulations were mostly observed around the tumor periphery with comparatively lower deep-tissue penetration, which could be due to the necrotic cell death inside the tumors as observed in their respective H&E staining images.

Tumor and whole-body dosimetry analysis of the nanocarriers was performed by drawing volumes of interest (VOIs) in SPECT/CT images. Since the mice were injected with a high dose of 1 mCi for better SPECT image resolution, all the tumors received a good therapeutic absorbed dose (29.3±6.18 Gy, 793.8±167.2 mSv/MBq) irrespective of nanocarriers and tumor model; however, all mice died of an overdose on day 10 post-injection. Importantly, the tumor dosimetry results demonstrated more than 5.2-fold higher dose delivery (153.7±7.74 Gy, 4155±209.3 mSv/MBq) in PSMA+PC3-Pip by the targeted nanocarrier. It has been observed that patients receiving as low as 14 Gy tumor-absorbed dose of [¹⁷⁷Lu]PSMA-617 demonstrated more than 50% reduction of prostate specific antigen. This suggests that the PSMA targeted nanocarrier could have a considerable therapeutic efficacy at as low as 91 μCi dose in PSMA+Pc3-Pip tumors. Calculation of human-equivalent organ and whole-body effective doses demonstrated the highest risk of radiation exposure to lungs, but mostly around 1 mSv/MBq.

Considering the very high toxicity of 1 mCi dose, the mice models bearing PC3-Pip xenografts were subjected to therapy study with three different single-dose treatments (500, 250, and 125 μCi) of [¹⁷⁷Lu]PEG-(DOTA)₁(ACUPA)₃. 250 μCi of [¹⁷⁷Lu]PSMA-617 was treated as a positive control, whereas saline containing 0.2 mg ascorbic acid was used as a negative control. Both 250 and 125 μCi doses of [¹⁷⁷Lu]PEG-(DOTA)₁(ACUPA)₃ significantly delayed the tumor growth (89 and 100 days median survival, respectively) compared to the vehicle (42 days). Highly efficient antitumor response was observed in 500 μCi dose of [¹⁷⁷Lu]PEG-(DOTA)₁(ACUPA)₃ without any regrowth of tumor up to 138 days. Whereas the single dose antitumor response of 250 μCi [¹⁷⁷Lu]PSMA-617 (46 days median survival) was consistent with prior literature (51 days median survival). The vehicle group demonstrated noticeable delayed tumor growth with 42 days of median survival compared to prior literature, which could be due to ascorbic acid content in the vehicle.

Apart from the subcutaneous models, the therapeutic efficacy of the targeted nanocarrier was also evaluated in PC3-Pip metastatic tumor models developed by intracardiac cell injection. The antitumor response before and after treatment of 250 μCi [¹⁷⁷Lu]PEG-(DOTA)₁(ACUPA)₃ was monitored by [⁶⁸Ga]PSMA-11 based μPET/CT imaging. Efficient antitumor response was observed in the treated group with no sign of any metastatic tumor, while the control mice without any drug treatment developed metastatic tumors in the neck region, including at axillary lymph nodes and salivary glands, and demonstrated strong accumulation of [⁶⁸Ga]PSMA-11. Besides, the organ biodistribution on day 35 and 51 demonstrated noticeable suppression of tumor burden of major organs, including liver, lungs, and spleen in [¹⁷⁷Lu]PEG-(DOTA)₁(ACUPA)₃ treated group as compared to the control group.

All the PC3-Pip tumor bearing mice that survived 138 days post drug treatment were subjected to chronic toxicity analysis. As observed in the imaging experiment, 1 mCi dose was extremely high for the mice, and all mice died on day 10 post-injection of the nanocarriers with rapid weight loss. However, none of the mice in the therapy cohort, treated with 500 μCi of [¹⁷⁷Lu]PEG-(DOTA)₁(ACUPA)₃ or less, reached the endpoint as a result of weight loss. Although, the nude mice are known to have lower registrant towards radioactivity, 500 μCi or less doses are well tolerated by the mice. Temporary body weight loss was observed in all the cohorts post drug injection due to C. bovis bacterial infection (FIG. 51C). However, the mice recovered body weight very quickly after the treatment of antiseptic chlorhexidine. Laboratory analyses for liver and kidney function tests and blood count measurements demonstrated elevated levels of alanine transaminase, alkaline transaminase, blood urea nitrogen, and lymphocytes in the treated group as compared to the vehicle group, which indicated poor kidney and liver function with inflammatory condition.

### Conclusion

In conclusion, a PSMA-targeted StarPEG theranostic nanocarrier, [¹⁷⁷Lu]PEG-(DOTA)₁(ACUPA)₃, was designed and synthesized for the radioligand imaging and therapy of prostate cancer, which demonstrated excellent suppression of both subcutaneous and metastatic PSMA+PC3-Pip xenografts. The single-dose therapeutic efficacy of the nanocarrier was compared to that of [¹⁷⁷Lu]PSMA-617, which demonstrated significant improvement in the therapeutic efficacy of [¹⁷⁷Lu]PEG-(DOTA)₁(ACUPA)₃ in preclinical models with clinical translation possibilities in the future.

### REFERENCES

1) Bray, F.; Ferlay, J.; Soerjomataram, I.; Siegel, R.; Torre, L.; Jemal, A. Global cancer statistics 2018: GLOBOCAN estimates of incidence and mortality worldwide for 36 cancers in 185 countries. Ca-a Cancer Journal For Clinicians 2018, 68 (6), 394-424, Article. DOI: 10.3322/caac.21492.
2) Sung, H.; Ferlay, J.; Siegel, R.; Laversanne, M.; Soerjomataram, I.; Jemal, A.; Bray, F. Global cancer statistics 2020: GLOBOCAN estimates of incidence and mortality worldwide for 36 cancers in 185 countries. Ca-a Cancer Journal For Clinicians 2021, 71 (3), 209-249, Article. DOI: 10.3322/caac.21660.
3) Maurer, T.; Eiber, M.; Schwaiger, M.; Gschwend, J. Current use of PSMA-PET in prostate cancer management. Nature Reviews Urology 2016, 13 (4), 226-235, Review. DOI: 10.1038/nrurol.2016.26.
4) Giesel, F.; Knorr, K.; Spohn, F.; Will, L.; Maurer, T.; Flechsig, P.; Neels, O.; Schiller, K.; Amaral, H.; Weber, W.; et al. Detection Efficacy of F-18-PSMA-1007 PET/CT in 251 Patients with Biochemical Recurrence of Prostate Cancer After Radical Prostatectomy. Journal of Nuclear Medicine 2019, 60 (3), 362-368, Article. DOI: 10.2967/jnumed.118.212233.
5) Morris, M.; Rowe, S.; Gorin, M.; Saperstein, L.; Pouliot, F.; Josephson, D., Wong, J.; Pantel, A.; Cho, S.; Gage, K.; et al. Diagnostic Performance of F-18-DCFPyL-PET/CT in Men with Biochemically Recurrent Prostate Cancer: Results from the CONDOR Phase III, Multicenter Study. Clinical Cancer Research 2021, 27 (13), 3674-3682, Article. DOT: 10.1158/1078-0432.CCR-20-4573.
6) Fendler, W.; Calais, J.; Eiber, M.; Flavell, R.; Mishoe, A.; Feng, F.; Nguyen, H.; Reiter, R.; Rettig, M.; Okamoto, S.; et al. Assessment of Ga-68-PSMA-11 PET Accuracy in Localizing Recurrent Prostate Cancer: A Prospective Single-Arm Clinical Trial. Jama Oncology 2019, 5 (6), 856-863, Article. DOI: 10.1001/jamaoncol.2019.0096.
7) Rahbar, K.; Ahmadzadehfar, H.; Kratochwil, C.; Haberkorn, U.; Schafers, M.; Essler, M.; Baum, R.; Kulkarni, H.; Schmidt, M.; Drzezga, A.; et al. German Multicenter Study Investigating Lu-177-PSMA-617 Radioligand Therapy in Advanced Prostate Cancer Patients. Journal of Nuclear Medicine 2017, 58 (1), 85-90, Article. DOI: 10.2967/jnumed.116.183194.
8) Rahbar, K.; Ahmadzadehfar, H.; Kratochwil, C.; Haberkorn, U.; Schafers, M.; Essler, M.; Baum, R.; Kulkarni, H.; Schmidt, M.; Drzezga, A.; et al. German Multicenter Study Investigating Lu-177-PSMA-617 Radioligand Therapy in Advanced Prostate Cancer Patients. Journal of Nuclear Medicine 2020, 61, 257S-262S, Article. DOI: 10.2967/jnumed.120.252122a.
9) Sartor, O.; de Bono, J.; Chi, K.; Fizazi, K.; Herrmann, K.; Rahbar, K.; Tagawa, S.; Nordquist, L.; Vaishampayan, N.; El-Haddad, G.; et al. Lutetium-177-PSMA-617 for Metastatic Castration-Resistant Prostate Cancer. New England Journal of Medicine 2021, Article|Early Access. DOI: 10.1056/NEJMoa2107322.
10) Meher, N.; Seo, K.; Wang, S.; Bidkar, A.; Fogarty, M.; Dhrona, S.; Huang, X.; Tang, R.; Blaha, C.; Evans, M.; et al. Synthesis and Preliminary Biological Assessment of Carborane-Loaded Theranostic Nanoparticles to Target Prostate-Specific Membrane Antigen. Acs Applied Materials & Interfaces 2021, 13 (46), 54739-54752, Article. DOI: 10.1021/acsami.1c16383.
11) Lesniak, W.; Boinapally, S.; Banerjee, S.; Azad, B.; Foss, C.; Shen, C.; Lisok, A.; Wharram, B.; Nimmagadda, S.; Pomper, M. Evaluation of PSMA-Targeted PAMAM Dendrimer Nanoparticles in a Murine Model of Prostate Cancer. Molecular Pharmaceutics 2019, 16 (6), 2590-2604, Article. DOI: 10.1021/acs.molpharmaceut.9b00181.
12) Afsharzadeh, M.; Hashemi, M.; Babaei, M.; Abnous, K.; Ramezani, M. PEG-PLA nanoparticles decorated with small-molecule PSMA ligand for targeted delivery of galbanic acid and docetaxel to prostate cancer cells. Journal of Cellular Physiology 2020, 235 (5), 4618-4630, Article. DOI: 10.1002/jcp.29339.
13) Bradley, C. Efficacy of a PSMA-targeted nanoparticle. Nature Reviews Urology 2018, 15 (10), 589-590, Editorial Material. DOI: 10.1158/1078-0432.CCR-18-0106.
14) Von Hoff, D.; Mita, M.; Ramanathan, R.; Weiss, G.; Mita, A.; LoRusso, P.; Burris, H.; Hart, L.; Low, S.; Parsons, D.; et al. Phase I Study of PSMA-Targeted Docetaxel-Containing Nanoparticle BIND-014 in Patients with Advanced Solid Tumors. Clinical Cancer Research 2016, 22 (13), 3157-3163, Article. DOI: 10.1158/1078-0432.CCR-15-2548.
15) Flores, O.; Santra, S.; Kaittanis, C.; Bassiouni, R.; Khaled, A.; Khaled, A.; Grimm, J.; Perez, J. PSMA-Targeted Theranostic Nanocarrier for Prostate Cancer. Theranostics 2017, 7 (9), 2477-2494, Article. DOI: 10.7150/thno.18879.
16) Lim, J.; Guan, B.; Nham, K.; Hao, G.; Sun, X.; Simanek, E. Tumor Uptake of Triazine Dendrimers Decorated with Four, Sixteen, and Sixty-Four PSMA-Targeted Ligands: Passive versus Active Tumor Targeting. Biomolecules 2019, 9 (9), Article. DOI: 10.3390/biom9090421.
17) Hrkach, J.; Von Hoff, D.; Ali, M.; Andrianova, E.; Auer, J.; Campbell, T.; De Witt, D.; Figa, M.; Figueiredo, M.; Horhota, A.; et al. Preclinical Development and Clinical Translation of a PSMA-Targeted Docetaxel Nanoparticle with a Differentiated Pharmacological Profile. Science Translational Medicine 2012, 4 (128), Article. DOI: 10.1126/scitranslmed.3003651.
18) Xenaki, K.; Oliveira, S.; Henegouwen, P. Antibody or Antibody Fragments: Implications for Molecular Imaging and Targeted Therapy of Solid Tumors. Frontiers in Immunology 2017, 8, Article. DOI: 10.3389/fimmu.2017.01287.
19) Maeda, H. Toward a full understanding of the EPR effect in primary and metastatic tumors as well as issues related to its heterogeneity. Advanced Drug Delivery Reviews 2015, 91, 3-6, Review. DOI: 10.1016/j.addr.2015.01.002.
20) Vera, D.; Fontaine, S.; VanBrocklin, H.; Hearn, B.; Reid, R.; Ashley, G.; Santi, D. PET Imaging of the EPR Effect in Tumor Xenografts Using Small 15 nm Diameter Polyethylene Glycols Labeled with Zirconium-89. Molecular Cancer Therapeutics 2020, 19 (2), 673-679, Article. DOI: 10.1158/1535-7163.MCT-19-0709.
21) Singh, Y.; Gao, D.; Gu, Z.; Li, S.; Stein, S.; Sinko, P. Noninvasive Detection of Passively Targeted Poly(ethylene glycol) Nanocarriers in Tumors. Molecular Pharmaceutics 2012, 9 (1), 144-155, Article. DOI: 10.1021/mp2003913.
22) Kobayashi, H.; Watanabe, R.; Choyke, P. Improving Conventional Enhanced Permeability and Retention (EPR) Effects; What Is the Appropriate Target? Theranostics 2014, 4 (1), 81-89, Review. DOI: 10.7150/thno.7193.
23) Maeda, H.; Wu, J.; Sawa, T.; Matsumura, Y.; Hori, K. Tumor vascular permeability and the EPR effect in macromolecular therapeutics: a review. Journal of Controlled Release 2000, 65 (1-2), 271-284, Article|Proceedings Paper. DOI: 10.1016/S0168-3659(99)00248-5.
24) Bjornmalm, M.; Thurecht, K.; Michael, M.; Scott, A.; Caruso, F. Bridging Bio-Nano Science and Cancer Nanomedicine. Acs Nano 2017, 11 (10), 9594-9613, Review. DOI: 10.1021/acsnano.7b04855.
25) Yu, M.; Zheng, J. Clearance Pathways and Tumor Targeting of Imaging Nanoparticles. Acs Nano 2015, 9 (7), 6655-6674, Review. DOI: 10.1021/acsnano.5b01320.
26) Goos, J.; Cho, A.; Carter, L.; Dilling, T.; Davydova, M.; Mandleywala, K.; Puttick, S.; Gupta, A.; Price, W.; Quinn, J.; et al. Delivery of polymeric nanostars for molecular imaging and endoradiotherapy through the enhanced permeability and retention (EPR) effect. Theranostics 2020, 10 (2), 567-584, Article. DOI: 10.7150/thno.36777.
27) Heneweer, C.; Holland, J.; Divilov, V.; Carlin, S.; Lewis, J. Magnitude of Enhanced Permeability and Retention Effect in Tumors with Different Phenotypes: Zr-89-Albumin as a Model System. Journal of Nuclear Medicine 2011, 52 (4), 625-633, Article. DOI: 10.2967/jnumed.110.083998.
28) Fujimori, K.; Covell, D.; Fletcher, J.; Weinstein, J. A Modeling Analysis of Monoclonal-Antibody Percolation Through Tumors—A Binding-Site Barrier. Journal of Nuclear Medicine 1990, 31 (7), 1191-1198, Article.
29) Miao, L.; Newby, J.; Lin, C.; Zhang, L.; Xu, F.; Kim, W.; Forest, M.; Lai, S.; Milowsky, M.; Wobker, S.; et al. The Binding Site Barrier Elicited by Tumor Associated Fibroblasts Interferes Disposition of Nanoparticles in Stroma-Vessel Type Tumors. Acs Nano 2016, 10 (10), 9243-9258, Article. DOI: 10.1021/acsnano.6b02776.
30) Perk, L.; Vosjan, M.; Visser, G.; Budde, M.; Jurek, P.; Kiefer, G.; van Dongen, G. p-Isothiocyanatobenzyl-desferrioxamine: a new bifunctional chelate for facile radiolabeling of monoclonal antibodies with zirconium-89 for immuno-PET imaging. European Journal of Nuclear Medicine and Molecular Imaging 2010, 37 (2), 250-259, Article. DOI: 10.1007/s00259-009-1263-1.
31) Deri, M.; Zeglis, B.; Francesconi, L.; Lewis, J. PET imaging with Zr-89: From radiochemistry to the clinic. Nuclear Medicine and Biology 2013, 40 (1), 3-14, Article. DOI: 10.1016/j.nucmedbio.2012.08.004.
32) Nanabala, R.; Anees, M.; Sasikumar, A.; Joy, A.; Pillai, M. Preparation of [Ga-68]PSMA-11 for PET-CT imaging using a manual synthesis module and organic matrix based Ge-68/Ga-68 generator. Nuclear Medicine and Biology 2016, 43 (8), 463-469, Article. DOI: 10.1016/j.nucmedbio.2016.05.006.
33) Barenholz, Y. Doxil®—The first FDA-approved nano-drug: Lessons learned. Journal of Controlled Release 2012, 160 (2), 117-134, Review. DOI: 10.1016/j.jconrel.2012.03.020.
34) Barenholz, Y.; Braddock, M. Doxil®—the First FDA-approved Nano-drug: from Basics via CMC, Cell Culture and Animal Studies to Clinical Use. Nanomedicines: Design, Delivery and Detection 2016, 51, 315-345, Article|Book Chapter. Barenholz, Y.; Peer, D. Doxil®—The First FDA-Approved Nano-Drug: From an Idea to a Product. Handbook of Harnessing Biomaterials in Nanomedicine: Preparation, Toxicity, and Applications 2012, 335-398, Article Book Chapter.
35) Jewett, J.; Bertozzi, C. Cu-free click cycloaddition reactions in chemical biology. Chemical Society Reviews 2010, 39 (4), 1272-1279, Review. DOI: 10.1039/b901970g.
36) Wang, M.; McNitt, C.; Wang, H.; Ma, X.; Scarry, S.; Wu, Z.; Popik, V.; Li, Z. The efficiency of F-18 labelling of a prostate specific membrane antigen ligand via strain-promoted azide-alkyne reaction: reaction speed versus hydrophilicity. Chemical Communications 2018, 54 (56), 7810-7813, Article. DOI: 10.1039/c8cc03999b.
37) Wang, S.; Blaha, C.; Santos, R.; Huynh, T.; Hayes, T.; Beckford-Vera, D.; Blecha, J.; Hong, A.; Fogarty, M.; Hope, T.; et al. Synthesis and Initial Biological Evaluation of Boron-Containing Prostate-Specific Membrane Antigen Ligands for Treatment of Prostate Cancer Using Boron Neutron Capture Therapy. Molecular Pharmaceutics 2019, 16 (9), 3831-3841, Article. DOI: 10.1021/acs.molpharmaceut.9b00464.
38) Wang, S., Li, J.; Hua, J.; Su, Y.; Beckford-Vera, D.; Zhao, W.; Jayaraman, M.; Huynh, T.; Zhao, N.; Wang, Y.; et al. Molecular Imaging of Prostate Cancer Targeting CD46 Using ImmunoPET. Clinical Cancer Research 2021, 27 (5), 1305-1315, Article. DOI: 10.1158/1078-0432.CCR-20-3310.
39) Mesters, J.; Henning, K.; Hilgenfeld, R. Human glutamate carboxypeptidase II inhibition: structures of GCPII in complex with two potent inhibitors, quisqualate and 2-PMPA. Acta Crystallographica Section D-Structural Biology 2007, 63, 508-513, Article. DOI: 10.1107/S090744490700902X.
40) Maresca, K.; Hillier, S.; Femia, F.; Keith, D.; Barone, C.; Joyal, J.; Zimmerman, C.; Kozikowski, A.; Barrett, J.; Eckelman, W.; et al. A Series of Halogenated Heterodimeric Inhibitors of Prostate Specific Membrane Antigen (PSMA) as Radiolabeled Probes for Targeting Prostate Cancer. Journal of Medicinal Chemistry 2009, 52 (2), 347-357, Article. DOI: 10.1021/jm800994j.
41) Machulkin, A.; Ivanenkov, Y.; Aladinskaya, A.; Veselov, M.; Aladinskiy, V.; Beloglazkina, E.; Koteliansky, V.; Shakhbazyan, A.; Sandulenko, Y.; Majouga, A. Small-molecule PSMA ligands. Current state, SAR and perspectives. Journal of Drug Targeting 2016, 24 (8), 679-693, Review. DOI: 10.3109/1061186X.2016.1154564.
42) Barrett, J.; Coleman, R.; Goldsmith, S.; Vallabhajosula, S.; Petry, N.; Cho, S.; Armor, T.; Stubbs, J.; Maresca, K.; Stabin, M.; et al. First-in-Man Evaluation of 2 High-Affinity PSMA-Avid Small Molecules for Imaging Prostate Cancer. Journal of Nuclear Medicine 2013, 54 (3), 380-387, Article. DOI: 10.2967/jnumed.112.111203.
43) Derks, Y.; Rijpkema, M.; Amatdjais-Groenen, H.; Loeff, C.; de Roode, K.; Kip, A.; Laverman, P.; Lutje, S.; Heskamp, S.; Lowik, D. Strain-Promoted Azide-Alkyne Cycloaddition-Based PSMA-Targeting Ligands for Multimodal Intraoperative Tumor Detection of Prostate Cancer. Bioconjugate Chemistry 2022, 33 (1), 194-205, Article. DOI: 10.1021/acs.bioconjchem.1c00537.
44) Kovar, J.; Cheung, L.; Simpson, M.; Olive, D. Pharmacokinetic and Biodistribution Assessment of a Near Infrared-Labeled PSMA-Specific Small Molecule in Tumor-Bearing Mice. Prostate Cancer 2014, 2014, Article. DOI: 10.1155/2014/104248.
45) Baranski, A.; Schafer, M.; Bauder-Wust, U.; Roscher, M.; Schmidt, J.; Stenau, E.; Simpfendorfer, T.; Teber, D.; Maier-Hein, L.; Hadaschik, B.; et al. PSMA-11-Derived Dual-Labeled PSMA Inhibitors for Preoperative PET Imaging and Precise Fluorescence-Guided Surgery of Prostate Cancer. Journal of Nuclear Medicine 2018, 59 (4), 639-645, Article. DOI: 10.2967/jnumed.117.201293.
46) Chen, Z.; Penet, M.; Nimmagadda, S.; Li, C.; Banerjee, S.; Winnard, P.; Artemov, D.; Glunde, K.; Pomper, M.; Bhujwalla, Z. PSMA-Targeted Theranostic Nanoplex for Prostate Cancer Therapy. Acs Nano 2012, 6 (9), 7752-7762, Article. DOI: 10.1021/nn301725w.
47) Kwon, Y.; Chung, H.; Lee, S.; Lee, S.; Jeong, B.; Kim, H. Synthesis of novel multivalent fluorescent inhibitors with high affinity to prostate cancer and their biological evaluation. Bioorganic & Medicinal Chemistry Letters 2018, 28 (4), 572-576, Article. DOI: 10.1016/j.bmcl.2018.01.047.
48) Banerjee, S.; Pullambhatla, M.; Shallal, H.; Lisok, A.; Mease, R.; Pomper, M. A Modular Strategy to Prepare Multivalent Inhibitors of Prostate-Specific Membrane Antigen (PSMA). Oncotarget 2011, 2 (12), 1244-1253, Article. Reich, D.; Wurzer, A.; Wirtz, M.; Stiegler, V.; Spatz, P.; Pollmann, J.; Wester, H.; Notni, J. Dendritic poly-chelator frameworks for multimeric bioconjugation. Chemical Communications 2017, 53 (17), 2586-2589, Article. DOI: 10.1039/c6cc10169 k.
49) Wurzer, A.; Pollmann, J.; Schmidt, A.; Reich, D.; Wester, H.; Notni, J. Molar Activity of Ga-68 Labeled PSMA Inhibitor Conjugates Determines PET Imaging Results. Molecular Pharmaceutics 2018, 15 (9), 4296-4302, Article. DOI: 10.1021/acs.molpharmaceut.8b00602.
50) Adams, G.; Schier, R.; McCall, A.; Simmons, H.; Horak, E.; Alpaugh, R.; Marks, J.; Weiner, L. High affinity restricts the localization and tumor penetration of single-chain Fv antibody molecules. Cancer Research 2001, 61 (12), 4750-4755, Article. Kirpotin, D.; Drummond, D.; Shao, Y.; Shalaby, M.; Hong, K.; Nielsen, U.; Marks, J.; Benz, C.; Park, J. Antibody targeting of long-circulating lipidic nanoparticles does not increase tumor localization but does increase internalization in animal models. Cancer Research 2006, 66 (13), 6732-6740, Article. DOI: 10.1158/0008-5472.CAN-05-4199.
51) Lee, H.; Hoang, B.; Fonge, H.; Reilly, R.; Allen, C. In Vivo Distribution of Polymeric Nanoparticles at the Whole-Body, Tumor, and Cellular Levels. Pharmaceutical Research 2010, 27 (11), 2343-2355, Article. DOI: 10.1007/s11095-010-0068-z.
52) Khandare, J. J.; Chandna, P.; Wang, Y.; Pozharov, V. P.; Minko, T. Novel Polymeric Prodrug with Multivalent Components for Cancer Therapy. The Journal of Pharmacology and Experimental Therapeutics 2006, 317 (3), 929-937, Article. DOI: 10.1124/jpet.105.098855.
53) Lim, J.; Guan, B.; Nham, K.; Hao, G.; Sun, X.; Simanek, E. E. Tumor Uptake of Triazine Dendrimers Decorated with Four, Sixteen, and Sixty-Four PSMA-Targeted Ligands: Passive versus Active Tumor Targeting. Biomolecules 2019, 9 (421), 1-15, Article. DOI: 10.3390/biom9090421.
54) Beckford-Vera, D. R.; Fontaine, S. D.; VanBrocklin, H. F.; Hearn, B. R.; Reid, R.; Ashley, G. W. PET Imaging of the EPR Effect in Tumor Xenografts Using Small 15 nm Diameter Polyethylene Glycols Labeled with Zirconium-89. Molecular Cancer Therapeutics 2020, 19, 673-679, Article. DOI: 10.1158/1535-7163.mct-19-0709.
55) N. Meher, G. Ashley, A. Bidkar, S. Dhrona, C. Fong, S. Fontaine, D. Vera, D. Wilson, Y. Seo, D. Santi, H. VanBrocklin and R. Flavell, Acs Applied Materials & Interfaces, 2022.
56) 2. N. Meher, K. Seo, S. Wang, A. Bidkar, M. Fogarty, S. Dhrona, X. Huang, R. Tang, C. Blaha, M. Evans, D. Raleigh, Y. Jun, H. VanBrocklin, T. Desai, D. Wilson, T. Ozawa and R. Flavell, Acs Applied Materials & Interfaces, 2021, 13, 54739-54752.
57) 3. R. Nanabala, M. Anees, A. Sasikumar, A. Joy and M. Pillai, Nuclear Medicine and Biology, 2016, 43, 463-469.
58) 4. N. Meher, H. F. VanBrocklin, D. M. Wilson and R. R. Flavell, Pharmaceuticals, 2023, 16, 315.
59) 5. J. Jewett and C. Bertozzi, Chemical Society Reviews, 2010, 39, 1272-1279.
60) 6. C. Choy, X. Ling, J. Geruntho, S. Beyer, J. Latoche, B. Langton-Webster, C. Anderson and C. Berkman, Theranostics, 2017, 7, 1928-1939.
61) 7. S. Maus, E. d. Blois, S. J. Ament, M. Schreckenberger and W. A. P. Breeman, International Journal of Diagnostic Imaging, 2014, 1, 1-12.
62) 8. E. A. M. Ruigrok, N. van Vliet, S. U. Dalm, E. de Blois, D. C. van Gent, J. Haeck, C. de Ridder, D. Stuurman, M. W. Konijnenberg, W. M. van Weerden, M. de Jong and J. Nonnekens, European Journal of Nuclear Medicine and Molecular Imaging, 2021, 48, 1350.
63) 9. S. Wang, C. Blaha, R. Santos, T. Huynh, T. Hayes, D. Beckford-Vera, J. Blecha, A. Hong, M. Fogarty, T. Hope, D. Raleigh, D. Wilson, M. Evans, H. VanBrocklin, T. Ozawa and R. Flavell, Molecular Pharmaceutics, 2019, 16, 3831-3841.
64) 10. S. Wang, J. Li, J. Hua, Y. Su, D. Beckford-Vera, W. Zhao, M. Jayaraman, T. Huynh, N. Zhao, Y. Wang, Y. Huang, F. Qin, S. Shen, D. Gioeli, R. Dreicer, R. Sriram, E. Egusa, J. Chou, F. Feng, R. Aggarwal, M. Evans, Y. Seo, B. Liu, R. Flavell and J. He, Clinical Cancer Research, 2021, 27, 1305-1315.
65) 11. J. Mesters, K. Henning and R. Hilgenfeld, Acta Crystallographica Section D—Structural Biology, 2007, 63, 508-513.
66) 12. V. J. Tschan, F. Borgna, S. D. Busslinger, M. Stirn, J. M. M. Rodriguez, P. Bernhardt, R. Schibli and C. Müller, 2022, 49, 3639-3650.
67) 13. D. Vera, S. Fontaine, H. VanBrocklin, B. Hearn, R. Reid, G. Ashley and D. Santi, Molecular Cancer Therapeutics, 2020, 19, 673-679.
68) 14. Y. Singh, D. Gao, Z. Gu, S. Li, S. Stein and P. Sinko, Molecular Pharmaceutics, 2012, 9, 144-155.
69) 15. H. Kobayashi, R. Watanabe and P. Choyke, Theranostics, 2014, 4, 81-89.
70) 16. J. Goos, A. Cho, L. Carter, T. Dilling, M. Davydova, K. Mandleywala, S. Puttick, A. Gupta, W. Price, J. Quinn, M. Whittaker, J. Lewis and T. Davis, Theranostics, 2020, 10, 567-584.
71) 17. C. Heneweer, J. Holland, V. Divilov, S. Carlin and J. Lewis, Journal of Nuclear Medicine, 2011, 52, 625-633.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An imaging agent of Formula (I): 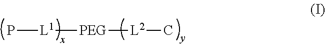
wherein:
PEG is branched polyethylene glycol;
L¹ and L² are independently selected linkers;
P is a prostate-specific membrane antigen (PSMA) targeted ligand;
C is a chelator;
x is an integer from 1 to 8;
y is an integer from 1 to 8,
such that x+y is less than or equal to 8,
wherein each C is independently selected from a chelating agent and a chelating agent binding a radioisotope suitable for positron emission tomography (PET) and/or single-photon emission computed tomography (SPECT).

2. The imaging agent of claim 1, wherein x+y is 4.

3. The imaging agent of any previous claim, wherein PEG is a polyethylene glycol of about 2 kD to about 100 kD.

4. The imaging agent of any previous claim, wherein PEG is a polyethylene glycol of about 40 kD.

5. The imaging agent of any previous claim, wherein L¹ and L² are independently selected from one or more of a bond, substituted or unsubstituted alkyl, substituted or unsubstituted heteroalkyl, substituted or unsubstituted cycloalkyl, substituted or unsubstituted aryl, substituted or unsubstituted heteroaryl, and substituted or unsubstituted acyl.

6. The imaging agent of any previous claim, wherein L¹ and L² are independently selected from

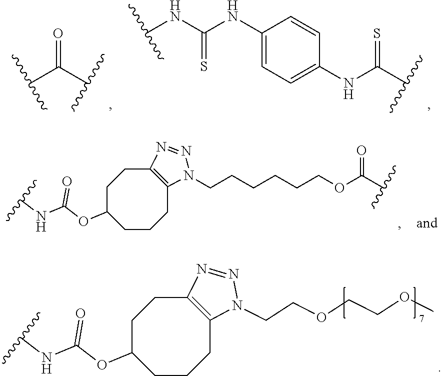

7. The imaging agent of any previous claim, wherein the chelator is selected from a desferrioxamine, a tetraaza macrocycle, or a multidentate plur-hydroxypyridinone.

8. The imaging agent of any previous claim, wherein the chelator is desferrioxamine-B (DFB) or 1,4,7,10-tetraazacyclododecane-1,4,7,10-tetraacetic acid (DOTA).

9. The imaging agent of any previous claim, wherein the radioisotope suitable for positron emission tomography (PET) and/or single-photon emission computed tomography (SPECT) is an ion selected from ⁸⁹Zr, ^{99m}Tc, ¹¹¹In, ¹⁷⁷Lu, ²²⁵Ac, ⁴⁴Sc, ⁶⁸Ga, ⁶⁴Cu, ⁶²Zn, ⁸⁶Y, ¹³⁴Ce, ¹⁴⁹Tb, ¹⁵²Tb, ¹⁵⁵Tb, and ²⁰³Pb.

10. The imaging agent of claim any previous claim, wherein the radioisotope is an ion of ⁸⁹Zr, ²²⁵Ac, or 177Lu.

11. The imaging agent of any previous claim, wherein the prostate-specific membrane antigen (PSMA) targeted is a compound of Formula (II): 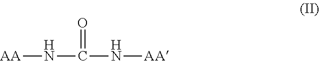
wherein:
AA and AA′ are independently selected amino acids connected through their NH₂ moieties by a urea linkage.

12. The imaging agent of any previous claim, wherein the prostate-specific membrane antigen (PSMA) targeted ligand is (S)-2-(3-((S)-5-amino-1-carboxypentyl)ureido)pentanedioic acid (ACUPA).

13. The imaging agent of any previous claim, wherein the imaging agent of Formula (T) is a compound selected from Formulae 101, 102, 103, 104, and a pharmaceutically acceptable salt thereof: 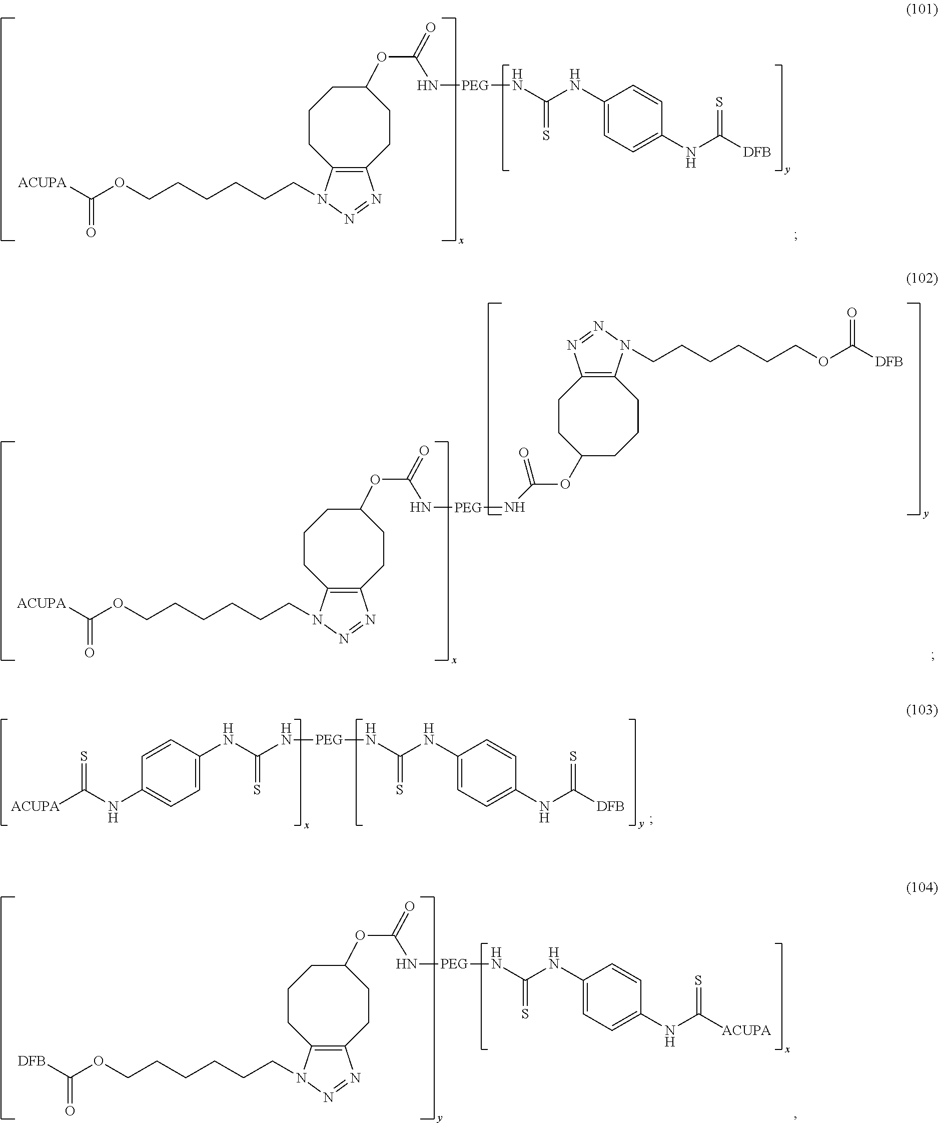
wherein:
PEG represents a branched polyethylene glycol;
x is an integer of 1 to 8;
y is an integer from 1 to 8,
such that x+y is less than or equal to 8,
wherein one desferrioxamine-B (DFB) moiety binds a radioisotope.

14. The imaging agent of claim 13, wherein the radioisotope is a ⁸⁹Zr ion.

15. The imaging agent of any previous claim, wherein the imaging agent of Formula (101) is a compound of any one of formula 1001-1028, or a pharmaceutically acceptable salt thereof, wherein one desferrioxamine-B (DFB) moiety binds a ⁸⁹Zr ion:
(101)

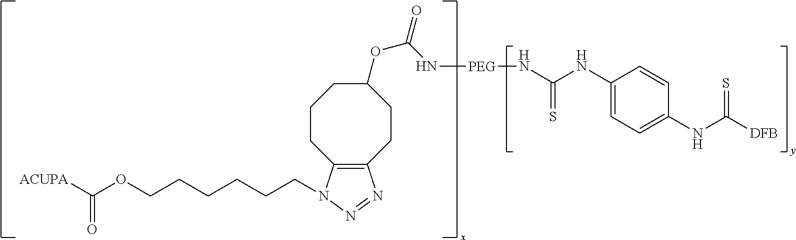

| Compound No. | x | y |
|---|---|---|
| 1001 | 1 | 1 |
| 1002 | 1 | 2 |
| 1003 | 1 | 3 |
| 1004 | 1 | 4 |
| 1005 | 1 | 5 |
| 1006 | 1 | 6 |
| 1007 | 1 | 7 |
| 1008 | 2 | 1 |
| 1009 | 2 | 2 |
| 1010 | 2 | 3 |
| 1011 | 2 | 4 |
| 1012 | 2 | 5 |
| 1013 | 2 | 6 |
| 1014 | 3 | 1 |
| 1015 | 3 | 2 |
| 1016 | 3 | 3 |
| 1017 | 3 | 4 |
| 1018 | 3 | 5 |
| 1019 | 4 | 1 |
| 1020 | 4 | 2 |
| 1021 | 4 | 3 |
| 1022 | 4 | 4 |
| 1023 | 5 | 1 |
| 1024 | 5 | 2 |
| 1025 | 5 | 3 |
| 1026 | 6 | 1 |
| 1027 | 6 | 2 |
| 1028 | 7 | 1 |

16. The imaging agent of any previous claim, wherein the imaging agent of formula (102) is a compound of any one of formula 1029-1056, or a pharmaceutically acceptable salt thereof, wherein one desferrioxamine-B (DFB) moiety binds a ⁸⁹Zr ion:
(102)

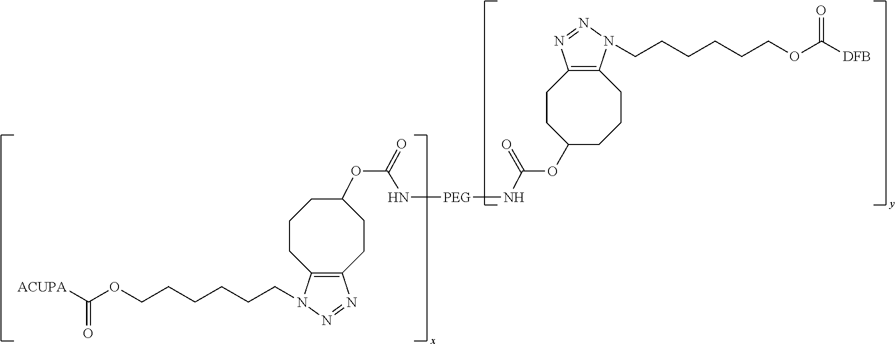

| Compound No. | x | y |
|---|---|---|
| 1029 | 1 | 1 |
| 1030 | 1 | 2 |
| 1031 | 1 | 3 |
| 1032 | 1 | 4 |
| 1033 | 1 | 5 |
| 1034 | 1 | 6 |
| 1035 | 1 | 7 |
| 1036 | 2 | 1 |
| 1037 | 2 | 2 |
| 1038 | 2 | 3 |
| 1039 | 2 | 4 |
| 1040 | 2 | 5 |
| 1041 | 2 | 6 |
| 1042 | 3 | 1 |
| 1043 | 3 | 2 |
| 1044 | 3 | 3 |
| 1045 | 3 | 4 |
| 1046 | 3 | 5 |
| 1047 | 4 | 1 |
| 1048 | 4 | 2 |
| 1049 | 4 | 3 |
| 1050 | 4 | 4 |
| 1051 | 5 | 1 |
| 1052 | 5 | 2 |
| 1053 | 5 | 3 |
| 1054 | 6 | 1 |
| 1055 | 6 | 2 |
| 1056 | 7 | 1 |

17. The imaging agent of any previous claim, wherein the imaging agent of formula (103) is a compound ofany one of formula 1057-1084, or a pharmaceutically acceptable salt thereof, wherein one desferrioxamine-B (DFB) moiety binds a ⁸⁹Zr ion:
(103)

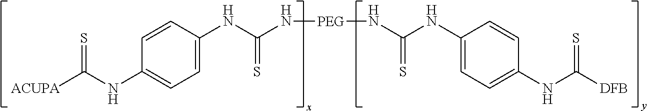

| Compound No. | x | y |
|---|---|---|
| 1057 | 1 | 1 |
| 1058 | 1 | 2 |
| 1059 | 1 | 3 |
| 1060 | 1 | 4 |
| 1061 | 1 | 5 |
| 1062 | 1 | 6 |
| 1063 | 1 | 7 |
| 1064 | 2 | 1 |
| 1065 | 2 | 2 |
| 1066 | 2 | 3 |
| 1067 | 2 | 4 |
| 1068 | 2 | 5 |
| 1069 | 2 | 6 |
| 1070 | 3 | 1 |
| 1071 | 3 | 2 |
| 1072 | 3 | 3 |
| 1073 | 3 | 4 |
| 1074 | 3 | 5 |
| 1075 | 4 | 1 |
| 1076 | 4 | 2 |
| 1077 | 4 | 3 |
| 1078 | 4 | 4 |
| 1079 | 5 | 1 |
| 1080 | 5 | 2 |
| 1081 | 5 | 3 |
| 1082 | 6 | 1 |
| 1083 | 6 | 2 |
| 1084 | 7 | 1 |

18. The imaging agent of any previous claim, wherein the imaging agent of formula (104) is a compound of any one of formula 1085-1112, or a pharmaceutically acceptable salt thereof, wherein one desferrioxamine-B (DFB) moiety binds a ⁸⁹Zr ion:
(104)

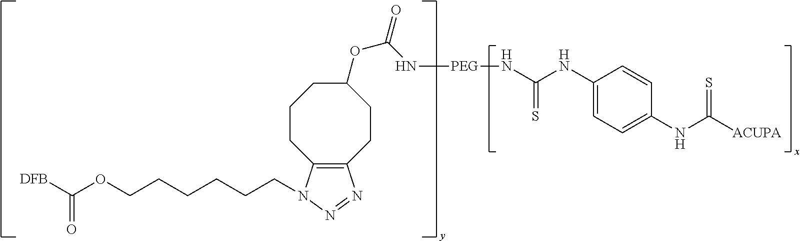

| Compound No. | x | y |
|---|---|---|
| 1085 | 1 | 1 |
| 1086 | 1 | 2 |
| 1087 | 1 | 3 |
| 1088 | 1 | 4 |
| 1089 | 1 | 5 |
| 1090 | 1 | 6 |
| 1091 | 1 | 7 |
| 1092 | 2 | 1 |
| 1093 | 2 | 2 |
| 1094 | 2 | 3 |
| 1095 | 2 | 4 |
| 1096 | 2 | 5 |
| 1097 | 2 | 6 |
| 1098 | 3 | 1 |
| 1099 | 3 | 2 |
| 1100 | 3 | 3 |
| 1101 | 3 | 4 |
| 1102 | 3 | 5 |
| 1103 | 4 | 1 |
| 1104 | 4 | 2 |
| 1105 | 4 | 3 |
| 1106 | 4 | 4 |
| 1107 | 5 | 1 |
| 1108 | 5 | 2 |
| 1109 | 5 | 3 |
| 1110 | 6 | 1 |
| 1111 | 6 | 2 |
| 1112 | 7 | 1 |

19. The imaging agent of any previous claim, wherein the imaging agent is selected from: 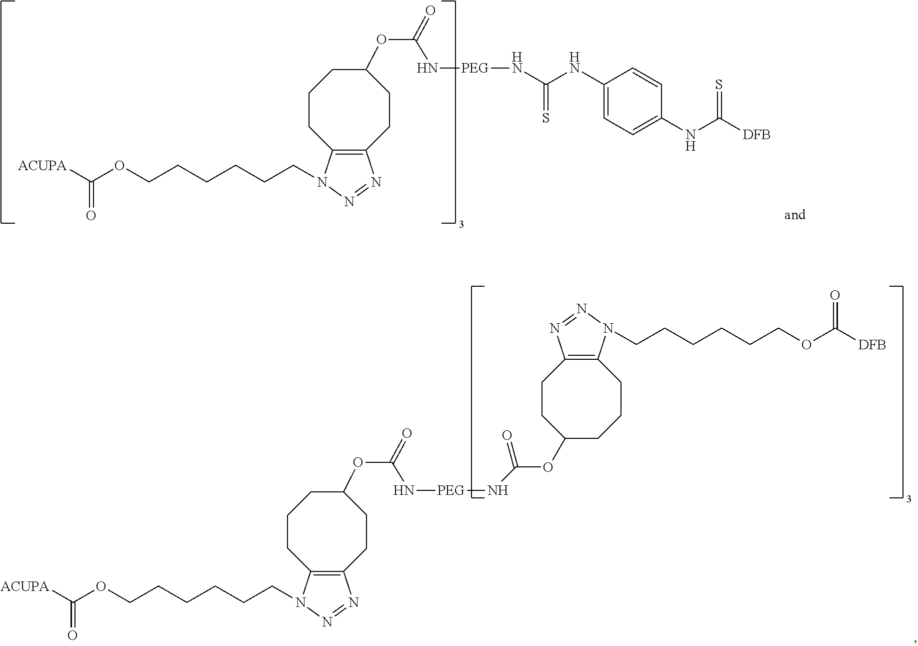
wherein one desferrioxamine-B (DFB) moiety binds a ⁸⁹Zr ion.

20. The imaging agent of any one of claims 1 to 13, wherein the imaging agent of Formula (I) is a compound of Formula 105 or a pharmaceutically acceptable salt thereof: 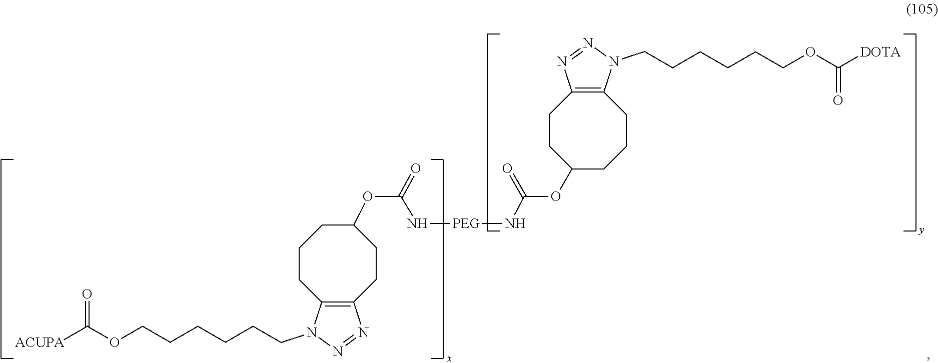
wherein:
PEG represents a branched polyethylene glycol;
x is an integer of 1 to 8;
y is an integer from 1 to 8,
such that x+y is less than or equal to 8,
wherein one 1,4,7,10-tetraazacyclododecane-1,4,7,10-tetraacetic acid (DOTA) moiety binds a radioisotope.

21. The radioisotope according to claim 20, wherein the radioisotope is a ¹⁷⁷Lu ion or a ²²⁵Ac ion.

22. The imaging agent of claim 20 or 21, wherein the imaging agent of Formula (105) is a compound of any one of formula 1113-1140, or a pharmaceutically acceptable salt thereof, wherein one 1,4,7,10-tetraazacyclododecane-1,4,7,10-tetraacetic acid (DOTA) moiety binds a ¹⁷⁷Lu ion or a ²²⁵Ac ion:
(105)

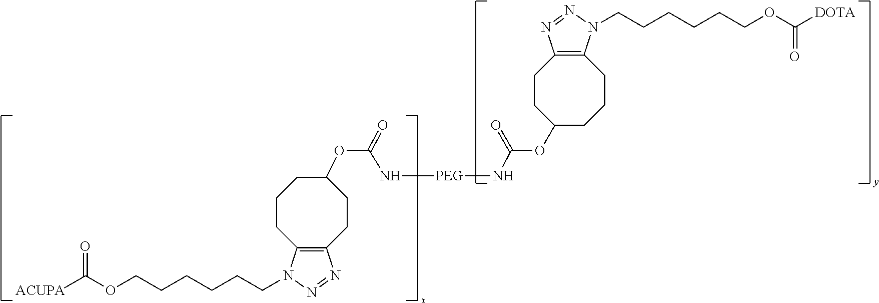

| Compound No. | x | y |
|---|---|---|
| 1113 | 1 | 1 |
| 1114 | 1 | 2 |
| 1115 | 1 | 3 |
| 1116 | 1 | 4 |
| 1117 | 1 | 5 |
| 1118 | 1 | 6 |
| 1119 | 1 | 7 |
| 1120 | 2 | 1 |
| 1121 | 2 | 2 |
| 1122 | 2 | 3 |
| 1123 | 2 | 4 |
| 1124 | 2 | 5 |
| 1125 | 2 | 6 |
| 1126 | 3 | 1 |
| 1127 | 3 | 2 |
| 1128 | 3 | 3 |
| 1129 | 3 | 4 |
| 1130 | 3 | 5 |
| 1131 | 4 | 1 |
| 1132 | 4 | 2 |
| 1133 | 4 | 3 |
| 1134 | 4 | 4 |
| 1135 | 5 | 1 |
| 1136 | 5 | 2 |
| 1137 | 5 | 3 |
| 1138 | 6 | 1 |
| 1139 | 6 | 2 |
| 1140 | 7 | 1 |

23. The imaging agent of any one of claims 20 to 22, wherein the imaging agent is: 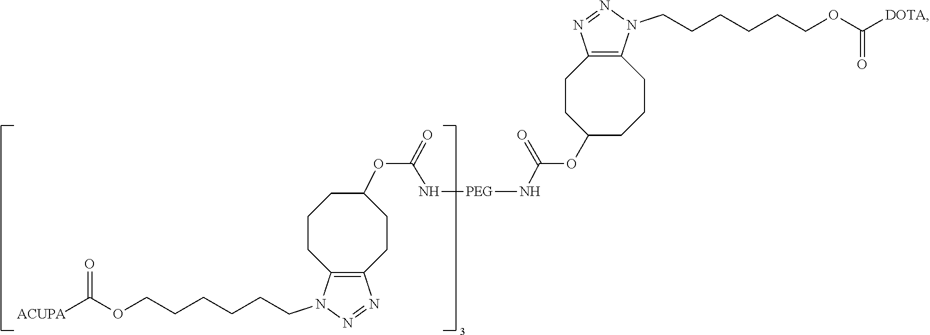
wherein the DOTA moiety binds a ¹⁷⁷Lu ion.

24. The imaging agent of any previous claim, wherein prostate-specific membrane antigen (PSMA)-expressing cells exhibit cell binding and internalization of the imaging agent relative to cells that do not express PSMA.

25. The imaging agent of any previous claim, wherein the imaging agent exhibits enhanced permeability and retention (EPR) in prostate-specific membrane antigen (PSMA)-expressing tumors relative to tumors that do not express PSMA.

26. The imaging agent of any previous claim, wherein the imaging agent exhibits targeted accumulation in prostate-specific membrane antigen (PSMA)-expressing tumors relative to tumors that do not express PSMA.

27. The imaging agent of any previous claim, wherein the imaging agent exhibits deep-tumor penetration in prostate-specific membrane antigen (PSMA)-expressing tumors relative to tumors that do not express PSMA.

28. The imaging agent of any previous claim, wherein the imaging agent exhibits a dissociation constant (K_{d}) from about 30 nM to about 800 nM.

29. The imaging agent of any previous claim, wherein the imaging agent exhibits a dissociation constant (K_{d}) from about 52 nM to about 66 nM.

30. The imaging agent of any previous claim, wherein the imaging agent exhibits IC₅₀ values from about 359 nM to about 706 nM.

31. The imaging agent of any previous claim, wherein the imaging agent exhibits IC₅₀ values from about 459 nM to about 575 nM.

32. The imaging agent of any previous claim, wherein the imaging agent exhibits IC₅₀ values from about 687 nM to about 1380 nM.

33. A prodrug of the imaging agent of any one of claims 1-32.

34. The prodrug of claim 33, wherein the prodrug delivers a chemotherapeutic.

35. The prodrug of claim 33 or 34, wherein the chemotherapeutic delivered by the prodrug is selected from a vinca alkaloid, an anthracycline, an epidophyllotoxin, a taxane, an antimetabolite, an alkylating agent, an antibiotic, a Cox-2 inhibitor, an antimitotic, an antiangiogenic, and an apoptotic agent.

36. The prodrug of any one of claims 33-35, wherein the prodrug delivers a therapeutic radionuclide.

37. The prodrug of any one of claim 33-36, wherein the therapeutic radionuclide delivered by the prodrug is selected from ¹⁷⁷Lu, ²¹²Bi, ²¹³Bi, ²¹¹At, ⁶⁴Cu, ⁶⁷Cu, ⁹⁰Y, ³²P, ³³P, ⁴⁷Sc, ¹¹¹Ag, ⁶⁷Ga, ¹⁴²Pr, ¹⁵³Sm, ¹⁴⁹Tb, ¹⁵⁵Tb, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, ¹⁹⁹Au, ²¹¹Pb, ⁴⁷Sc, ²¹²Pb, ²²⁵Ac, and ²²⁷Th.

38. A pharmaceutical composition comprised of the imaging agent of any one of claims 1-37, a prodrug of the imaging agent, or a combination thereof, and a pharmaceutically acceptable carrier.

40. A method of imaging a solid tumor in a subject which method comprises administering to said subject the imaging agent of any of claims 1-32 or 38 wherein said imaging agent exhibits enhanced permeability and retention (EPR) and effects the concentration of said imaging agent in said tumor and detecting the imaging agent by positron emission tomography (PET) and/or single-photon emission computed tomography (SPECT).

41. The method of claim 40, wherein the tumor is a prostate-specific membrane antigen (PSMA)-expressing tumor.

42. The method of claim 40 or 41, wherein the method correlates the imaging and treatment of the tumor.

43. The method of claim 42, wherein the method comprises monitoring the accumulation of said imaging agent in the tumor and monitoring the volume of said tumor.

44. A corresponding image generated by the method of claim 40.

45. A method of treating a solid tumor which method comprises administering to a subject in need thereof a therapeutically effective amount of the prodrug of any of claims 33-37 wherein said prodrug exhibits enhanced permeability and retention (EPR) and effects the concentration of said prodrug in said tumor and detecting the prodrug by positron emission tomography (PET) and/or single-photon emission computed tomography (SPECT).

46. The method of claim 45, wherein the tumor is a prostate-specific membrane antigen (PSMA)-expressing tumor.

47. The method of any one of claims 40-46, wherein the subject is a human, rat, mouse, cat, dog, horse, sheep, cow, monkey, avian, or amphibian.

48. A kit that includes one or more imaging agents according to any of claims 1 to 38.
